(19) United States
(12) Patent Application Publication
(10) Pub. No.: US 2012/0124685 A1
(43) Pub. Date: May 17, 2012

(54) COMPOSITIONS AND METHODS FOR GENE EXPRESSION AND CHROMATIN PROFILING OF INDIVIDUAL CELL TYPES WITHIN A TISSUE

(76) Inventors: Steven Henikoff, Seattle, WA (US); Roger B. Deal, Decatur, GA (US); Gilbert Lee Henry, Leesburg, VA (US)

(21) Appl. No.: 13/234,109

(22) Filed: Sep. 15, 2011

Related U.S. Application Data
(63) Continuation-in-part of application No. PCT/US11/40375, filed on Jun. 14, 2011.
(60) Provisional application No. 61/354,663, filed on Jun. 14, 2010.

(51) Int. Cl. C12Q 1/68; A01K 67/027; A01H 5/00; C12N 15/63; C12N 5/10

(52) U.S. Cl. 800/14; 435/320.1; 435/419; 435/6.1; 435/325; 435/352; 435/350; 435/351; 435/363; 435/366; 800/298; 800/13

(57) ABSTRACT

The present invention provides compositions, methods, and kits for generating and isolating tagged nuclei of specific cell types with a high yield and purity. The compositions, methods, and kits provided herein comprise expressing in a cell a nuclear envelope tagging fusion polypeptide comprising a nuclear envelope targeting domain and an affinity reagent binding region. In some embodiments, expression of the fusion polypeptide is under the control of a cell type-specific promoter. Some embodiments also comprise expressing in a cell a biotin ligase, wherein the affinity reagent binding region comprises a biotin ligase accepting site, and wherein at least one of the nuclear envelope tagging fusion polypeptide and the biotin ligase is expressed under the control of a cell type-specific promoter.

### CROSS-REFERENCE TO RELATED APPLICATION

This application is a continuation-in-part of PCT Patent Application No. PCT/US2011/040375, filed on Jun. 14, 2011, which claims the benefit of U.S. Provisional Application No. 61/354,663, filed on Jun. 14, 2010, the entire disclosures of which are hereby incorporated by reference herein.

### STATEMENT OF GOVERNMENT LICENSE RIGHTS

This invention was made in part with Government support under NIH #1F32GM083449-01 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is: 37731_Seq_Final_{—}2011-09-15.txt. The text file is 221 KB; was created on Sep. 15, 2011; and is being submitted via EFS-Web with the filing of the specification.

### FIELD OF THE INVENTION

This invention relates to methods, reagents, and kits for selectively isolating nuclei from a cell type of interest suitable for use in analysis of gene expression and chromatin profiling of individual cell types within a tissue.

### BACKGROUND

Growth and development of multicellular organisms requires the production of many specialized cell types that make up the tissues and organs of the adult body. The generation of a differentiated cell from an undifferentiated progenitor involves epigenetic reprogramming of the stem cell genome to establish the appropriate lineage-specific transcription program. Initial establishment and subsequent maintenance of this transcriptional program is effected through chromatin-based gene silencing and activation mechanisms involving the dynamic interplay of transcription factors, post-translational modification of histones, the deposition of histone variants, DNA methylation, and nucleosome remodeling (Brien, G. L., and A. P. Bracken, “Transcriptomics: Unravelling the Biology of Transcription Factors and Chromatin Remodelers During Development and Differentiation,” Semin. Cell Dev. Biol. 20:835-841, 2009; Muller, C., and A. Leutz, “Chromatin Remodeling in Development and Differentiation,” Curr. Opin. Genet. Dev. 11:167-174, 2001; Ng, R. K., and J. B. Gurdon, “Epigenetic Inheritance of Cell Differentiation Status,” Cell Cycle 7:1173-1177, 2008). Defining precisely how cellular differentiation is imposed and maintained is a central goal of developmental biology, and is also critical to understanding how the process can go awry, leading to disease states such as cancer. Despite the importance of this problem, knowledge of the mechanics of differentiation processes in vivo is still quite limited, in large part due to the technical difficulty associated with isolating pure cell types from a tissue for transcriptional and epigenomic profiling.

Current methods for the study of pure individual cell types include the use of cultured cell lines (Mito, Y., et al., “Genome-Scale Profiling of Histone H3.3 Replacement Patterns,” Nat. Genet. 37:1090-1097, 2005; Rao, R. R. and S. L. Stice, “Gene Expression Profiling of Embryonic Stem Cells Leads to Greater Understanding of Pluripotency and Early Developmental Events,” Biol. Reprod. 71:1772-1778, 2004; Rivolta, M. N. and M. C. Holley, “Cell Lines in Inner Ear Research,” J. Neurobiol. 53:306-318, 2002), ex vivo differentiation from progenitor cells (Bhattacharya, B., et al., “A Review of Gene Expression Profiling of Human Embryonic Stem Cell Lines and Their Differentiated Progeny,” Curr. Stem Cell Res. Ther. 4:98-106, 2009; Trion, S., et al., “Directed Differentiation of Pluripotent Stem Cells: From Developmental Biology to Therapeutic Applications,” Cold Spring Harb. Symp. Quant. Biol. 73:101-110, 2008), laser capture microdissection (LCM) of sectioned tissues (Brunskill, E. W., et al., “Atlas of Gene Expression in the Developing Kidney at Microanatomic Resolution,” Dev. Cell 15:781-791, 2008; Jiao, Y., et al., “A Transcriptome Atlas of Rice Cell Types Uncovers Cellular, Functional and Developmental Hierarchies,” Nat. Genet. 41:258-263, 2009; Nakazono, M., et al., “Laser-Capture Microdissection, a Tool for the Global Analysis of Gene Expression in Specific Plant Cell Types: Identification of Genes Expressed Differentially in Epidermal Cells or Vascular Tissues of Maize,” Plant Cell 15:583-596, 2003), and fluorescence-activated cell sorting (FACS) of fluorescently labeled cell lines or protoplasts (Birnbaum, K., et al., “A Gene Expression Map of the Arabidopsis Root,” Science 302:1956-1960, 2003; de la Cruz, A. F., and B. A. Edgar, “Flow Cytometric Analysis of Drosophila Cells,” Methods Mol. Biol. 420:373-389, 2008; Gifford, M. L., et al., “Cell-Specific Nitrogen Responses Mediate Developmental Plasticity,” Proc. Natl. Acad. Sci. USA 105, 803-808, 2008; Zhang, Y., et al., “Identification of Genes Expressed in C. elegans Touch Receptor Neurons,” Nature 418:331-335, 2002). Of these techniques, LCM and FACS are the only ones applicable to in vivo studies, but both are limited in that they involve extensive tissue manipulation, require complex and highly expensive equipment, and offer relatively low throughput. Several new methods, such as cell type-specific chemical modification of RNA (Miller, M. R., et al. “TU-Tagging: Cell Type-Specific RNA Isolation From Intact Complex Tissues,” Nat. Methods 6:439-441, 2009) and affinity tagging of ribosomal proteins or poly(A)-binding proteins (Heiman, M., et al., “A Translational Profiling Approach for the Molecular Characterization of CNS Cell Types,” Cell 135:738-748, 2008; Mustroph, A., et al., “Profiling Translatomes of Discrete Cell Populations Resolves Altered Cellular Priorities During Hypoxia in Arabidopsis,” Proc. Natl. Acad. Sci. USA, 2009; Roy, P. J., et al., “Chromosomal Clustering of Muscle-Expressed Genes in Caenorhabditis elegans,” Nature 418:975-979, 2002) have also been successfully employed to measure the gene expression profiles of individual cell types, but these approaches cannot be used to study chromatin features.

Therefore, a need exists for a simple and broadly applicable method for studying gene expression and chromatin regulation in individual cell types to make the study of cell differentiation and function more accessible.

### SUMMARY

This summary is provided to introduce a selection of concepts in a simplified form that are further described below in the Detailed Description. This summary is not intended to identify key features of the claimed subject matter nor is it intended to be used as an aid in determining the scope of the claimed subject matter.

In one aspect, the present invention provides a vector for selectively labeling nuclei in a cell type of interest comprising a nucleic acid sequence encoding a fusion polypeptide comprising (a) a nuclear envelope targeting region and (b) an affinity reagent binding region. In some embodiments, the affinity reagent binding region comprises a biotin ligase accepting site. In some embodiments, the affinity binding region comprises an epitope recognized by an antibody.

In another aspect, the present invention provides a cell comprising a vector for selectively labeling the cell type of interest, the vector comprising a nucleic acid sequence encoding a fusion polypeptide comprising (a) a nuclear envelope targeting region; and (b) an affinity reagent binding region, wherein the fusion polypeptide is incorporated into the nuclei of the cell. In some embodiments, the cell is in a tissue, culture, or part of a transgenic organism.

In another aspect, the invention provides a kit for selectively labeling nuclei in a cell type of interest, the kit comprising: (a) a vector comprising a first expression cassette comprising a nucleic acid sequence encoding a fusion polypeptide comprising: (i) a nuclear envelope targeting region; and (ii) an affinity reagent binding region; and (b) a capture molecule capable of specifically binding to the affinity binding region, or a modification thereof. In some embodiments, the affinity binding region comprises a biotin ligase accepting site. In some embodiments, the kit further comprises a second expression cassette for expressing a biotin ligase polypeptide. In some embodiments, the capture reagent is bound to a magnetic particle.

In another aspect, the invention provides a method for generating in vivo biotinylated nuclei in a cell type of interest. The method according to this aspect comprises recombinantly expressing in the cell a fusion polypeptide comprising (i) a nuclear envelope targeting region and (ii) an affinity reagent binding region, wherein one of the fusion polypeptide or a molecule that modifies the fusion polypeptide is under the control of a promoter specific to the cell type of interest.

In another aspect, the invention provides a method for selectively isolating nuclei from a cell type of interest present in a plurality of cells wherein at least a portion of the cells recombinantly express a fusion polypeptide comprising (i) a nuclear envelope targeting region and (ii) an affinity reagent binding region, wherein at least one of the fusion polypeptide or a molecule that modifies the fusion protein is under the control of a promoter specific to the cell type of interest. The method comprises: (a) lysing the plurality of cells under conditions suitable to generate a cell lysate comprising a plurality of intact nuclei; (b) contacting the cell lysate with a capture molecule that specifically binds to the affinity reagent binding region, or a modified form thereof, under conditions suitable to bind the nuclei comprising the fusion polypeptide; and (c) isolating the nuclei bound to the capture molecule.

In another aspect, the present invention provides a method of generating in vivo biotinylated nuclei in a cell type of interest. The method comprises recombinantly co-expressing in the cell: (a) a fusion polypeptide comprising (i) a nuclear envelope targeting region; and (ii) a biotin ligase accepting site; and (b) a biotin ligase; wherein the co-expression of the recombinant fusion polypeptide and the biotin ligase produces biotinylated nuclei in the cell of interest. In some embodiments, the nucleic acid sequences encoding the fusion polypeptide and biotin ligase are present on the same vector, and wherein the co-expressing comprises introducing one or more copies of the vector encoding the fusion polypeptide and biotin ligase into the cell type of interest, or a progenitor of the cell type of interest. In other embodiments, the nucleic acid sequences encoding the fusion polypeptide and biotin ligase are present on separate vectors, and wherein the co-expressing comprises introducing one or more copies of the vector encoding the fusion polypeptide and introducing one or more copies of the vector encoding biotin ligase into the cell type of interest, or a progenitor of the cell type of interest. In some embodiments, the cell type of interest is in a mixture of multiple cell types. In some embodiments, the method further comprises isolating biotinylated nuclei from the cells using a capture molecule that specifically binds to biotin.

In another aspect, the present invention provides a method of selectively isolating nuclei from a cell type of interest wherein at least a portion of the cells co-express (i) a recombinant fusion polypeptide comprising a nuclear envelope targeting region and a biotin ligase accepting site, and (ii) a biotin ligase, wherein expression of at least one of the recombinant fusion polypeptide or the biotin ligase is under the control of a promoter that is specific for the cell type of interest, and wherein the co-expression of the recombinant fusion polypeptide and the biotin ligase selectively produces biotinylated nuclei in the cell type of interest. The method comprises: (a) lysing the plurality of cells from the mixture under conditions suitable to generate a cell lysate comprising a plurality of intact nuclei; and (b) contacting the cell lysate with a capture molecule that specifically binds to biotin under conditions suitable to bind the biotinylated nuclei; and (c) isolating the biotinylated nuclei bound to the capture molecule. In some embodiments, the cell type of interest is in a mixture of multiple cell types, such as a cell culture or tissue. In some embodiments, the capture molecule is bound to a magnetic particle. In some embodiments, the capture molecule is selected from the group consisting of: streptavidin or a fragment thereof, avidin or a fragment thereof, and an anti-biotin antibody or a fragment thereof. In some embodiments, the method further comprises extracting nucleic acids from the isolated biotinylated nuclei. In some embodiments, the method further comprises performing gene expression analysis on the isolated nucleic acids. In some embodiments, the method further comprises performing analysis of the chromatin structure of the nucleic acids.

Finally, in another aspect, the present invention provides a method of visually tagging nuclei in a cell type of interest comprising introducing a vector comprising a nucleic acid sequence encoding a fusion polypeptide comprising (a) a nuclear envelope targeting region and (b) a fluorescent protein, into the cell type of interest. In some embodiments, the cell type of interest is eukaryotic. In some embodiments, the nuclear envelope targeting region selectively targets the outer nuclear membrane. In some embodiments, the nuclear envelope targeting region selectively targets the inner nuclear membrane. In some embodiments, the vector is a viral vector. In some embodiments, the cell type of interest is a neuron, such as a post-mitotic neuron.

The compositions, kits and methods of the present invention are useful, for example, for isolating the nuclei of a cell type of interest from a mixture of a plurality of cell types. The resulting purified nuclei can be used to perform transcriptional profiling and epigenomic profiling. Therefore, the compositions, kits and methods of the present invention provide a time and cost-effective approach for generating gene expression and epigenomic data for a cell type of interest to make the study of cell differentiation and function more accessible.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a schematic illustration of a nucleic acid construct used to transgenically express a nuclear tagging fusion (NTF) protein to label the nuclear envelope for the isolation of nuclei tagged in specific cell types, as described in Example 1;

FIG. 1B is a schematic illustration of a nucleic acid construct used to transgenically express an NTF protein to label the nuclear envelope, further including optional spacer regions and a visualization tag, as described in Example 1;

FIG. 1C is a schematic illustration of an embodiment of a nucleic acid construct used to transgenically express biotin ligase, as described in Example 1;

FIG. 1D is a schematic illustration of an embodiment of the invention in which the nuclear envelope is labeled with a transgenically expressed NTF protein comprising a nuclear envelope targeting region and an affinity reagent binding region, as described in Example 1;

FIG. 1E is a schematic illustration of an embodiment of the invention in which the nuclear envelope is labeled with a transgenically expressed NTF protein comprising a nuclear envelope targeting region and an affinity reagent binding region, wherein the affinity reagent binding region comprises a biotin ligase accepting site which is biotinylated by a biotin ligase, as described in Example 1;

FIG. 1F is a schematic illustration of an embodiment of the invention in which the nuclear envelope is labeled with a transgenically expressed NTF protein comprising a nuclear envelope targeting region, a first spacer region, a visualization tag, a second spacer region, and an affinity reagent binding region, wherein the affinity reagent binding region comprises a biotin ligase accepting site which is biotinylated by a biotin ligase, as described in Example 1;

FIG. 1G is a schematic illustration of a device useful to isolate nuclei labeled in accordance with the methods of the invention, as described in Example 1;

FIG. 2A is a confocal projection image of the differentiation zone of an ADF8p:NTF/ACT2p:BirA transgenic root showing expression of the NTF protein in hair cells. The Green Fluorescent Protein (GFP) domain provides a visualization signal that is shown as the lighter gray globular shapes (illustrative examples indicated by dashed circles). Propidium iodide staining of cell walls is shown as the linear wall architecture of the cells; as described in Example 1;

FIG. 2B is a confocal projection of the differentiation zone of an GL2p:NTF/ACT2p:BirA transgenic root showing expression of the NTF protein in non-hair cells. The GFP domain provides a visualization signal that is shown as the lighter gray globular shapes (illustrative examples indicated by dashed circles). Propidium iodide staining of cell walls is shown as the linear wall architecture of the cells; as described in Example 1;

FIG. 2C is a confocal section of the post-meristematic region of a GL2p:NTF/ACT2p:BirA transgenic root. The signal from the GFP domain appears on the circular nuclear envelopes, as indicated by arrows, as described in Example 1;

FIG. 2D is a fluorescence micrograph of nuclei (one is shown in inset) isolated from ADF8p:NTF/ACT2p:BirA transgenic roots and incubated with streptavidin Dynabeads®. The GFP and beads are shown as the brighter shades and are indicated with arrows. The DAPI staining of DNA is shown as the darker shade, as described in Example 1;

FIG. 2E is a streptavidin western blot of whole cell extracts (input) and anti-GFP immunoprecipitates (IP) from roots of ACT2p:BirA, ADF8p:NTF/ACT2p:BirA, and GL2p:NTF/ACT2p:BirA transgenic plants, wherein the top and bottom bands in each lane are endogenous biotinylated proteins and the middle band is the 42 kD NTF protein, as described in Example 1;

FIG. 3A is a streptavidin western blot of total protein obtained from the supernatant and pelleted nuclei from GL2p:NTF/ACT2p:BirA transgenic plants before and after two cycles of washing with nuclei purification buffer (NPB). The location of the NTF protein is indicated with the arrow, as described in Example 1;

FIG. 3B is a micrograph of total nuclei extracted from ADF8p:NTF/ACT2p:BirA transgenic Arabidopsis thaliana roots after incubation with streptavidin-coated Dynabeads®, with an exemplary bead-bound nucleus indicated with a circle, as described in Example 1;

FIG. 3C is a micrograph of total nuclei extracted from GL2p:NTF/ACT2:BirA transgenic A. thaliana roots after incubation with streptavidin-coated Dynabeads®, with exemplary bead-bound nucleus indicated with a circle, as described in Example 1;

FIG. 3D is a micrograph of total nuclei extracted from ACT2p:BirA transgenic A. thaliana roots after incubation with streptavidin-coated Dynabeads®, as described in Example 1;

FIG. 3E is a micrograph of total nuclei extracted from GL2p:NTF/ACT2p:BirA transgenic A. thaliana roots after incubation with streptavidin-coated Dynabeads® pre-treated with free biotin, as described in Example 1;

FIG. 4A is a fluorescence activated cell sorting (FACS) scatterplot of red versus green (GFP) fluorescence signals from 20,000 sorting events of non-transgenic protoplasts, wherein the boxed area shows the gate used for sorting GFP-positive protoplasts, as described in Example 2;

FIG. 4B is a fluorescence activated cell sorting (FACS) scatterplot of red versus green (GFP) fluorescence signals from 20,000 sorting events of protoplasts from the GL2p:NTF/ACT2p:BirA transgenic line, wherein the boxed area shows the gate used for sorting GFP-positive protoplasts, as described in Example 2;

FIGS. 4C and 4D are brightfield images of FACS-purified protoplasts from GL2p:NTF/ACT2p:BirA transgenic roots, as described in Example 2;

FIGS. 4E and 4F are GFP images of the same cells illustrated in FIGS. 4C and 4D, respectively, indicating the relative purity of the non-hair cell nuclei as isolated by FACS, as described in Example 2;

FIG. 5 is a scatter plot of nuclear RNA versus total RNA hybridization signals derived from the average of two replicates of tiling array data covering the entire sequenced portion of the A. thaliana genome. The whole genome expression profiles were performed using total RNA and nuclear RNA obtained from the differentiated root hair zone of 7 day old plants, as described in Example 2;

FIG. 6A graphically illustrates RT-PCR analysis of selected INTACT (isolation of nuclei tagged in specific cell types) hair (H) cell-enriched genes in wild type and gl2-8 roots, wherein all epidermal cells are H cells. The data represent the average of two biological replicates +/−SD. Asterisks indicate P values <0.05 and P values higher than 0.05 are indicated on the graph, as described in Example 2;

FIG. 6B graphically illustrates observed versus expected percentage of genes in each Gene Ontology (GO) annotation category for H cell-enriched genes, wherein Chi-square P values are indicated as ***<0.001, **<0.01, and *<0.03, as described in Example 2;

FIG. 6C graphically illustrates observed versus expected percentage of genes in each Gene Ontology (GO) annotation category for non-hair (NH) cell-enriched genes, wherein Chi-square P values are indicated as ***<0.001, **<0.01, and *<0.03, as described in Example 2;

FIG. 7A graphically illustrates euchromatic chromatin landscapes of the histone H3 modifications, H3K4me3 and H3K27me3, in hair (H) and non-hair (NH) cells. Chromosome 1 genes are shown schematically in the top line, wherein genes encoded in the top strand are indicated above the line and genes encoded in the bottom strand are below the line. Asterisks indicate genes where H3K4me3 and H3K27me3 overlap in both cell types. Each chromatin landscape is the average of two biological replicates, displayed on the same log-ratio scale, as described in Example 3;

FIG. 7B is a heat map of the H3K4me3 histone modification chromatin landscape relative to gene ends in hair (H) cells (−1 kb to +1 kb relative to transcription start and end sites). Genes are ranked according to expression levels in hair (H) cells, from the highest expression level at the top of the heat map to the lowest expression level at the bottom of the heat map. Yellow indicates positive log 2 ratios of H3K4me3, black indicates zero log 2 ratios, and blue represents negative log 2 ratios (with representative areas of yellow and blue as indicated), as described in Example 3;

FIG. 7C is a heat map of the H3K27me3 histone modification chromatin landscape relative to gene ends in hair (H) cells (−1 kb to +1 kb relative to transcription start and end sites). Genes are ranked according to expression levels in hair (H) cells, from the highest expression level at the top of the heat map to the lowest expression level at the bottom of the heat map. Yellow indicates positive log 2 ratios of H3K27me3, black indicates zero log 2 ratios, and blue represents negative log 2 ratios (with representative areas of yellow and blue as indicated), as described in Example 3;

FIG. 8A is a heat map of the H3K4me3 histone modification chromatin landscape relative to gene ends in non-hair (NH) cells (−1 kb to +1 kb relative to transcription start and end sites). Genes are ranked according to expression levels in non-hair (NH) cells, from the highest expression level at the top of the heat map to the lowest expression level at the bottom of the heat map. Yellow indicates positive log 2 ratios of H3K4me3, black indicates zero log 2 ratios, and blue represents negative log 2 ratios (with representative areas of yellow and blue as indicated), as described in Example 3;

FIG. 8B is a heat map of the H3K27me3 histone modification chromatin landscape relative to gene ends in non-hair (NH) cells (−1 kb to +1 kb relative to transcription start and end sites). Genes are ranked according to expression levels in non-hair (NH) cells, from the highest expression level at the top of the heat map to the lowest expression level at the bottom of the heat map. Yellow indicates positive log 2 ratios of H3K27me3, black indicates zero log 2 ratios, and blue represents negative log 2 ratios (with representative areas of yellow and blue as indicated), as described in Example 3;

FIG. 9A is a heat map showing H3K4me3 and H3K27me3 differences between hair (H) and non-hair (NH) cell types (H cell profile minus NH cell profile) around the 5′ end of genes (−1 kb to +1 kb from transcription start site) for the 946 H cell-enriched genes. The H cell-enriched genes are ranked according to fold difference in expression level between H and NH cells, with the highest fold difference level at the top of the heat map to the lowest fold difference level at the bottom of the heat map. Blue represents higher modification levels in NH cells while yellow indicates lower levels in NH cells, black represents no difference, and gray indicates no data where analysis was stopped when another genomic feature was encountered. Illustrative areas of blue and yellow dominance in the clusters are indicated, as described in Example 3;

FIG. 9B is a heat map showing H3K4me3 and H3K27me3 differences between hair (H) and non-hair (NH) cell types (H cell profile minus NH cell profile) around the 5′ end of genes (−1 kb to +1 kb from transcription start site) for the 118 NH cell-enriched genes. The NH cell-enriched genes are ranked according to fold difference in expression level between H and NH cells, with the highest fold difference level at the top of the heat map to the lowest fold difference level at the bottom of the heat map. Yellow represents higher modification levels in H cells, blue indicates lower modification levels in H cells, black represents no difference, and gray indicates no data where analysis was stopped when another genomic feature was encountered. Illustrative areas of blue and yellow dominance are indicated, as described in Example 3;

FIG. 9C is the same heat map illustrated in FIG. 9A clustered into 3 groups (kₘₑₐₙₛ=3) over −1 kb to +1 kb, wherein white horizontal bars delineate the three clusters and illustrative areas of blue and yellow dominance are indicated, as described in Example 3;

FIG. 9D is the same heat map illustrated in FIG. 9B clustered into 3 groups (kₘₑₐₙₛ=3) over −1 kb to +1 kb, wherein white bars delineate the three clusters and illustrative areas of blue and yellow dominance are indicated, as described in Example 3;

FIG. 9E graphically illustrates the euchromatic chromatin landscape of the histone H3 modifications, H3K4me3 and H3K27me3, in hair (H) and non-hair (NH) cells on the H cell-enriched gene At5g70450 (indicated by dotted box). Genes above the top line are encoded on the top strand while those below the line are encoded on the bottom strand, as described in Example 3;

FIG. 9F graphically illustrates the euchromatic chromatin landscape of the histone H3 modifications, H3K4me3 and H3K27me3, in hair (H) and non-hair (NH) cells on the H cell-enriched gene At3g49960 (indicated by dotted box). Genes above the top line are encoded on the top strand while those below the line are encoded on the bottom strand, as described in Example 3;

FIG. 9G graphically illustrates the euchromatic chromatin landscape of the histone H3 modifications, H3K4me3 and H3K27me3, in hair (H) and non-hair (NH) cells on the NH cell-enriched gene At1g66800 (indicated by dotted box). Genes above the top line are encoded on the top strand while those below the line are encoded on the bottom strand, as described in Example 3;

FIG. 9H graphically illustrates the euchromatic chromatin landscape of the histone H3 modifications, H3K4me3 and H3K27me3, in hair (H) and non-hair (NH) cells on the NH cell-enriched gene At5g42591 (indicated by dotted box). Genes above the top line are encoded on the top strand while those below the line are encoded on the bottom strand, as described in Example 3;

FIG. 10A is a heat map showing H3K4me3 and H3K27me3 differences between hair (H) and non-hair (NH) cell types (H cell profile minus NH cell profile) around the 5′ end of genes (−1 kb to +1 kb from transcription start site) for the 946 H cell-enriched genes. The H cell-enriched genes were clustered into 5 groups using k-means clustering, delineated by the white horizontal lines. Yellow represents higher modification levels in H cells, blue indicates lower levels in H cells, black represents no difference, and gray indicates no data where analysis was stopped when another genomic feature was encountered. Illustrative areas of blue and yellow dominance are indicated, as described in Example 3;

FIG. 10B is a heat map showing H3K4me3 and H3K27me3 differences between hair (H) and non-hair (NH) cell types (H cell profile minus NH cell profile) around the 5′ end of genes (−1 kb to +1 kb from transcription start site) for the 946 H cell-enriched genes. The H cell-enriched genes were clustered into 10 groups using k-means clustering, delineated by the white horizontal lines. Yellow represents higher modification levels in H cells, blue indicates lower levels in H cells, black represents no difference, and gray indicates no data where analysis was stopped when another genomic feature was encountered. Illustrative areas of blue and yellow dominance in the clusters are indicated, as described in Example 3;

FIG. 11A is a schematic illustration of a nucleic acid construct encoding a nuclear tagging fusion (NTF) protein used to label and isolate nuclei of Caenorhabditis elegans germline cells using the INTACT method, as described in Example 4;

FIG. 11B is a schematic illustration of a nucleic acid construct encoding a biotin ligase used to label and isolate nuclei of C. elegans germline cells using the INTACT method, as described in Example 4;

FIG. 12A is a fluorescence micrograph illustrating the localization of expressed NPP-9:mCherry:BLRP fusion protein in the nuclear envelopes of transgenic C. elegans germline cells. Autofluorescence in gut granules is also visible, as described in Example 4;

FIG. 12B is a streptavidin western blot of transgenic C. elegans whole cell extracts obtained from cells expressing the NPP-9:mCherry:BLRP NTF protein only or cells co-expressing the NTF protein and biotin ligase (BirA). The predicted size of the biotinylated fusion protein is indicated with an arrow, as described in Example 4;

FIG. 13A is a micrograph of DAPI stained total nuclei isolated from transgenic C. elegans with the NPP-9:mCherry:BLRP and BirA vectors, as described in Example 4;

FIG. 13B is a fluorescent micrograph of total nuclei isolated from transgenic C. elegans with the NPP-9:mCherry:BLRP vector, wherein bright spots indicate the presence of the NTF protein in the nuclear envelopes, as described in Example 4;

FIG. 13C is a western blot stained with anti-mCherry and anti-histone H3 antibodies of precipitates from transgenic C. elegans cells either expressing the NPP-9:mCherry:BLRP NTF protein only or co-expressing the NTF protein and biotin ligase (BirA), as described in Example 4;

FIG. 14A is a fluorescent micrograph of nuclei isolated from NPP-9::mCherry::BLRP transgenic C. elegans using streptavidin-coated Dynabeads® in connection with a flow apparatus incorporating a magnetic field, as described in Example 4;

FIG. 14B is a DAPI-stained micrograph of nuclei isolated from NPP-9::mCherry::BLRP transgenic C. elegans using streptavidin-coated Dynabeads® in connection with a flow apparatus incorporating a magnetic field, as described in Example 4;

FIG. 14C is a fluorescent micrograph of nuclei isolated from NPP-9::mCherry::BLRP and BirA::GFP transgenic C. elegans using streptavidin-coated Dynabeads® in connection with a flow apparatus incorporating a magnetic field, as described in Example 4;

FIG. 14D is a DAPI-stained micrograph of nuclei isolated from NPP-9::mCherry::BLRP and BirA::GFP transgenic C. elegans using streptavidin-coated Dynabeads® in connection with a flow apparatus incorporating a magnetic field, as described in Example 4;

FIG. 14E is a high magnification fluorescent micrograph of nuclei isolated from NPP-9::mCherry::BLRP and BirA::GFP transgenic C. elegans using streptavidin-coated Dynabeads® in connection with a flow apparatus incorporating a magnetic field, as described in Example 4;

FIG. 14F is a high magnification DAPI-stained micrograph of the nuclei illustrated in FIG. 14E, which were isolated from NPP-9::mCherry::BLRP and BirA::GFP transgenic C. elegans using streptavidin-coated Dynabeads® in connection with a flow apparatus incorporating a magnetic field, as described in Example 4;

FIG. 15A is a schematic illustration of a nucleic acid construct encoding a nuclear tagging fusion (NTF) protein used to label and isolate nuclei of Drosophila melanogaster somitic cells according to the INTACT method, as described in Example 5;

FIG. 15B is a schematic illustration of a nucleic acid construct encoding a biotin ligase used to label nuclei of D. melanogaster germline cells using the INTACT method, as described in Example 4;

FIG. 16 is a micrograph of a transgenic D. melanogaster embryo expressing both NTF protein and BirA ligase. The micrograph shows mCherry fluorescence from the NTF protein in the somitic cells. The inset is a higher magnification image showing the localization of the mCherry fluorescence to the nuclear envelope, as described in Example 5;

FIG. 17A is a DAPI-stained micrograph of nuclei isolated from transgenic D. melanogaster embryos expressing both the NTF protein and BirA ligase from the twist promoter. DNA in the nuclei is indicated with the intense signal, as described in Example 5.

FIG. 17B is a micrograph of the same nuclei illustrated in FIG. 17A, after incubation with fluorescing anti-FLAG antibodies. The NTF protein-tagged nuclei are indicated with the fluorescence signal, as described in Example 5;

FIG. 17C is a fluorescence micrograph of the same nuclei illustrated in FIG. 17A, after incubation with fluorescence-tagged streptavidin. The NTF protein-tagged nuclei are indicated with the fluorescence signal, as described in Example 5; and

FIG. 17D is a fluorescence micrograph of the same nuclei illustrated in FIG. 17A showing the mCherry fluorescence of the NTF protein-tagged nuclei, as described in Example 5;

FIGS. 18A-1 and FIG. 18A-2 provide schematic illustrations of two embodiments of a nucleic acid construct used to transgenically express a nuclear tagging fusion (NTF) protein based on the Sun-1 protein and containing a domain encoding SUN domain (SD) to embed the protein in the inner nuclear membrane INM of the nuclear envelope. In the first embodiment (A-1), a domain encoding GFP at the 3′ end relative to the SUN-encoding domain results in an affinity reagent binding region and visualization tag being C-terminal to the SUN domain and localizing in the lumen (L) of the nuclear membrane. In the second embodiment (A-2), the visualization tag is a tdTomato domain incorporating a sequence encoding an epitope tag to serve as the affinity reagent binding region, as described in Example 6;

FIG. 18B is an illustration of an embodiment of the present invention in which a nuclear tagging protein comprising a SUN domain is embedded in the INM of the nuclear envelope. Two embodiments are shown, comprising either a tdTomato+epitope tag or a 2XGFP, as described in Example 6;

FIG. 19A is a schematic illustration of a nucleic acid construct used to transgenically express a nuclear tagging fusion (NTF) protein based on the Nesprin-3 protein and containing a domain encoding KASH domain (KD) to embed the protein in the outer nuclear membrane ONM of the nuclear envelope. The GFP proteins are encoded at the 5′ end relative to the KASH-encoding domain, resulting in the GPF domains being N-terminal to the SUN domain and localizing in the cytoplasm of the cell, as described in Example 6;

FIG. 19B is an illustration of an embodiment of the present invention in which a nuclear tagging protein comprising a KASH domain is embedded in the ONM of the nuclear envelope, as described in Example 6;

FIG. 20A is a fluorescence micrograph illustrating the nuclear membrane localization (green fluorescence indicated by the arrow) of Sun-2XGFP nuclear tagging fusion protein in a rat primary hippocampal cultured cell electroporated at P0. The expression of the tagging fusion protein was driven by the CMV promoter. The image was acquired at P6 using an IX81 Olympus Disk Spinning Unit Confocal microscope (bar=10 μm), as described in Example 6;

FIG. 20B is a fluorescence micrograph illustrating the nuclear localization (red fluorescence indicated by the arrow) of Sun-tdTomato-3XMYC nuclear tagging fusion protein in a rat primary hippocampal cultured cell electroporated at P0. The expression of the tagging fusion protein was driven by the CMV promoter. The image was acquired at P6 using an IX81 Olympus Disk Spinning Unit Confocal microscope (bar=10 μm), as described in Example 6;

FIG. 20C is a fluorescence micrograph illustrating the nuclear localization (green fluorescence indicated by the arrow) of LacZ-nls-GFP protein in a rat primary hippocampal cultured cell electroporated at P0. The expression of the fusion protein was driven by the CMV promoter. The image was acquired at P6 using an IX81 Olympus Disk Spinning Unit Confocal microscope (bar=10 μm), as described in Example 6;

FIG. 21A is a fluorescence micrograph illustrating the localization of Sun-tdTomato-3XMYC after chronic expression in Lentivirus-infected striatal neurons. A Zeiss LSM 510 microscope was used to collect the image from 40 μm thick cryosections, as described in Example 6;

FIG. 21B is a fluorescence micrograph illustrating the localization of Sun-tdTomato-3XMYC after chronic expression in Lentivirus-infected striatal neurons, wherein the cells were also infected with Lentivirus expressing GFP. Images from both fluorescence channels are overlayed. A Zeiss LSM 510 microscope was used to collect the images from 40 μm thick cryosections, as described in Example 6;

FIG. 22A is a representative transmission electron micrograph of a nucleus from a cultured COS cell isolated in the presence of 0.5% NP40. The white arrow in the inset indicates the INM. The nucleus lacks an ONM. n=20 for the extraction. Images were obtained with a FEI Tecnai G2 transmission electron microscope (bar=1 μm), as described in Example 6;

FIG. 22B is a representative transmission electron micrograph of a nucleus from a cerebellar neuron isolated from in vivo in the presence of 0.5% NP40. The white arrow in the inset indicates the INM. The nucleus lacks an ONM. n=20 for the extraction. Images were obtained with a FEI Tecnai G2 transmission electron microscope (bar=1 μm), as described in Example 6;

FIG. 22C is a representative transmission electron micrograph of a nucleus from a cerebellar neuron isolated from in vivo in the absence of any detergent. The white arrow in the inset indicates the INM, whereas the dark arrow indicates the ONM. n=20 for the extraction. Images were obtained with a FEI Tecnai G2 transmission electron microscope (bar=1 μm), as described in Example 6;

FIG. 23A is a schematic representation of an embodiment of the nuclear immunopurification procedure, wherein the cells are first lysed and the nuclei are immunopurified, followed by biochemical manipulation, such as Micrococcal nuclease or DNaseI treatment, as described in Example 6;

FIG. 23B is a schematic representation of the nuclear immunopurification procedure, wherein the cells are first permeabilized, followed by biochemical manipulation, such as Micrococcal nuclease or DNaseI treatment. Finally, the cells are lysed and the nuclei are immune-purified, as described in Example 6;

FIG. 24A illustrates the extracted nucleosomal DNA obtained at increasing salt concentrations (50-400 mM) from ˜10⁶ COS cells tagged with Sun-2XGFP, and subsequently immunopurified and subjected to Micrococcal nuclease, as described in Example 6;

FIG. 24B illustrates the extracted nucleosomal DNA obtained at increasing salt concentrations (50-400 mM) from ˜10⁶ COS cells and subjected to Micrococcal nuclease, as described in Example 6;

FIGS. 25A and B are fluorescence micrographs illustrating the tagged nuclei in the ventral nerve cord (VNC) of 3rd instar of D. melanogaster larvae. The nerve cells expressed nuclear tagging fusion proteins incorporating the C. elegans SUN domain protein Unc-84 fused with GFP. Panel B merges the image of panel A with the DAPI stain image, as described in Example 7;

FIGS. 25C and D are fluorescence micrographs illustrating the tagged nuclei in the ventral nerve cord (VNC) of 3rd instar of D. melanogaster melanogaster larvae. The nerve cells expressed nuclear tagging fusion proteins incorporating the D. melanogaster KASH domain protein klarsicht fused with GFP. Panel B merges the image of panel A with the DAPI stain image, as described in Example 7;

FIGS. 25E and F are fluorescence micrographs illustrating the tagged nuclei in the ventral nerve cord (VNC) of 3rd instar of D. melanogaster melanogaster larvae. The nerve cells expressed nuclear tagging fusion proteins incorporating the C. elegans SUN domain protein Unc-84 fused with GFP. Panel B merges the image of panel A with the DAPI stain image, as described in Example 7;

FIGS. 25 G and H are fluorescence micrographs illustrating the tagged nuclei in the ventral nerve cord (VNC) of 3rd instar of D. melanogaster melanogaster larvae. The nerve cells expressed nuclear tagging fusion proteins incorporating the C. elegans SUN domain protein Unc-84 fused with tdTomato. Panel B merges the image of panel A with the DAPI stain image, as described in Example 7;

FIGS. 26A and B are fluorescence micrographs of illustrating the frontal and ventral views of a D. melanogaster brain exhibiting cell-type specific expression of the Unc-84-2XGFP nuclear tagging fusion protein in fruitless neurons (illustrative signal indicated by white arrow), as described in Example 7;

FIGS. 26C and D are fluorescence micrographs of illustrating the frontal and ventral views of a D. melanogaster brain exhibiting cell-type specific expression of the Unc-84-2XGFP nuclear tagging fusion protein in Kenyon cells of the mushroom body (illustrative signal indicated by white arrow), as described in Example 7;

FIGS. 26E and F are fluorescence micrographs of illustrating the frontal and ventral views of a D. melanogaster brain exhibiting cell-type specific expression of the Unc-84-2XGFP nuclear tagging fusion protein in a sub-population of cells in the antennal lobe (illustrative signal indicated by white arrow), as described in Example 7; and

FIGS. 26 G and H are fluorescence micrographs of illustrating the frontal and ventral views of a D. melanogaster brain exhibiting cell-type specific expression of the Unc-84-2XGFP nuclear tagging fusion protein in octopaminergic neurons (illustrative signal indicated by white arrow), as described in Example 7.

### DESCRIPTION OF THE SEQUENCE LISTING

SEQ ID NO:1 Arabidopsis RAN GTPASE ACTIVATING PROTEIN 1 (RanGAP1) WPP domain DNA

SEQ ID NO:2 Arabidopsis RAN GTPASE ACTIVATING PROTEIN 1 (RanGAP1) WPP domain amino acid

SEQ ID NO:3 enhanced Green Fluorescent Protein (eGFP) DNA

SEQ ID NO:4 enhanced Green Fluorescent Protein (eGFP) amino acid

SEQ ID NO:5 biotin ligase recognition peptide DNA

SEQ ID NO:6 biotin ligase recognition peptide amino acid

SEQ ID NO:7 shortened biotin ligase recognition peptide DNA

SEQ ID NO:8 shortened biotin ligase recognition peptide amino acid

SEQ ID NO:9 DNA encoding the full length nuclear tagging fusion (NTF) protein, as used in EXAMPLE 1

SEQ ID NO:10 full length amino acid of the nuclear tagging fusion (NTF) protein, as used in EXAMPLE 1

SEQ ID NO:11 E. coli biotin holoenzyme synthetase (BirA) DNA

SEQ ID NO:12 E. coli biotin holoenzyme synthetase (BirA) amino acid

SEQ ID NO:13 A. thaliana ACTIN DEPOLYMERIZING FACTOR 8 (ADF8) promoter

SEQ ID NO:14 A. thaliana GLABRA 2 (GL2) promoterA

SEQ ID NO:15 A. thaliana ACTION 2 (ACT2) promoter

SEQ ID NO:16 mCherry fluorescent protein DNA

SEQ ID NO:17 mCherry fluorescent protein amino acid

SEQ ID NO:18 C. elegans H3.3 (his-72) promoter sequence (Chromosome III, 12368042 to 12369042, −strand)

SEQ ID NO:19 C. elegans H3.3 (his-72) 3′UTR sequence (Chromosome III, 12366572 to 12367571, −strand)

SEQ ID NO:20 C. elegans pie-1 promoter sequence (Chromosome III, 12424364 to 12426776, +strand)

SEQ ID NO:21 C. elegans pie-1 3′ UTR sequence (Chromosome III, 12428972 to 12429871, +strand)

SEQ ID NO:22 C. elegans NPP-9 domain DNA with introns

SEQ ID NO:23 C. elegans NPP-9 domain amino acid

SEQ ID NO:24 DNA encoding the full length nuclear tagging fusion (NTF) protein, as used in EXAMPLE 4

SEQ ID NO:25 full length amino acid of the nuclear tagging fusion (NTF) protein, as used in EXAMPLE 4

SEQ ID NO:26 3X FLAG affinity tag domain nucleic acid

SEQ ID NO:27 3X FLAG affinity tag domain amino acid

SEQ ID NO:28 D. melanogaster RanGAP domain DNA with introns

SEQ ID NO:29 D. melanogaster RanGAP domain amino acid

SEQ ID NO:30 DNA encoding the full length nuclear tagging fusion (NTF) protein, as used in EXAMPLE 5

SEQ ID NO:31 full length amino acid of the nuclear tagging fusion (NTF) protein, as used in EXAMPLE 5

SEQ ID NO:32 D. melanogaster twist promoter

SEQ ID NOS:33-86 primer sequences

SEQ ID NO:87 biotin ligase recognition peptide DNA

SEQ ID NO:88 biotin ligase recognition peptide amino acid

SEQ ID NO:89 amino acid sequence of linker used in the nuclear tagging fusion proteins based on Sun-1 and Nesprin-3, as described in EXAMPLE 6

SEQ ID NO:90 DNA encoding the mouse Nesprin-3 protein, as used in EXAMPLE 6

SEQ ID NO:91 full length amino acid of the mouse Nesprin-3 protein, as used in EXAMPLE 6

SEQ ID NO:92 DNA encoding the mouse Sun-1 protein, as used in EXAMPLE 6

SEQ ID NO:93 full length amino acid of the mouse Sun-1 protein, as used in EXAMPLE 6

SEQ ID NO:94 DNA encoding the D. melanogaster klarsicht protein (klar), as used in EXAMPLE 7

SEQ ID NO:95 full length amino acid of the D. melanogaster klarsicht (klar) protein, as used in EXAMPLE 7

SEQ ID NO:96 DNA encoding the C. elegans Unc-84 protein, as used in EXAMPLE 7

SEQ ID NO:97 full length amino acid of the C. elegans Unc-84 protein, as used in EXAMPLE 7

SEQ ID NO:98 DNA encoding the C. elegans Unc-83 protein, as used in EXAMPLE 7

SEQ ID NO:99 full length amino acid of the C. elegans Unc-83 protein, as used in EXAMPLE 7

### DETAILED DESCRIPTION

Unless defined otherwise, all technical and scientific terms used herein have the meaning commonly understood by one of ordinary skill in the art to which this invention belongs. Practitioners are particularly directed to Sambrook, J., and Russell, D. W., eds., Molecular Cloning: A Laboratory Manual, 3rd ed., Cold Spring Harbor Laboratory Press, Cold Spring Harbor, N.Y. (2001), which is incorporated herein by reference, for definitions and terms of the art.

The following definitions are presented to provide clarity with respect to the terms as they are used in the specification and claims to describe the present invention.

As used herein, the term “gene” refers to a nucleic acid (e.g., DNA or RNA) sequence that comprises coding sequences necessary for the production of an RNA and/or a polypeptide, or its precursor as well as noncoding sequences (untranslated regions) surrounding the 5′ and 3′ ends of the coding sequences. The term “gene” encompasses both cDNA and genomic forms of a gene. A functional polypeptide can be encoded by a full length coding sequence or by any portion of the coding sequence as long as the desired activity or functional properties (e.g., enzymatic activity, ligand binding, signal transduction, antigenic presentation) of the polypeptide are retained. The sequences which are located 5′ of the coding region and which are present on the mRNA are referred to as 5′ untranslated sequences (“5′UTR”). The sequences which are located 3′ or downstream of the coding region and which are present on the mRNA are referred to as 3′ untranslated sequences, or (“3′UTR”).

As used herein, the terms “polypeptide” or “protein” are used interchangeably to refer to polymers of amino acids of any length. A polypeptide or amino acid sequence “derived from” a designated protein refers to the origin of the polypeptide.

As used herein, the term “promoter” refers to a region, or combination of regions, of DNA within a gene that facilitates the transcription of the gene. These regions typically provide binding sites for transcription factors, which participate in the assembly of the transcriptional complex.

As used herein, the term “operatively linked” refers to a juxtaposition wherein the components so described are in a relationship permitting them to function in their intended manner. For example, a promoter sequence is operatively linked to a coding sequence if the promoter sequence promotes transcription of the coding sequence.

As used herein, the term “antibody” encompasses antibodies and antibody fragments thereof, derived from any antibody-producing vertebrate (e.g., mouse, rat, rabbit, camelid, and primate, including human), that specifically bind to a polypeptide target of interest, or portions thereof.

As used herein, the term “vector” is a nucleic acid molecule, preferably self-replicating, which transfers and/or replicates an inserted nucleic acid molecule into and/or between host cells. Exemplary vectors include plasmid vectors and viral vectors. An example of viral vector is a Lentiviral vector.

As used herein, the terms indicating “percent identity” or “percent identical,” refer to the percentage of nucleotides in a nucleic acid sequence or amino acid residues in a polypeptide sequence that are identical with the nucleic acid sequence or amino acid sequence of a specified molecule, after aligning the sequences to achieve the maximum percent identify. For example, the Vector NTI Advance™ 9.0 may be used for sequence alignment.

As used herein, the term “variant,” in reference to a nucleic acid or polypeptide of any length, refers to a related nucleic acid or polypeptide that has between 90% and 99% identity with the nucleic acid or polypeptide of reference over the length of the reference nucleotide or amino acid sequence, such as 90%, 91%, 92%, 93%, 94%, 95%, 96%, 97%, 98%, and 99% identity or with the reference nucleotide or amino acid sequence. Furthermore, the related nucleic acid or polypeptide possesses the equivalent functional qualities of the reference nucleic acid or protein. For example, a polypeptide that is a variant of biotin ligase recognition peptide can have between 90% to 99% identity with the sequence of the reference biotin ligase recognition peptide, wherein the variant polypeptide is capable of recognition and biotinylation by biotin ligase. In another example, a polypeptide that is a variant of a nuclear envelope targeting region polypeptide can have between 90% and 99% identity with the sequence of the reference nuclear envelope targeting region polypeptide, wherein the variant polypeptide is capable of being translocated and attached to the nuclear envelope of the cell. In yet another example, a variant nucleic acid promoter sequence can have between 90% and 99% identity with the reference promoter sequence, wherein the variant promoter sequence is capable of initiating transcription with the same or similar transcription factors as the reference promoter sequence.

The present invention provides a cost- and time-effective method to isolate the nuclei of a cell type of interest to enable genomic analyses of the cell-type.

In one embodiment, the invention utilizes a vector comprising a nucleic acid sequence encoding a fusion polypeptide comprising (a) a nuclear envelope targeting region and (b) an affinity reagent binding region.

As used herein, the term “affinity reagent binding region” refers to an amino acid sequence that is capable of directly binding to, or being bound by, a capture affinity reagent (e.g., an antibody that selectively binds to an epitope in the affinity reagent binding region), and also encompasses an amino acid sequence that is modified, such as by a post-translational modification (e.g., biotinylated in vivo), wherein the modified (e.g., biotinylated) version of the amino acid sequence is capable of binding to an affinity reagent (e.g. avidin and streptavidin).

As used herein, the terms “affinity reagent”, “capture reagent”, and “capture molecule” are used interchangeably to refer to reagents that bind to affinity reagent binding regions with sufficient specificity and avidity to facilitate the isolation of any molecule or cell structure, namely nuclei, with an affinity reagent binding region incorporated therein.

In some embodiments, the affinity region comprises an epitope tag and the affinity binding reagent is an antibody that selectively binds to the epitope tag. In some embodiments, the affinity binding region comprises a “biotin ligase accepting site,” also referred to as a “biotin ligase recognition peptide (BLRP),” that is biotinylated in vivo with a biotin ligase and the affinity binding reagent is a capture molecule capable of specifically binding to biotin. In some embodiments, the in vivo biotinylated nuclei of the cell type of interest are subsequently purified utilizing a biotin capture molecule.

Various embodiments of this invention, also referred to herein as “INTACT” (isolation of nuclei tagged in specific cell types), allow for the production and isolation of cell-type specific nuclei that are tagged (i.e., labeled) with a nuclear tagging fusion (“NTF”) polypeptide comprising an affinity binding region (e.g., comprising an epitope tag or biotin ligase accepting site for biotinylation) and a nuclear envelope targeting domain.

In an exemplary embodiment, isolation of tagged nuclei was accomplished by the co-expression of Escherichia coli biotin ligase BirA and a nuclear tagging fusion (NTF) protein in two Arabidopsis thaliana root epidermis cell types, as described in EXAMPLE 1. In the exemplary embodiment described in EXAMPLE 1, the NTF protein comprised the following three regions: (1) the WPP domain of Arabidopsis RAN GTPASE ACTIVATING PROTEIN 1 (RanGAP1), which is necessary and sufficient for envelope association (Rose, A., and I. Meier, “A Domain Unique to Plant RanGAP Is Responsible for Its Targeting to the Plant Nuclear Rim,” Proc. Natl. Acad. Sci. USA 98:15377-15382, 2001), (2) the green fluorescent protein (GFP) for visualization, and (3) the affinity binding region comprising the biotin ligase recognition peptide (BLRP), which acts as a substrate for the E. coli biotin ligase BirA (Beckett, D., et al., “A Minimal Peptide Substrate in Biotin Holoenzyme Synthetase-Catalyzed Biotinylation,” Protein Sci. 8:921-929, 1999). Cell type-specific expression of the NTF protein was driven in A. thaliana root epidermis hair cells using ACTIN DEPOLYMERIZING FACTOR 8 (ADF8) promoter (Ruzicka, D. R., et al., “The Ancient Subclasses of Arabidopsis Actin Depolymerizing Factor Genes Exhibit Novel and Differential Expression,” Plant J. 52:460-472, 2007), and in non-hair cells using GLABRA2 (GL2) promoter (Masucci, J. D., et al., “The Homeobox Gene GLABRA2 is Required for Position-Dependent Cell Differentiation in the Root Epidermis of Arabidopsis thaliana,” Development 122:1253-1260, 1996). As described in EXAMPLES 1-3, the method provided a high yield and purity of nuclei from each cell type of interest, facilitating a robust analyses of the genome-wide gene expression and chromatin structures for each cell type.

To demonstrate the applicability of the INTACT method to all eukaryotic organisms, NTF protein and BirA ligase were co-expressed specifically in germline cells of Caenorhabditis elegans resulting in the successful production and isolation of tagged germline cell nuclei, as described in EXAMPLE 4. Similarly, NTF protein and BirA ligase were successfully co-expressed specifically in somitic cells of Drosophila melanogaster embryos, as described in EXAMPLE 5. Furthermore, NTF proteins incorporating SUN or KASH domains, in connection with either GFP or tdTomato/epitope tag, were expressed mice (as described in EXAMPLE 6) and D. melanogaster (EXAMPLE 7).

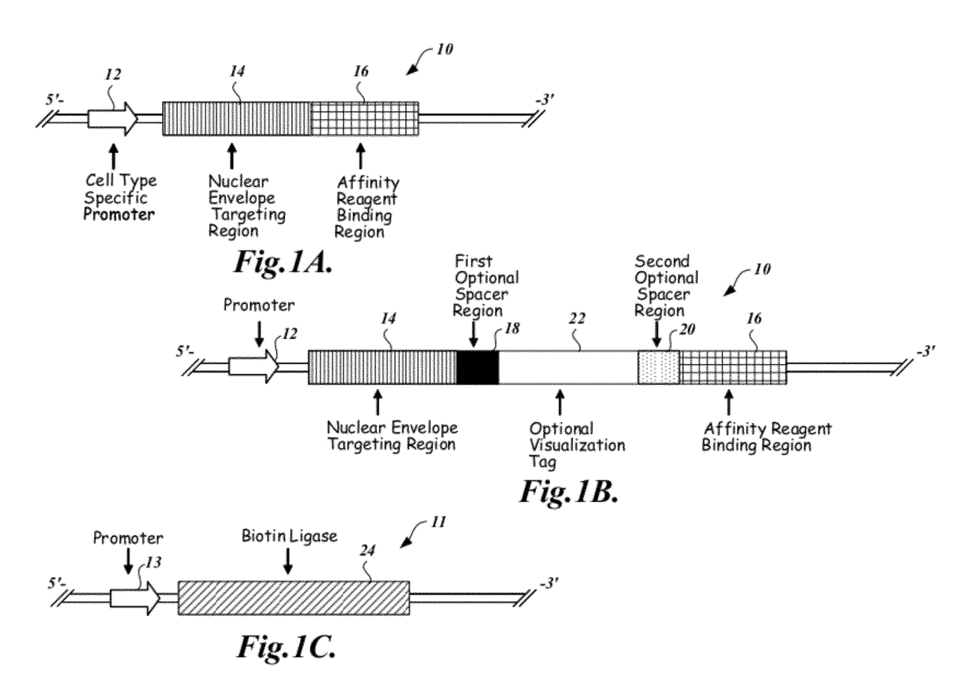

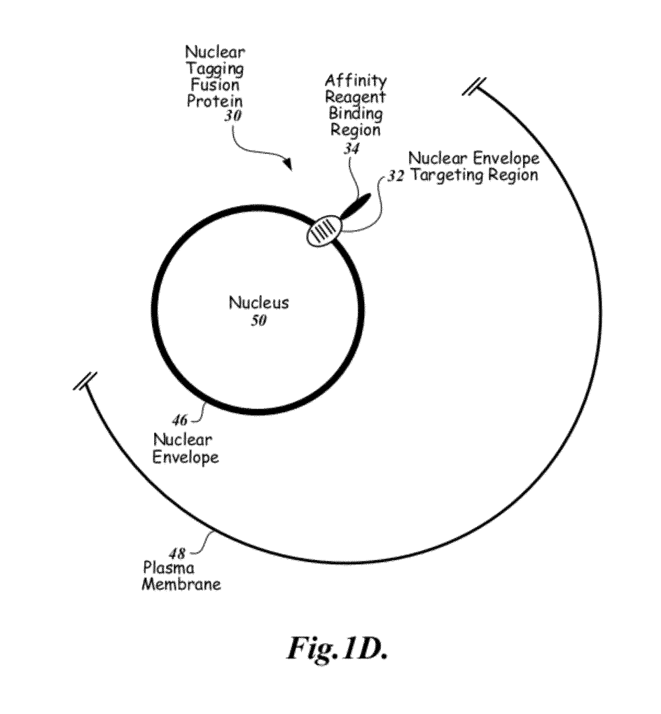

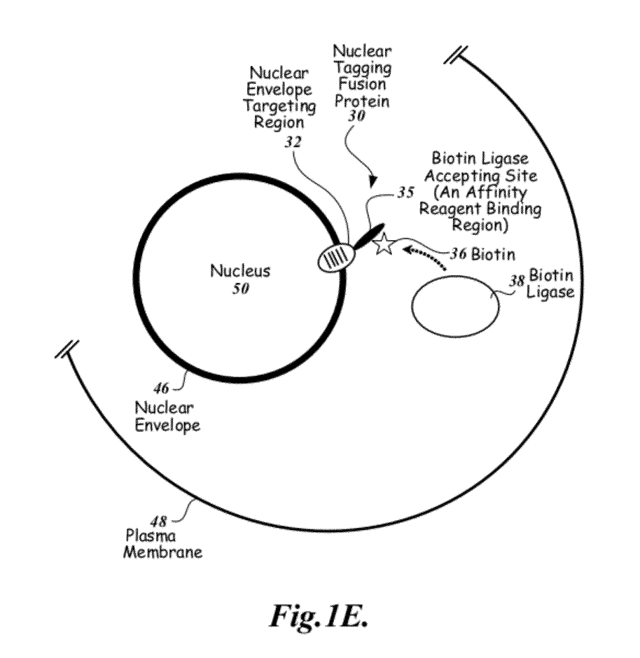

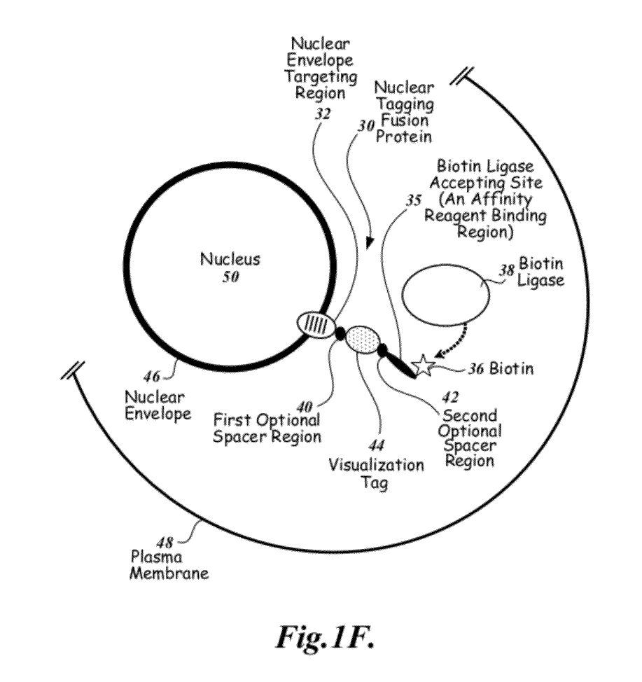

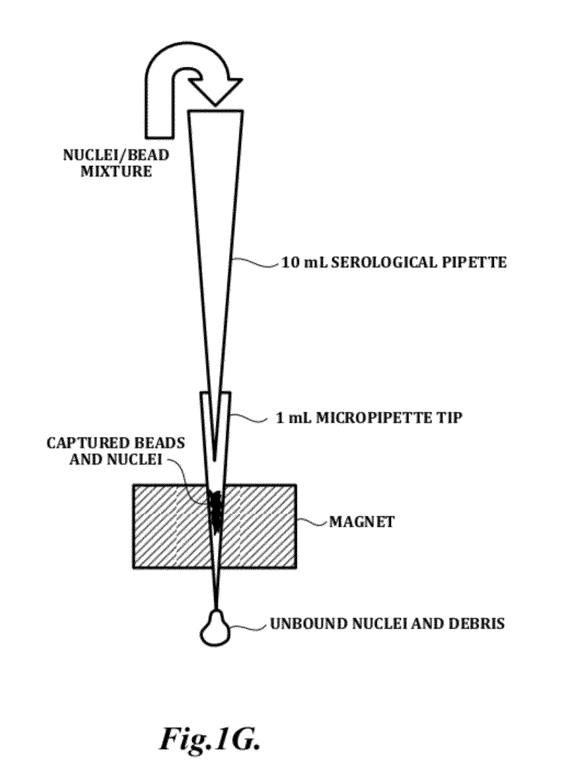

In accordance with the foregoing, in one embodiment, the present invention provides a vector 10 for selectively labeling nuclei in a cell type of interest comprising a nucleic acid sequence encoding a nuclear tagging fusion (NTF) polypeptide 30 comprising (a) a nuclear envelope targeting region 32; and (b) an affinity reagent binding region 34. In the embodiment of the vector shown in FIG. 1A, the vector 10 includes a nucleotide sequence 14 encoding a nuclear envelope targeting region 32. The encoded nuclear envelope targeting region 32 can be any amino acid sequence that causes the translocation of the translated fusion polypeptide 30 to the nuclear envelope 46 of the cell type of interest to facilitate the incorporation of the fusion protein 30 into the nuclear envelope 46. The nuclear envelope targeting region 32 is preferably chosen to correspond with the intra-nuclear transport infrastructure of the organism of interest. In some embodiments, the nuclear targeting region is associated with a transmembrane domain that becomes embedded in the nuclear envelope bilayer and anchors the fusion protein in place. However, in some embodiments, the transmembrane domain does not need to be incorporated into the sequence of the nuclear envelope targeting region, but rather may be a distinct domain in the fusion protein.

For example, in one embodiment as described in EXAMPLES 1-3, the vector 10 was designed for use in plant cells and comprised a nucleic acid sequence encoding the WPP domain of the Arabidopsis RAN GTPASE ACTIVATING PROTEIN 1 (RanGAP1), set forth herein as SEQ ID NO: 1. As described in EXAMPLE 1, the expressed NTF protein that included the amino acid sequence of the RanGAP1 WPP domain, set forth herein as SEQ ID NO:2, successfully caused the translocation and incorporation of the fusion protein to the nuclear envelope of A. thaliana epidermal root cells. In some embodiments, the vector 10 comprises a nucleic acid sequence encoding a nuclear envelope targeting region with an amino acid sequence of SEQ ID NO:2, or a variant thereof. Because the RanGAP1 WPP domain is relatively conserved in plants, use of this domain is predicted to be useful in employing this system in many other, if not all, types of plants.

For embodiments using cell-types from non-plant organisms, the C-terminus of the endogenous RanGAP protein may be used, or any number of nuclear pore proteins may be used. For example, in one embodiment for the nematode Caenorhabditis elegans, NPP-9, a C. elegans homolog of mammalian Nup358/RanBP2 was used to target the NTF protein 30 to the nuclear envelope, as described in EXAMPLE 4. The amino acid sequence for the NPP-9 domain is set forth herein as SEQ ID NO:23, and is encoded by the nucleic acid set forth herein as SEQ ID NO:23. Additionally, in an embodiment for Drosophila melanogaster, the D. melanogaster RanGAP protein was used to target the NTF protein 30 to the nuclear envelope, as described in EXAMPLE 5. The amino acid sequence for the D. melanogaster RanGAP domain is set forth herein as SEQ ID NO:29, and is encoded by the nucleic acid set forth herein as SEQ ID NO:28. Accordingly, some embodiments comprise a nucleic acid encoding polypeptides that are variants with at least 90% identity of SEQ ID NOS: 23 and 29.

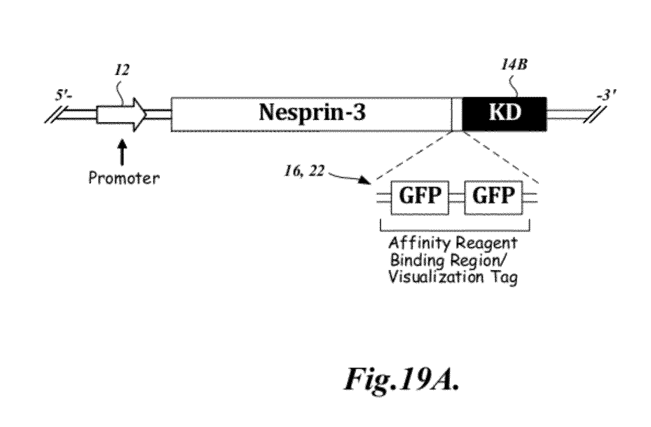

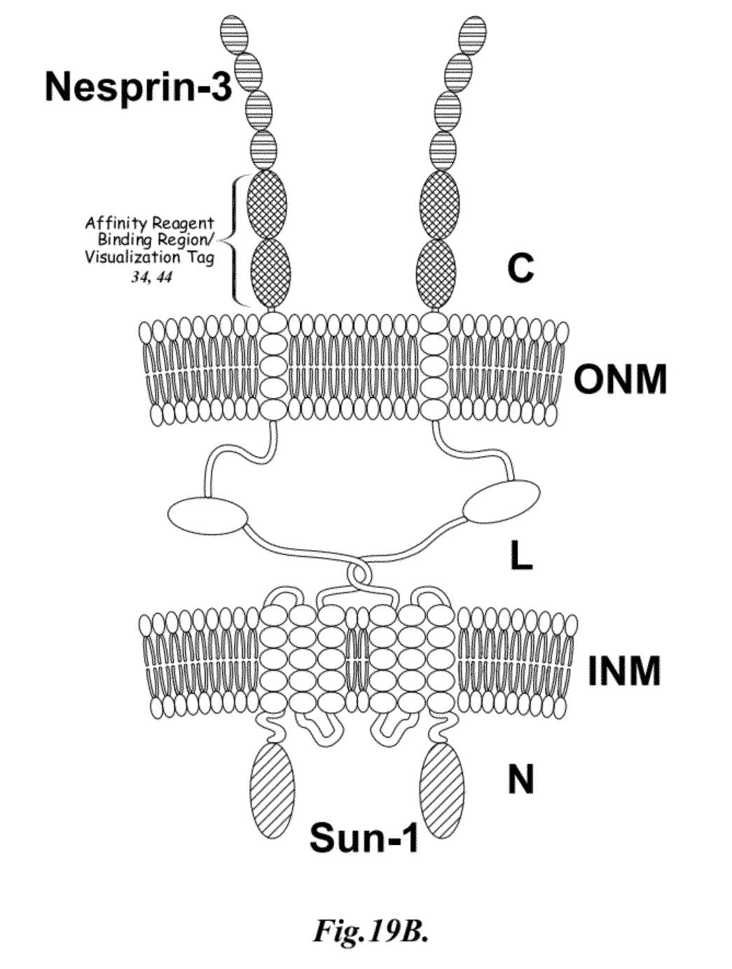

The nuclear envelope is a double lipid bilayer composed of an inner nuclear membrane (INM) and outer nuclear membrane (ONM) separated by a space referred to as the lumen (L) (see FIG. 19B). Therefore, in some embodiments, the nuclear envelope targeting region can comprise a polypeptide that causes the specific translocation of the NTF protein to one of the lipid bilayers of the nuclear envelope. In some embodiments, the nuclear envelope targeting region causes the NTF to be embedded in the ONM. As described in EXAMPLES 6 and 7, NTF proteins incorporating members of the KASH domain family of proteins were shown to tag the ONM of nuclei in mice and Drosophila, respectively. Accordingly, in some embodiments, the nuclear envelope targeting region comprises a KASH domain. Illustrative KASH domains include the sequence from amino acid residue 947 to amino acid residue 975 of SEQ ID NO:91 and the sequence from amino acid residue 512 to amino acid residue 567 of SEQ ID NO:95. In some embodiments, the KASH domain has a polypeptide sequence with at least 90% identity to the sequence from amino acid residue 947 to amino acid residue 975 of SEQ ID NO:91 or the sequence from amino acid residue 512 to amino acid residue 567 of SEQ ID NO:95, or any naturally occurring homolog thereof.

In other embodiments, the nuclear envelope targeting region causes the NTF to be embedded in the INM. Embodiments that incorporate nuclear envelope targeting regions specific for the INM are useful to accommodate culture or extraction techniques that may compromise the ONM of the nuclear envelope. For example, some detergents may disrupt the ONM, as described in EXAMPLE 6. In this regard, NTF proteins incorporating members of the SUN domain family of proteins were shown to tag the INM of nuclei in mice and Drosophila, respectively, as described in EXAMPLES 6 and 7. Accordingly, in some embodiments, the nuclear envelope targeting region comprises a SUN domain. Illustrative SUN domains include the sequence from amino acid residue 771 to amino acid residue 911 of SEQ ID NO:93, and the sequence from amino acid residue 971 to amino acid residue 1108 of SEQ ID NO:97. In some embodiments, the KASH domain has a polypeptide sequence with at least 90% identity to the sequence from amino acid residue 771 to amino acid residue 911 of SEQ ID NO:93, or the sequence from amino acid residue 971 to amino acid residue 1108 of SEQ ID NO:97, or any naturally occurring homolog thereof. An additional representative SUN domain is incorporated in the sequence from amino acid residue 425 to amino acid residue 460 of the D. melanogaster klaroid protein, the amino acid sequence of which has the Genbank accession number NM_{—}136396.3, hereby incorporated herein by reference (as accessed on Sep. 15, 2011).

As illustrated in FIG. 1A, the vector construct 10 encoding the NTF 30 protein also includes a nucleic acid sequence 16 encoding an affinity reagent binding region 34. In some embodiments, the affinity reagent binding region 34 of the expressed NTF protein 30 comprises an epitope tag (e.g., immunological tag) that is capable of being specifically bound by an affinity (capture) reagent, such as an antibody or fragment thereof. An epitope tag refers to a contiguous sequence of amino acids that are specifically bound by an immunological capture reagent, such as an antibody or fragment thereof. Illustrative, non-limiting examples of suitable epitope tags include c-myc, HA, FLAG-tag, GST, 6HIS, VSVg, V5, HSV, and AU1 and others that are well known in the art. As will be recognized by persons of ordinary skill in the art, the epitope tags can be optionally multimerized to create repeating units of the epitope tag.

A person of ordinary skill in the art will recognize that the affinity reagent binding region 34 can also be an epitope located within a detectable polypeptide, such as a fluorescent protein or other visualization tag. Thus, in some embodiments, the affinity (capture) reagent, such as an antibody, can be used against an epitope contained in a fluorescence protein or other visualization tag that is included in the fusion protein, as described herein. In another embodiment, the capture reagent that specifically binds to the affinity reagent binding region 34 may be labeled with a molecule capable of emitting detectable light or energy. In some embodiments, the immunological capture agent may also be bound to a bead. Numerous types of antibody-bound beads are commercially available.

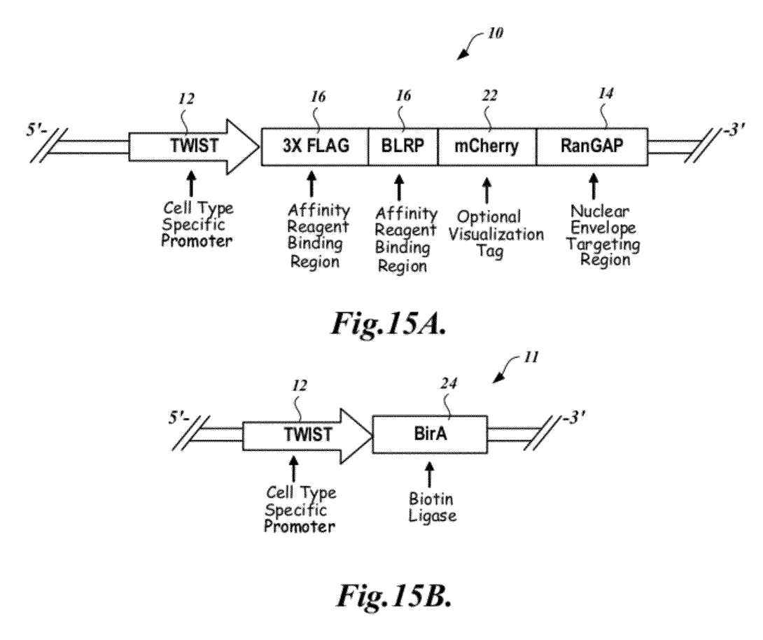

To ensure access of the affinity capture reagent to the affinity reagent binding region 34 (e.g., epitope tag or biotin ligase accepting site) of the NTF protein 30 of a tagged nucleus, it is preferred that the vector 10 encodes an NTF protein 30 such that the relative positions of the translated nuclear targeting region 32 and affinity reagent binding region 34 will result in the positioning of the affinity binding region 34 in the extra-nuclear space of the cell upon the incorporation of the NTF protein 30 to the nuclear envelope 46. For example, FIG. 1D is a schematic illustration of an embodiment of the INTACT system in which the nuclear envelope 46 is labeled with a transgenically expressed NTF protein 30. The nuclear envelope targeting region 32 is illustrated as being embedded in the nuclear envelope 46. The affinity reagent binding region 34 is disposed in the extra nuclear space contained within the plasma membrane 48 of the cell. Accordingly, in cases where the nuclear envelope targeting region 32 results in the C terminal end of the NTF 30 protein being positioned in the extra-nuclear space, the NTF protein 30 is encoded by the vector 10 in a manner in which the translated affinity reagent binding region 34 (e.g., epitope tag or biotin ligase accepting site) is at the C terminal end of the NTF protein 30 relative to the location of the nuclear envelope targeting region 32. For example, FIG. 1A illustrates a vector 10 construct encoding the NTF protein 30 with the nucleotide sequence 14 encoding the nuclear envelope targeting region 32 at the 5′ end of the construct relative to the nucleotide sequence 16 encoding the affinity binding region 34 (e.g., epitope tag or biotin ligase accepting site). A person of ordinary skill in the art would recognize that in cases where the nuclear envelope targeting region 32 results in the N terminal end of the NTF protein 30 being situated in the extra-nuclear space, the vector 10 encoding the NTF polypeptide comprises the nucleotide sequence 14 encoding the nuclear envelope targeting region located at the 3′ end of the protein relative to the location of the nucleotide sequence 16 encoding the affinity reagent binding region (e.g., epitope tag or biotin ligase accepting site). An example of this embodiment is described in Example 5 and illustrated in FIG. 15A, wherein the nucleic acid 14 encoding the nuclear envelope targeting region (e.g. RanGAP) is located at the 3′ end of the vector 10 relative to the domains encoding for visualization tags 22 (e.g. mCherry) and/or affinity reagent binding regions 16 (e.g. sequence encoding biotin ligase binding polypeptide and/or the sequence encoding the FLAG epitope tag).

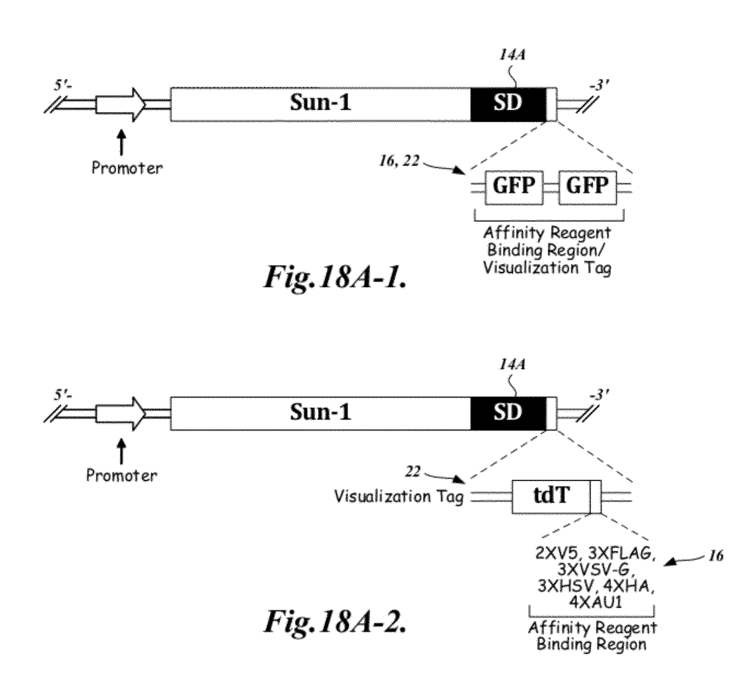

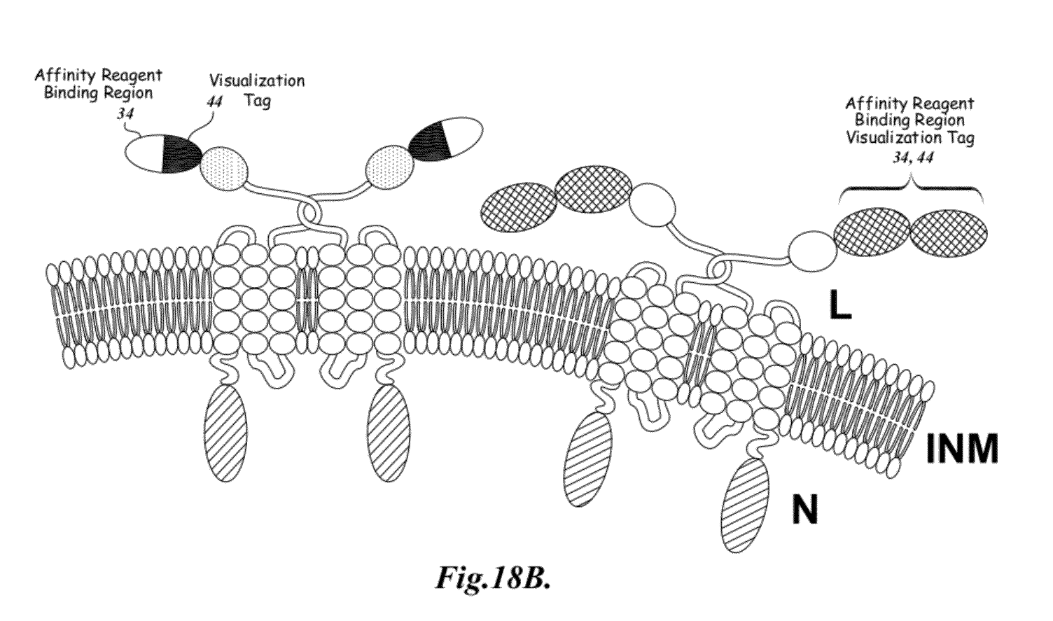

In other embodiments, the NTF protein comprises a nuclear envelope targeting region, such as a SUN domain, that causes the translocation of the NTF in the INM. In such embodiments, it is preferred that the affinity reagent binding region is positioned such that it resides in the lumen between the INM and ONM upon embedding of the NTF in the INM. For example, as described in EXAMPLE 6 and illustrated in FIGS. 18A and B, GFP or epitope tags were incorporated into the Sun-1 protein at a position C-terminal to the SUN domain, which resulted their positioning in the lumen.

In some embodiments, the encoded affinity binding region 34 comprises a biotin acceptor site 35 for a biotin ligase. The encoded biotin acceptor site 35 is capable of becoming biotinylated in vivo in the presence of a biotin holoenzyme synthetase. In vivo biotinylation is a highly specific post-translational modification mediated by endogenous biotin ligases (Cronan, J. E., et al., J. Biol. Chem. 265:10327-33, 1990). In one embodiment of the vector 10, the encoded biotin ligase acceptor site 35 is a target for the E. coli biotin carboxyl carrier protein (BCCP), a subunit of acetyl-CoA carboxylase (Samols, et al., J. Biol. Chem. 263:6461-4, 1988). Escherichia coli biotin holoenzyme synthetase (BirA) is encoded by the nucleic acid sequence set forth herein as SEQ ID NO:11 (Barker and Campbell, J. Mol. Biol. 146:451-67, 1981), and has the polypeptide sequence set forth herein as SEQ ID NO:12. The BirA enzyme is an exemplary enzyme that catalyzes biotin activation by covalently joining biotin with ATP to form biotin-5′-adenylate, with subsequent transfer to the epsilon amino group of a specific BCCP lysine residue (Barker and Campbell, J. Mol. Biol. 146:469-92, 1981b). Because in vivo biotinylation is highly specific for the BCCP lysine, it can be achieved without modification of critical lysine residues belonging to antibody recognition sequences and thus without functional loss of the recognition domains. Accordingly, in one embodiment, as described in EXAMPLES 1-3, the vector 10 comprises the nucleotide sequence set forth herein as SEQ ID NO:5, which encodes a biotin ligase accepting site 35, set forth in herein as SEQ ID NO:6. In other embodiments, the vector 10 comprises any nucleic acid sequence encoding a biotin ligase accepting site 35 with an amino acid sequence of SEQ ID NO:6, or variant thereof. In other embodiments, as described in EXAMPLES 4 and 5, the vector 10 comprises the nucleotide sequence set forth herein as SEQ ID NO:87, which encodes a biotin ligase accepting site 35, with an amino acid sequence set forth in herein as SEQ ID NO:88. In other embodiments, the vector 10 comprises a nucleic acid sequence encoding a biotin ligase accepting site 35 comprising an amino acid sequence of SEQ ID NO:88, or variant thereof.

It is noted that while BirA typically recognizes a large protein domain, Schatz and colleagues have identified short peptides (Schatz, P. J., Biotechnology 11:1138-43, 1993; Beckett, et al., Protein Sci. 8:921-9, 1999) that efficiently mimic BCCP biotin acceptor function. Accordingly, in some embodiments, the vector 10 comprises a nucleic acid sequence that encodes a shortened biotin ligase accepting site comprising the amino acid sequence GLNDIFEAQKIEWHE, set forth herein as SEQ ID NO:8. An example of a nucleic acid sequence encoding the shortened biotin ligase accepting site of SEQ ID NO:8 is set forth herein as SEQ ID NO:7. In some embodiments, the vector 10 comprises a nucleic acid sequence that encodes a shortened biotin ligase accepting site that is a variant of SEQ ID NO:8.

As described herein, the embodiments of the vectors, kits and methods incorporating an in vivo biotinylated fusion protein and biotin capture reagent allow for high yields of purified nuclei and purity of nucleic acid from cell-type specific cells. This likely due to the fact that the interaction between biotin and streptavidin is orders of magnitude stronger than typically observed for antigen/antibody interactions. Therefore, such embodiments allow for the isolation of a high percentage of the tagged nuclei and the selective purification of nucleic acids from the tagged nuclei.

In some embodiments, such as is illustrated in FIGS. 1A and B, the vector 10 further comprises a cell-type specific promoter 12 operatively linked to the nucleic acid encoding the NTF protein 30. As is well-known in the art, a promoter permits the binding of transcription factors and assembly of the transcription complex to facilitate the transcription of the sequence, thus permitting generation of the NTF protein. In preferred embodiments, the promoter 12 is located in the vector 10 near or adjacent to the 5′ end of the sequence encoding the NTF protein. Because of the vast variety of promoters in eukaryotic organisms, promoters can be selected that are specific to discrete cell-types within a tissue. Therefore, any known cell-specific promoter sequence from the eukaryotic organism of choice can be incorporated into the vector 10 to facilitate the transcription and subsequent translation of the NTF protein 30 sequence exclusively within the cell type of interest, and not within other cells within the same tissue or organism.

In some embodiments, the target organism is a plant. In one illustrative embodiment, as described in EXAMPLE 1, the vector 10 includes the promoter 12 for ACTIN DEPOLYMERIZING FACTOR 8 (ADF8) (Ruzicka et al., 2007), presented herein as SEQ ID NO:13, resulting in expression of the NTF protein 30 exclusively in hair cells of the A. thaliana root epidermis. Accordingly, in some embodiments wherein the cell type of interest is derived from a plant root epidermis hair cell, the vector comprises a promoter nucleic acid sequence that is a variant of SEQ ID NO:13 (ADF8) and has at least 90% identity thereto. In another embodiment, also described in EXAMPLE 1, the vector 10 included the promoter 12 for GLABRA 2 (GL2) (Masucci et al., 1996), presented herein as SEQ ID NO:14, resulting in expression of the NTF protein exclusively in the non-hair cells of the A. thaliana root epidermis. Accordingly, in some embodiments wherein the cell type of interest is derived from a plant root epidermis cell, the vector comprises a promoter sequence that is a variant of SEQ ID NO:14 (GL2) and has at least 90% identity thereto.

As will be apparent to persons of ordinary skill in the art, promoter sequences 12 for cell-type specific transcription of the NTF encoding nucleic acid can be selected from the organism of choice. For example, in embodiments in which the cell type of interest is a D. melanogaster cell type, known promoters specific for the D. melanogaster cell type may be used. For example, in the embodiment described in EXAMPLE 5 and illustrated in FIG. 15A, the D. melanogaster twist promoter sequence, set forth herein as SEQ ID NO:32, was used to drive the expression of the NTF protein-encoding vector 10 in somitic cells of the D. melanogaster. Accordingly, in some embodiments, the vector comprises a promoter sequence that is a variant of SEQ ID NO:32 (twist) and has at least 90% identity thereto.

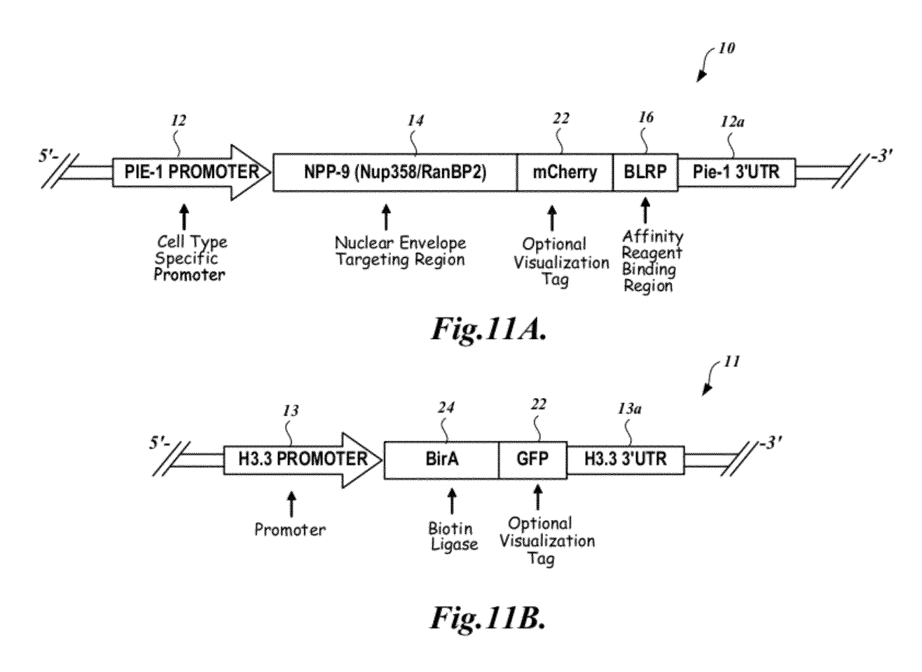

In some embodiments, the cell-type specific promoter 12 comprises the incorporation of 3′ UTR sequence to further facilitate the cell-type specific transcription of the vector sequence. For example, in the embodiment described in EXAMPLE 4, the promoter sequence 12 comprises the sequence for C. elegans pie-1 promoter, set forth herein as SEQ ID NO:20, and contains additional 3′ UTR sequence 12a C. elegans pie-1 3′ UTR, set forth herein as SEQ ID NO:21, were used for germline specific expression of the transgenic constructs. As illustrated in FIG. 11A, to accomplish germline specific expression, the pie-1 promoter 12 was disposed at the 5′ position on the vector 10 relative to the sequence encoding the NTF protein 14, 22, 16, whereas the pie-1 3′ UTR sequence 12a was disposed at the 3′ position on the vector 10 relative to the sequence 14, 22, 16 encoding the NTF protein. Accordingly, in some embodiments, the vector comprises a promoter sequence that is a variant of SEQ ID NO:20 (pie-1) and has at least 90% identity thereto.

As described above, some embodiments of the vector 10 encode an affinity reagent binding region comprising a biotin ligase accepting site 35. In further embodiments, the vector 10 encoding the NTF protein 30 in a first expression cassette also comprises a nucleic acid sequence 24 encoding a biotin ligase 38 in a second expression cassette 11, wherein the encoded biotin ligase 38 is capable of ligating biotin 36 to the biotin ligase accepting site 35 in the encoded NTF protein 30. As described above, biotin ligase 38 catalyzes biotin activation by covalently joining biotin 36 with ATP to form biotin-5′-adenylate, with subsequent transfer to the epsilon amino group of a specific lysine residues within a specific amino acid sequence recognized by the ligase 38. In some embodiment, biotin ligase 38 is E. coli biotin ligase BirA, the polypeptide sequence of which is set forth herein as SEQ ID NO:11, and is encoded by the nucleic acid sequence 24 set forth herein as SEQ ID NO:12. Accordingly, in some embodiments, the vector comprises a nucleotide sequence encoding an amino acid sequence of Accordingly, in some embodiments, the biotin ligase 38 is encoded by a nucleotide sequence 24 SEQ ID NO:11, or any variant thereof. In some embodiments, the expression cassette 11 encodes a variant biotin ligase 38 with an amino acid sequence with at least 90% identity to SEQ ID NO:12.

In preferred embodiments, the expression of at least one of the NTF polypeptide 30 or biotin ligase polypeptide 38 is controlled by a cell type-specific promoter 12. As described above, any known cell type-specific promoter sequence 12 can be incorporated into the vector(s) 10 to facilitate the transcription, and to enable the subsequent translation, of the sequence to which it is operatively linked in the cell type of interest. In some embodiments, only one of the sequences encoding the NTF protein sequence or the biotin ligase is operatively linked to a cell type-specific promoter 12, whereas the other is operatively linked to a constitutive promoter 13. In other embodiments, the sequences encoding both the fusion protein sequence (i.e., first expression cassette) and the biotin ligase (i.e., second expression cassette 11) are operatively linked to the same or different promoters 12 that is/are specific for the same cell type. In some embodiments, a single cell type specific promoter sequence 12 drives the expression of 1) an NTF protein comprising a nuclear targeting region, an affinity reagent binding region comprising a biotin ligase accepting site, and 2) a biotin ligase (i.e., in a unitary expression cassette). Consequently, in each of the embodiments described, only the cell type of interest will co-express both the NTF protein sequence and the biotin ligase to result in nuclei biotinylated in vivo.

In other embodiments, the sequence 24 encoding biotin ligase 38 is operatively linked to a distinct promoter sequence 13 (i.e. in a second expression cassette 11). In some embodiments, the sequence 24 encoding the biotin ligase 38 in the second expression cassette 11 is operatively linked to a cell type specific promoter sequence 12, which can be the same as, or different from, the promoter sequence 12 driving expression of the NTF protein 30. In other embodiments, expression of the biotin ligase 38 (i.e., second expression cassette) is under the control of a constitutive promoter 13 that is not cell-type specific and the expression of the NTF polypeptide 30 is under the control of a cell type-specific promoter 12. For example, in the embodiments illustrated in FIGS. 1A, B, and C, the sequences encoding the NTF protein are operatively linked to a cell type-specific promoter 12 (FIGS. 1A and B), whereas the sequence 24 encoding biotin ligase 38 (BirA) is under the control of a constitutive promoter 13 that can be universally expressed (cell type non-specific) (FIG. 1C). In this regard, the diagonal lines at the ends of the linear schematics represented in FIGS. 1A, B, and C are intended to illustrate that additional sequences may be included on the vector 10, including the other sequences represented by the schematics in FIGS. 1A, B, and C.

In some embodiments, the encoded NTF polypeptide 30 further comprises a visualization tag region 44, which is useful in permitting the visual confirmation of the NTF protein 30 being incorporated into the nuclear envelope 46 of the cells of interest that contain the vector 10. In some embodiments, the affinity reagent binding region 34 comprises the visualization tag, which thus serves a dual purpose of allowing for visualization and binding to an affinity binding reagent. As illustrated in FIG. 1F, in other embodiments, the vector encodes NTF protein 30 comprising a visualization tag region 44 in addition to a distinct affinity binding reagent region 34, such as a biotin ligase accepting site 35. An encoded visual tag 44 may comprise any sequence known in the art to facilitate visualization of expressed proteins. In some embodiments, the visualization tag region encodes an epitope tag in the NTF polypeptide 30. In another embodiment, as described in EXAMPLE 1, the visualization tag region 44 is the green fluorescent protein (GFP), the polypeptide sequence of which is set forth herein as SEQ ID NO:4, and is encoded by the sequence set forth herein as SEQ ID NO:3. Upon translation, the GFP polypeptide emits bright green light when exposed to blue light, enabling visualization of the fusion protein using fluorescence microscopy. The present invention also contemplates the incorporation into the NTF protein 30 of any of the numerous related GFP variants known in the art to similarly fluoresce upon stimulation, such as blue fluorescent protein, cyan fluorescent protein, and yellow fluorescent protein. In another embodiment, as described in EXAMPLES 4 and 5, the visualization tag 44 is mCherry, for example, as set forth herein as SEQ ID NO:17, and encoded by the nucleic acid sequence set forth herein as SEQ ID NO:16. Persons of ordinary skill in the art will recognize that the nucleotide domains for the nuclear envelope targeting region 14, the affinity reagent binding region 16, and the visualization tag 22 can be in any order relative to each other in the vector construct 10, so long as the order will result in the placement of the translated affinity reagent binding region 34 in the extra nuclear space (after the NTF protein 30 is attached to/embedded in the nuclear membrane 46), as described above.

In some embodiments, the encoded NTF polypeptide 30 further comprises one or more spacer regions 40 that separate the nuclear envelope targeting region 32 and the affinity reagent binding region 34 (e.g., epitope tag or biotin ligase accepting site 35) of the fusion protein. For example, in the embodiment of the vector 10 illustrated in FIG. 1B, a first optional spacer region 18 separates the nucleic acid sequences encoding the nuclear envelope targeting region 14 and the nucleic acid sequence encoding the optional visualization tag 22. Furthermore, a second optional spacer region 20 separates the nucleic acid sequence encoding the optional visualization tag 22 and the nucleic acid sequence encoding the affinity reagent binding region 16. FIG. 1F illustrates the translated NTF protein 30 of a similar embodiment after it has been incorporated into the nuclear envelope 46. Two optional spacer regions 40, 42 are also illustrated in FIG. 1F. Each encoded spacer region 40, 42 may be comprised of one or more contiguous amino acids. Without being bound by theory, in preferred embodiments, the spacer region(s) 40, 42 provide flexibility and additional length to the NTF protein 30 to facilitate the incorporation of the NTF protein to the nuclear envelope 46 and exposure of the biotin ligase accepting site 35 to potential biotinylation by a biotin ligase 38, such as BirA. In some embodiments, the one or more encoded spacer region(s) 40, 42 comprise from one to about 100 amino acids, such as from one to about 50 amino acids, such as from one to 10 amino acids, or from at least 10 to about 20 or more amino acids, suc has from 20 to about 30 amino acids, from 30 to about 40 amino acids, or from 40 to about 50 amino acids. In the exemplary embodiment described in EXAMPLE 1, the nuclear targeting region (WPP) is separated from the biotin ligase accepting site by two spacer regions, the first being three alanine residues situated between the WPP domain and the GFP, and the second being five alanine residues situated between the GFP and the biotin ligase accepting site. Another exemplary embodiment described in EXAMPLES 6 and 7, involves the construction of the NTF, wherein the GFP or tdTomato/epitope tag is inserted into a 42 amino acid residue linker sequence. This results in a 21 amino acid residue linker on both the N-Terminal and C-terminal sides of the visualization tag/affinity reagent binding region.

In some embodiments, the vector 10 encodes the NTF protein 30 and the biotin ligase 38. In this regard, the vector 10 may encode the NTF protein 30 and the biotin ligase 38 in the same (i.e. unitary) expression cassette driven by a cell type-specific promoter 12. Alternatively, the vector 10 may encode the NTF protein 30 and the biotin ligase 38 in the separate (i.e. first and second) expression cassettes, wherein at least one of the first and second expression cassettes 11 is driven by a cell type-specific promoter 12. In other embodiments, the vector 10 encodes the NTF protein 30, and a separate (i.e. second vector) encodes the biotin ligase 38 in a second expression cassette 11. As above, at least one of the first and second expression cassettes 11 is driven by a cell type-specific promoter 12.

One of ordinary skill in the art will recognize that in accordance with some embodiments of the invention, the vector(s) provided by the present invention for producing labeled nuclei (e.g., epitope tagged or in vivo biotinylated nuclei) may optionally include additional sequences known by those of skill in the art that facilitate the functionality of the vector in the cell type of interest. For example, vectors can include additional known sequences such as an origin of replication, selectable markers and sequences to facilitate transcription and translation of the fusion protein and biotin ligase. Such sequences also include polyadenylation tails, UTR sequences and Kozak sequences. See Sambrook, J., and Russell, D. W., eds., Molecular Cloning: A Laboratory Manual, 3rd ed., Cold Spring Harbor Laboratory Press, Cold Spring Harbor, N.Y. (2001). In some embodiments, the vector is a plasmid. In other embodiments, the vector is a viral vector. In a further embodiment, the viral vector is a Lentivirus vector.

It is demonstrated that the use of the present invention is widely applicable to eukaryotic organisms, including plants and animals, as described in EXAMPLES 1-5. Accordingly, in some embodiments, the vector(s) are useful for producing labeled (e.g., epitope tagged or biotinylated) nuclei in a cell type of interest that is derived from a eukaryotic organism. As used herein, the term “derived from” is used to indicate the originating organism that gave rise to the cell type of interest. In preferred embodiments, the cell type of interest is a specific type of differentiated cell that has developed within the originating organism at some temporal point in the organism's development, and is distinct from other cell types within the same organism. At the time of expression of the fusion protein (or co-expression with biotin ligase) encoded by the vector(s) of the present invention, the cell-type of interest may be incorporated into an intact tissue of the living originating organism, or maintained in an appropriate cell culture environment. Accordingly, in some embodiments, the vector comprising the NTF polypeptide is useful for producing labeled (e.g., epitope tagged or in vivo biotinylated) nuclei in a cell type of interest that is derived from a multicellular eukaryotic organism.

In some embodiments, the vector 10 encoding the NTF polypeptide 30 is useful for producing labeled (e.g., epitope tagged or in vivo biotinylated) nuclei in a cell type of interest that is derived from a plant, such as A. thaliana. In some embodiments, the vector 10 encoding the NTF polypeptide 30 is useful for producing labeled (e.g., epitope tagged or in vivo biotinylated) nuclei in a cell type of interest that is derived from an animal. In other embodiments, the vector 10 encoding the NTF polypeptide 30 is useful for producing labeled (e.g., epitope tagged or in vivo biotinylated) nuclei in a cell type of interest that is derived from an arthropod, such as D. melanogaster. In some embodiments, the vector 10 encoding the NTF polypeptide 30 is useful for producing labeled (e.g., epitope tagged or in vivo biotinylated) nuclei in a cell type of interest that is derived from a nematode, such as C. elegans. In some embodiments, the vector 10 encoding the NTF fusion 30 polypeptide is useful for producing labeled (e.g., in vivo biotinylated) nuclei in a cell type of interest that is derived from a mammal, such as rodents, dogs, cats, cats, horses, or primates including humans. In a further embodiment, the vector 10 encoding the NTF fusion 30 polypeptide is useful for producing labeled nuclei in a cell type of interest that is derived from a mouse.

In another aspect, the present invention provides a cell comprising a vector 10 for selectively labeling the cell type of interest, the vector 10 comprising a nucleic acid sequence encoding a nuclear tagging fusion (NTF) polypeptide 30 comprising (a) a nuclear envelope targeting region 32, and (b) an affinity reagent binding region 34, wherein the expressed NTF polypeptide 30 is incorporated into the nucleus 50 of the cell. Exemplary embodiments of the vector 10 have been described above. In some embodiments, the affinity reagent binding region 34 is an epitope tag, as described above. In some embodiments, the affinity reagent binding region 34 of the NTF polypeptide 30 comprises a biotin acceptor site 35 and the cell further comprises a vector 10 encoding a biotin ligase 38, such as BirA.

In one embodiment, the cell is part of a transgenic organism. In another embodiment, the cell is in a tissue. The tissue can be in a living organism or be maintained under appropriate culture conditions to permit the further development of the cell within the tissue. As used herein, the term “tissue” is used to describe an intermediate level of cellular organization between individual cells and a whole organism. The tissue is comprised of multiple cells, often of varying types, that may cooperate or function in concert to perform a united task. Accordingly, a cell contemplated in this embodiment is likely to be surrounded by different cell types with distinct developmental histories. In yet another embodiment, the invention provides a cell comprising the vector or vectors described above, wherein the cell is in culture. As used herein, the term “culture” is intended to mean any environment outside the organism of origin wherein conditions are maintained to facilitate the continuation of cell functions.

In another aspect, the invention provides a kit for selectively labeling nuclei 50 in a cell type of interest, the kit comprising: (a) a vector 10 comprising a first expression cassette comprising a nucleic acid sequence encoding a nuclear tagging fusion (NTF) polypeptide 30 comprising: (i) a nuclear envelope targeting region 32; and (ii) an affinity reagent binding region 34; and (b) a capture molecule (i.e., affinity reagent) capable of specifically binding to the affinity reagent binding region, or a modification thereof. In some embodiments, the affinity reagent binding region 34 comprises an epitope tag. In some embodiments, the affinity reagent binding 34 region comprises a biotin ligase accepting site 35. In some embodiments, the kit further comprises a second expression cassette 11 for expressing a biotin ligase polypeptide 38. In some embodiments the first and second expression cassettes are on the same vector 10. In other embodiments the first and second expression cassettes are on different vectors. In some embodiments, the capture reagent is bound to a magnetic particle. Various elements of the kit are described above in the context of the vector.

In some embodiments, the sequence encoding the NTF polypeptide is operatively linked to a cell type-specific promoter 12 for a cell type of interest. In embodiments comprising a second expression cassette 11 encoding a biotin ligase 38, the sequence encoding at least one of the NTF polypeptide or the biotin ligase polypeptide is operatively linked to a cell type-specific promoter 12. In some embodiments, the first, second, or both expression cassettes are adapted to receive a promoter 12 to be operationally linked to the sequence encoding the fusion protein and/or the sequence encoding the biotin ligase. For example, the expression cassette can include an insertion site flanked by one or more restriction enzyme recognition sequences for insertion of a promoter sequence, such as a particular cell-type specific promoter, using standard cloning techniques known by those of skill in the art.

In some embodiments, the first and second expression cassettes are provided on the same vector 10. In other embodiments, the first and second expression cassettes are provided in separate vectors.

The components of the kit may be adapted to function in cells of any eukaryotic organism of interest. Organisms of interest can include fungi, plants, and animals. Animals of interest include arthropods, nematodes, and mammals. One of ordinary skill in the art will recognize that functionality for any organism of interest requires selection of the appropriate nucleic acid sequence 14 encoding a nuclear envelope targeting domain 32, as described herein. In some embodiments, the first expression cassette encoding the NTF polypeptide is adapted to receive a nucleic sequence 14 encoding a nuclear envelope targeting domain 32 useful for translocation of the NTF polypeptide 30 to the nuclear envelope 46 of the organism of interest. As above, the expression cassette for the NTF polypeptide 30 can include an insertion site flanked by one or more restriction enzyme recognition sequences for insertion of a sequence encoding a nuclear envelope targeting region sequence using standard cloning techniques known by those of skill in the art.

In further embodiments, the kits of the invention further comprise an affinity reagent, i.e., capture molecule, that specifically binds to an epitope, such as one of any known epitope tags, in the affinity reagent binding region. Affinity reagents include antibodies or fragments thereof.

In some embodiments, the kit further comprises an affinity reagent, i.e., capture molecule, that specifically binds to biotin to facilitate the isolation of the in vivo biotinylated nuclei of a cell type of interest. The capture molecule can be any molecule known to specifically bind to biotin. Suitable examples include streptavidin, avidin, or antibodies specific for biotin, or functional fragments of any of the aforesaid molecules.

In some embodiments, the kit comprises a capture molecule that is bound to a magnetic particle to facilitate the isolation for the in vivo biotinylated nuclei of a cell type of interest.

In some embodiments of the kit, the affinity reagent binding region comprises at least one fluorescent protein domain. Such fluorescent protein domains are known in the art, and include GFP, dtTomato, and mCherry.

In some embodiments of the kit, the nuclear envelope targeting region comprises a SUN domain, a KASH domain, a WPP domain, an NPP-9 domain, a Nup358/RanBP2, or RanGAP domain, as described in EXAMPLES 1-7.

In some embodiments, the kit further comprises a device to facilitate isolation of the tagged (i.e., epitope tagged or in vivo biotinylated) nuclei of a cell type of interest. In one embodiment illustrated in FIG. 1G, the device comprises a tube or series of tubes permitting a controlled flow of cell lysates along the length. Along part of the length of a tube, a magnetic field is applied from a magnet, which restricts flow of tagged (i.e., epitope tagged or in vivo biotinylated) nuclei bound to magnetic particles while unbound nuclei and cellular debris from the cell lysate exist in the tube. Accordingly, in some embodiments, the kit further comprises a magnet, flow tubes and/or collection receptacles.

Each kit is preferably provided in suitable packaging and may also contain reagents useful for selectively isolating tagged (e.g., epitope tagged or in vivo biotinylated) nuclei in a cell type of interest, such as, for example, transfection reagents, selective media, control inserts, sequencing primers and PCR amplification primers, dNTPs, high fidelity polymerase and buffer, reagents for cell lysis, rinse buffers, reagents for DNA extraction, detection reagents, instructions, and the like. In some embodiments, the kit includes cells transformed with one or more vector(s) of the kit. Cells can include eukaryotic cells of various origins, for example, plant, arthropod, nematode, or mammalian cells.

In another aspect, the present invention provides a method of generating in vivo biotinylated nuclei in a cell type of interest comprising recombinantly co-expressing in the cell (a) a nuclear tagging fusion (NTF) polypeptide 30 comprising (i) a nuclear envelope targeting region 32; and (ii) a biotin ligase accepting site 35; and (b) a biotin ligase 38; wherein the co-expression of the recombinant NTF polypeptide 30 and the biotin ligase 38 produces biotinylated nuclei in the cell type of interest. The methods of this embodiment of the invention may be carried out using the vectors and kits described herein.

In accordance with the foregoing, the co-expression of the recombinant NTF polypeptide 30 and the biotin ligase 38 produces biotinylated nuclei in the cell. Without intending to be bound by theory, the nucleic acid sequences encoding both the NTF polypeptide and the biotin ligase are transcribed into mRNA by virtue of the assembly of transcription factors and transcription complex proteins, including RNA Polymerase as facilitated by the operatively linked promoters. The mRNA is used as a translation template by the cells' endogenous ribosomes. The NTF polypeptide 30, by virtue of the nuclear envelope targeting region 32, is transported and incorporated into the nuclear envelope 46. See, for example, the embodiment illustrated in FIG. 1E, wherein the nuclear targeting region 32 is embedded in the nuclear envelope 46 of the nucleus 50. Meanwhile, the biotin ligase accepting site 35 is recognized by the biotin ligase 38 that is co-expressed in the same cell of interest. As previously described, biotin 36 is ligated to the target lysine residue of the biotin ligase accepting site 35 of the affinity reagent binding region (indicated by a dashed arrow), which is situated in the extra-nuclear space of the cell. Thus, the cell nucleus 50 is biotinylated in vivo by virtue of having incorporated into its nuclear envelope 46 at least one polypeptide covalently ligated to a biotin molecule 36. The term “in vivo” is used to convey that the biotinylation occurs in the living cell. In some embodiments, the cell recombinantly co-expressing the NTF polypeptide 30 and biotin ligase 38 is in a transgenic organism. In other embodiments, the cell recombinantly co-expressing the NTF polypeptide 30 and biotin ligase 38 is maintained in culture.

As described herein, the vector 10 encoding the NTF polypeptide 30 can optionally encode additional domains, such as one or more spacer regions 40, 42 and/or one or more visualization tags 44. FIG. 1F illustrates an embodiment wherein the NTF polypeptide 30 further comprises a first optional spacer region 40 and a second optional spacer region 42 on either side of a visualization tag domain 44. As described above, the spacer regions 40, 42 can be useful to provide flexibility and additional length to the NTF polypeptide 30 to facilitate the incorporation of the NTF protein 30 to the nuclear envelope 46 and to enhance exposure of the affinity reagent binding region 34 to the affinity reagent (i.e., capture reagent). The visualization tag 44 can be useful, for example, to visualize the localization of the NTF protein 30 to the nuclear envelope 46, and/or assess the purity nuclei isolated according to the methods described herein. In the illustrative embodiment illustrated in FIG. 1F, the affinity reagent binding region 34 contains a biotin ligase accepting site 35, which is recognized by biotin ligase 38, which ligates a biotin molecule 36 thereto.

Conventional cloning techniques may be used to insert a sequence encoding a known nuclear envelope targeting region in frame with a sequence encoding the affinity reagent binding region, such as a biotin ligase accepting site, within an expression vector, to obtain a sequence encoding a fusion protein. See, e.g., Sambrook et al., Molecular Cloning: A Laboratory Manual, 3d Ed., Cold Spring Harbor Press, Plainsview, N.Y. (2000). Examples of sequences encoding nuclear envelope targeting regions, affinity reagent binding regions comprising epitope tags, and biotin ligase accepting sites are provided herein. Similarly, conventional cloning techniques may be used to insert sequences encoding a biotin ligase into an expression vector, as described herein.

The nucleotide sequences encoding the NTF polypeptide 30 and the biotin ligase 38 are each operatively linked to promoter sequence(s) within the vector(s) that facilitates the binding of transcription factors and assembly of the transcription complex to generate mRNA transcripts of the sequences and subsequently generate the corresponding polypeptide gene products. In a preferred embodiment, expression of at least one of the NTF polypeptide and the biotin ligase is under the control of a promoter 12 specific to the cell type of interest. In a further embodiment, expression of both of the NTF polypeptide and the biotin ligase are under the control of a promoter 12 specific to the cell type of interest, which may be the same or different promoter 12. Use of a promoter 12 specific to a cell type of interest in this manner ensures that the co-expression of the NTF protein and biotin ligase will be exclusive to the cell type of interest, and not in neighboring cells with distinct developmental histories.

In one embodiment, the nucleotide sequences encoding the NTF polypeptide 30 comprising a nuclear envelope targeting region 32 and an affinity reagent binding region 34 are introduced into the cell, or a progenitor of the cell type of interest. In one embodiment, the nucleotide sequences encoding the NTF polypeptide 30 and the biotin ligase 38 are present in the same expression vector 10, wherein the co-expressing comprises introducing one or more copies of the vector encoding the NTF polypeptide 30 and biotin ligase 38 into the cell, or a progenitor of the cell type of interest. In another embodiment, the nucleotide sequences encoding the NTF polypeptide 38 and the biotin ligase 38 are present on separate expression vectors, wherein the co-expressing comprises introducing one or more copies of the vector 10 encoding the NTF polypeptide 30 and one or more copies of the vector encoding biotin ligase 38 in a second expression cassette 11 into the cell, or a progenitor of the cell type of interest.

The term “introduce” is used herein to describe any act of causing the vector 10 to be present in a cell at any time in the course of the cell's development. In some embodiments of the method, at least one copy of the expression vector or vectors is introduced into an existing cell of the type of interest by direct use of conventional transformation techniques (e.g., DNA transfection). In another embodiment, at least one copy of the expression vector or vectors is introduced into a cell type of interest by transforming the vector or vectors into a progenitor cell of the cell type of interest. Consequently, by virtue of DNA replication and cell division, the progenitor cells give rise to a plurality of cells of the cell type of interest, each cell of which contains at least one copy of the vector or vectors (i.e., genetically modified cells). The progeny cells of the progenitor cells may comprise a multitude of cell-types with distinct developmental histories in addition to the cell type of interest. In one embodiment, the progenitor cell is a stem cell. In another embodiment, the progenitor cell is an embryo.

Alternatively, in some embodiments, the vector or vectors introduced into a progenitor cell is not duplicated in its entirety during the course of cell replication. In contrast, the elements of the vector or vectors, including the sequences encoding the NTF polypeptide 30 and biotin ligase 38 and their operatively linked promoters, are transferred into the genome of the cell. In accordance with the foregoing, in a further embodiment, at least one copy of the expression vector or vectors is introduced into a cell type of interest by transforming the vector or vectors into a progenitor cell of the cell type of interest, and by virtue of DNA replication and cell division, the progenitor cell gives rise to a plurality of cells of the cell type of interest, each cell of which contains at least one copy of the sequences encoding the NTF polypeptide 30 and biotin ligase 38 and their operatively linked promoters.

In another embodiment, the NTF protein 38 further comprises a visualization tag 44. As described, this is useful to perform visual confirmation of the proper expression and nuclear envelope-localization of the fusion protein. Visual confirmation may be performed using standard microscopy techniques. The visualization tag 44 may be one of many conventional and well-known polypeptide sequences known to emit light or other detectable energy, as described above.

In another embodiment, the cell type of interest is present in a mixture of multiple cell types. The different cell types in the mixture are understood to have distinct developmental histories, although they may be the progeny of a common progenitor cell. As a consequence of the distinct developmental histories, the different cell types exhibit different phenotypes and possess unique repertoires of gene transcription factors. In one embodiment, all of the cell types are the progeny of a common progenitor cell that received the vector or vectors encoding the NTF polypeptide 30 and biotin ligase 38. In another embodiment, the mixture of cell types may be in a cell culture, as distinct from the organism of origin. In another embodiment, the mixture is a tissue, which is an organized conglomeration of cells of different types that cooperate to perform a function in the organism of origin.

In some embodiments, the cell type of interest is of plant, nematode, arthropod, or mammalian origin. For example, in the embodiments described in EXAMPLE 1, the cell types of interest were hair cell and non-hair cells in the root epidermis of the plant A. thaliana. In the embodiment described in EXAMPLE 4, the cell type of interest was germline cells in C. elegans. In the embodiment described in EXAMPLE 5, the cell type of interest was somitic cells in D. melanogaster embryos.

In another embodiment, as described herein, the method further comprises isolating labeled (e.g., biotinylated) nuclei from the cells using a capture molecule that specifically binds to the affinity reagent binding region, or a modified (e.g., biotinylated) form thereof.

In another aspect, the invention provides a method for selectively isolating nuclei from a cell type of interest present in a plurality of cells. The method according to this aspect comprises (a) recombinantly expressing in a plurality of cells of a cell type of interest a nuclear tagging fusion (NTF) polypeptide 30 comprising (i) a nuclear envelope targeting region 32 and (ii) an affinity reagent binding region 32, wherein the NTF polypeptide is under the control of a promoter specific to the cell type of interest 12; (b) lysing the plurality of cells of step (a) under conditions suitable to generate a cell lysate comprising a plurality of intact nuclei; (c) contacting the cell lysate with a capture molecule that specifically binds to the affinity reagent binding region, or a modification thereof, under conditions suitable to bind the nuclei comprising the fusion polypeptide; and (d) isolating the nuclei bound to the capture molecule. The methods of this embodiment of the invention may be carried out using the vectors and kits described herein.

In another aspect, the invention provides a method for selectively isolating nuclei from a cell type of interest present in a plurality of cells, wherein at least a portion of the cells recombinantly express a fusion polypeptide, the fusion polypeptide comprising (i) a nuclear envelope targeting region 32 and (ii) an affinity reagent binding region 32, wherein the NTF polypeptide is under the control of a promoter specific to the cell type of interest 12. The method according to the aspect comprises (a) lysing the plurality of cells under conditions suitable to generate a cell lysate comprising a plurality of intact nuclei; (b) contacting the cell lysate with a capture molecule that specifically binds to the affinity reagent binding region, or a modification thereof, under conditions suitable to bind the nuclei comprising the fusion polypeptide; and (c) isolating the nuclei bound to the capture molecule. The methods of this embodiment of the invention may be carried out using the vectors and kits described herein.

In some embodiments of the method, the nuclei of the cell type of interest are isolated from a mixture of multiple cell types obtained from at least one of a plant, a nematode, an arthropod, or a mammal. In some embodiments, the nuclei are isolated from a mixture or plurality of cells obtained from a mammal, such as a mouse.

In some embodiments, the nuclear envelope targeting regions comprises a SUN domain, a KASH domain, a WPP domain, an NPP-9 domain, a Nup358/RanBP2, or RanGAP domain, as described in EXAMPLES 1-7.

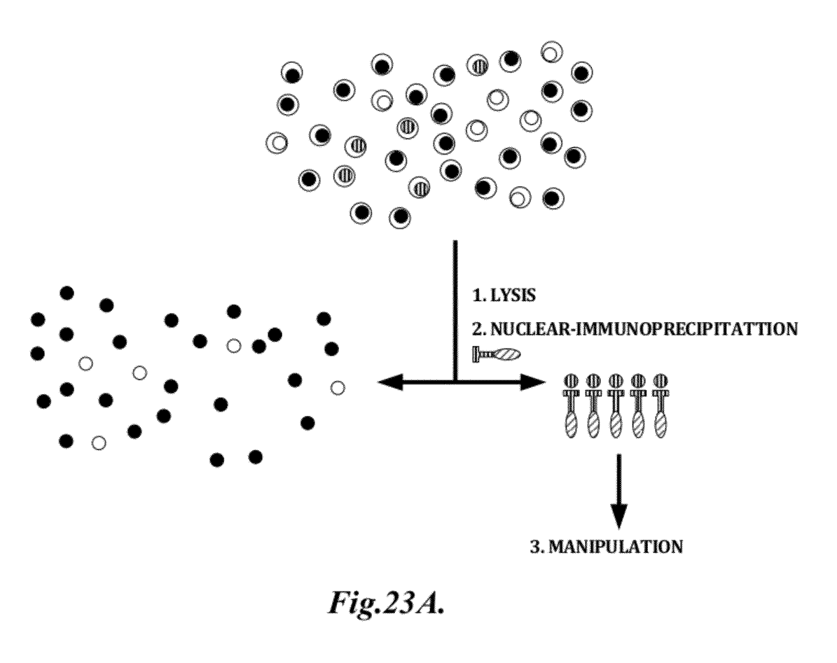

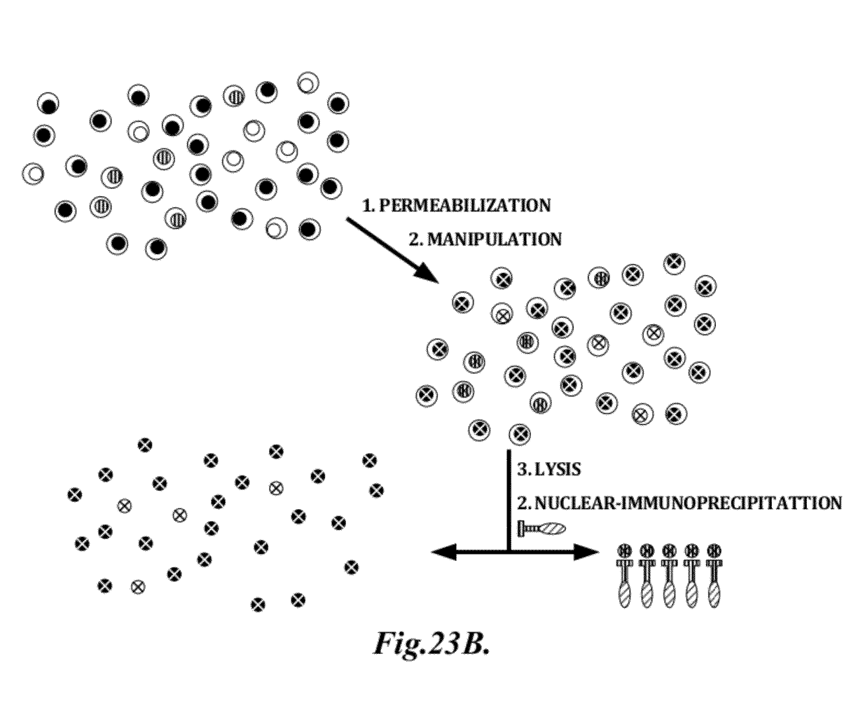

In some embodiments, the method further comprises permeabilizing the cells and subjecting the nucleic acids therein to biochemical manipulation before the cell lysis step, as illustrated in FIG. 23B. Relevant biochemical manipulations include digestion by nucleases. As explained in EXAMPLE 6, this approach is advantageous for time sensitive techniques such as DNaseI hyper-sensitivity mapping

In some embodiments, the method comprises introducing a viral vector, encoding the nuclear tagging fusion protein into the host organisms to induce recombinant expression of the fusion protein. Any viral vector suitable to induce expression within a host cell is contemplated. One example is a Lentivirus vector. The host organism can be any eukaryotic organism for which nuclear envelope targeting regions are known (and incorporated into the NTF protein). Illustrative eukaryotic organisms include plants, nematodes, arthropods, and mammals. More specifically, illustrative model organisms include A. thaliana, C. elegans, D. melanogaster, and Mus musculus.

In some embodiments, the viral vector is introduced into a progenitor cell of the cell type of interest. In other embodiments, the viral vector is introduced into a post-mitotic cell. As used herein, the term “post-mitotic” is used to refer to the cell cycle state where the cell will no longer undergo further division. In some embodiments, the post mitotic cell is a neuron. An exemplary description of this embodiment is described in EXAMPLE 6.

In another aspect, the present invention provides a method of selectively isolating nuclei from a cell type of interest wherein at least a portion of the cells co-express (i) a recombinant nuclear tagging fusion (NTF) polypeptide 30 comprising a nuclear envelope targeting region 32 and a biotin ligase accepting site 35, and (ii) a biotin ligase 38, wherein expression of at least one of the recombinant NTF polypeptide 30 or the biotin ligase 38 is under the control of a promoter 12 that is specific for the cell type of interest, and wherein the co-expression of the recombinant NTF polypeptide 30 and the biotin ligase 38 produces biotinylated nuclei only in the cell type of interest. The method comprises: (a) lysing the plurality of cells from the mixture under conditions suitable to generate a cell lysate comprising a plurality of intact nuclei from the plurality of cells; (b) contacting the cell lysate with a capture molecule that specifically binds to biotin under conditions suitable to bind the biotinylated nuclei; and (c) isolating the biotinylated nuclei bound to the capture molecule.

Cells may be lysed by any conventional method sufficient to interrupt the continuity of the cell's outer plasma membrane (illustrated in FIGS. 1D, E, and F, as 48) but that does not interrupt the integrity of the nuclear envelope and any fusion polypeptides, such as biotinylated fusion polypeptides, incorporated therein. The outer cell plasma membrane 48 must be sufficiently interrupted and degraded so as to allow access to the intact nuclear membrane and proteins incorporated therein. The cells may be lysed by any one or a combination of well-known conventional techniques. In some embodiments, the cells are lysed mechanically. For instance, the cells may be repeatedly forced through a narrow space, such as with a homogenizer. Alternatively, the cells may be ground by rotating blades, or subjected to compression between a mortar and pestle. In yet other embodiments, cells may be subjected to freeze-thaw cycles. In further embodiments, cells are first frozen before they are subjected to any of the techniques described. In yet other embodiments, the cells are lysed chemically, such as with suitable hydrolytic enzymes. For instance, the cells may be suspended in hypotonic buffer. Often, it is preferable to also treat cells with protease inhibitors to maintain the integrity of proteins embedded in the nuclear membrane. In the embodiment described in EXAMPLE 1, roots from A. thaliana were harvested from the plant, frozen in liquid nitrogen, ground to a fine powder, and resuspended in a nuclei purification buffer (NPB) (pH=7) containing: 20 mM MOPS, 40 mM NaCl, 90 mM KCl, 2 mM EDTA, 0.5 mM EGTA, 0.5 mM spermidine, 0.2 mM spermine, and Roche Complete protease inhibitors per manufacturer's instructions.

It is preferred that nuclei are rinsed to rid the solution of cellular debris from the lysate. The nuclei can be rinsed in NPB, pelleted by centrifugation, and resuspended in NPB multiple times. In preferred embodiments, the nuclei are finally resuspended in a low volume to enhance the interaction between the nuclei and capture molecules, such as streptavidin-containing molecules that specifically bind to biotin. For example, as described in EXAMPLE 1, nuclei from an initial 3 grams of plant root tissue were finally resuspended in 1 mL of NPB after introduction.

In accordance with an embodiment of the method provided by the present invention, a capture molecule is contacted with the cell lysate under conditions suitable for binding to the biotinylated nuclei. As described herein, the capture molecule can be any molecule that specifically binds to biotin that is attached to the fusion protein. In some embodiments, the capture molecule is streptavidin, or a fragment thereof. In some embodiments, the capture molecule is avidin, or a fragment thereof. In some embodiments, the capture molecule is an anti-biotin antibody, or a fragment thereof.

In some embodiments, the capture molecule is immobilized on a solid substrate, such as a tissue culture plate or filter and the cell lysate is passed over the immobilized capture molecule. Interactions between the biotinylated nuclei and immobilized capture molecules effectively immobilize the biotinylated nuclei and allow the non-biotinylated nuclei to be rinsed away. After isolation, the nuclei may be collected, for example, by interrupting the interaction between the biotinylated nuclei and the capture molecule, and collecting the supernatant.

In other embodiments, the capture molecule is not immobilized on a solid substrate. In a preferred embodiment, the capture molecule is bound to a magnetic particle. For instance, as described in EXAMPLE 1, streptavidin-coated Dynabeads®(Invitrogen M-280) were contacted to the cell lysate at ˜1.5×10⁷ beads/mL of resuspended nuclei. The mixture was agitated by rotation at 4° C. for 30 minutes to maximize the binding of the streptavidin-coated beads to the biotinylated nuclei. In one embodiment, the biotinylated nuclei are subsequently isolated from the mixture by passing the mixture through a magnetic field at least one time. It is preferred that the mixture be diluted by about ten-fold to lower the concentration. In the embodiment described in EXAMPLE 1, the suspension was passed through a pipette placed in the groove of a MiniMACS™ separator magnet (Miltenyi Biotec, catalog #130-042-102). The suspension was allowed to pass through the pipette at approximately 0.75 mL per minute. The magnetic field captured the bead-bound biotinylated nuclei while allowing the non-biotinylated nuclei and other debris to pass. In some embodiments, the process is repeated by resuspending the isolated nuclei. The pipette is removed from the magnetic field and the nuclei are resuspended by repeatedly drawing NPB or other suitable buffer repeatedly in and out of the pipette. The process can be thus repeated as described.

In some embodiments, the method provided by the present invention further comprises extracting the nucleic acids from the isolated (e.g., tagged or biotinylated) nuclei. Conventional techniques and reagents, including many commercially available kits, are available for extracting of DNA and RNA. Isolated nucleic acids are useful for subsequent genomic analyses of the cell type of interest, including analyses of gene expression and chromatin regulation. Illustrative analyses are described in EXAMPLES 2 and 3.

In another aspect, the invention provides a method of visually tagging nuclei in a cell type of interest. The method comprises introducing a vector comprising a nucleic acid sequence encoding a fusion polypeptide into a cell-type of interest. The polypeptide comprises (a) a nuclear envelope targeting region and (b) a fluorescent protein. The methods of this aspect of the invention may be carried out using the vectors and kits described herein

In some embodiments, the vector is a plasmid. In some embodiments, the vector is a viral vector. In a further embodiment, the viral vector is a Lentivirus.

In some embodiments, the cell type of interest is eukaryotic. Eukaryotic cells include fungal, plant, and animal cells. Animal cells include the non-limiting categories: poriforan, cniderian, platyhelminth, nematode, annelid, mollusk, arthropod, echinoderm and vertebrate cells. In some embodiments, the vertebrate cells are mammalian, such as mouse cells. Additional specific examples of animal groups are described above.

The cell type of interest may be in culture or in vivo within a host organism.

In some embodiments of this aspect, the nuclear envelope targeting region selectively targets the outer nuclear membrane (ONM). For example, in embodiments described in EXAMPLES 6 and 7, a KASH domain was incorporated into an NTF protein. Upon expression, the NTF protein localized to the ONM providing a tag on nuclei that enabled their visualization and isolation.

In some embodiments of this aspect, the nuclear envelope targeting region selectively targets the inner nuclear membrane (INM). For example, in embodiments described in EXAMPLES 6 and 7, a SUN domain was incorporated into an NTF protein. Upon expression, the NTF protein localized to the ONM providing a tag on nuclei that enabled their visualization and isolation. Specific localization of the NTF proteins incorporating the SUN (and KASH) domain to the INM (or ONM) are described in more detail in EXAMPLE 6.

In accordance with this aspect, the cell type of interest can be any cell type for which a functional promoter and nuclear envelope targeting region is known. Thus, the cell type of interest can be from any lineage. For example, in some embodiments, the cell type of interest is a nerve cell.

In some embodiments, the method further comprises isolating the tagged nuclei. For example, the expressed fusion protein can comprise an affinity reagent binding region as described above. The tagged nuclei can be isolated or purified utilizing any of the methods, kits or reagents described above. In some embodiments, the tagged nuclei are isolated under conditions that preserve both the INM and ONM. This can be accomplished, for example, through the use of very mild detergents, or with reagents that omit or lack detergents, as described in EXAMPLE 6. In some embodiments, the nuclei are isolated under conditions that preserve only the INM, as described in EXAMPLE 6.

In some embodiments, the cells are permeabilized prior to isolation of the tagged nuclei. The permeabilization is useful to introduce reagents into the cells that can biochemically manipulate the genomic DNA or chromatin before the cells are lysed and the nuclei are isolated, as described in EXAMPLE 6.

The compounds, kits, and methods of the present method as described herein are useful for isolating the nuclei from a cell type of interest. In some embodiments, the cells of the cell type of interest exist in a mixture of multiple cell types that exhibit distinct phenotypes and developmental histories. Consequently, the present invention provides a cost effective and robust alternative to present methods for analyzing genome expression and chromatin regulation in a specific cell type. The method reduces the need for expensive and highly technical equipment, avoids undue manipulation of the biological sample, and results in a highly pure sample of genomic material from a cell type of interest, making the study of cell differentiation and function more accessible.

The following examples merely illustrate the best mode now contemplated for practicing the invention, but should not be construed to limit the invention. All literature citations are expressly incorporated by reference.

### Example 1

This Example describes the development of a method and reagents for isolation of nuclei tagged in specific cell types (INTACT) in the model system Arabidopsis thaliana.

Rationale:

As a proof-of-concept, in this Example, the INTACT system was employed to study the two cell types of the Arabidopsis root epidermis: hair (H) cells and non-hair (NH) cells. These two cell types originate from a common progenitor and make up the entire epidermal layer of the root, arising in alternating vertical cell files along the axis of this organ. The hair cells form long tubular outgrowths that are involved in water and nutrient uptake, anchorage, and interaction with soil microbes, while the non-hair cells do not produce such outgrowths (see Grierson, C., and J. Schiefelbein, “Root hairs,” in The Arabidopsis Book, C. R. Somerville and E. M. Meyerowitz, eds. (Rockville, Md.: American Society of Plant Biologists), 2002). The formation of these cell types has been extensively studied at the genetic and cell biological levels (Ishida, T., et al., “A Genetic Regulatory Network in the Development of Trichomes and Root Hairs,” Annu. Rev. Plant Biol. 59:365-386, 2008), and many genes that are expressed preferentially in each cell type have been identified (Birnbaum et al., Science 2003; Brady, S. M., et al., “A High-Resolution Root Spatiotemporal Map Reveals Dominant Expression Patterns,” Science 318:801-806, 2007; Won, S.-K., et al., “cis-Element- and Transcriptome-Based Screening of Root Hair-Specific Genes and Their Functional Characterization in Arabidopsis,” Plant Physiology 150:1459-1473, 2009), providing a point of comparison for the gene expression studies using the INTACT method, as described in EXAMPLE 2.

Methods:

Constructs and Transgenic Plants for INTACT

The vector used for INTACT, illustrated schematically in FIG. 1B, encoded a nuclear tagging fusion (NTF) protein comprising a nuclear envelope targeting sequence 14, a luminescent visualization tag 22, and a recognition affinity reagent binding region 16. In the embodiment of the INTACT vector used in this example, the encoded nuclear envelope targeting protein used for INTACT consisted of a fusion of the WPP domain of Arabidopsis RanGAP1 (At3g63130; amino acids 1-111, set forth herein as SEQ ID NO:2, inclusive, encoded by the sequence set forth herein as SEQ ID NO:1) (Rose and Meier, 2001) at the N-terminus of the NTF polypeptide. The encoded WPP domain was followed by the enhanced green fluorescent protein (eGFP) (Zhang, G., et al., “An Enhanced Green Fluorescent Protein Allows Sensitive Detection of Gene Transfer in Mammalian Cells,” Biochem. Biophys. Res. Commun. 227:707-711, 1996) with the polypeptide sequence set forth herein as SEQ ID NO:4, encoded by the nucleic acid sequence set forth herein as SEQ ID NO:3. The eGFP domain was followed by the biotin ligase recognition peptide (BLRP), a biotin ligase accepting site with the amino acid sequence set forth herein as SEQ ID NO:6 (Beckett et al., 1999) at the C-terminus of the encoded NTF polypeptide. The BLRP was encoded by the nucleic acid sequence set forth herein as SEQ ID NO:5. In the embodiment shown in FIG. 1B, the nuclear envelope targeting region 14 (here, WPP domain of RanGAP1) was separated from the visualization tag, GFP, 22 by an optional first spacer region 18 comprising 3 alanine residues, and the visualization tag, GFP, 22 was separated from the affinity reagent binding region, BLRP, 16 by a second optional spacer region 20 comprising 5 alanine residues. The combination of the described domains provided an NTF protein comprising the amino acid sequence set forth as SEQ ID NO:10. The sequence for the gene construct 10 encoding the NTF protein, set forth herein as SEQ ID NO:9, was cloned under control of a cell type specific promoter 12, i.e., the ADF8 (At4g00680) promoter, the sequence of which is set forth herein as SEQ ID NO:13, as described in Ruzicka et al., Plant J. 52:460-472, 2007, incorporated herein by reference, for hair cell expression. Furthermore, the gene construct 10 encoding the NTF protein was cloned under control of the GL2 (At1g79840) promoter 12, the sequence of which is set forth herein as SEQ ID NO:14, as described in Masucci et al., Development 122:1253-1260 (1996), incorporated herein by reference, for non-hair cell expression.

Each of these constructs encoding the NTF protein were co-transformed into Arabidopsis ecotype Col-0 along with a vector comprising a second expression cassette 11 encoding the E. coli biotin ligase 38 (BirA) (the polypeptide of which is set forth herein as SEQ ID NO:12, and is encoded by the nucleic acid sequence set forth herein as SEQ ID NO:11). Expression of the BirA gene was driven from the constitutive ACT2 (At3g18780) promoter (the nucleic acid sequence of which is set forth herein as SEQ ID NO: 15), as described in An, Y. Q., et al., “Strong, Constitutive Expression of the Arabidopsis Act2/Act8 Actin Subclass in Vegetative Tissues,” Plant J. 10:107-121, 1996, incorporated herein by reference. See also Zilberman, D., et al., “Histone H2A.Z and DNA Methylation Are Mutually Antagonistic Chromatin Marks,” Nature 456:125-129, 2008 (see FIG. 1C). However, it is noted that in some embodiments, a cell type specific promoter can be used to drive expression of BirA.

First-generation double transgenic plants were selfed to produce plants that were homozygous for both the NTF and BirA transgenes. Multiple individual NTF/BirA double transgenic lines showing the expected expression patterns were combined and used in all subsequent experiments.

Plant Growth and Harvesting of Root Tissue

Plants were grown under fluorescent light for 16 hours per day at 22° C. on agar-solidified ½ strength MS media; Murashige, T., and F. Skoog, “A Revised Medium for Rapid Growth and Bioassays With Tobacco Tissue Culture,” Plant Physiol. 15:473-497, 1962. Plates were kept in a nearly vertical orientation such that the roots grew along the surface of the media. When the plants reached 7 days of age, a 1.25 cm section of the roots, from within the fully differentiated root hair zone but below the position of the first lateral roots, was harvested with a razor blade. This region of root tissue was used in all experiments.

Purification of Biotinylated Nuclei

For each purification, 3 g of root tissue was frozen in liquid nitrogen, ground to a fine powder and resuspended in 10 mL of nuclei purification buffer (NPB) containing: 20 mM MOPS, 40 mM NaCl, 90 mM KCl, 2 mM EDTA, 0.5 mM EGTA, 0.5 mM spermidine, 0.2 mM spermine, pH=7) containing Roche Complete® protease inhibitors. Nuclear suspensions were then filtered through 70 μM nylon mesh and pelleted at 1000×g for 5 minutes at 4° C. Nuclei were washed with 1 mL of NPB, pelleted again, and finally resuspended in 1 mL of NPB. Twenty-five microliters of Invitrogen M-280 streptavidin-coated Dynabeads® (˜1.5×10⁷ beads) were added to the nuclear suspensions and this mixture was rotated at 4° C. for 30 minutes to allow binding of beads to the biotinylated nuclei.

The 1 mL suspension of beads and nuclei was diluted to 10 mL volume with NPB containing 0.1% Triton X-100 (NPBt) and drawn into a plastic 10 mL serological pipette. A MiniMACS™ separator magnet (Miltenyi Biotec, catalog #130-042-102) was then used to capture the Dynabeads®-bound nuclei using a flow-based setup, as shown in FIG. 1G. This was accomplished by inserting a 1 mL micropipette tip into the groove running the length of the magnet and then inserting the narrow end of the serological pipette, containing the nuclei and bead suspension, into the wide end of the 1 mL pipette tip and allowing the suspension to flow past the magnet at a rate of 0.75 mL/min. As the suspension flowed past the magnet, beads and nuclei were captured on the wall of the 1 mL pipette tip, and all of the solution was allowed to drain out. Beads and nuclei were then eluted from the wall of the tip by placing it on a pipette and repeatedly drawing 1 mL of NPBt into and out of the tip. This suspension was again brought up to a final volume of 10 mL with NPBt, and the magnetic purification was repeated just as before. Beads and nuclei were again released into 1 mL of NPBt, then collected by centrifugation, decanted and used immediately or resuspended in 20 μL NPB and frozen at −20° prior to use. The 1 mL pipette tips used in the purification were pre-treated with NPB+1% BSA for 10 minutes to prevent the beads from sticking too firmly to the wall of the tip.

Typically, 3 g of tissue yielded 1−3×10⁵ nuclei. This amount was used for each RNA isolation or chromatin immunoprecipitation experiment, as described below. Purity and yield of nuclei after purification were determined by staining of total nuclei with DAPI prior to purification and subsequent counting of the number of bead-bound nuclei and unbound nuclei in the purified preparation, considering bead-bound nuclei to be the target nuclei and non bead-bound nuclei as contaminating nuclei from other cell types.

Analysis of Nuclear Tagging Fusion Protein Retention on the Nuclear Surface

Total nuclei were isolated from GL2p:NTF/ACT2p:BirA transgenic roots and were washed twice with nuclei purification buffer (NPB) to test for dissociation of NTF from the non-hair cell nuclei by streptavidin western blotting. Nuclei were initially extracted in 1 mL of NPB and pelleted by centrifugation. Total protein from 10% percent of this supernatant fraction and 10% of the pelleted nuclei was loaded on a 12% polyacrylamide gel (input). Nuclei were then resuspended in 1 mL NPB with mixing for 5 min, pelleted again, and the wash was repeated. Total protein from 10% of the nuclei from each wash (washed nuclei), and 100% of total protein from each wash supernatant (wash supernatant; prepared by trichloroacetic acid precipitation of protein from the entire supernatant) were loaded on the same gel. Streptavidin western blotting was performed as described below.

For imaging analysis, total nuclei were extracted from the roots of each indicated line and were mixed for 30 minutes with streptavidin-coated Dynabeads®. The same number of total nuclei were used in each case. Nuclei-bead mixtures were then mounted on glass slides and viewed at 20× magnification under a light microscope.

Immunoprecipitation and Western Blotting

Whole cell extracts were prepared from transgenic roots by grinding in liquid N₂ and resuspension in 2 volumes of RIPA buffer (50 mM Tris, 150 mM NaCl, 1% NP-40, 0.5% sodium deoxycholate, 0.1% sodium dodecyl sulfate, pH=7.5) containing Roche Complete® protease inhibitors. This extract was cleared by centrifugation to give the input fraction. An aliquot of input was treated with an anti-GFP polyclonal antibody (Santa Cruz Biotechnology, catalog #GFP-FL), followed by incubation with protein A agarose (Millipore, catalog #16-157) to immunoprecipitate the NTF protein. Bead-bound proteins were washed twice for 5 minutes with RIPA buffer and eluted with 2X SDS loading buffer (100 mM Tris, 10% sodium dodecyl sulfate, 30% glycerol, 1% β-mercaptoethanol, 0.2% bromophenol blue, pH=7.5). Input and immunoprecipitated fractions were electrophoresed on a 12% SDS polyacrylamide gel and transferred to a nitrocellulose membrane. The membrane was blocked in PBSt (11.9 mM sodium phosphate, 137 mM NaCl, 2.7 mM KCl, 0.1% Triton X-100, pH=7.4) with 10% milk for 30 minutes, washed twice for 5 minutes with PBSt, and incubated with a 1:2000 dilution of streptavidin-HRP (GE, catalog #RPN1231) in PBSt with 1% BSA for 30 minutes. The membrane was then washed three times for 5 minutes with PBSt and biotinylated proteins were detected using ECL detection reagents (Pierce, catalog #34075).

Fluorescence-Activated Cell Sorting (FACs) of Non-Hair Cell Protoplasts

As a control for comparison, Arabidopsis non-hair cells were isolated from root extracts using fluorescence-activated cell sorting (FACS) according to a methodology previous described by Birnbaum, K., et al., “Cell Type-Specific Expression Profiling in Plants Via Cell Sorting of Protoplasts From Fluorescent Reporter Lines,” Nat. Methods 2:615-619, 2005, incorporated herein by reference in its entirety.

Results:

The present inventors developed a novel system for tagging nuclei using an outer nuclear envelope-tagging fusion (NTF) protein. In the present embodiment, the NTF served as a substrate for biotinylation. As shown in FIG. 1B, the encoded nuclear tagging fusion (NTF) protein comprises three parts: (1) a nuclear envelope targeting sequence 14, such as the WPP domain of Arabidopsis RAN GTPASE ACTIVATING PROTEIN 1 (RanGAP1), which is necessary and sufficient for envelope association (see Rose et al., 2001); (2) a visualization tag 22, such as the green fluorescent protein (GFP); and (3) an affinity reagent binding region 16, such as the biotin ligase recognition peptide (BLRP), which acts as a substrate for the E. coli biotin ligase BirA (Beckett et al., 1999). Thus, in this specific embodiment, as illustrated in FIGS. 1E and F, expression of the NTF and BirA in the same cell type result in the production of biotinylated nuclei exclusively in that cell type. In some embodiments, the fusion gene encoding NTF is driven by a cell type-specific promoter, such as a promoter specific for expression of the NTF protein in hair cells using the ACTIN DEPOLYMERIZING FACTOR 8 (ADF8) promoter (Ruzicka et al., 2007) in one transgenic line, and a promoter specific for expression in non-hair cells, such as the GLABRA2 (GL2) promoter (Masucci et al., 1996) in another line. In the present Example, both of these transgenic lines also expressed BirA from the constitutive ACTIN2 (ACT2) promoter (An et al., 1996) to provide biotinylation of the NTF in the hair or non-hair cell types.

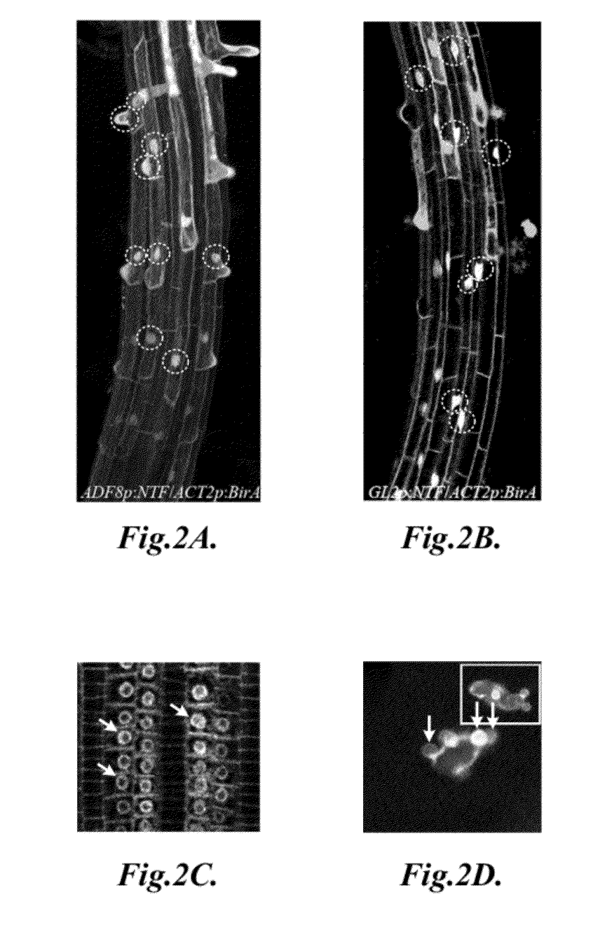

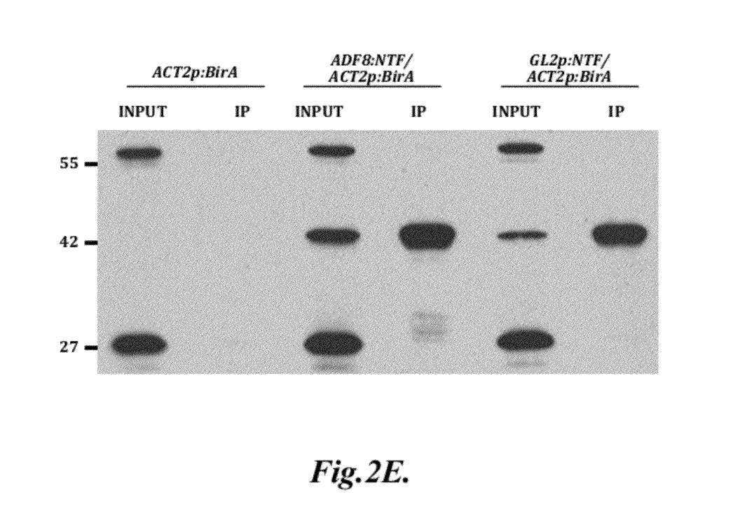

Fluorescence microscopic examination of the ADF8p:NTF/ACT2p:BirA and GL2p:NTF/ACT2p:BirA transgenic lines showed that both promoters were expressed exclusively in the expected cell type and that the NTF did indeed accumulate on the nuclear envelope (FIGS. 2A-C). Specifically, FIG. 2A is a confocal projection image of the differentiation zone of an ADF8p:NTF/ACT2p:BirA transgenic root showing expression of the NTF protein in hair cells. FIG. 2B is a confocal projection of the differentiation zone of an GL2p:NTF/ACT2p:BirA transgenic root showing expression of the NTF protein in non-hair cells. For both figures, illustrative examples of the GFP signal are indicated by dashed circles showing localization of the NTF in the nuclear membranes. Propidium iodide staining of cell walls is shown in red, appearing in the present gray-scale figure generally as the linear wall architecture of the cells. FIG. 2C is a confocal section of the post-meristematic region of a GL2p:NTF/ACT2p:BirA transgenic root, with arrows indicating illustrative GFP signal localizing on the nuclear envelopes.

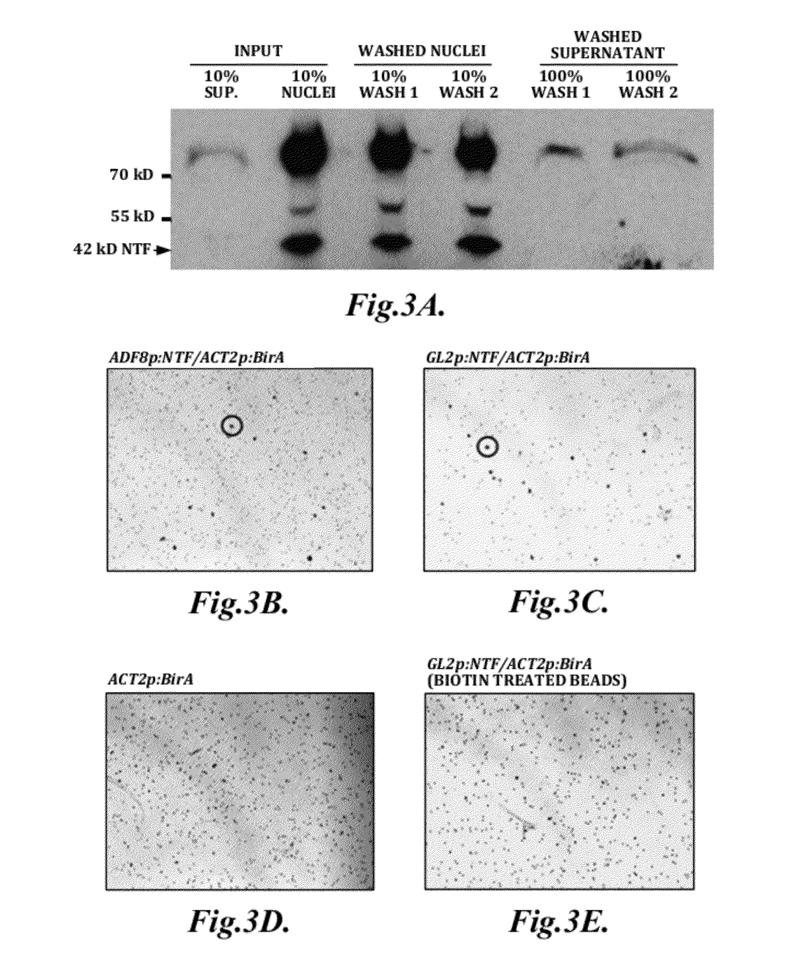

Furthermore, as shown in FIG. 2D and FIG. 3A, it was observed that nuclei isolated from these lines retained the NTF on their surface. Specifically, FIG. 2D is a fluorescence micrograph of a nucleus isolated from ADF8p:NTF/ACT2p:BirA transgenic roots and incubated with streptavidin Dynabeads®. The beads, illustrated with arrows, remain bound to the nucleus. FIG. 3A, discussed in more detail below, is a streptavidin western blot that demonstrates that streptavidin-bound beads remain bound to tagged nuclei even after several washes.

As a further confirmation that the NTF was biotinylated, streptavidin western blotting was performed on whole cell extracts, on anti-GFP immunoprecipitates (IP) from the roots of each transgenic line, and on extracts from a line expressing only ACT2p:BirA. As shown in FIG. 2E, a biotinylated protein of the expected 42 kD size was detected only in plants that expressed the NTF, and this protein could be immunoprecipitated with an anti-GFP antibody. Thus, the NTF was expressed properly in each line and was found to be biotinylated.

To isolate labeled nuclei from hair and non-hair cells, total nuclei from the fully differentiated root hair zone of young seedlings in each transgenic line were extracted and the nuclei were incubated with streptavidin-coated magnetic beads. A simple liquid flow-based system was employed to capture the bead-bound nuclei on a magnet as the solution of bound and unbound nuclei flowed past. This apparatus was constructed from common laboratory supplies and a Dynal Mini-MACS™ magnet, as diagrammed in FIG. 1G. Using two successive rounds of flow purification enabled isolation of an average (+/−SD) of 150,000+/−45,000 hair cell nuclei from ADF8p:NTF/ACT2p:BirA and 250,000+/−65,000 non-hair cell nuclei from GL2p:NTF/ACT2p:BirA, starting with 3 grams of root segments from each line. The consistently higher yield of non-hair cell nuclei from the GL2p:NTF/ACT2p:BirA line was expected, given that there are generally 10-14 non-hair cell files in the epidermis and only 8 hair cell files (see Dolan, L., et al., “Cellular Organisation of the Arabidopsis thaliana Root,” Development 119:71-84, 1993; Grierson and Schiefelbein, Root Hairs, Arabidopsis Book:1-22 (2003)). The average purity (+/−SD) of the nuclei obtained was found to be 92.8+/−1.6% for hair cell nuclei and 95+/−2.2% for non-hair cell nuclei.

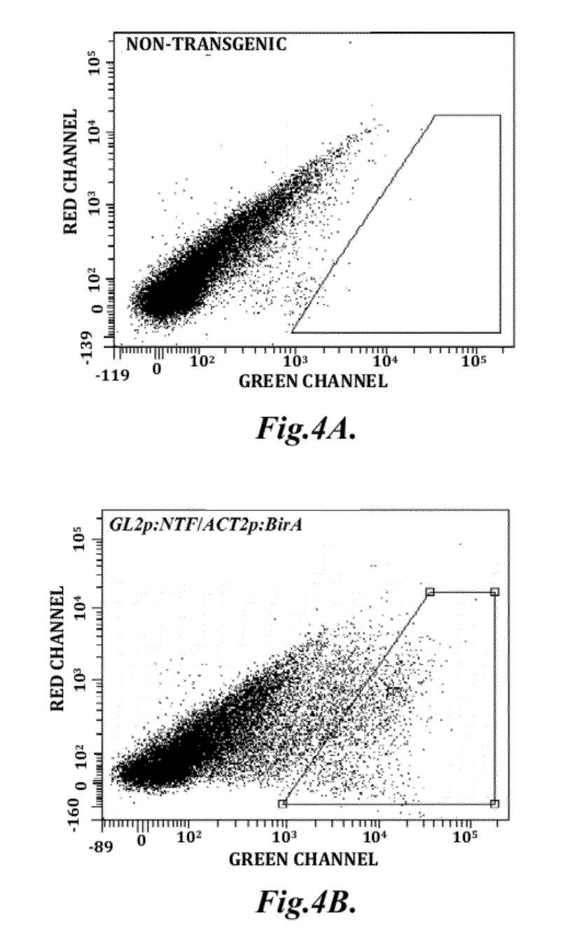

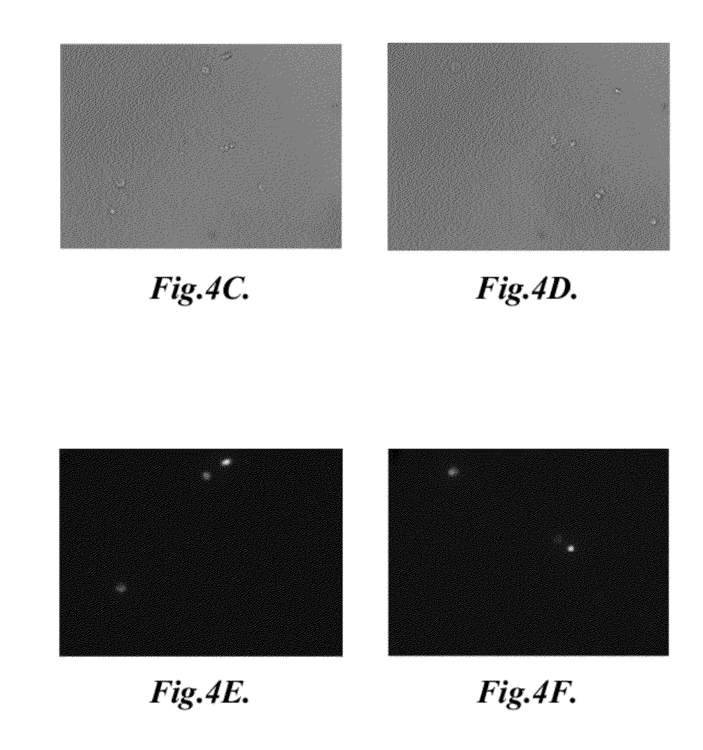

As a control for comparison to the INTACT method, GFP-positive non-hair cell protoplasts were also sorted using fluorescence-activated sorting (FACS) according to the methodology previously described by Birnbaum, et al., 2005. FIGS. 4A and B are FACS scatterplots, wherein the boxed area is the gate used for sorting GFP-positive protoplasts. FIGS. 4C-F are brightfield and GFP micrographs of the FACS-purified protoplasts. As demonstrated in FIGS. 4A-F, in contrast to the INTACT method described above, purity of non-hair cell protoplasts in the FACS-purified preparations was found to be 48+/−5% based on the number of GFP-positive versus GFP-negative protoplasts, as determined by microscopic examination. The purity measurements in the FACS control method are considered to be accurate because membrane disruption and cytoplasmic leaking from protoplasts during sorting do not affect GFP fluorescence. This is because the NTF is tethered to the nuclear envelope and does not appear to dissociate, as described below and illustrated in FIG. 3. Despite the conservative GFP gate settings used, insufficiently pure hair or non-hair cell protoplasts were obtained, which prohibited expression profiling for comparison to our INTACT-derived expression profiles described in EXAMPLE 2 below.

As described, it was also demonstrated that nuclei purified from the ADF8p:NTF/ACT2p:BirA hair cell and GL2p:NTF/ACT2p:BirA non-hair cell lines could be specifically bound by streptavidin-coated magnetic beads, which resisted dissociation even after multiple washes with nuclei purification buffer, and shown in FIGS. 3A and 3B. In this regard, as shown in FIG. 3A, no NTF could be detected in the wash fractions and the amount found in the washed nuclei fractions did not decrease detectably, indicating that NTF does not dissociate from the nuclear envelope under the conditions used. As shown in FIGS. 3B and C, large dark spots indicate bead-bound nuclei and small spots are single beads. Bead-bound nuclei are present in the ADF8p:NTF/ACT2p:BirA (FIG. 3B) and GL2p:NTF/ACT2:BirA (FIG. 3C) nuclei preparations, but not in those from a line carrying only ACT2p:BirA alone (FIG. 3D) or in GL2p:NTF/ACT2p:BirA preparations in which the beads were pre-treated with free biotin (FIG. 3E).

Discussion:

In order to circumvent the limitations of current methods and to make the study of cell differentiation and function more accessible, a simple and generally applicable method was developed for studying gene expression and chromatin in individual cell types. To avoid the need for dissociating or mechanically separating cells, a strategy was developed to transgenically tag nuclei in specific cell types and then isolate them from the total pool of nuclei derived from a tissue by affinity isolation targeting the tag.

It has been shown that the nuclear and total cellular mRNA pools are generally comparable, making nuclei a reasonable source of mRNA for gene expression measurements (see Barthelson, R. A., et al. “Comparison of the Contributions of the Nuclear and Cytoplasmic Compartments to Global Gene Expression in Human Cells,” BMC Genomics 8:340, 2007; Jacob, Y., et al., “The Nuclear Pore Protein AtTPR Is Required for RNA Homeostasis, Flowering Time, and Auxin Signaling,” Plant Physiol. 144:1383-1390, 2007). Thus, affinity purified nuclei can be used for the measurement of the gene expression and chromatin profiles of individual cell types. The present strategy to achieve this was to express an expression cassette encoding a nuclear tagging fusion (NTF) protein comprising a nuclear envelope targeting sequence, green fluorescent protein (GFP), and the biotin ligase recognition peptide (BLRP), in the presence of E. coli biotin ligase (BirA) in individual cell types (i.e., under the control of a cell-type specific promoter) in order to generate biotinylated nuclei specifically in those cells. These nuclei could then be purified from the total nuclear pool by virtue of the interaction between biotin and streptavidin. This strategy is referred to herein as INTACT, for isolation of nuclei tagged in specific cell types.

The data provided herein demonstrate that the novel INTACT method is easy to perform, does not require sophisticated instrumentation or specialized skills, and can produce large quantities of the desired nuclei at very high purity, in contrast to FACS and LCM-based methods for cell isolation. For example, INTACT provided recovery of >10⁵ nuclei at nearly 100% purity, whereas <10% of hair cell-specific protoplasts with only 50% purity were recovered using FACS based on GFP fluorescence (see FIGS. 4A-F). INTACT is also clearly suitable for isolating nuclei from relatively rare cell types, given that hair and non-hair cells each represent only about 10% of cells in the primary root (Dolan et al., 1993). Given the high specificity and avidity of the biotin-streptavidin interaction, it is also possible to isolate nuclei from cells with even lower abundance in sufficient quantities simply by starting with a larger amount of whole tissue. In addition, this approach is applicable to any organism that can be transformed, and is limited only by the need for a suitable nuclear envelope-targeting domain and a promoter that is expressed in the cell type of interest and not in nearby cells. The RanGAP1 WPP domain is likely to be useful for many other, if not all, plant cell types. For adaptation of the method to non-plant systems, the C-terminus of RanGAP, or nuclear pore complex proteins are useful in place of the WPP domain for nuclear targeting. Thus, INTACT represents a universal strategy for cell type-specific profiling.

Conclusion:

These results demonstrate that the INTACT method results in high yield and high purity of cell-specific nuclei for each cell type tested. The average purity (+/−SD) of the nuclei obtained was found to be 92.8+/−1.6% for hair cell nuclei and 95+/−2.2% for non-hair cell nuclei, which was considerably greater than the purity observed with the use of FACS to isolate GFP-positive protoplasts.

### Example 2

This Example describes gene expression profiling of the INTACT-purified nuclei from hair cells and non-hair cells of the A. thaliana root epidermis, generated as described in EXAMPLE 1.

Rationale:

As described above in EXAMPLE 1, the formation of hair and non-hair cells of the A. thaliana root epidermis has been extensively studied at the genetic and cell biological levels (Ishida et al., 2008), and many genes that are expressed preferentially in each cell type have been identified (Birnbaum et al., 2003; Brady et al., 2007; Won et al., 2009), providing a point of comparison for gene expression studies using the INTACT method.

Methods:

Generation and Purification of Biotinylated Nuclei

Biotinylated nuclei from hair and non-hair cells of A. thaliana were generated and purified as described in EXAMPLE 1.

Gene Expression Profiling Using Nuclear RNA

Total RNA was isolated from purified nuclei (obtained as described in EXAMPLE 1), using the Qiagen RNeasy® Micro kit. RNA was first treated with RNase-free Dnase I and then cDNA was prepared and amplified using the Sigma Whole Transcriptome Amplification Kit (Sigma, catalog #WTA2). This synthesis/amplification method begins with a cDNA synthesis using primers with a random 3′ end and defined 5′ end, followed by PCR using primers that match the 5′ end of the primers used for cDNA synthesis. The amplified cDNA was labeled in a random priming reaction using Cy dye-containing random 9mers as directed in the Roche NimbleGen® protocol supplied with the arrays. Sheared genomic DNA was labeled with the complementary Cy dye and was then co-hybridized along with labeled cDNA to a custom-designed Arabidopsis 1.9 million feature tiling array obtained from Roche NimbleGen®, which was described previously (Bernatavichute, Y. V., et al. “Genome-Wide Association of Histone H3 Lysine Nine Methylation With CHG DNA Methylation in Arabidopsis thaliana,” PLoS ONE 3:e3156, 2008). This array covers the entire sequenced portion of the Arabidopsis genome with an isothermal probe design. All array hybridizations and scanning were performed by the Genomics Shared Resource lab at the Fred Hutchinson Cancer Research Center.

Two biological replicates of the experiment were performed for each cell type and the raw log₂ ratio data from each of these were processed by conversion to standard deviates on a probe-by-probe basis. An expression score was then calculated for each gene by averaging the log₂ ratios of the first 100 exonic probes, starting at the 3′ end of the gene and moving toward the 5′ end. In order to define the set of genes enriched in each cell type we compared the data sets from each cell type using the program CyberT® (described in Baldi, P., and A. D. Long, “A Bayesian Framework for the Analysis of Microarray Expression Data: Regularized t-Test and Statistical Inferences of Gene Changes,” Bioinformatics 17:509-519, 2001). Within CyberT®, a Bayesian analysis was performed using with a window size of 101 and a confidence level of 10. Genes were classified as enriched in a given cell type if they showed a fold difference between cell types of >1.3 and a Bayes p value of <0.02.

Gene Ontology (GO) analysis was performed on each set of cell type-enriched genes using the GeneCodis 2.0 program (Carmona-Saez, P., et al., “GENECODIS: A Web-Based Tool for Finding Significant Concurrent Annotations in Gene Lists,” Genome Biol 8:R3, 2007; Nogales-Cadenas, R., et al., “GeneCodis: Interpreting Gene Lists Through Enrichment Analysis and Integration of Diverse Biological Information,” Nucleic Acids Res. 37:W317-322, 2009) with a hypergeometric test and false discovery rate calculation to correct the p values for multiple testing. The full set of genes present on the array was used as the background set in these analyses. Chi squared tests were also performed on the observed versus expected percentage of genes in selected GO categories.

Comparison of Whole Genome Expression Profiles from Total and Nuclear RNA Pools

Whole-genome expression profiling was performed using total and nuclear RNA pools from the differentiated root hair zone (same root segment used for INTACT purifications) of 7 day old non-transgenic plants. RNA isolated from whole root segments and nuclei was converted to cDNA, amplified, labeled, and hybridized to tiling arrays as described above. The whole genome expression profiles for each RNA source were compared on a scatterplot. A linear trend line was fit to the data to obtain an R value.

qRT-PCR Analysis

Wild Type (WT) Col-0 and gl2-8 mutant seedlings (T-DNA insertion line SALK_{—}130213) (Alonso, J. M., et al., “Genome-Wide Insertional Mutagenesis of Arabidopsis thaliana,” Science 301:653-657, 2003) were grown on plates of agar-solidified ½ strength MS as described above, and RNA was prepared from the root hair zone of 7-day-old seedlings using the Qiagen RNeasy® Plant Mini kit. Each RNA sample was treated with RNase-free DNAse I and cDNA was prepared using the Superscript® III kit (Invitrogen, catalog #18080-051) with oligo dT primers according to the manufacturer's instructions. Real-time PCR was performed on an Applied Biosystems 7900HT instrument using SYBR green detection chemistry. Relative quantities of each transcript were calculated using the 2^{ddct} method (Livak, K. J., and T. D. Schmittgen, “Analysis of Relative Gene Expression Data Using Real-Time Quantitative PCR and the 2(-Delta Delta C(T)) Method,” Methods 25:402-408, 2001) with At1g13320 serving as the endogenous control transcript in each case (Czechowski, T., et al., “Genome-Wide Identification and Testing of Superior Reference Genes for Transcript Normalization in Arabidopsis,” Plant Physiol. 139:5-17, 2005). Primer sequences are given below in TABLE 1.

TABLE 1
Primer sequences and results are provided for RT-PCR testing of putative hair cell genes.
Primer sequences are provided for 27 putative hair cell-enriched genes tested by RT-PCR in
wild-type and gl2-8 roots, and are set forth herein as SEQ ID NOS: 33-86. The far right
column indicates whether the gene had significantly higher expression (p < 0.07) in gl2-8
compared to wild-type roots, as expected for true hair cell genes (Y = yes, N = no.)

| | | SEQ | | SEQ ID | Higher in |
|---|---|---|---|---|---|
| Gene | Forward Primer | ID NO: | Reverse Primer | NO: | gl2-8? |
| AT1G04160 | CAGACAAGCTGTTGGGTTCCTG | 33 | AAGTTGTTGGACACTAAGGATCGG | 34 | Y |
| AT1G12040 | ACCACCGTGTCCTGAATCATCTC | 35 | TTTGTGTCACGGGTGCGTAG | 36 | Y |
| AT1G12560 | AAGACTCCAACGCTGGTGGTTG | 37 | TCCTTTGGCATGGCACTCTTCG | 38 | Y |
| AT1G18410 | AGGACGACAAACATTGCAAAGGC | 39 | TCTTCTTCATGCCTTGAGAACTCG | 40 | Y |
| AT1G70710 | TGTGGCGAATTAACTGCTTCCC | 41 | TCCGAGAATGTAATCCACCTGACG | 42 | N |
| AT2G03720 | ACAAGAACACCCGTAGGACAAGC | 43 | CGCCGTTTCAACCACCACTATC | 44 | Y |
| AT2G24980 | CTCCCAGCTATGAACACAAAGGC | 45 | AGTTTACCTTTGGCGATGGAGTG | 46 | Y |
| AT2G37670 | AGACGGTCAGGGTGCACTTATC | 47 | CGCTTGTGATTTCTTGTTGCTCTG | 48 | Y |
| AT2G39390 | CGAAGACGCAATGGCGAGAATC | 49 | TAGCGACACGAAGGAGAGCAAG | 50 | Y |
| AT2G44110 | AGCTGATGCTTCCTTGGGATGG | 51 | TGGGTTGGTCTTGCAATGAGTCG | 52 | Y |
| AT3G04630 | TGCTGAGAGAGTTGGAGCTCAG | 53 | AGAGGAGCATCCTGCTGCTTTG | 54 | N |
| AT3G10740 | CCTTCTCACAGCCAGAGAAGGTTG | 55 | CGGGAGAACAACGGTCATATCCTC | 56 | N |
| AT3G12540 | ACAACGCCTGCGTTATGAATGG | 57 | ACCTCCCACGTCAATTGTTGCC | 58 | Y |
| AT3G54580 | TCATTTGCGCTCTAGGAGTTGTC | 59 | TGGTGGAGAGCTATCTGTGTATGG | 60 | Y |
| AT3G54870 | TGCGTTAGCTGAAGGCAGTTCTC | 61 | AAGTGAAGTCCTCGCAGAACCC | 62 | Y |
| AT3G62680 | TCCCTTGGCGTTGTACGGATAC | 63 | TCCGACGCTAAAGAGCTCCAAG | 64 | Y |
| AT4G28530 | TGCACGAGTTCCGTCTTGAGTG | 65 | TGCACAAGACCCAGTCTTCCTTAG | 66 | Y |
| AT4G31250 | CGCTAATCTCCTCCATGCTAACCG | 67 | AGCCAATCCTCTCGTAACTCCTC | 68 | Y |
| AT4G34580 | TGACTTGAAACCTGCTCATGTCG | 69 | ACAAGTGCTTCTTCCAAAGCCTTC | 70 | Y |
| AT5G05500 | GATTTACGCCGCTGGTCCATTG | 71 | TCAGTAAGTGGGTGGTGCAGTC | 72 | Y |
| AT5G19790 | AGATACGGATGTGGCTCGGAAC | 73 | AGACATGCAGCTTCATCGTAGGC | 74 | Y |
| AT5G22880 | TCTCCAGCAAAGCCATGGGAATC | 75 | AGCTTCGAAGACTCACCAGCAAG | 76 | N |
| AT5G48870 | TTCACAGCTTCTTCCTTCAGAGC | 77 | CCAACGAGCTCCTTATCTCCTTTC | 78 | N |
| AT5G49520 | GGTTTGCGTTTCTGACGAAGAGC | 79 | TGGTGCAACGGTAATAGCTTCTGG | 80 | Y |
| AT5G52010 | AGAGCATTGGGAAGGCATGCTG | 81 | TCTCTCCCTTCTCAACACCACTCC | 82 | N |
| AT5G54050 | TGAAGTGGAGCCATGGTTTAGGAC | 83 | GGTCAACGCAGTCTTTGTGCATC | 84 | Y |
| AT5G58010 | GGTTCCCAACACCAACAAGACG | 85 | ACTGATCCTGCACCTCCCAATC | 86 | Y |

Results:

Overall, 21 out of the 27 tested genes (78%) were confirmed to have higher expression in gl2-8 roots, as illustrated in TABLE 1. Genes that showed increased expression in the mutant are likely to be true hair cell-specific genes, but those that do not show an increase are not necessarily false positives. Some hair cell-specific transcripts might not have a higher relative abundance in the gl2 mutant because the hair-like cells induced in the mutant may express only a subset of the entire hair cell transcriptome, and some genes that are hair cell-specific (as compared to non-hair cells) may be expressed in other root cell types. The latter scenario could prevent detection of increased abundance in the mutant due to signals arising from other root cell types.

After successfully isolating nuclei from fully differentiated hair and non-hair cells, gene expression profiles of each cell type were measured using nuclear RNA. cDNA was prepared and amplified from the total nuclear RNA of each cell type. The cDNA was Cy dye-labeled and hybridized to Roche NimbleGen® whole-genome tiling microarrays along with fragmented genomic DNA labeled with the complementary Cy dye. Expression scores for the 26,992 annotated genes represented on the array were calculated using data from each of two biological replicates per cell type, and these datasets were then compared. A gene was defined as preferentially expressed in a given cell type if it showed a fold difference between cell types of >1.3 with a Bayes p value of <0.02 (Baldi and Long, 2001). Using these criteria, 946 genes were identified that were enriched in hair cells and 118 genes were identified that were enriched in non-hair cells.

To determine whether the hair and non-hair cell-enriched genes identified by INTACT correspond to genes identified using other methods, the identified cell type-enriched gene lists were compared to those obtained in previous expression studies. Nineteen of 24 confirmed hair cell-specific genes identified by Won et al., 2009, were present in the hair cell-enriched gene list generated by the INTACT method, and none were found in the non-hair gene set. Therefore, most of the previously confirmed hair cell-enriched genes were found using INTACT, and these genes were found throughout the range of expression levels in the present dataset, indicating that INTACT can identify cell type-specific genes regardless of expression level, as shown in TABLE 2.

TABLE 2
Expression levels of confirmed hair cell
genes identified using the INTACT method.

| | Rank by expression in |
|---|---|
| Confirmed hair cell gene | hair cell (total = 26992) |
| AT1G54970 | 1970 |
| AT1G70460 | 2308 |
| AT3G10710 | 2873 |
| AT3G62680 | 2984 |
| AT5G67400 | 3155 |
| AT1G12560 | 3617 |
| AT1G12040 | 3641 |
| AT1G12950 | 3774 |
| AT1G34760* | 3882 |
| AT1G62980 | 3947 |
| AT4G02270 | 5235 |
| AT1G69240 | 6127 |
| AT4G22080 | 7168 |
| AT1G30850 | 7308 |
| AT1G62440* | 7985 |
| AT1G16440 | 8004 |
| AT2G45890* | 8040 |
| AT4G38390 | 9209 |
| AT5G22410 | 10007 |
| AT4G29180 | 10350 |
| AT1G51880 | 10388 |
| AT4G25220 | 12866 |
| AT1G05990* | 14687 |
| AT1G63450 | 18909 |

All 26,992 genes present on the array were ranked by expression level in the hair (H) cell expression profile, from highest to lowest (1-26992, respectively). The table shows the expression rank of each of the 24 confirmed H cell-specific genes (Won et al., 2009), indicating that cell type-specific genes can be detected throughout the range of expression levels. Asterisks denote the four genes not categorized as hair cell-specific in our analysis.

The INTACT cell type-enriched gene lists were compared to genes identified from earlier studies that performed expression profiling using FACS-purified protoplasts of hair and non-hair cells (Birnbaum et al., 2003; Brady et al., 2007). Only about 20% of the genes previously defined as specific to each cell type were present in the corresponding INTACT gene lists. In addition, only 11 of the 24 confirmed hair cell-specific genes were found in the FACS-based hair cell-enriched gene list. The discrepancies between INTACT and FACS-based expression profiles of each cell type could be attributable to technical differences between the studies, such as cDNA amplification methods, microarray platforms used, and methods for defining cell-type specific expression. However, a major source of variation may also arise from differences in the purity of target cells or nuclei achieved with each of the methods, as described in EXAMPLE 1. While the INTACT method is shown here to give nearly 100% purity of the desired nuclei, in contrast, a published FACS protocol (Birnbaum et al., 2005) was unable to achieve a purity of greater than 50% for hair or non-hair cell protoplasts from the present transgenic lines (see FIG. 4). Thus, differences in the expression profiles could also result from a higher level of contamination from other cell types that seems to be inherent to FACS purification of plant protoplasts.

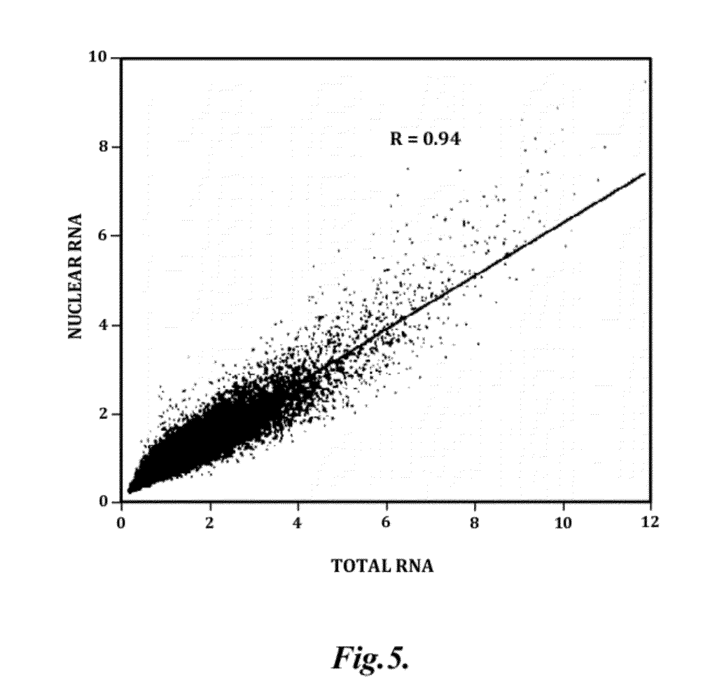

Another possible explanation for the discrepancies between INTACT and FACS-based expression profiles is that differences in the total and nuclear RNA pools could be prevalent in the tissue used for these experiments. In order to address this issue, whole-genome expression profiling was performed for nuclear and total RNA from the same tissue used for INTACT purification of hair and non-hair cell nuclei. FIG. 5 is a scatter plot of nuclear RNA versus total RNA hybridization signals derived from the average of two replicate tiling arrays. As shown, a very high degree of similarity (R=0.94) in the composition of these two RNA pools was demonstrated. Therefore, expression profiles derived from pure nuclei or protoplasts of a given cell type are comparable.

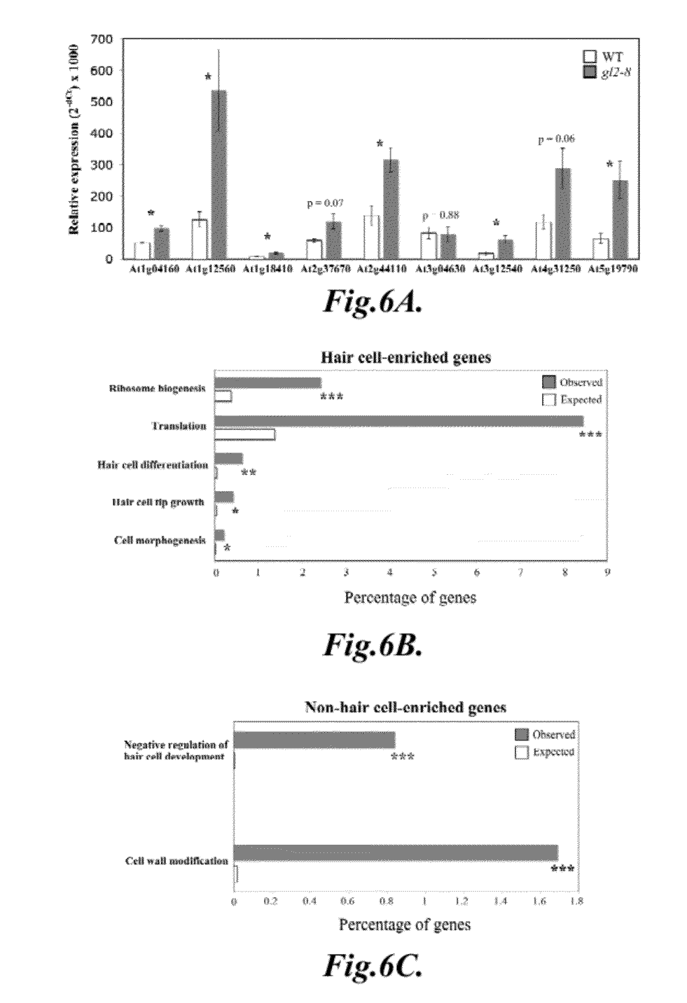

As an independent measure of the accuracy of the present expression profiles, 27 genes were selected from the hair cell-enriched set and analyzed for expression levels in wild-type and gl2-8 mutant roots. Given that all epidermal cells are converted to hair cells in a gl2 mutant (Di Cristina, M., et al., “The Arabidopsis Athb-10 (GLABRA2) is an HD-Zip Protein Required for Regulation of Root Hair Development,” Plant J. 10:393-402, 1996; Masucci et al., 1996), it is reasoned that true hair cell-specific genes should show higher relative expression levels in gl2-8 roots as compared to wild-type roots. In total, 21 of the 27 genes (78%) tested were found to have a higher relative expression level in gl2-8 roots, and 10 of these 21 were found only in the INTACT hair cell dataset and not in the FACS-based dataset (Brady et al., 2007) (see TABLE 1 above). Expression levels for a representative subset of the tested genes is shown in FIG. 6A.

While not wishing to be bound by theory, it is hypothesized that the inability to detect increases in expression for 6/27 hair-cell enriched genes has a biological basis, given the high purity of the present cell-type specific population of nuclei obtained by the INTACT method. It is unknown how closely the hair-like cells induced in the mutant resemble normal hair cells in terms of their global gene expression profile. It is possible that these hair-like cells express only a part of the hair cell transcriptome, certainly enough to cause polarized growth and secondary cell wall thickening, but perhaps not all of it. Therefore, genes that are at significantly higher levels in gl2-8 are very likely to be hair cell-specific, but those that do not increase are not necessarily false positives. Furthermore, because the present expression profile comparisons were only between hair and non-hair cells, genes are categorized as hair-cell specific only relative to non-hair cells, but some of these genes might also be expressed in other root cell types. In the case of such genes, an expression increase in the mutant could be obscured by signals from other root cell types.

To test for biological functions known to be associated with the hair and non-hair cell types, each cell type-enriched gene set was analyzed for overrepresentation of Gene Ontology (GO) terms (Ashburner, M., et al., “Gene Ontology: Tool for the Unification of Biology,” The Gene Ontology Consortium. Nat. Genet. 25:25-29, 2000). FIGS. 6B and C graphically illustrates observed versus expected percentage of genes in each Gene Ontology (GO) annotation category for H cell-enriched genes and NH cell-enriched genes, respectively. In the hair cell gene set, a significant enrichment of multiple GO terms was located at all levels, including those associated with protein translation, actin and tubulin cytoskeletal systems, cell wall modification, and hair cell differentiation and growth (FIG. 6B and TABLE 3 below). Within the non-hair cell gene set, significant overrepresentation of GO terms was observed for cell wall modification and negative regulation of hair cell specification (FIG. 6C and TABLE 3 below). Thus, in each case, overrepresentation of terms was detected that correspond to biological functions known to be relevant to each cell type (Grierson and Schiefelbein, 2003; Masucci et al., 1996).

TABLE 3
Functional classification of cell type-enriched genes.

| | | | | # of genes | # of genes | |
|---|---|---|---|---|---|---|
| | | | | in query set | in | |
| | | | | (out of 946 | reference | |
| | Classification | | | H cell/118 | set (26992 | Corrected |
| Cell Type | scheme | Category | Subcategory | NH cell) | genes) | p value |
| Hair cell | Gene | Biological | Ribosome biogenesis | 23 | 96 | 1.9 × 10−13 |
| | Ontology (GO) | Process | (GO: 0042254) | | | |
| | | | Translation | 80 | 365 | 1.5 × 10−40 |
| | | | (GO: 0006412) | | | |
| | | | Response to hydrogen | 6 | 33 | 8.0 × 10−4 |
| | | | peroxide | | | |
| | | | (GO: 0042542) | | | |
| | | | Proton transport | 4 | 12 | 5.0 × 10−4 |
| | | | (GO: 0015992) | | | |
| | | | Root hair cell | 6 | 11 | 7.2 × 107 |
| | | | differentiation | | | |
| | | | (GO: 0048765) | | | |
| | | | Root hair cell tip | 4 | 8 | 9.3 × 10−5 |
| | | | growth | | | |
| | | | (GO: 0048768) | | | |
| | | | Cell morphogenesis | 2 | 2 | 1.2 × 10−3 |
| | | | involved in | | | |
| | | | differentiation | | | |
| | | | (GO: 0000904) | | | |
| | | Molecular | Hydrolase activity, | 7 | 40 | 4.3 × 10−4 |
| | | Function | acting on glycosyl | | | |
| | | | bonds | | | |
| | | | (GO: 0016798) | | | |
| | | | Structural constituent | 88 | 364 | 2.7 × 10−48 |
| | | | of ribosome | | | |
| | | | (GO: 0003735) | | | |
| | | | Actin binding | 8 | 54 | 5.5 × 10−4 |
| | | | (GO: 0003779) | | | |
| | | | Microtubule motor | 9 | 67 | 5.3 × 10−4 |
| | | | activity | | | |
| | | | (GO: 0003777) | | | |
| | | | Peroxidase activity | 13 | 80 | 3.9 × 10−6 |
| | | | (GO: 0004601) | | | |
| | | Cellular | Ribosome | 63 | 218 | 8.1 × 10−40 |
| | | Component | (GO: 0005840) | | | |
| | | | Membrane | 77 | 1371 | 3.2 × 10−5 |
| | | | (GO: 0016020) | | | |
| | | | Plasma membrane | 102 | 1894 | 9.6 × 10−6 |
| | | | (GO: 0005886) | | | |
| | | | Anchored to | 24 | 237 | 3.5 × 10−6 |
| | | | membrane | | | |
| | | | (GO: 0031225) | | | |
| | | | Anchored to plasma | 7 | 66 | 8.2 × 10−7 |
| | | | membrane | | | |
| | | | (GO: 0046658) | | | |
| | | | Vacuole | 35 | 523 | 2.2 × 10−4 |
| | | | (GO: 0005773) | | | |
| | | | Cell wall | 36 | 321 | 9.2 × 10−10 |
| | | | (GO: 0005618) | | | |
| | | | Plant-type cell wall | 24 | 260 | 1.7 × 10−5 |
| | | | (GO: 0009505) | | | |
| | KEGG | | Ribosome (3010) | 64 | 215 | 8.6 × 10−40 |
| | Pathways | | | | | |
| | | | Oxidative | 14 | 112 | 4.1 × 10−4 |
| | | | phosphorylation (190) | | | |
| | | | Phenylalanine | 11 | 81 | 9.8 × 10−4 |
| | | | metabolism (360) | | | |
| | | | Methane metabolism | 12 | 81 | 4.0 × 10−4 |
| | | | (680) | | | |
| Non-hair | Gene | Biological | Cell wall modification | 2 | 5 | 9.3 × 10−3 |
| cell | Ontology (GO) | Process | (GO: 0042545) | | | |
| | | | Choline biosynthetic | 1 | 1 | 0.054 |
| | | | Choline biosynthetic | 1 | 1 | 0.054 |
| | | | process | | | |
| | | | (GO: 0042425) | | | |
| | | | Iron chelate transport | 1 | 1 | 0.054 |
| | | | (GO: 0015688) | | | |
| | | | Negative regulation of | 1 | 1 | 0.054 |
| | | | trichoblast fate | | | |
| | | | specification | | | |
| | | | (GO: 0010062) | | | |
| | | Molecular | None significantly | | | |
| | | Function | enriched | | | |
| | | Cellular | None significantly | | | |
| | | Component | enriched | | | |
| | KEGG | | None significantly | | | |
| | Pathways | | enriched | | | |

Hair and non-hair cell type-enriched genes were analyzed for overrepresentation of Gene Ontology (GO) terms and Kyoto Encyclopedia of Genes and Genomes (KEGG) pathway terms using the Genecodis 2.0 program as described in the Methods section, above.

Discussion:

Gene expression profiling using INTACT-purified hair and non-hair cell nuclei revealed a large number of genes that are preferentially expressed in each of these cell types. Among the genes classified herein as hair cell-enriched, most of the reporter-confirmed hair cell-specific genes were identified in the gl2-8 mutant roots as compared to wild-type roots. Additionally, increased expression was observed for many of the putative hair cell genes in the gl2-8 mutant roots as compared to wild-type roots. Analysis of overrepresentation of GO terms within the present gene sets revealed genes that were previously characterized as being involved in the specification of each of these cell types. In the case of hair cells, the GO terms analysis revealed an overabundance of genes involved in structural and physiological processes known to be important for the function of this cell type, such as translation, energy generation, cell expansion, vacuole function, and cytoskeletal dynamics. Furthermore, because nuclear and total RNA pools have a very similar composition, and INTACT provides nuclei at nearly 100% purity, the expression profiles generated from INTACT-purified nuclei should accurately represent the transcriptome of the cell type from which they were purified.

Conclusion:

These results demonstrate that the INTACT method results in high yield and purity of nuclei cell-specific nuclei populations that are suitable for gene expression analysis across the entire genome. Using the INTACT method, hundreds of genes were identified that are preferentially expressed in hair cell and non-hair cells of A. thaliana root epidermis, including nearly all of the previously confirmed hair cell-specific genes.

### Example 3

This Example describes chromatin profiling of the INTACT-purified nuclei from hair cells and non-hair cells of the A. thaliana root epidermis.

Rationale:

As described above in EXAMPLE 1, the formation of hair and non-hair cells of the A. thaliana root epidermis has been extensively studied at the genetic and cell biological levels (Ishida et al., 2008), and many genes that are expressed preferentially in each cell type have been identified (Birnbaum et al., 2003; Brady et al., 2007; Won et al., 2009). Additionally, as described in EXAMPLE 2, the use of the INTACT method enabled the identification of 946 genes that were enriched in hair cells and 118 genes enriched in non-hair cells using whole-genome tiling microarrays. These data provide an opportunity to examine the relationship of preferentially expressed genes with chromatin structure.

Methods:

Chromatin Profiling by Chromatin Immunoprecipitation

For chromatin immunoprecipitation (ChIP) experiments, excised root tissue were treated with 1% formaldehyde in NPB for 15 minutes prior to extraction and purification of biotinylated nuclei as described above. The ChIP protocol used herein is based on that of Gendrel et al (Gendrel, A. V., et al., “Profiling Histone Modification Patterns in Plants Using Genomic Tiling Microarrays,” Nat. Methods 2:213-218, 2005), but was modified for smaller amounts of starting material. Purified nuclei were lysed in 120 μL of nuclei lysis buffer (50 mM Tris, 10 mM EDTA, 1% sodium dodecyl sulfate, pH=8) and sonicated using a Diagenode Bioruptor® to yield chromatin fragments with an average size of ˜500 bp. Sonicated chromatin was cleared by centrifugation and diluted to 1.3 mL final volume with ChIP dilution buffer (16.7 mM Tris, 1.2 mM EDTA, 1.1% Triton X-100, 167 mM NaCl, pH=8). Diluted chromatin was pre-treated with 20 μL (bed volume) of protein A agarose beads (Millipore, catalog #16-157) for 30 minutes at 4° C. and then cleared by centrifugation. This chromatin was then divided into 2-3 aliquots of equal volume and 1-3 μg of antibody was added to each aliquot. The following antibodies were used in the experiments: H3, Abcam ab1791; H3K4me3, Abcam ab8580; H3K27me3, Millipore 07-449. Antibodies were incubated with chromatin at 4° C. overnight on a rocking platform, then 20 μL (bed volume) of protein A agarose beads were added with rocking at 4° C. for an additional 2 hours. Beads were washed once for 5 minutes at 4° C. in 0.5 mL of each of the following buffers: low salt wash buffer (20 mM Tris, 150 mM NaCl, 0.1% sodium dodecyl sulfate, 1% Triton X-100, 2 mM EDTA, pH=8), high salt wash buffer (20 mM Tris, 500 mM NaCl, 1% sodium deoxycholate, 1% NP-40, 1 mM EDTA, pH=8), LiCl wash buffer (10 mM Tris, 250 mM LiCl, 0.1% sodium dodecyl sulfate, 1% Triton X-100, 2 mM EDTA, pH=8), and TE (10 mM Tris, 1 mM EDTA, pH=7.5). Chromatin was eluted from the beads in 200 μL of elution buffer (100 mM NaHCO₃, 1% sodium dodecyl sulfate) with vortexing for 5 minutes, then NaCl was added to 0.5 M and eluted chromatin was heated to 100° C. for 15 minutes to reverse crosslinks. DNA was isolated by treating the chromatin with RNase A, Proteinase K, and purification using the Qiagen MinElute® kit. Amplification of ChIP DNA was performed with the Sigma Single Cell Whole Genome Amplification kit (Sigma, catalog # WGA4) as directed, and the amplified material was labeled with Cy3 or Cy5 dye as described above. For each experiment, the H3K4me3 or H3K27me3 ChIP DNA was co-hybridized to the tiling array (same array as used for expression analysis) along with H3 ChIP DNA from the same starting chromatin to equalize for nucleosome occupancy.

Two biological replicates of each ChIP were performed and the log₂ ratios from each replicate array were converted to standard deviates, averaged, and smoothed using triangular smoothing as described previously (Ooi, S. L., et al., “A Native Chromatin Purification System for Epigenomic Profiling in Caenorhabditis elegans,” Nucleic Acids Res, 38(4):e26, 2010). These data were used for all analyses. Cluster analysis was performed with Cluster 3 (Eisen, M. B., et al., “Cluster Analysis and Display of Genome-Wide Expression Patterns,” Proc. Natl. Acad. Sci. USA 95:14863-14868, 1998) and results were viewed using Java Treeview 1.1.0 (Saldanha, A. J., “Java Treeview—Extensible Visualization of Microarray Data,” Bioinformatics 20:3246-3248, 2004). End analysis was performed as previously described (Henikoff, S., et al., “Genome-Wide Profiling of Salt Fractions Maps Physical Properties of Chromatin,” Genome Research 19:460-469, 2009), and the analysis of each gene was stopped at the point where another genomic feature (gene or transposable element) was encountered. All microarray data are available from GEO (Accession Number GSE19654).

Results:

In order to gain insight into the chromatin changes that accompany the differentiation of hair and non-hair cells from a common progenitor, two different histone modifications were profiled in each cell type: the transcription-associated mark trimethylation of H3 lysine 4 (H3K4me3) (Santos-Rosa, H., et al., “Active Genes Are Tri-Methylated at K4 of Histone H3,” Nature 419:407-411, 2002) and the Polycomb silencing-associated mark trimethylation of H3 lysine 27 (H3K27me3) (Nekrasov, M., et al., “Pcl-PRC2 Is Needed to Generate High Levels of H3-K27 Trimethylation at Polycomb Target Genes,” EMBO J. 26:4078-4088, 2007).

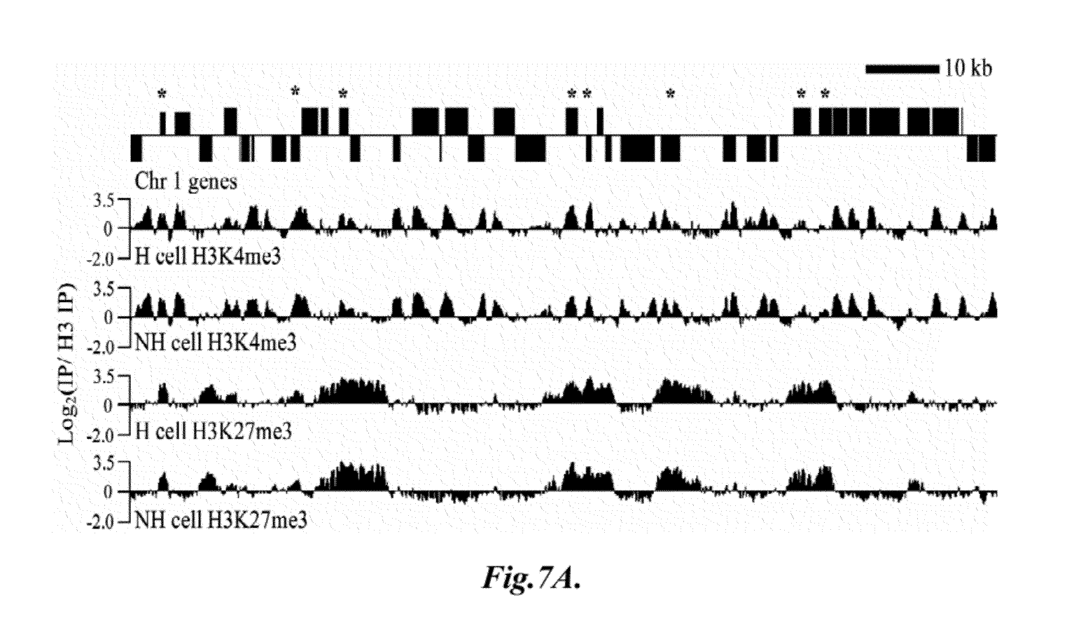

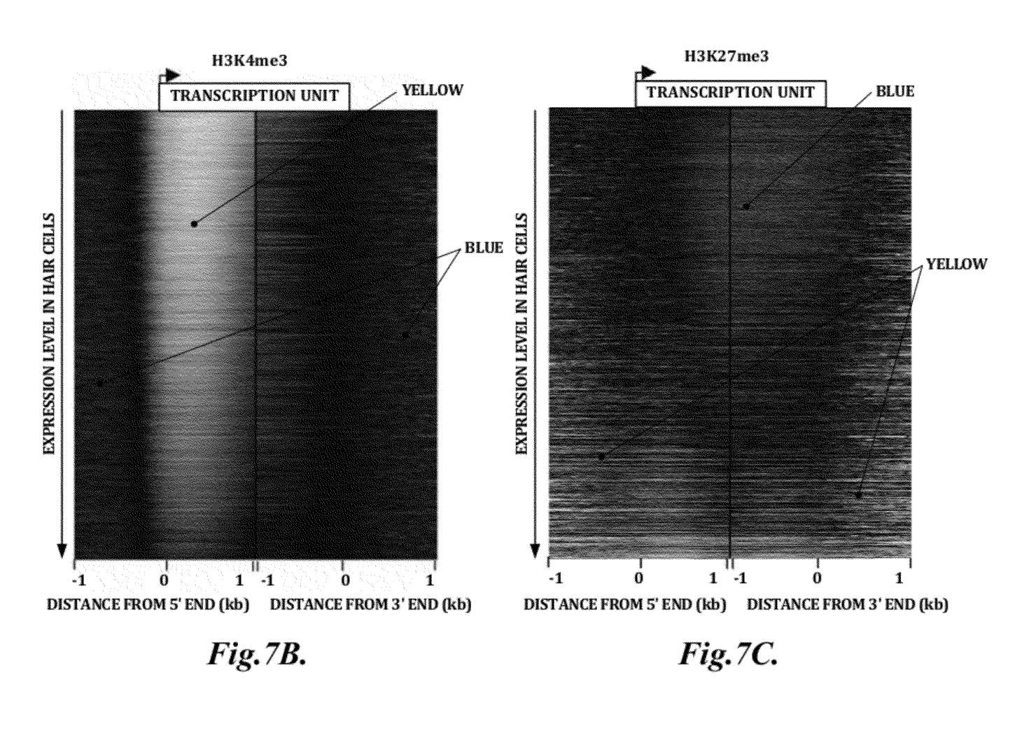

Chromatin immunoprecipitation (ChIP) was performed by shearing crosslinked chromatin from purified hair and non-hair cell nuclei to an average size of 500 bp, followed by immunoprecipitation with an antibody against either H3K4me3 or H3K27me3. To equalize for nucleosome occupancy, a sample of each input chromatin was also immunoprecipitated with an antibody against the C-terminus of H3, which should precipitate all nucleosomes irrespective of their post-translational modifications. Each amplified and labeled H3K4me3 or H3K27me3 ChIP DNA was co-hybridized to tiling arrays along with amplified and labeled H3ChIP DNA from the same input chromatin. Two biological replicates of each ChIP were performed for each of the two cell types. FIG. 7A graphically illustrates the resulting euchromatic chromatin landscapes of the histone H3 modifications, H3K4me3 and H3K27me3, in a region of chromosome 1 of hair (H) and non-hair (NH) cells. Chromosome 1 genes are shown schematically in the top line, wherein genes encoded in the top strand are indicated above the line and genes encoded in the bottom strand are below the line. Chromatin landscapes are expressed in a log ratio scale for each modification in each cell type. As shown in FIG. 7A, examination of a gene-rich region of chromosome 1 indicated that the ChIP experiments were highly reproducible and showed a high level of similarity between cell types for both modifications.

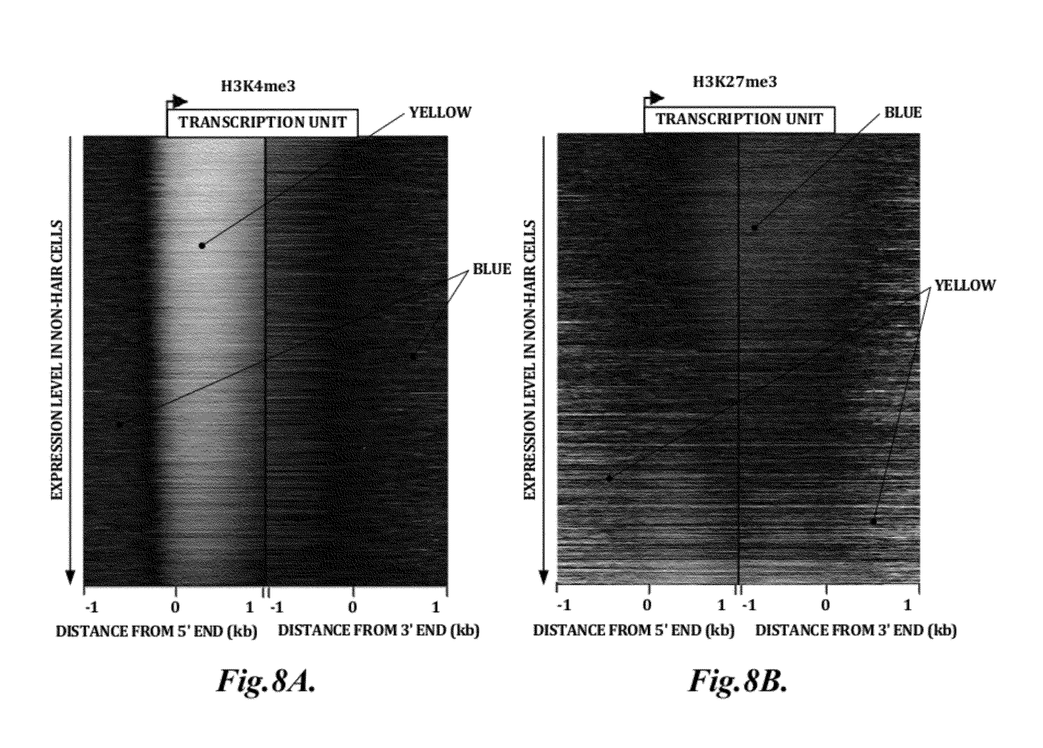

To visualize the relationship between gene expression and each of the modifications, heat maps were generated by aligning the profiles for each modification at the 5′ and 3′ ends of each annotated gene on the array, and then ranking genes by decreasing expression level in the corresponding cell type. FIG. 7B is a grey-scale illustration of the heat map generated for the H3K4me3 histone modification landscape relative to gene ends in hair (H) cells (−1 kb to +1 kb relative to transcription start and end sites). Similarly, FIG. 8A is a grey-scale illustration of the heat map generated for the H3K4me3 histone modification landscape relative to gene ends in non-hair (NH) cells (−1 kb to +1 kb relative to transcription start and end sites). The area of most intense yellow representation, indicating positive log 2 ratios for H3K4me3, is maximal just downstream of the transcription start site, and decreases with decreasing gene expression level in both hair (H) and non-hair (NH) cells (see area indicated as “yellow” FIG. 7B and FIG. 8A, respectively; the area indicated as “blue” represents data points of negative log 2 ratio for H3K27me3.), as described previously in other organisms (Bernstein, B. E., et al., “Genomic Maps and Comparative Analysis of Histone Modifications in Human and Mouse,” Cell 120:169-181, 2005; Krogan, N. J., et al., “The Paf1 Complex Is Required for Histone H3 Methylation by COMPASS and Dot1p: Linking Transcriptional Elongation to Histone Methylation,” Mol. Cell. 11:721-729, 2003; Roh, T.-Y., et al., “The Genomic Landscape of Histone Modifications in Human T Cells,”Proc. Natl. Acad. Sci. USA 103:15782-15787, 2006).

Regarding the H3K27me3 histone modification, FIG. 7C is a grey-scale illustration of the heat map generated for the H3K27me3 histone modification landscape relative to gene ends in hair (H) cells (−1 kb to +1 kb relative to transcription start and end sites). Similarly, FIG. 8B is a grey-scale illustration of the heat map generated for the H3K4me3 histone modification landscape relative to gene ends in non-hair (NH) cells (−1 kb to +1 kb relative to transcription start and end sites). The area of most intense “yellow” representation, indicating positive log 2 ratios for H3K27me3, is indicated. In contrast to the results for H3K4me3 described above, H3K27me3 is generally excluded from the most highly expressed genes, is found in promoters of genes with mid-level expression, and covers the entire body of genes with the lowest expression levels in both cell types (see area indicated as “yellow” in FIG. 7C and FIG. 8B). The area indicated as “blue” represents data points of negative log 2 ratio for H3K27me3.

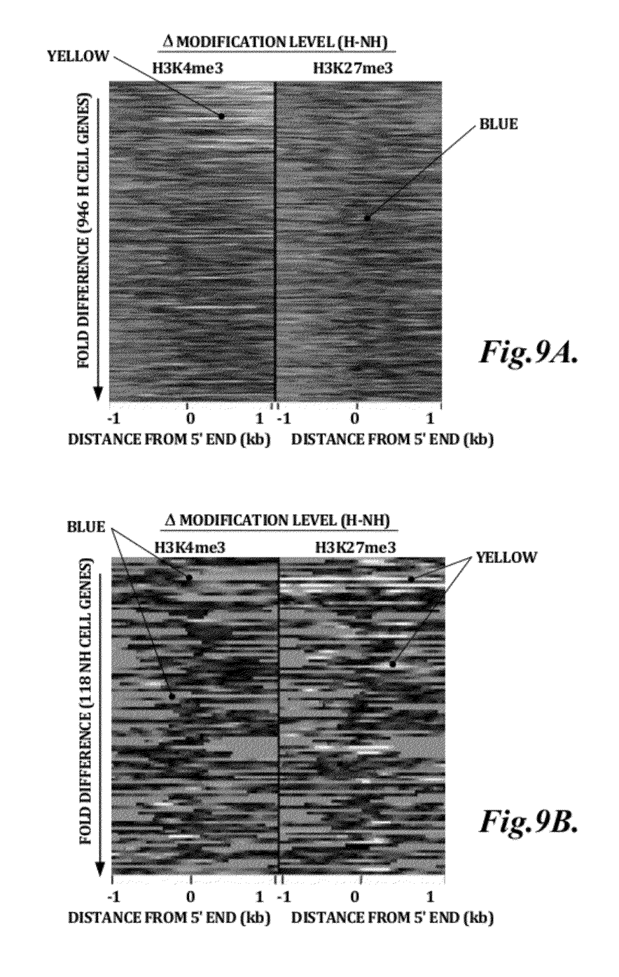

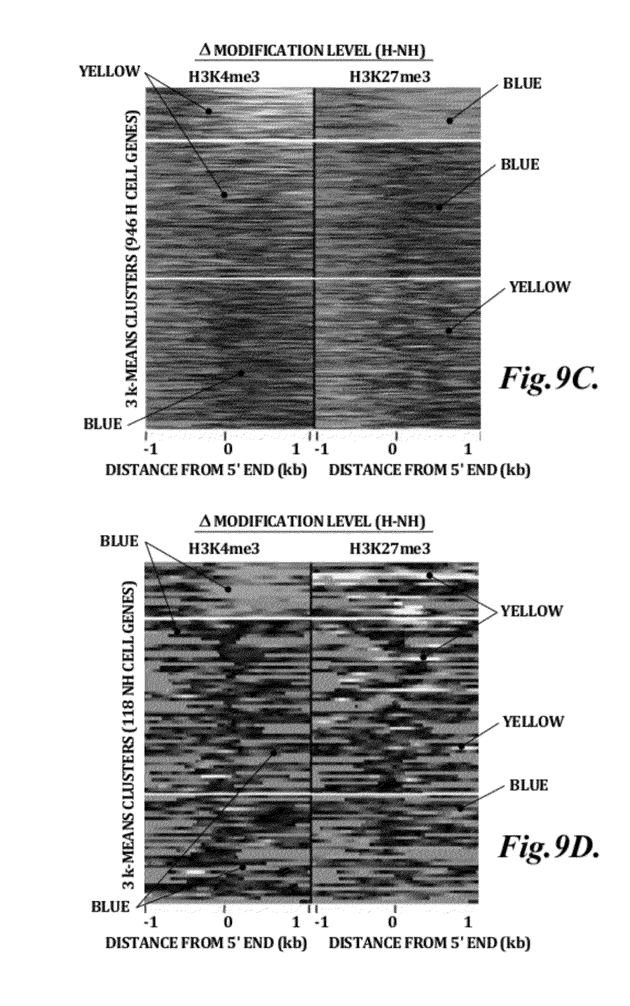

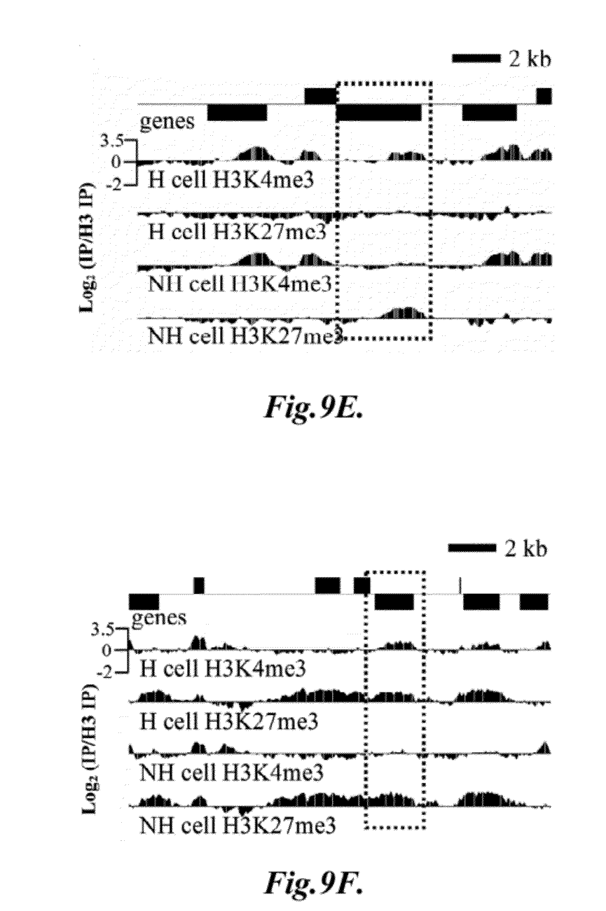

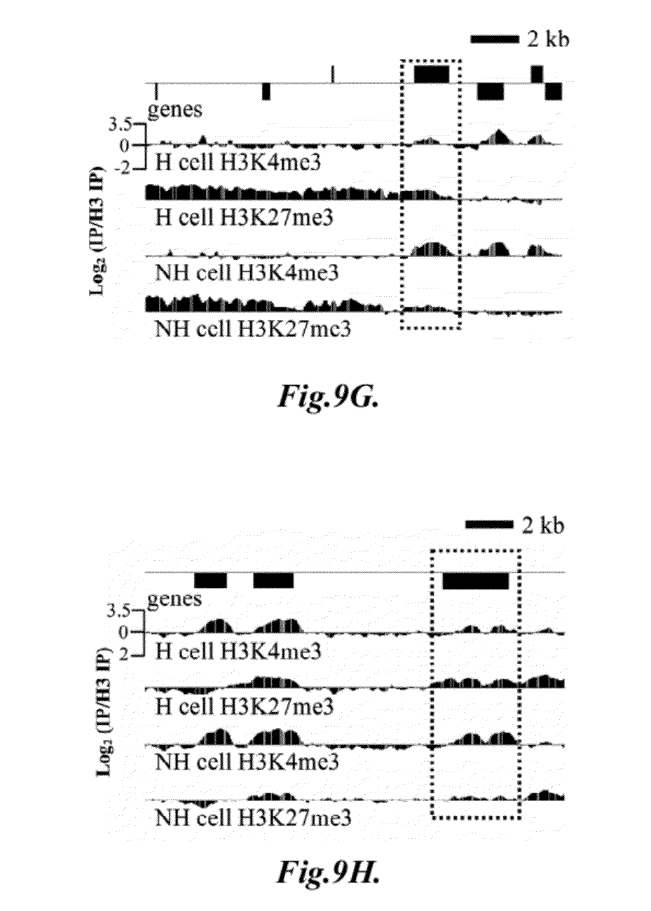

FIG. 9A is a grey-scale illustration of a heat map showing H3K4me3 and H3K27me3 differences between hair (H) and non-hair (NH) cell types (H cell profile minus NH cell profile) around the 5′ end of genes (−1 kb to +1 kb from transcription start site) for the 946 H cell-enriched genes. The H cell-enriched genes were ranked according to fold difference in expression level between H and NH cells, with the highest fold difference level at the top of the heat map to the lowest fold difference level at the bottom of the heat map. The heat map densities indicated as “yellow” are more highly occupied by the epitope (histone modification) than over the annotated genome as a whole, whereas those densities indicated as “blue” are less occupied by the epitope than the genome as a whole. The predominance of “yellow” signal in the H3K4me3 column indicates higher H3K4me3 modification levels in highly expressed H-enriched genes in the H cells. Conversely, the predominance of “blue” signal in the H3K27me3 column indicates higher H3K27me3 modification levels in highly expressed H-enriched genes in the NH cells. However, many genes showed an overlap of H3K4me3 and H3K27me3 (FIG. 9A and TABLE 4), as has been described in mammalian stem cell lines and isolated primary cells (Bernstein et al., 2006; Roh et al., 2006).

TABLE 4
Characterization of Chromatin Profiles in Each Cell Type.

| H3 Histone | | | Non Hair |
|---|---|---|---|
| Modification | | Hair Cell | cell |
| H3K4me3 | Total number of peaks | 14443 | 15054 |
| | Avg. peak length +/− SD | 1527 +/− 1066 | 1494 +/− 1030 |
| | (bp) | | |
| | Number genes containing | 16930 | 17600 |
| | a peak (at least 400 bp | | |
| | overlap with gene) | | |
| H3K27me3 | Total number of peaks | 6416 | 6496 |
| | Avg. peak length +/− SD | 3608 +/− 3848 | 3600 +/− 3885 |
| | (bp) | | |
| | Number genes containing | 7352 | 7389 |
| | a peak (at least 400 bp | | |
| | overlap with gene) | | |
| Regions | Total number of overlap | 2111 | 2260 |
| with | domains (at least 500 bp | | |
| H3K4me3 | overlap between | | |
| and | H3K4me3 and | | |
| H3K27me3 | H3K27me3 peaks) | | |
| | Avg. overlap length +/− | 1038 +/− 565 | 1059 +/− 590 |
| | SD (bp) | | |
| | Number genes containing | 1937 | 2090 |
| | a H3K4me3/H3K27me3 | | |
| | domain (at least 400 bp | | |
| | overlap with gene) | | |

Peaks were identified in the ChIP data using the PeakPicker program within the CARPET suite of tiling array analysis tools (Cesaroni, M., et al., “CARPET: A Web-Based Package for the Analysis of Chip-Chip and Expression Tiling Data,” Bioinformatics 24: 2918-2920, 2008) with the following parameter settings: window size of 1000 bp, minimum log p value of 3.5, maximum distance between two probes of 200 bp, and a minimum distance between peaks of 500 bp. Peaks were assigned to genes using the TAIR 8 genome annotation and at least 400 bp overlap with a gene body was required for each modification. Transposons and pseudogenes were excluded from the analysis.

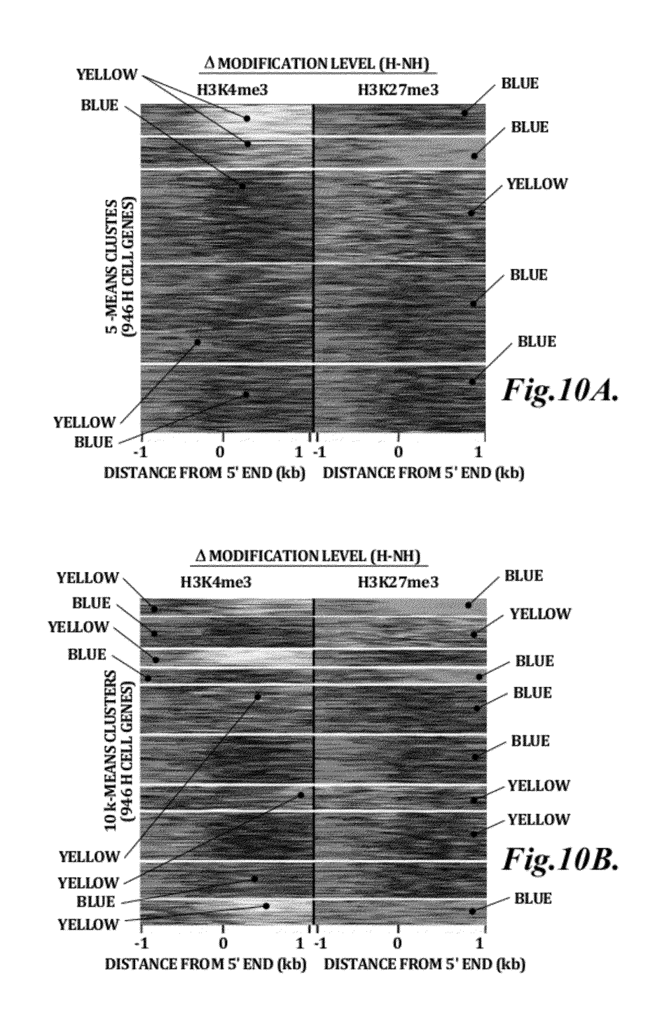

In order to determine whether differences in the H3K4me3 and H3K27me3 profiles between cell types might correspond to genes that were preferentially expressed in each cell type, each non-hair (NH) cell profile was subtracted from the corresponding hair (H) cell profile. Heat maps were generated from the subtracted profiles for each modification by aligning them at the 5′ ends of genes and ranking each list of cell type-enriched genes based on the fold difference in expression level between the cell types, from largest to smallest. H3K4me3 is enriched at active genes and depleted from inactive genes, and the heat maps show high H3K4me3 levels in coding regions of active relative to inactive genes. Conversely, H3K27me3 is enriched at inactive genes and depleted from active genes, and the heat maps show low levels of H3K27me3 in the coding regions of active relative to inactive genes. Cell type-enriched genes with the largest fold differences between cell types often showed both higher H3K4me3 and lower H3K27me3 levels in the cell type where they were preferentially expressed (FIGS. 9A-B). k-means clustering of the same heat maps into 3 clusters showed that many genes enriched in a given cell type show this pattern, and this was particularly evident in hair cells. However, many of the cell-type enriched genes show no distinct chromatin differences between cell types, while others show subtle chromatin differences in the opposite direction. Comparing differences between hair and non-hair cells, it is observed that where H3K4me3 increases, H3K27me3 decreases, and vice-versa, which is expected if chromatin features conform with expression differences between the cell types. This indicates that a change in the balance of H3K4me3 and H3K27me3 identifies some, but not all, genes with preferential expression in a given cell type (FIGS. 9C-D). Using larger numbers of clusters showed that the class of genes with higher H3K4me3 and lower H3K27 remained a coherent group (FIGS. 10A and B). This higher-level clustering also revealed that there were genes on which only H3K4me3 was higher or only H3K27me3 was lower in the cell type where the gene was preferentially expressed (FIGS. 10A and B).

FIGS. 9E-H graphically illustrates the euchromatic chromatin landscape of the histone H3 modifications, H3K4me3 and H3K27me3, in hair (H) and non-hair (NH) cells on the H cell-enriched genes At5g70450 and At3g49960, and on the NH cell enriched genes At1g66800 and At5g42591, respectively. As shown in FIGS. 9E-H, examination of the H3K4me3 and H3K27me3 chromatin landscapes over individual hair or non-hair cell-enriched genes showed that these genes often display differences in both modifications.

Discussion:

The preferential expression of a gene in one cell type often correlates with major differences between the cell types in the trimethylation of histone H3 at lysines 4 and 27, demonstrating that chromatin differences exist between hair and non-hair cells, which can be readily monitored in nuclei purified using this method. The INTACT method is simple, fast, and should be widely applicable.

Profiling of two histone modifications, H3K4me3 and H3K27me3, in hair and non-hair cell nuclei, showed that it is possible to produce robust and highly reproducible ChIP data from the number of nuclei obtained using INTACT. Both of these histone modifications showed distributions similar to those recently described in Arabidopsis (Oh, S., et al., “Genic and Global Functions for Paf1C in Chromatin Modification and Gene Expression in Arabidopsis,” PLoS Genet. 4:e1000077, 2008; Zhang, X., et al., “Genome-Wide Analysis of Mono-, di- and Trimethylation of Histone H3 Lysine 4 in Arabidopsis thaliana,” Genome Biol. 10:R62, 2009; Zhang, X., et al., “Whole-Genome Analysis of Histone H3 Lysine 27 Trimethylation in Arabidopsis,” PLoS Biol 5:e129, 2007). In addition, it is demonstrated that in each cell type the level of H3K4me3 within a gene decreases with decreasing expression level and the H3K27me3 modification increases, decreasing expression (FIGS. 7 and 8), as expected. These correlations between expression levels and well-studied chromatin modifications serve as an independent confirmation of the accuracy of the present gene expression profiles for each cell type.

Previous profiling of H3K4me3 and H3K27me3 in Arabidopsis suggested that many plant genes have overlapping regions of H3K4me3 and H3K27me3, as observed in mammalian cells, but because whole plant tissues were used in these experiments it was not clear whether these overlaps were in individual cells or were an artifact of the amalgamation of signals from multiple cell types (Oh et al., 2008; Zhang et al., 2009; Zhang et al., 2007). By profiling chromatin landscapes at cell type-resolution we are able to show that these modifications do indeed coexist in the same cell type, as has been observed in mammalian cells (Bernstein, B. E., et al., “A Bivalent Chromatin Structure Marks Key Developmental Genes in Embryonic Stem Cells,” Cell 125:315-326, 2006; Roh et al., 2006).

A comparison of each histone modification profile by subtraction of the non-hair cell profile from that of the hair cell showed that the largest expression differences between cell types often corresponded to an increase in H3K4me3 and a decrease in H3K27me3 in the cell type showing preferential expression of a given gene. This suggests that a balance between the activities of Trithorax group protein-mediated H3K4 trimethylation and Polycomb group protein-mediated trimethylation of H3K27 is involved in establishing cell type-specific expression. However, many differentially expressed genes showed little difference in histone modification levels between cell types over cell type-enriched genes, indicating that there are mechanisms for generating cell type-specific expression that are unrelated to the H3K4me3/H3K27me3 balance.

Conclusion:

These results demonstrate that the INTACT method results in high yield and purity of nuclei cell-specific nuclei populations that are suitable for robust and highly reproducible chromatin analysis.

### Example 4

This Example describes the application of the INTACT method to produce and isolate in vivo biotinylated nuclei in germline cells of Caenorhabditis elegans.

Rationale:

As proof of applicability of the INTACT method to non-plant eukaryote organisms, transgenes for a nuclear tagging fusion protein and biotin ligase were co-expressed in germline cells of C. elegans and the resulting nuclei were isolated.

Methods:

Constructs and Transgenic Nematodes for INTACT

Vectors encoding a nuclear tagging fusion (NTF) protein and a biotin ligase were constructed as illustrated schematically in FIGS. 11A and B, respectively. In the embodiment of the INTACT vector used in this example, the encoded nuclear envelope tagging (NTF) fusion polypeptide comprised of the NPP-9 domain, the C. elegans homolog of mammalian Nup358/RanBP2, at the N-terminus of the translated polypeptide to serve as the nuclear targeting region. The amino acid sequence of the NPP-9 domain is set forth herein as SEQ ID NO:23. The nucleic acid 14 encoding the NPP-9 domain was disposed at the 5′ end of the vector 10 encoding the NTF protein. A nucleotide sequence including introns that encodes the NPP-9 domain is set forth herein as SEQ ID NO:22, and corresponds to the sequence used as part of the vector 10 encoding the full length NTF protein. The encoded NPP-9 domain was followed by the mCherry domain to serve as the visualization tag. The polypeptide sequence for the mCherry domain 22 is set forth herein as SEQ ID NO:17, and is encoded in the vector 10 by a nucleic acid sequence 22 set forth herein as SEQ ID NO:16. The encoded mCherry domain in the NTF protein was followed by the affinity reagent binding region, specifically, a biotin ligase recognition peptide (BLRP) comprising an amino acid sequence set forth herein as SEQ ID NO:88, encoded in the vector 10 by the nuclei acid sequence 16 set forth herein as SEQ ID NO:87. Finally, the BLRP domain was followed by a 3X FLAG epitope tag domain, comprising a full amino acid sequence set forth herein as SEQ ID NO:27. The full 3X FLAG epitope tag was encoded in the vector 10 by a nucleic acid sequence set forth herein as SEQ ID NO:26.

Ultimately, the full length amino acid sequence of the NTF polypeptide is set forth herein as SEQ ID NO:25, encoded in the vector 10 by the nucleic acid sequence set forth as SEQ ID NO:24. The sequence encoding the fusion protein was operatively linked to the pie-1 promoter 12, which is specific for C. elegans germline cells. Specifically, the pie-1 promoter 12 (SEQ ID NO:20) was disposed in the vector at the 5′ end of the NTF encoding sequence, and the pie-1 3′UTR 12a (SEQ ID NO:21) was disposed in the vector at the 3′ end of the NTF encoding sequence.

In the embodiment illustrated in this example, a separate expression vector comprising a second expression cassette 11 with the gene 24 encoding E. coli biotin ligase (BirA), previously described herein in EXAMPLE 1 (amino acid sequence set forth herein as SEQ ID NO:12, encoded by the nuclei acid sequence set forth herein as SEQ ID NO:11). The nucleotide sequence 24 encoding the BirA ligase was followed on its 3′ end with an optional sequence 22 encoding a visualization tag, specifically GFP. This provides a simple mechanism to confirm expression of the BirA ligase. As illustrated in FIG. 11B, the BirA gene was operatively linked to the H3.3 histone promoter, which is a constitutive promoter 13 in C. elegans cells. As illustrated, the BirA gene 24 was flanked at the 5′ end by the H3.3 histone promoter (set forth herein as SEQ ID NO:18), and the optional sequence 22 encoding the GFP was flanked at the 3′ end by the H3.3 3′UTR 13a (set forth herein as SEQ ID NO:19).

Each of the constructs illustrated in FIGS. 11A and B were co-transformed into C. elegans. The nematode worms were cultured under standard conditions (see e.g., Brenner, S., “The Genetics of Caenorhabditis elegans,” Genetics 77:71-94, 1974). The constructs were each inserted into the C. elegans genome using microparticle bombardment of adult worms, as described in Berezikov (Berezikov, E., et al., “Homologous Gene Targeting in Caenorhabditis elegans by Biolistic Transformation,” Nucleic Acids Res. 32:e40, 2004). Strains with stably integrated transgenes were crossed to combine the BirA and the NPP-9 transgenes.

Purification of Biotinylated Nuclei/Immunoprecipitation and Western Blotting

For purification of nuclei, whole worms were frozen in liquid nitrogen, ground into a fine powder, and cells were lysed as previously described in EXAMPLE 1 in reference to plant cells.

Whole cell extracts were prepared, and nuclei were isolated as described in EXAMPLE 1. Fusion protein was immunoprecipitated and electrophoresed as previously described in EXAMPLE 1 in reference to plant cells, except that an mCherry fluorescence was used to detect the presence of tagged nuclei isolated using the miniMACS™ separator magnet instead of GFP fluorescence, also described in EXAMPLE 1.

Results:

The INTACT method was demonstrated herein, in EXAMPLES 1-3, to be effective in causing the biotinylation of nuclei for two plant cell types, facilitating their purification and robust genomic analyses. As described in this example, the INTACT method resulted in transgenically expressed NTF and biotinylated protein being localized in the nuclear envelope of C. elegans cell types of interest. In the embodiment presented in this example, a fusion protein comprised of a NPP-9 domain that served as a nuclear envelope targeting region, an mCherry domain that served as a visualization tag region, and a biotin ligase accepting site that served as the affinity reagent binding region. The nucleic acid encoding the NTF protein was expressed under the control of the pie-1 promoter and pie-1 3′UTR sequence, a promoter sequence specific for gene expression in C. elegans germline cells.

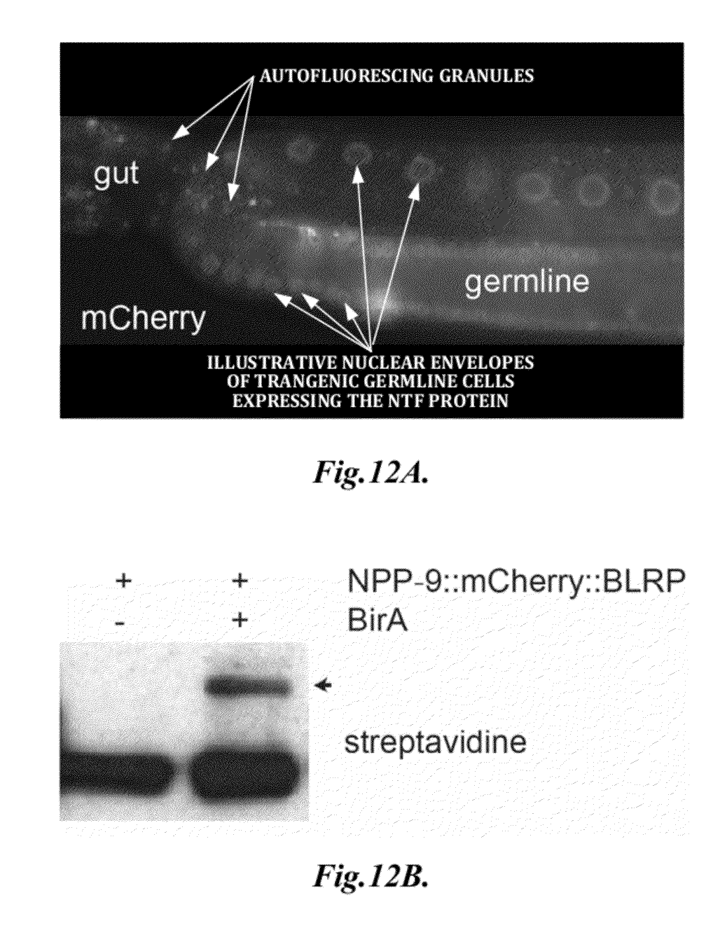

FIG. 12A is a fluorescence micrograph of a live C. elegans worm transgenic with NPP-9:mCherry:BLRP. As illustrated with mCherry fluorescence, the expressed NPP-9:mCherry:BLRP fusion protein localized in the nuclear envelopes of transgenic C. elegans germline cells. Illustrative tagged nuclear envelopes are indicated. Autofluorescence of the gut granulose is also visible, as indicated.

To determine whether transgenically expressed fusion proteins were biotinylated in vivo when co-expressed with biotin ligase (BirA), NTF protein was immunoprecipitated from C. elegans that did or did not also transgenically co-express biotin ligase BirA. The immunoprecipitated NTF protein was blotted and probed using streptavidin-HRP. Referring to FIG. 12B, biotinylated fusion protein of the expected size was detected only in worms that co-expressed the fusion protein and BirA, indicated by the arrow. In contrast, worms expressing only the fusion protein did not have biotinylated fusion protein. The lower bands on the blots represent endogenous biotinylated product.

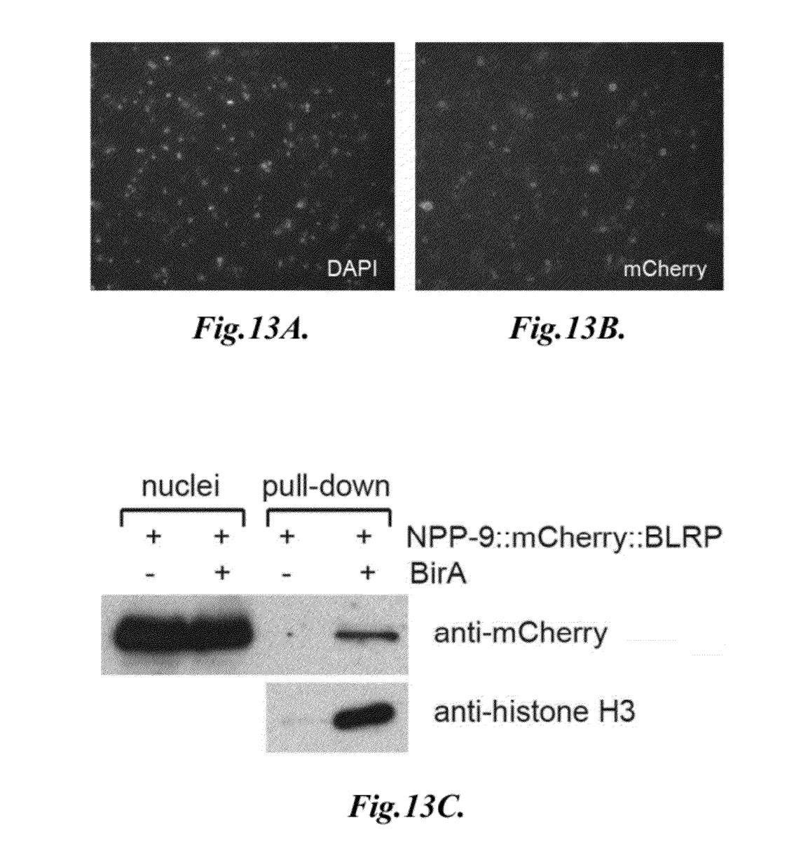

To determine whether the intact nuclei recovered from whole nuclei extractions retained fusion protein on their surface, cells from C. elegans transgenic for the NTF protein and BirA were lysed and intact nuclei were isolated, as described in EXAMPLE 1 in regard to plant cells. Recovered cells were stained with DAPI, and visualized for the presence of DNA and mCherry staining. FIG. 13A is a micrograph of DAPI stained total nuclei isolated from transgenic C. elegans with the NPP-9:mCherry: BLRP and BirA vectors. As illustrated, whole nuclei extracts stained with DAPI reveal all nuclei in the field of view that were recovered from the lysate. FIG. 13B is a fluorescent micrograph of the same total nuclei isolated from transgenic C. elegans with the NPP-9:mCherry:BLRP vector, as illustrated in FIG. 13A. As illustrated, a large fraction of the total nuclei population in the field of view fluoresce (relative to FIG. 13A), indicating the presence of the fusion protein on their surface. Therefore, intact nuclei recovered from whole nuclei extractions according to the INTACT method retain fusion protein on their surface.

To determine whether the intact nuclei isolated from transgenic C. elegans lysates were biotinylated (via the NTF polypeptide tag), immunoprecipitates were assessed from C. elegans expressing the fusion protein alone, or co-expressing the fusion protein and BirA. The precipitates were assessed before or after streptavidin “pull-down”, which was accomplished by incubation with streptavidin-coated Dynabead® and the application of a magnetic field, as described in EXAMPLE 1. FIG. 13C is a western blot of cells lysates from transgenic C. elegans cells, either expressing the NPP-9:mCherry:BLRP NTF protein only, or co-expressing the NPP-9:mCherry:BLRP NTF protein and biotin ligase (BirA) stained with anti-mCherry and anti-histone H3 antibodies. As illustrated, cells receiving the vector encoding the NTF protein produced detectable levels of fusion protein as illustrated with an anti-mCherry antibody, regardless of co-expression with BirA. In contrast, after application of the isolation technique utilizing an incubation period with streptavidin-coated Dynabeads® and a magnetic capture flow apparatus (described in EXAMPLE 1), only cells receiving both the fusion protein vector and BirA vector had detectable levels of fusion protein (see “pull down” column). Use of an anti-histone H3 antibody confirmed that the “pull down” technique recovered whole nuclei by virtue of detecting histone H3. The analysis also confirmed that the technique recovers nuclei only from cells that co-express the fusion protein and BirA, indicated by the lack of signal from cells expressing the fusion protein only.

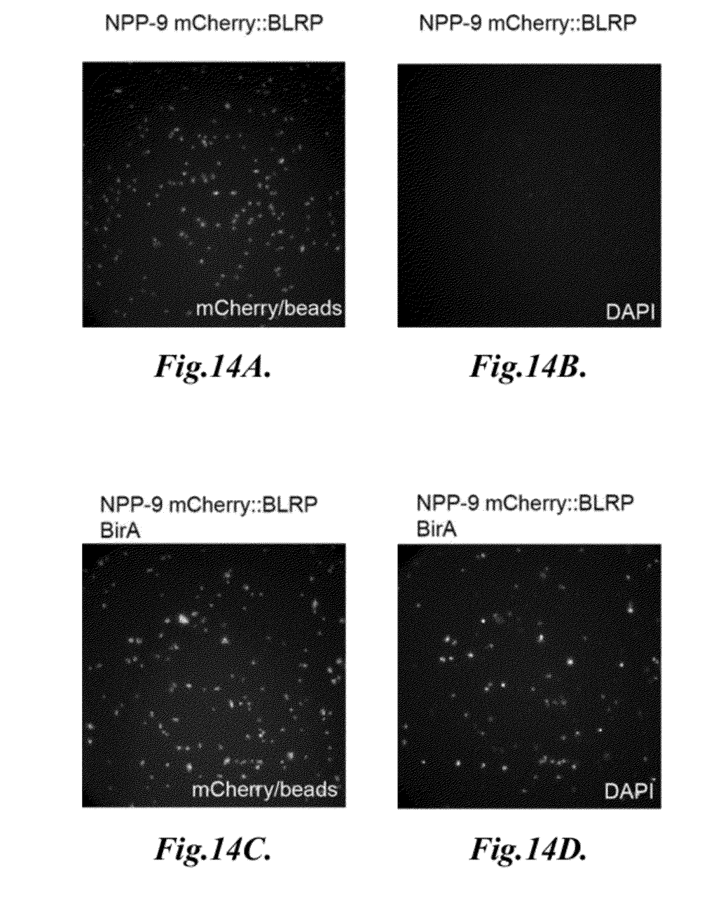

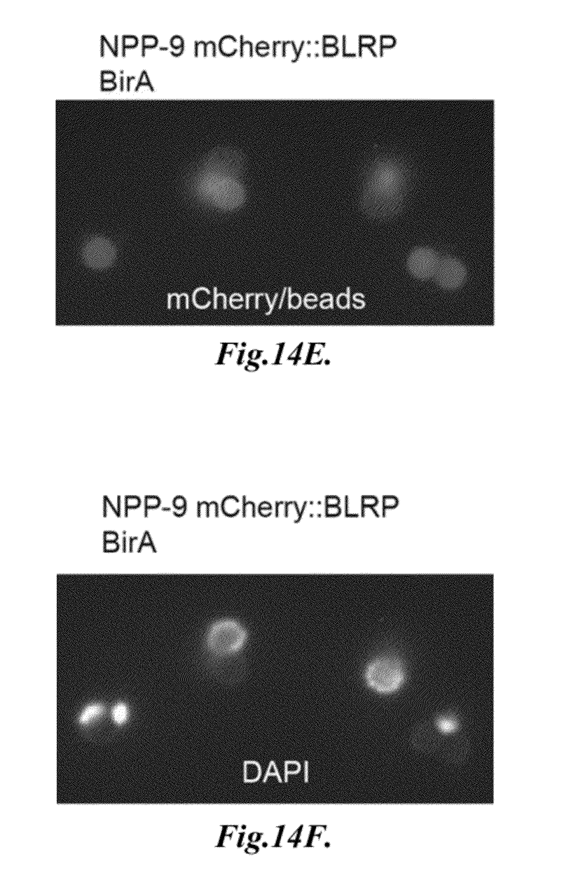

FIGS. 14A-F illustrate microscopic analyses of nuclei isolated using the magnetic capture flow apparatus (described above and in EXAMPLE 1 and illustrated in FIG. 1G). NTF protein bound to nuclei is not detected from cells that transgenically expressed the fusion protein but not BirA. In this regard, autofluorescence is visible from magnetic beads in the mCherry micrograph illustrated in FIG. 14A, whereas no nuclei are visible in the same isolate sample when viewed for DAPI staining, as illustrated in FIG. 14B. In contrast, fluorescent nuclei are visible as bright spots in the sample isolated from cells co-expressing the NTF protein and BirA (FIG. 14C). This presence of nuclei is confirmed because the DAPI foci indicate the presence of nuclei by virtue of staining of DNA in the nuclei (FIG. 14D). Thus, only nuclei were recovered from cells transgenically co-expressing the NTF protein and BirA were isolated by the INTACT method. Detailed DAPI and m-Cherry views of the isolated nuclei are illustrated in FIGS. 14E and F.

Discussion:

It is demonstrated herein that the NTF protein comprising a nuclear envelope targeting region and biotin accepting site can be selectively expressed in a cell type of interest (germline cells) in live C. elegans. By virtue of the nuclear envelope targeting region, the NTF protein can be incorporated into the nuclear envelope and is retained therein even after cell lysis and isolation of the nuclei. Furthermore, it is demonstrated herein that the nuclei tagged with the NTF are biotinylated when the cells also co-express BirA. Thus, the in vivo biotinylated nuclei can be easily isolated from a cell lysate with high yields and purity for relatively low cost and without highly technical equipment.

Conclusion:

These results demonstrate that the INTACT method, incorporating promoter and nuclear envelope targeting regions for the nematode, C. elegans, results in a high yield and purity of the cell type of interest. This confirms that the INTACT method is applicable to animal systems as well as plants.

### Example 5

This Example describes the application of the INTACT method to produce and isolate in vivo biotinylated nuclei in Drosophila melanogaster.

Rationale:

As additional proof of applicability of the INTACT method to non-plant eukaryote organisms, transgenes for a nuclear tagging fusion protein NTF and biotin ligase were co-expressed in D. melanogaster, and the resulting biotinylated nuclei were detected in the specific cell type of interest.

Methods:

Constructs and Transgenic Nematodes for INTACT

Vectors encoding a nuclear tagging fusion protein and a biotin ligase were constructed, and are illustrated schematically in FIGS. 15A and B, respectively. In this embodiment of the INTACT method, the vector 10 encoding the nuclear envelope tagging fusion (NTF) polypeptide contained two distinct affinity reagent binding regions 16, (i.e. a nucleic acid encoding the 3X FLAG epitope tag and BLRP). The nucleic acid sequence encoding the 3X FLAG epitope tag is set forth herein as SEQ ID NO:26, and is disposed at the 5′ end of the fusion gene construct 10. This resulted in an NTF protein with three tandem repeats of the FLAG epitope tag at the N terminus. The polypeptide sequence for the full 3X FLAG epitope tag is set forth herein as SEQ ID NO:27. In the vector construct 10, the FLAG-encoding sequence was followed at its 3′ end with the sequence encoding the biotin ligase recognition peptide (BLRP), as described previously in EXAMPLE 4 (the nucleic acid sequence set forth herein as SEQ ID NO:87, encoding a polypeptide domain with an amino acid sequence set forth herein as SEQ ID NO:88). In the vector construct 10, the BLRP-encoding sequence was followed at its 3′ end by a nucleotide sequence 22 encoding mCherry 22, set forth herein as SEQ ID NO:16 (encoding the amino acid sequence set forth as SEQ ID NO:17). The mCherry domain can serve as a visualization tag, in addition to an affinity reagent binding region, as described in EXAMPLE 4. In the vector construct 10, the sequence encoding mCherry was followed at its 3′ end with a sequence encoding Drosophila RanGap 14, set forth herein as SEQ ID NO:28, which includes non-coding intron sequences. The resulting amino acid sequence of the Drosophila RanGap is set forth herein as SEQ ID NO:29. It is notable that in this embodiment, the sequence of the first expression cassette encoding the NTF protein resulted in an NTF protein with the nuclear envelop targeting region (i.e., Drosophila RanGAP) at the C-terminus because this specific nuclear envelope targeting region embeds in the nuclear membrane in such a manner that exposes the N-terminus of the protein to the extra-nuclear space. The full length nucleic acid sequence encoding the NTF polypeptide is set forth herein as SEQ ID NO:30. The corresponding polypeptide sequence for the NTF polypeptide is set forth herein as SEQ ID NO:31.

The sequence encoding the fusion protein was operatively linked to the twist promoter 12, which is specific for somitic cells in D. melanogaster embryos. Specifically, the twist promoter was disposed in the vector at the 5′ end of the NTF encoding sequence. The sequence of the twist promoter is set forth herein as SEQ ID NO:32.

As described in EXAMPLES 1 and 4, the embodiment illustrated in this example incorporated a separate expression vector comprising a second expression cassette 11 containing the gene 24 encoding the E. coli biotin ligase (BirA) (amino acid sequence set forth herein as SEQ ID NO:12, encoded by the nuclei acid sequence set forth herein as SEQ ID NO:11). As illustrated in FIG. 15B, the BirA gene 12 was operatively linked to the twist promoter 12, which is the same somitic cell-specific promoter used in the vector encoding the NTF protein.

The nucleic acid sequence encoding the fusion protein, shown in FIG. 15A, was inserted into the twist-BirA Casper vector, shown in FIG. 15B. The plasmid containing both twist-BirA and twist-FLAG-blrp-mCherry-RanGAP was then transformed into D. melanogaster flies using a microinjection service (Genetic Services, Inc., Cambridge, Mass.). Live embryos transgenic for NTF protein and BirA ligase were visualized for mCherry fluorescence using standard fluorescence and fluorescence confocal microscopy.

In an alternative approach, the vectors shown in FIG. 15A (encoding the fusion protein, and 15B (encoding BirA), could be used to co-transform flies; or each vector (15A and 15B) could be used to transform flies to generate lines that could then be crossed to create a double transgenic line.

Nuclei were isolated from embryos transgenic for NTF protein and BirA. Briefly, Drosophila whole embryos were dechorionated with bleach. Nuclear extracts were made by disrupting the cells' plasma membranes in nuclear buffer using a douncing homogenizer. Nuclei were washed, and collected by centrifugation prior to incubation with beads, as described in EXAMPLE 1. Isolated nuclei samples were treated with DAPI stain, incubated with anti-FLAG antibody, and incubated with streptavidin conjugated to a fluorescent tag. As is commonly known, these treatments can be applied simultaneously or in a series, commonly in the order listed herein, under standard conditions known for fluorescent antibodies. Staining/fluorescence was visualized using standard fluorescence microscopy techniques targeting each of the treatments applied. For example, visualization of fluorescence can be performed using any of several different fluorescent microscopes, including Nikon E800, Zeiss LSM Confocal, and Deltavision.

Results:

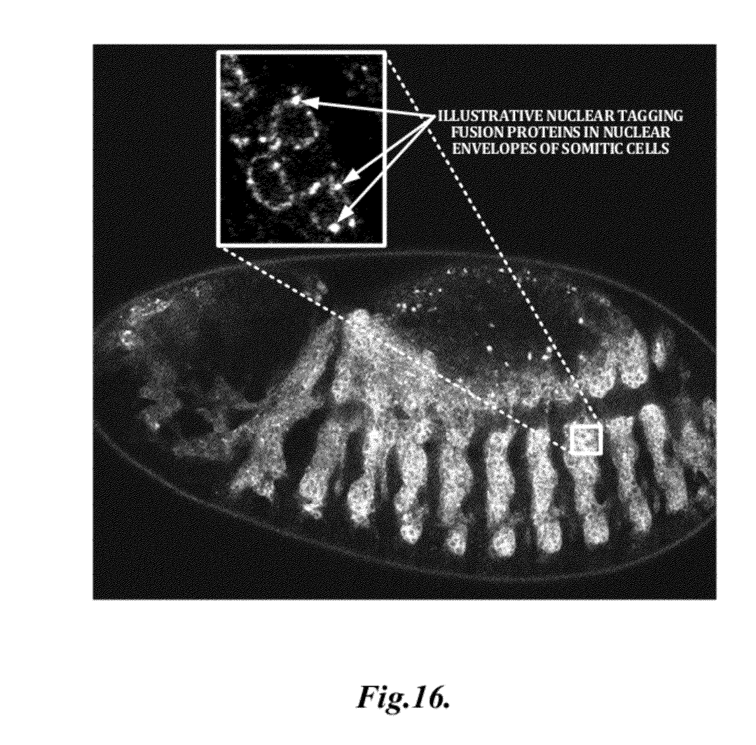

As demonstrated in this example, the INTACT method successfully resulted in the co-expression of transgenic NTF protein in somitic cells of the D. melanogaster embryos under the control of the Drosophila twist promoter. FIG. 16 is a fluorescence micrograph of a D. melanogaster embryo transgenic for both NTF protein and BirA. The micrograph shows mCherry fluorescence from the NTF protein in the somitic cells of the embryo. The inset illustrates the localization of the NTF protein at the nuclear envelope of the somitic cells. This demonstrates the ability to tag the nuclear envelope of D. melanogaster somitic cells by transgenically co-expressing therein an NTF protein comprising a nuclear envelope targeting protein (RanGAP) and one or more affinity reagent binding regions (for example, a FLAG epitope tag, a BLRP, and/or the mCherry domain).

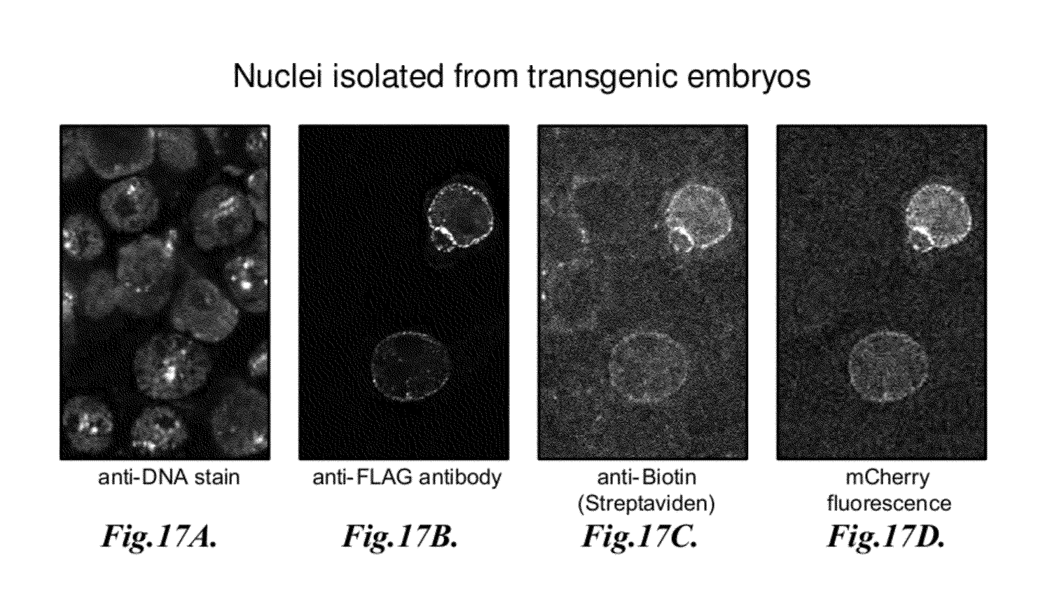

In order to verify the localization and retention of the NTF protein in the nuclear envelopes of the D. melanogaster somitic cells, the nucleus isolates were visualized for the presence of DNA, the FLAG epitope, biotinylation, and mCherry fluorescence. FIG. 17A is a DAPI-stained micrograph of nuclei isolated from transgenic D. melanogaster embryos expressing both NTF protein and BirA from the twist promoter. DNA in the nuclei is indicated, confirming the isolation of intact nuclei from the cell extracts. FIG. 17B is a fluorescence micrograph of the same nuclei illustrated in FIG. 17A, after incubation with fluorescing anti-FLAG antibodies. The fluorescence signal indicates that two of the nuclei in the field of view are tagged with the FLAG epitope at the outer surface, as would be expected with an embedded NTF protein according to the INTACT method. FIG. 17C is a fluorescence micrograph of the same nuclei illustrated in FIG. 17A, after incubation with fluorescence-tagged streptavidin. The same pattern of fluorescence is observed as in FIG. 17B, indicating that the same nuclei are tagged with the NTF protein. Furthermore, the fact that incubation with streptavidin resulted in signal indicates that the NTF protein embedded in the nuclei were biotinylated. This confirms that the cell successfully expressed functional BirA ligase from the vector. Finally, FIG. 17D is a fluorescence micrograph of the same nuclei illustrated in FIG. 17A, showing the mCherry fluorescence of the NTF protein-tagged nuclei. Combined, these results confirmed the successful tagging of the nuclear envelope with the NTF protein, successful biotinylation of the NTF protein by the transgenic BirA ligase, and the subsequent ability to visualize the tagged nuclei through the detection of any of a number of a plurality of visualization tags incorporated into the NTF protein.

Conclusion:

These results demonstrate that the INTACT method, incorporating promoter and nuclear envelope targeting regions for D. melanogaster, results in the cell-specific tagging of nuclei. This provides further confirmation that the INTACT method is applicable to a variety of animal systems, as well as plants.

### Example 6

This Example describes the application of a nuclear immunopurification method to rapidly and efficiently purify in vitro- and in vivo-labeled nuclei in mice.

In this example, the nuclear tagging protein is an integral membrane protein fused to a fluorescent protein module that allows tagged nuclei to be visualized at any point during the isolation procedure. The tagging protein is easily diversified by the addition of standard affinity reagent binding region tags, thus allowing the user to label multiple genetically distinct types of nuclei in one experiment. The data described herein establishes the applicability of the INTACT procedure to enable the isolation of cell-type specific nuclei in mammals. Thus, the INTACT method simplifies the generation of cell-type specific genomic, biochemical, and cell biological data across eukaryotic cells of all lineages.

Rationale:

Many biological problems involve the study of functionally relevant cell types or cellular states. Though there are numerous schemes for defining cellular states, it is widely accepted that cell types are determined by the expression of cell-type specific combinations of proteins, RNAs, and epigenetic modifications of genomes (Arendt, D., “The Evolution of Cell Types in Animals: Emerging Principles From Molecular Studies,” Nat. Rev. Genet. 9:868-882, 2008; Christodoulou, F., et al., “Ancient Animal MicroRNAs and the Evolution of Tissue Identity,” Nature 463:1084-1088, 2010; Hemberger, M., et al., “Epigenetic Dynamics of Stem Cells and Cell Lineage Commitment: Digging Waddington's Canal,” Nat. Rev. Mol. Cell. Biol. 10:526-37, 2009; Zernicka-Goetz, M., et al., “Making a Firm Decision: Multifaceted Regulation of Cell Fate in the Early Mouse Embryo,” Nature Rev. Genet. 10:467-477, 2009). All of these factors can now be studied with high-throughput genomic and proteomic approaches that leverage the power of fully sequenced genomes.

Despite the ever-expanding array of techniques that can be used to analyze genomes, transcriptomes and proteomes, many of these methods are biochemical approaches that require millions of cells to obtain a robust signal. As a result, these genome-scale assays are most easily applied to either homogeneous populations of easily grown tissue culture cells or highly heterogeneous mixtures of cells obtained from whole tissues. A major challenge for the field is the development of techniques for the isolation of specific cell types from heterogeneous tissues or mixtures. The development of in situ measurement technologies solves this problem for some types of measurements (Levsky, J. M., et al., “Single-Cell Gene Expression Profiling,” Science 297:836-840, 2002). Other solutions include FACS sorting of heterogeneous populations of cells or the purification of proteins and their binding partners in a cell type specific manner through the use of various tagging approaches (Shilo, Y. and R. Aebersold., “Quantitative Proteome Analysis Using Isotope-Coded Affinity Tags and Mass Spectrometry,” Nat. Protoc. 1:139-145, 2006; Morin X., et al., “A Protein Trap Strategy to Detect GFP-Tagged Proteins Expressed From Their Endogenous Loci in Drosophila,” Proc. Natl. Acad. Sci. 98:15050-15055, 2001; Clyne P. J., et al., “Green Fluorescent Protein Tagging Drosophila Proteins at Their Native Genomic Loci With Small P Elements,” Genetics 165:1433-1441, 2003; Quñones-Coello A. T., et al., “Exploring Strategies for Protein Trapping in Drosophila,” Genetics 175:1089-1104, 2007; Buszczak M., et al., “The Carnegie Protein Trap Library: a Versatile Tool for Drosophila Developmental Studies,” Genetics 175:1505-1531, 2007; Huh W., et al., “Global Analysis of Protein Localization in Budding Yeast,” Nature 425:686-691, 2003).

A method for the isolation of intact nuclei from a specific cell type is desirable for many reasons. For example, the chromatin of isolated nuclei maintains much of its structure even when the outer cellular membrane is destroyed and the details of this structure can be probed by a variety of enzymatic manipulations. Examples include the classical nuclease mapping methods that have been used for many years as a means to position transcriptional enhancers, promoters and other important genomic structures (Enver, T., et al., “Simian Virus 40-Mediated C is Induction of the Xenopus Beta-Globin DNase I Hypersensitive Site,” Nature 318:680-3, 1985; Richard-Foy, H. and G. L. Hager, “Sequence-Specific Positioning of Nucleosomes Over the Steroid-Inducible MMTV Promoter,” EMBO J. 6:2321-2328, 1987; Weintraub, H., and M. Groudine, “Chromosomal Subunits in Active Genes Have an Altered Conformation,” Science 193:848-856, 1976; Wu C., “The 5.′ Ends of Drosophila Heat Shock Genes in Chromatin Are Hypersensitive to DNase I,” Nature 286:854-860, 1980). These techniques can be successfully expanded to whole genome resolution as a result of fully sequenced genomes and high-throughput analytical technologies, such as DNA microarrays and single molecule sequencing (Barski, A, et al., “High-Resolution Profiling of Histone Methylations in the Human Genome,” Cell 129:823-837, 2007; Bernstein B. E., et al., “Genomic Maps and Comparative Analysis of Histone Modifications in Human and Mouse,” Cell 120:169-181, 2005; Boyle A. P., et al., “High-Resolution Mapping and Characterization of Open Chromatin Across the Genome,” Cell 132:311.-322, 2008; Core, L. J., et al., “Nascent RNA Sequencing Reveals Widespread Pausing and Divergent Initiation at Human Promoters,” Science 322:1845-1848, 2008; Crawford, G. E., et al., “DNase-chip: A High-Resolution Method to Identify DNase I Hypersensitive Sites Using Tiled Microarrays,” Nat. Methods 3:503-509, 2006; Heintzman N. D., et al., “Distinct and Predictive Chromatin Signatures of Transcriptional Promoters and Enhancers in the Human Genome,” Nat. Genet. 39:311.-318, 2007; Henikoff, S., et al., “Genome-Wide Profiling of Salt Fractions Maps Physical Properties of Chromatin,” Genome Res. 19:460-469, 2008; Ren B., et al., “Genome-Wide Location and Function of DNA Binding Proteins,” Science 290:2306-9, 2000; Sabo, P. J., et al., “Genome-Scale Mapping of DNase I Sensitivity In Vivo Using Tiling DNA Microarrays,” Nat. Methods 3:511-518, 2006). Second, some structurally complex tissues or cell types are difficult to isolate with current technology. For example, structurally complex neurons are difficult to isolate without damaging the outer membrane. This makes FACS sorting of whole cells a challenge. The ability to isolate neuron-specific nuclei would simply efforts to study specific neuronal sub-types.

As described in EXAMPLES 1-5, the INTACT method was successfully applied to isolate cell-type specific nuclei in plants (A. thaliana; EXAMPLES 1-3), nematodes (C. elegans; EXAMPLE 4), and insects (D. melanogaster; EXAMPLE 5). This example further demonstrates that the INTACT method can be applied to mammalian cells to isolate cell-type specific nuclei. Specifically, this example describes methods and constructs that take advantage of the relative stability of isolated nuclei and permits isolation of the organelle from a specific homogeneous cell type. In the described nucleus immunopurification method, purified populations of genetically tagged nuclei are isolated on magnetic beads. To perform the immunopurification, a genetically encoded tag was developed that is positioned on the outside of the nucleus. The tag is a fusion protein where either GFP or tdTomato is fused to the nuclear integral membrane proteins Sun-1 or Nesprin-3 (Crisp M., et al., “Coupling of the Nucleus and Cytoplasm: Role of the LINC Complex,” J. Cell Biol. 172:41-53, 2006; Wilhelmsen, K., et al., “Nesprin-3, A Novel Outer Nuclear Membrane Protein, Associates With the Cytoskeletal Linker Protein Plectin,” J. Cell Biol. 171:799-810, 2005; Haque, F., et al., “SUN1 Interacts With Nuclear Laminin a and Cytoplasmic Nesprins to Provide a Physical Connection Between the Nuclear Lamina and the Cytoskeleton,” Mol. Cell. Biol. 26:3738-3751, 2006). Thus, nuclei can be tracked through the entire procedure, the integrity of the chromatin is preserved throughout the isolation process, multiple distinct classes of nuclei can be isolated in one experiment, and the method is simple to execute. As described supra, the only requirements of the broad applicability of the technique are 1) a cell type that will accept a transgene and for which a nuclear envelop targeting sequence is known, and 2) a promoter that can drive the expression of the nuclear tagging protein in the cell population of interest.

Methods:

Antibodies

GFP (Invitrogen A11122), MYC (Abcam ab9106), FLAG (Sigma F7425), HSV (Sigma H6030), VSV-G (Sigma V4888), HA (Abcam ab71113), AU1 (Abcam ab3401), and V5 (Sigma V8137).

DNA Constructs

A polynucleic acid encoding a synthetic polypeptide linker with the sequence LAAASGGGGSGGGGSLAAASEFSAAALSGGGGSGGGGSAAAL (SEQ ID NO:89), was inserted into the Nesprin-3 reading frame between amino acids 907 and 908 of the unmodified amino acid sequence. The unmodified amino acid sequence of Nesprin-3 has the Genbank Accession No. NP_{—}001036164.1, incorporated herein by reference, and is set forth herein as SEQ ID NO:91. The unmodified Nesprin-3 polypeptide is encoded by a nucleic acid that has the Genbank Accession No. NM_{—}001042699.1, incorporated by reference, and is set forth herein as SEQ ID NO:90. The same cassette was placed between amino acid 913 and the stop codon of Sun-1 polypeptide sequence. The unmodified Sun-1 amino acid sequence has the Genbank Accession No. NP_{—}077771.1, incorporated herein by reference, and is set forth herein as SEQ ID NO:93. The unmodified Sun-1 polypeptide is encoded by a nucleic acid that has the Genbank Accession No. NM_{—}024451.1, incorporated herein by reference, and is set forth herein as SEQ ID NO:92. A polynucleic acid encoding two copies of the super-folder GFP variant was then cloned into the centrally located EcoRI site of the linker (corresponding to the amino acids EF in the linker, underlined in the recitation above). The super-folder GFP variant is described in (Pedelacq, J-D., et al., “Engineering and Characterization of a Superfolder Green Fluorescent Protein,” Nat. Biotech. 24:79-88, 2005), which is expressly incorporated herein by reference in it entirety. The Sun-tdTomato constructs used the same linker strategy except that the incoming fluorescent protein carried a restriction site at its 3′ end that allowed the addition of various C-terminal epitope tags. Epitope tags were multimerized as follows: 3XMYC, 4×HA, 3X FLAG, 3XVSVg, 2XV5, 3X HSV, and 4×AU1, the nucleic acid and amino acid sequences of which are standard and well-known in the art.

Lentivirus Production

Lentivirus was produced in transfected 293/T17 cells using a third generation production scheme (Hanawa, H., et al., “Efficient Gene Transfer Into Rhesus Repopulating Hematopoietic Stem Cells Using a Simian Immunodeficiency Virus-Based Lentiviral Vector System,” Blood 103:4062-4069, 2004). After media harvest, the supernatant was concentrated first on a Vivacell 100 (Sartorius) concentrator followed by ultracentrifugation for 3 hours at 100,000 Xg. Viruses were untitered and as indicated in the text, Synapsin, Murine Stem Cell Virus (MSCV), and Cytomegalovirus (CMV) promoters were used to drive expression.

Assay Systems

Cos, Hela, 293, and N2a cells were transfected by the Fugene method (Roche). Transfected-detergent permeabilized cells were processed for immunohistochemistry using standard techniques. Rat primary hippocampal cultures were electroporated using the Amaxa-Nucleofector system (Lonza) at P0. Primary cultures were virus infected at P3-P4 using 1:100-1:200 dilutions of concentrated lentivirus. 500 nl of concentrated lentivirus was infused into the striatum of isoflurane anesthetized 8 week old C57BL/6 male mice using an Angle Two Stereotaxic system (myNeurol.ab) at −1.89ML, 0.50 AP, −4.00 DV (Bregma=0). Brains were processed using standard cryo-histological methods.

Magnetic Bead Preparation

The following conditions are per immunopurification reaction. 150 μls (4.5 mg) Protein G Dynabeads (Invitrogen 100.03D) were concentrated on a magnetic stand and resuspended in 600 μls of PBS/0.1% Tween20 containing 10-15 μg of purified antibody. 500 μls (5 mg) of Sheep Anti-Rabbit Dynabeads (Invitrogen 112.03D) were washed 3X in PBS/0.5% BSA and resuspended in 600 μls of PBS/0.5% BSA containing 10-30 μg of purified antibody. 250 μls (1.5 mg) of Biotin Binder Dynabeads (Invitrogen 110.47) were washed 3X in PBS/0.5% BSA and resuspended in 600 μls of PBS/0.5% BSA containing 5-15 μg of purified biotinylated antibody. The antibody was adsorbed to the bead for a minimum of 15 minutes at room temperature or indefinitely at 4° C. with constant agitation. After the completion of the binding reaction, the beads were washed 2-3X in the binding buffer minus antibody and resuspended in 500 μls of the immunopurification buffer.

Immunopurification of Nuclei

10⁶-10⁷ cells were swelled in 1 ml 10 mM β-Glycerophosphate pH 7, 2 mM MgCl₂, 1% Tween40 for 5 minutes on ice (Philpot, J. S, and J. E. Stanier, “The Choice of the Suspension Medium for Rat-Liver-Cell Nuclei,” Biochem. J. 63:214-223, 1956). After the addition of an equal volume of dH₂O, the incubation was continued for 5 minutes on ice (Cocco, L., et al., “Inositides in the Nucleus: Presence and Characterization of the Isozymes of Phospholipase β Family in NIH 3T3 Cells,” Biochim. Biophys. Acta. 1438:295-299, 1999). The suspension was then Dounce homogenized and equilibrated with an equal volume of 120 mM β-Glycerophosphate pH 7, 2 mM MgCl₂, 10-80% Glycerol. Nuclei were pelleted through a two-step sucrose cushion at 1000×g for 10 minutes at 4° C. The lower cushion was 500 mM Sucrose, 2 mM MgCl₂, 25 mM KCL, 65 mM β-Glycerophosphate pH 7, 5-40% Glycerol. The upper cushion was 340 mM Sucrose, 2 mM MgCl₂, 25 mM KCl, 65 mM β-Glycerophosphate pH 7, 5-40% Glycerol. 5% Glycerol is standard, but higher levels can be used. All solutions contain β-mercaptoethanol at 1 mM, sodium butyrate at 5 mM, and PMSF at 1 mM.

Whole tissue was disrupted using a Potter Elvehjem homogenizer in 250 mM Sucrose, 2 mM MgCl₂, 25 mM KCl, 65 mM β-Glycerophosphate pH 7. The sample was filtered through a 40 μm mesh, and brought to 0.5% NP40 and homogenized with another 4-6 tractions when nuclei containing only the INM (Inner Nuclear Membrane) was desired. To isolate nuclei containing both the ONM (Outer Nuclear Membrane) and INM, the sample was first filtered as above and then Dounce (tight pestle B) homogenized until nuclei were liberated. The lysate was then layered over a two-step sucrose cushion as previously described.

Pelleted nuclei were gently resuspended in immunopurification buffer: 340 mM Sucrose, 2 mM MgCl₂, 25 mM KCL, 65 mM β-Glycerophosphate pH 7, 5% Glycerol (lacking β-mercaptoethanol). Nuclei were then added to an equal volume of magnetic beads in the same buffer. The beads were in 5-10 fold excess over total nuclei. The binding reaction was run at 4° C. for 20 minutes with constant agitation. It was essential that the immunopurification mixture fill the reaction vessel because the presence of any air in the tube during the incubation may have caused the nuclei to clump, thus reducing the efficacy of the immunopurification. Immunoadsorbed nuclei were washed using a magnetic stand 5 times as follows: 1×5 mls immunopurification buffer, 4×1 ml immunopurification buffer. Adsorbed nuclei were then Micrococcal nuclease treated in 15 mM HEPES pH 7.5, 1 mM KCl, 2 mM MgCl₂, 1 mM CaCl₂, 340 mM Sucrose.

An alternate procedure involved first the permeabilization of 10⁶-10⁷ cells in 35 mM Hepes pH 7, 5 mM K2HPO₄, 80 mM KCl, 5 mM MgCl₂, 0.5 mM CaCl₂, 50 ug/ml lysolecithin for 1 minute at room temperature, followed by enzymatic treatment (DNaseI or Micrococcal Nuclease) in 35 mM Hepes pH 7, 5 mM K2HPO₄, 80 mM KCl, 5 mM MgCl₂, 2 mM CaCl₂ (Pfiefer, G. P. and A. D. Riggs, “Chromatin Differences Between Active and Inactive X Chromosomes Revealed by Genomic Footprinting of Permeabilized Cells Using DNase I and Ligation-Mediated PCR. Genes Dev. 5:1102-1113, 1991). After appropriate washes, the aforementioned nuclear isolation protocol was used to harvest nuclei for the immunopurification.

Nucleosome Extraction

10⁶ bead-bound or unbound nuclei were digested with 12.5 units of Micrococcal nuclease (Worthington) at 37° C. for 15 minutes in 15 mM Hepes pH 7, 1 mM KCl, 5 mM MgCl₂, 2 mM CaCl₂, 340 mM Sucrose. The reaction was terminated by the addition of 5 mM EGTA and nucleosomes were extracted on ice by a 50-400 mM NaCl series in 15 mM Hepes pH 7, 1 mM KCl, 5 mM MgCl₂, 2 mM EGTA, 340 mM Sucrose (Henikoff, S., et al., “Genome-Wide Profiling of Salt Fractions Maps Physical Properties of Chromatin,” Genome Res. 19:460-469, 2008; Sanders, M. M., “Fractionation of Nucleosomes by Salt Elution From Micrococcal Nuclease-Digested Nuclei,” J. Cell Biol. 79:97-109, 1978). Each extraction reaction was for 20 minutes.

Bead-Nuclei Imaging

Following each nuclear-immunopurification experiment one third of the input, bound and combined supernatant/wash material was loaded into an 8-well Lab-Tek chamber slide. After the nuclei and bead-nuclei complexes settled to an even monolayer, photomicrographs were taken at low magnification (4-10×) with a standard epifluorescence equipped microscope.

Results:

The Tagging Strategy

A great deal is known about the structural network that anchors the nucleus into the cytoskeleton of a eukaryotic cell. The outer nuclear membrane (ONM) is traversed by a family of single pass integral membrane proteins that contain a conserved KASH (Klarsicht, ANC-1, Syne Homology) domain that functions as a nuclear envelop targeting domain (see FIG. 19B; 14b) (Apel, E. D., et al., “Syne-1, a Dystrophin- and Klarsicht-Related Protein Associated With Synaptic Nuclei at the Neuro-Muscular Junction,” J. Biol. Chem. 275:31986-31995, 2000; Fischer-Vise, J. A. and K. L. Mosely, “Marbles Mutants: Uncoupling Cell Determination and Nuclear Migration in the Developing Drosophila Eye,” Development 120:2609-2618, 1994; Malone, C. J., et al., “The C. Elegans Hook Protein, ZYG-12, Mediates the Essential Attachment Between Centrosome and Nucleus,” Cell 115:825-836, 2003; Rosenberg-Hasson, Y., et al., “A Drosophila Dystrophin-Related Protein, MSP-300, is Required for Embryonic Muscle Morphogenesis,” Mech. Dev. 60:83-94, 1996; Starr, D. A., et al., “unc-83 Encodes a Novel Component of the Nuclear Envelope and Is Essential for Proper Nuclear Migration,” Development 128:5039-5050, 2001; Starr, D. A. and M. Han, “Role of ANC-1 in Tethering Nuclei to the Actin Cytoskeleton,” Science 298:406-409, 2002; Welte, M. A., et al., “Developmental regulation of vesicle transport in Drosophila embryos: forces and kinetics,” Cell 92:547-557, 1998). An illustrative KASH domain from mice, as used herein, comprises amino acids 947 to 975 of SEQ ID NO:91. These proteins provide a linkage to the filamentous networks of the cytoplasm (C) (Razafsky, D. and D. Hodzic, “Bringing KASH Under the SUN: The Many Faces of Nucleo-Cytoskeletal Connections,” J. Cell Biol. 186:461-472, 2009; Starr, D. A. and J. A. Fischer, “KASH 'n Karry: The Kash Domain Family of Cargo-Specific Cytoskeletal Adaptor Proteins,” BioEssays 27:1136-1146, 2005). The inner nuclear membrane (INM) contains a triple pass membrane protein that contains a conserved SUN (Sad1p, UNC-84) domain (FIGS. 18B and 19B; 14a) (Jaspersen, S. L., et al., “The Sad1-UNC-84 Homology Domain in Msp3 Interacts With Mps2 to Connect the Spindle Pole Body With the Nuclear Envelope,” J. Cell Biol. 174:665-675, 2006; Kracklauer, M. P., et al., “Drosophila Klaroid Encodes a SUN Domain Protein Required for Klarsicht Localization to the Nuclear Envelope and Nuclear Migration in the Eye,” Fly 1:75-85, 2007; Lee, K. L., et al., “Laminin-Dependent Localization of UNC-84, a Protein Required for Nuclear Migration in Caenorhabditis elegans,” Mol. Biol. Cell 13:892-901, 2002; Malone, C. J., et al., “UNC-84 Localizes to the Nuclear Envelope and Is Required for Nuclear Migration and Anchoring During C. elegans Development,” Development 126:3171-3181, 1999; Moriguchi, K., et al., “Functional Isolation of Novel Nuclear Proteins Showing a Variety of Subnuclear Localizations,” Plant Cell 17:389-403, 2005). An illustrative SUN domain from mice, as used herein, comprises amino acids 777 to 911 of SEQ ID NO:93. This family of proteins interacts with the laminin network of the nucleoplasm (N) (Crisp M., et al., “Coupling of the Nucleus and Cytoplasm: Role of the LINC Complex,” J. Cell Biol. 172:41-53, 2006; Hague, F., et al., “SUN1 Interacts With Nuclear Laminin a and Cytoplasmic Nesprins to Provide a Physical Connection Between the Nuclear Lamina and the Cytoskeleton,” Mol. Cell. Biol. 26:3738-3751, 2006; Lee, K. L., et al., “Laminin-Dependent Localization of UNC-84, a Protein Required for Nuclear Migration in Caenorhabditis elegans,” Mol. Biol. Cell 13:892-901, 2002; Hodzic, D. M., et al., “Sun2 Is a Novel Mammalian Inner Nuclear Membrane Protein,” J. Biol. Chem. 279:25805-25812, 2004; Wang, Q., et al., “Characterization of the Structures Involved in Localization of the SUN Proteins to the Nuclear Envelope and the Centrosome,” DNA Cell Biol. 25:554-562, 2006).

The KASH domain interacts with the SUN domain within the lumen (L) of the nuclear double lipid bilayer (FIG. 19B) (Crisp M., et al., “Coupling of the Nucleus and Cytoplasm: Role of the LINC Complex,” J. Cell Biol. 172:41-53, 2006; Padmakumar, V. C., et al., “The Inner Membrane Protein Sun1 Mediates the Anchorage of Nesprin-2 to the Nuclear Envelope,” J. Cell Sci. 118:3419-3430, 2005; Stewart-Hutchinson, P. J., et al., “Structural Requirements for the Assembly of LINC Complexes and Their Function in Cellular Mechanical Stiffness,” Exp. Cell Res. 314:1892-1905, 2008). The present approach exploits this topology by introducing both fluorescent protein and epitope tag domains within the luminal C-terminal region of the mouse SUN family member, Sun-1 (FIG. 18A) or the N-terminal cytosolic domain of the mouse KASH family member, Nesprin-3 (FIG. 19A) (Crisp M., et al., “Coupling of the Nucleus and Cytoplasm: Role of the LINC Complex,” J. Cell Biol. 172:41-53, 2006; Wilhelmsen, K., et al., “Nesprin-3, A Novel Outer Nuclear Membrane Protein, Associates With the Cytoskeletal Linker Protein Plectin,” J. Cell Biol. 171:799-810, 2005; Haque, F., et al., “SUN1 Interacts With Nuclear Laminin a and Cytoplasmic Nesprins to Provide a Physical Connection Between the Nuclear Lamina and the Cytoskeleton,” Mol. Cell. Biol. 26:3738-3751, 2006).

FIG. 18A provides schematic illustrations of two embodiments of the expression cassette encoding the Sun-1-based nuclear tagging fusion protein. The cassette illustrated in FIG. 18A-1 encodes the Sun-1 nucleoplasmic domain at the N-terminal end of the polypeptide, the SUN Domain (SD), which serves as the nuclear envelope targeting region 14a, and 2XGFP at the C-terminal end of the polypeptide. FIG. 18A-2 illustrates the embodiment where the visualization tag is encoded by a tdTomato sequence, which also contains a sequence encoding an epitope tag to serve as the affinity reagent binding region. The encoding sequence for the GFP domains serve dually as affinity reagent binding regions 16 and visualization tags 22. Two copies of the stabilized super-folder variant were used to enhance both the brightness of the resultant fusion protein and to increase its antigenicity (Pedelacq, J-D., et al., “Engineering and Characterization of a Superfolder Green Fluorescent Protein,” Nat. Biotech. 24:79-88, 2005). The nucleic acid encoding the tdTomato domain serves as a visualization tag 22, and the epitope tags serve as the affinity reagent binding region 16. All resulting tdTomato fusions include an epitope tag because high quality antibodies do not exist for the RFP monomer from which tdTomato is derived (Shaner, N. C., et al., “Improved monomeric red, orange and yellow fluorescent proteins derived from Discocoma sp. Red Fluorescent Protein,” Nat. Biotech. 22:1567-1572, 2004). A representation of an expressed Sun-1-based nuclear tagging fusion protein as it is located on the INM of the nuclear envelope is provided in FIG. 18B. The SUN domain, which spans the INM three times serves as the nuclear envelope targeting region 32. The C-terminal end of the polypeptide extends into the luminal space (L). This luminal portion of the protein contains the nuclear envelope targeting region 34, which, depending on the illustrated embodiment, is an epitope tag separate from a visualization tag 40 (here, tdTomato), or contains GFP domains that serve as both 34/40.

FIG. 19A is a schematic illustration of the expression cassette encoding the Nesprin-3-based nuclear tagging fusion protein. The cassette encodes the cytosolic Nesprin-3 domain at the N-terminal end of the protein, followed by a double copy of the GFP domain to serve dually as affinity reagent binding regions-16 and visualization tags 22, and the KASH domain (KD), which serves as the nuclear envelope targeting region 14b at the C-Terminal end of the encoded protein. The third member of the mouse Nesprin family was selected because it is encoded by a relatively small protein (975aa) (Wilhelmsen, K., et al., “Nesprin-3, A Novel Outer Nuclear Membrane Protein, Associates With the Cytoskeletal Linker Protein Plectin,” J. Cell Biol. 171:799-810, 2005). A representation of an expressed Nesprin-3-based nuclear tag fusion protein is provided in FIG. 19B. The KASH domain, which spans the ONM one time serves as the nuclear envelope targeting region. The N-terminal end of the protein, including the two GFP domains, extends into the cytosolic space. The GFP domains serve as the affinity reagent binding region 34 and as a visualization tag 40. As illustrated, the C-terminus of the Nesprin-3 protein interacts with the C-terminus of the Sun-1 protein in the lumen (L) of the nuclear envelope.

The precise location of the fusion protein junctions were determined by trial and error in the case of Nesprin-3. Ultimately, it was determined that a position between the transmembrane and C-terminal-most spectrin domain was the best location for the insertion of a tag. Moreover, GFP fluorescence is undetectable in fusions where the insertion is bounded by less than 10 linker amino acids on either side of the fluorescent protein. Null mutations in the C. elegans SUN homolog UNC-84 are fully rescued by C-terminal UNC-84-GFP fusions. Therefore, Sun-1 was fused to GFP and tdTomato in the exact same manner (Malone, C. J., et al., “UNC-84 Localizes to the Nuclear Envelope and Is Required for Nuclear Migration and Anchoring During C. elegans Development,” Development 126:3171-3181, 1999). Neither Sun-1 nor Nesprin-3 was truncated because there is evidence in the literature that such manipulations lead to dominant negative activity (Crisp M., et al., “Coupling of the Nucleus and Cytoplasm: Role of the LINC Complex,” J. Cell Biol. 172:41-53, 2006) However, in a subsequent experiment, described below in EXAMPLE 7, a KASH domain family protein from D. melanogaster was truncated by the first 164 amino acids and retained tagging function with apparently healthy cells.

Cellular Localization of Nuclear Tags

In preliminary expression tests using various transformed tissue culture cell lines (COA, HeLa, 293, and N2a), it was clearly evident that all of the tested nuclear tagging fusion proteins (Nesprin-3-2XGFP, Sun-2XGFP and Sun-tdTomato3XMYC) localized properly to the periphery of the nuclear envelope. Specifically, DsRed1 was co-expressed with Nesprin-2XGFP and Sun-2XGFP tags, and GFP was co-expressed with Sun-tTomato. The CMV promoter was used to drive expression in all cells. Image acquisition was at 24 hours post-transfection using an IX81 Olympus Disk Spinning Unit Confocal microscope. The resulting cells were observed for fluorescence of the Nesprin or Sun tags alone, and for a merger of fluorescence of the tags and the co-expressed reporter images. Analysis revealed clear and distinct labeling of the nuclear envelope for each nuclear tag construct in each cell line tested (not shown).

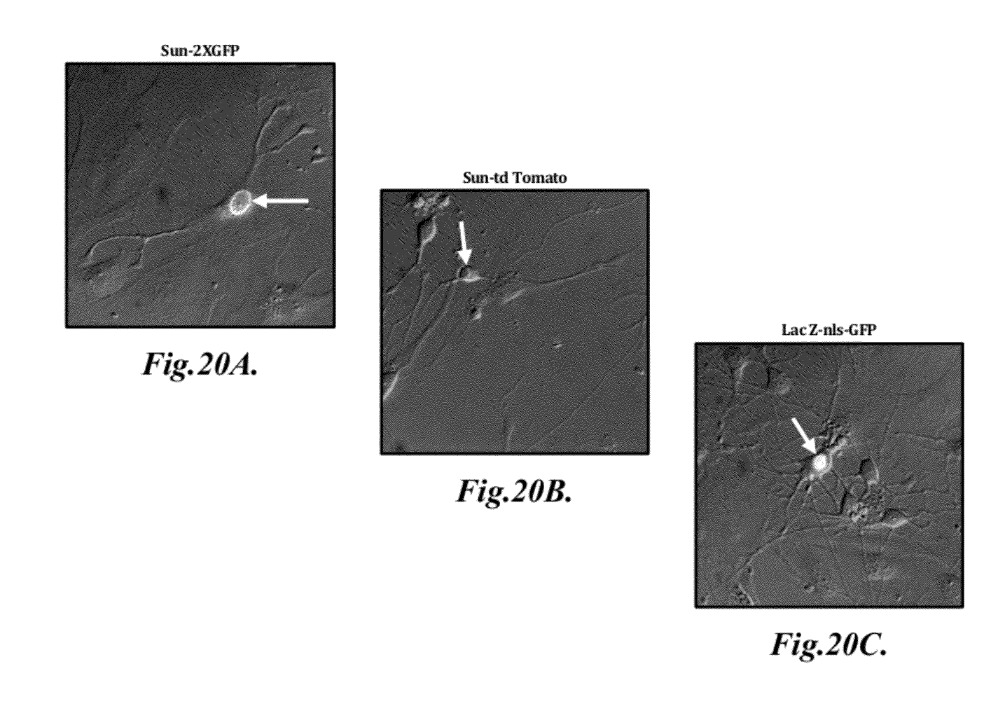

Furthermore, cell division is not required for proper localization of the nuclear tags. In this regard, post-mitotic neurons in rat primary hippocampal cultures received Sun-1 nuclear tagging proteins incorporating either 2xGFP or tdTomato visualization tags. Alternatively, cells were made to express an alternative polypeptide incorporating LacZ coupled with a nuclear localization sequence and a GFP visualization tag. The expression of the tagging proteins was driven by the CMV promoter. The primary neurons were transformed via electroporation. The resulting fluorescence patterns are illustrated in FIGS. 20A-C, respectively. As illustrated, the cells expressing the Sun-based nuclear envelope tagging proteins exhibited tight localization of the protein tags to the nuclear periphery (FIG. 20A for Sun-2xGFP; FIG. 20B for Sun-tdTomato, where the red fluorescence pattern is indicated with an arrow). In contrast, expression of the transgenic LacZ-nls-GFP protein resulted in homogenous fluorescent signal of the entire nucleus. See FIG. 20C. Similarly, nuclear localization of the expressed nuclear envelope tagging proteins was also exhibited in hippocampal primary cultures where expression was driven for ten days through the MSCV promoter in a Lentivirus expression system. Specifically, Sun-2xGFP, Sun-tdTomato-3xMYC, and GFP were expressed using the Lentivirus vector in infected primary hippocampal cultures. Nuclear envelope localization was observed for the nuclear envelope tagging proteins (not shown). There were no observed signs of cytotoxicity in either virus-infected or electroporated cell cultures.

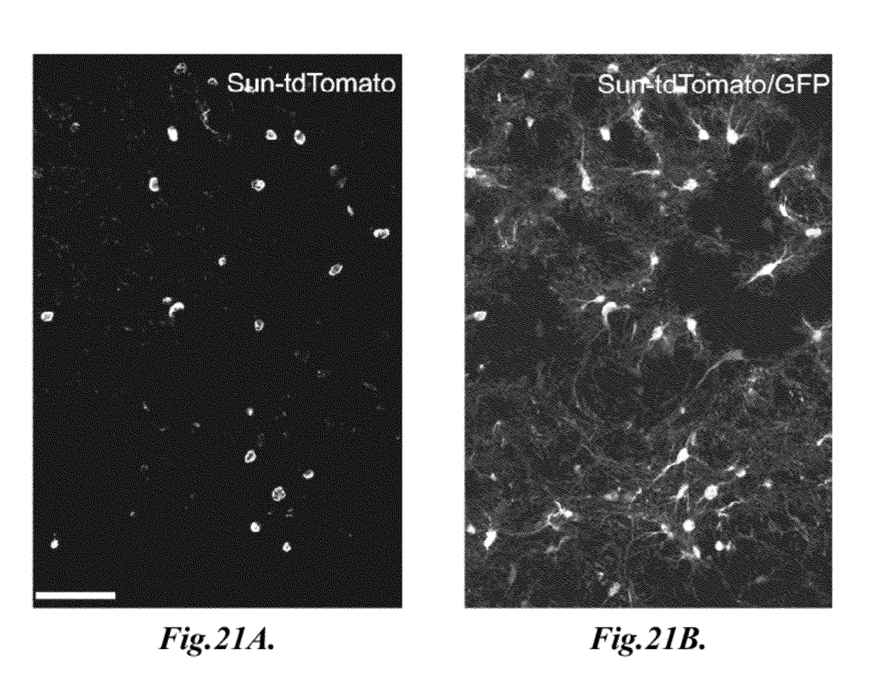

FIG. 21A illustrates striatal neurons obtained from adult mice infected in vivo with Lentiviral vectors encoding Sun-tdTomato. FIG. 21A illustrates striatal neurons obtained from adult mice infected in vivo with Lentiviral vectors encoding Sun-tdTomato and Lentiviral vectors encoding GFP. As illustrated, chronic expression of nuclear tags in the striatum of Lentivirus-infused mice resulted in highly localized nuclear fluorescence after two weeks of expression driven by the Synapsin promoter. No obvious behavioral perturbations were observed in infected animals. In general, expression from the Lentiviral vectors using either the Synapsin or MSCV promoters was lower than that obtained from transfected tissue culture cells or electroporated neurons, where the highly active immediate early promoter of CMV drives expression of the tag. In cases where very high expression was driven for long periods of time, a low level leakage of the nuclear tag into the ER was observed. Despite this issue it was clear that the nuclear tags, when expressed at appropriate levels, allowed the efficient and stable tagging of nuclei over extended periods of time in a variety of cell types, including cells expressing the tags in vitro and in vivo.

Next, it was determined whether the fusion tags localized to the correct nuclear membrane through selective permeabilization of tagged cells with the detergents Triton X-100 and Digitonin. In this regard, moderate levels of Triton X-100 will permeabilize all cellular membranes, whereas only the outer nuclear bilayer (ONM) is disrupted by low levels of Digitonin. See FIG. 19B for a diagram of the INM and ONM of the nuclear membrane. COS cells expressing Nesprin-2XGFP or Sun-2XGFP were permeabilized with either 0.2% Triton or 0.003% Digitonin. GFP was detected by observed immunofluorescence and via immuno-detection. It was clearly evident that for cells expressing Nesprin-2XGFP, the fluorescent protein tag was immuno-detected regardless of the detergent used in the permeabilization. This indicates that Nesprin-based tags can be detected with either detergent, as the epitope is essentially in the cytosol (Crisp M., et al., “Coupling of the Nucleus and Cytoplasm: Role of the LINC Complex,” J. Cell Biol. 172:41-53, 2006). In contrast, the GFP epitope when fused to Sun-1 was only immuno-detected in cells permeabilized with the stronger detergent Triton X-100. The detection of a luminal Sun tag required that cells be permeabilized with the strong detergent Triton X-100.

Purification of Nuclei

An important consideration in the development of nuclear-immunopurification procedure is the obvious problem that clumped nuclei can not be used in the assay. In general the inclusion of glycerol in many of the isolation buffers is advantageous to prevent aggregation (Philpot, J. S, and J. E. Stanier, “The Choice of the Suspension Medium for Rat-Liver-Cell Nuclei,” Biochem. J. 63:214-223, 1956). Thus, a β-Glycerophosphate based buffer system was used.

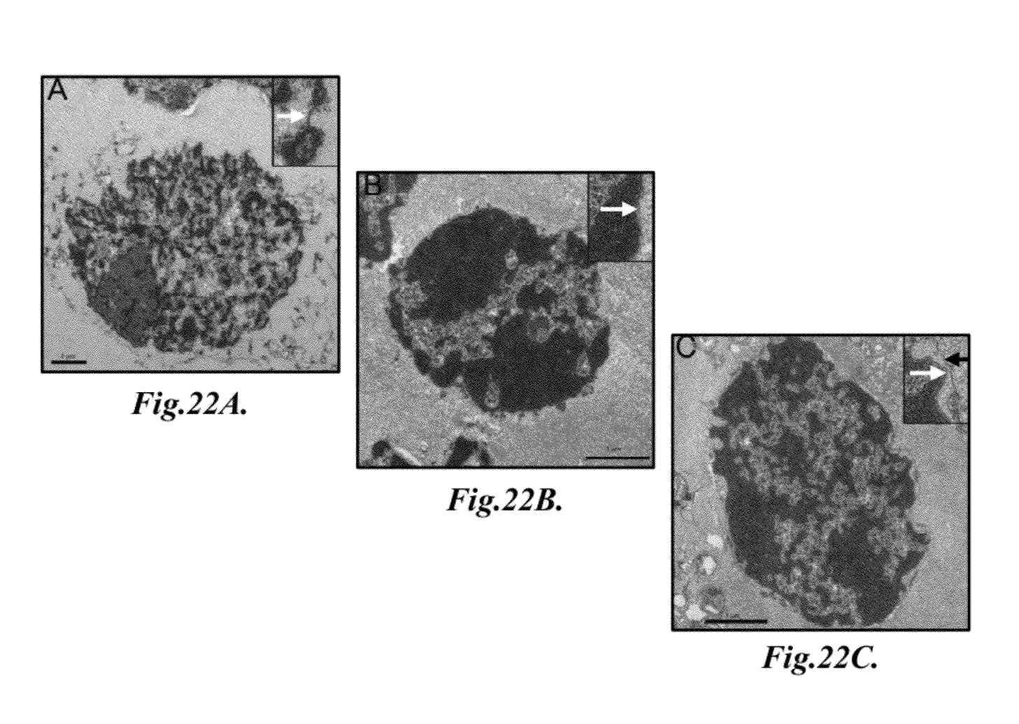

A second consideration is that clearly the differential localization of the Sun and Nesprin-based nuclear tagging proteins requires an isolation procedure that selectively preserves the architecture of the nuclear membranes. For the analysis of the present example, it was possible to isolate nuclei containing the INM only, or retaining both the ONM and the INM, from in vivo tissue sources. For example, FIG. 22B illustrates a representative transmission electron microscope image of a cerebellar nucleus isolated from in vivo tissue in the presence of 0.5% NP40. As illustrated, the nucleus lacks the ONM but retains the INM (white arrow in the inset image). FIG. 22C illustrates a representative transmission electron microscope image of a cerebellar nuclei isolated from in vivo in absence of a detergent. As illustrated, the nucleus retains both the ONM (dark arrow in the inset image) and INM (white arrow in the inset image). However, it was apparent that for the majority of tissue culture cell lines, isolation of nuclei with the ONM was not possible as the obligatory inclusion of detergent in the lysis buffer solubilized the ONM (FIG. 22A; white arrow in the inset image indicates the INM) (Blobel, G. and V. R. Potter, “Nuclei From Rat Liver: Isolation Method That Combines Purity With High Yield,” Science 154:1662-1665, 1966). Therefore, in cell culture, a Sun-based tag is preferably used, whereas, for in vivo expression of the tags, either the Sun- or Nesprin-based tags can be employed, depending on whether a detergent is employed.

A third issue is based on the finding that nuclei are very difficult to immunoprecipitate from crude cellular lysates. Thus, a procedure was developed that involves two steps: 1) the bulk purification of the organelle by density based sedimentation through high concentration sucrose, and 2) the selective immunopurification of tagged nuclei. Molecular manipulations of the nuclei can be performed before or after the immunopurification.

Nuclear-Immunopurification

The effectiveness of bead-conjugated antiGFP (or anti-epitope tag) to appropriately isolate and/or purify the tagged nuclei was assessed. A 1:1 mixture of Sun-2XGFP and Sun-tdTomato-3XMYC tagged COS cell nuclei were prepared from transfected cells. Nuclear-immunopurification using either anti-GFP-Dynabeads or anti-MYC-Dynabeads effectively separated the differentially tagged nuclei. In both experiments the beads (˜10⁷) were in 10-fold excess to nuclei (˜10⁶). Bound beads were washed 5 times, as described in the Methods section, and the total wash material was combined with that obtained from the supernatant of the immunopurification reaction. Magnetic beads pre-loaded with an anti-GFP antibody were observed to effectively demix a mixture of Sun-GFP and Sun-tdTomato-3XMYC tagged COS cell nuclei (not shown). The converse experiment produced concordant results: magnetic beads pre-loaded with an anti-epitope tag (MYC) antibody were observed to effectively demix a mixture of Sun-GFP and Sun-tdTomato-3XMYC tagged COS cell nuclei (not shown).

Furthermore, the variations of the Sun-tdTomato tag were generated to independently incorporate the epitope tags, HA, AU1, FLAG, HSV, V5, and VSV-G. Bead-bound antibodies against each epitope tag were assessed for the ability to appropriately isolate and/or purify the tagged nuclei from a 1:1 mixture of Sun-2XGFP and Sun-tdTomato-3X[epitope tag] tagged COS cell nuclei, as described above. Beads carrying antibody against each of the epitope tags effectively immunopurified the corresponding tagged nucleus with little if any enrichment for a control GFP-tagged nucleus included in the binding reaction (not shown). No differences in the stability or localization of the various Sun-tdTomato epitope tagged proteins were detected.

Nuclei to bead titrations were performed. A 1:1 mixture of Sun-2XGFP and Sun-tdTomato-3XMYC tagged COS cell nuclei was subjected to nuclear-immunopurification using either anti-GFP-Dynabeads or anti-MYC-Dynabeads. The combined immunopurification supernatant and washes for the corresponding nuclear-immunopurification experiment were observed. After the 1:1 mixture was generated, it was diluted to 1:5, 1:10, and 1:20. The 1:1, 1:5, 1:10 and 1:20 columns represent binding reactions containing 1×10⁷, 0.2×10⁷, 0.1×10⁷, and 0.05×10⁷ nuclei per 1×10⁷ beads. At higher dilutions, some cross-reactivity was observed between anti-GFP polyclonal antibodies and tdTomato in the “bound” sample (after the wash was removed). This cross-reactivity becomes problematic when dealing with non-saturating levels of nuclei. As indicated, the anti-GFP polyclonal used in this study inefficiently detects tdTomato. Thus, in practice, single tag experiments can be performed with either the red or green fluorescent tags; however, double label experiments are best performed using the Sun-dTomato-epitope tag variants.

It is apparent that the immunopurification protocol can be performed with a variety of magnetic beads. However, the preferred system is a Protein G coupled Dynabead that is adsorbed to the antibody of interest prior to the actual capture. A second option is to use preadsorbed Sheep Anti-Rabbit Dynabeads. A third option involves the biotinylation of a primary antibody coupled with the use of Streptavidin Dynabeads. The first option is preferred for this example simply because the adsorption protocol is rapid and there is no observed need to block the beads before the immunopurification.

Manipulating Nuclei

After nuclear-immunopurification, a downstream manipulation can be performed on bead-bound nuclei. See FIG. 23A, which illustrates a representative experimental scheme. An alternate approach is to first permeabilize cells, perform a manipulation of interest and then run the immunopurification reaction afterwards (Wilhelmsen, K., et al., “Nesprin-3, A Novel Outer Nuclear Membrane Protein, Associates With the Cytoskeletal Linker Protein Plectin,” J. Cell Biol. 171:799-810, 2005). See FIG. 23B, which illustrates the alternative experimental scheme. The latter approach is better suited to time sensitive techniques such as DNaseI hyper-sensitivity mapping.

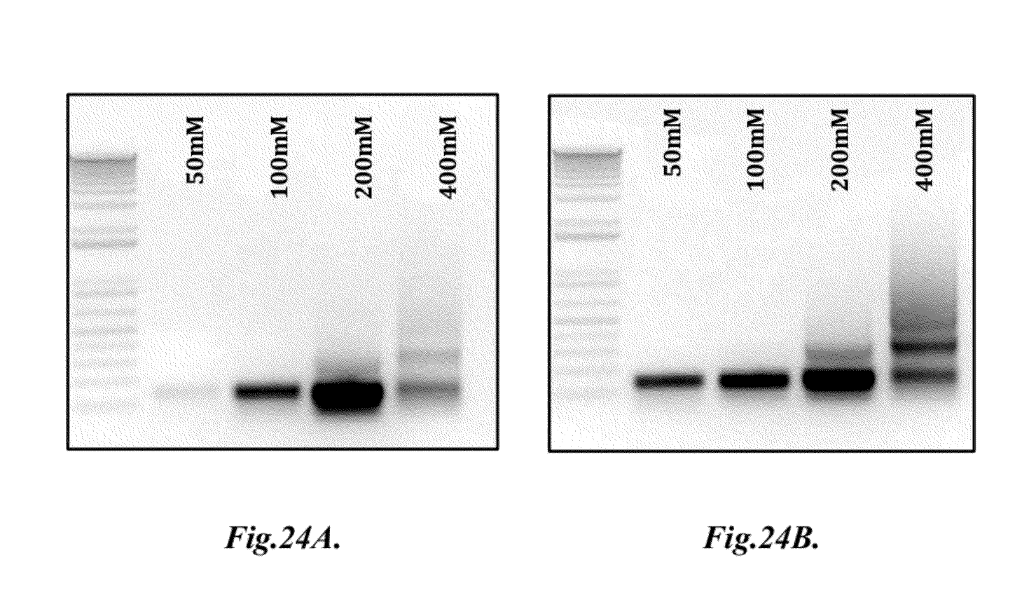

The chromatin of bead bound nuclei was successfully digested with micrococcal nuclease (FIGS. 24A and B). Furthermore, it is possible to differentially extract open and closed chromatin from digested nuclei by means of a simple salt extraction gradient (Henikoff, S., et al., “Genome-Wide Profiling of Salt Fractions Maps Physical Properties of Chromatin,” Genome Res. 19:460-469, 2008; Sanders, M. M., “Fractionation of Nucleosomes by Salt Elution From Micrococcal Nuclease-Digested Nuclei,” J. Cell Biol. 79:97-109, 1978). See FIGS. 24A and B, where salt concentrations are indicated for each column. Although the data presented in FIGS. 24A and B results from the experiment where the genomic manipulation was performed after nuclear-immunopurification, according to the experimental scheme illustrated in FIG. 23A, essentially the same data are obtained if the nuclease treatment is performed on permeabilized cells, according to the experimental scheme illustrated in FIG. 23B (not shown). Moreover, a major advantage of magnetizing the nucleus is that serial salt extraction experiments can be performed more rapidly and quantitatively as the transition from one step of a protocol to the next is based on concentration at a magnet rather than repeated cycles of resuspension-centrifugation (Henikoff, S., et al., “Genome-Wide Profiling of Salt Fractions Maps Physical Properties of Chromatin,” Genome Res. 19:460-469, 2008; Sanders, M. M., “Fractionation of Nucleosomes by Salt Elution From Micrococcal Nuclease-Digested Nuclei,” J. Cell Biol. 79:97-109, 1978).

Finally, it was observed that Dynabeads are weak ion exchangers and bind DNA at low (˜50 mM) salt concentrations. Thus, digested nucleosomes were inefficiently released from the bead-nucleus complex at low (<100 mM) levels of salt (compare FIGS. 24A and B). At higher levels of salt, the nucleosome elution profile is very similar to that obtained with unbound nuclei (compare FIGS. 24A and B).

Conclusion:

In conclusion, a generalized scheme for the isolation of genetically tagged nuclei is demonstrated. The only requirement for the nuclear-immunopurification system is that the target cell be genetically taggable. One advantage of this strategy is that the nucleus is effectively coated with magnetic beads. This inhibits clumping and lysis by avoiding the use of centrifugation steps. Thus, one advantageous application of this technology is that multi-step procedures that include the magnetic bead coating provided by nuclear-immunopurification maintain the nuclear structure during lengthy manipulations.

The data presented in this example demonstrates that the nuclear tagging approach of the INTACT method can be successfully adapted and applied in vivo to mice. Thus, the INTACT method can be applied to eukaryotic cells of any lineage, including mammalian cells. Use of cell-type specific promoters permits the production of cell-type specific genomic data. A nuclear tag can be introduced through the traditional transgenic approach, or, as shown in this example for mice, through a faster route such as a viral vector. This approach can be easily coupled with numerous analytical techniques, such as chromatin immunoprecipitation (CHIP) (Barski, A, et al., “High-Resolution Profiling of Histone Methylations in the Human Genome,” Cell 129:823-837, 2007; Ren B., et al., “Genome-Wide Location and Function of DNA Binding Proteins,” Science 290:2306-9, 2000, and as illustrated above in Example 3), DNaseI hypersensitivity (Boyle A. P., et al., “High-Resolution Mapping and Characterization of Open Chromatin Across the Genome,” Cell 132:311.-322, 2008; Crawford, G. E., et al., “DNase-chip: A High-Resolution Method to Identify DNase I Hypersensitive Sites Using Tiled Microarrays,”Nat. Methods 3:503-509, 2006; Sabo, P. J., et al., “Genome-Scale Mapping of DNase I Sensitivity In Vivo Using Tiling DNA Micro arrays,” Nat. Methods 3:511-518, 2006), and/or nuclear run-on (Core, L. J., et al., “Nascent RNA Sequencing Reveals Widespread Pausing and Divergent Initiation at Human Promoters,” Science 322:1845-1848, 2008). In combination with the aforementioned procedures, nuclear-immunopurification can facilitate the study of cell-type specific transcriptional enhancers, promoters and other genomic elements, enabling a deeper understanding of the mechanisms that control cell type-specific processes.

### Example 7

This example describes the design of nuclear tagging fusion proteins incorporating additional KASH and SUN domains, their in vivo expression in D. melanogaster resulting in the nuclear tagging of specific cell types, and the use of capture reagents to specifically isolate tagged nuclei.

Rationale:

The nucleus of a eukaryotic cell is a double lipid bilayer composed of both an inner nuclear membrane (INM) and an outer nuclear membrane (ONM). As described above, the KASH domain family of proteins are embedded in the ONM; while, the SUN domain family of proteins are embedded in the INM. As described in Example 6, nuclear tagging fusion proteins were constructed that incorporated either a KASH domain or a SUN domain. The nuclear tagging fusion proteins were successfully used to tag nuclei in mice, and permitted the purification of tagged nuclei for subsequence genomic analysis. As further proof that the KASH and SUN domain family members can serve as nuclear envelope targeting regions in the INTACT method, additional nuclear tagging fusion proteins using additional KASH and SUN domain family members were expressed in D. melanogaster, resulting in the in vivo tagging of nuclei. Additionally, using the GLY4/UAC D. melanogaster expression system, cell-type specific expression of the nuclear tagging fusion proteins was demonstrated. Finally, the ability to purify tagged nuclei from a mixture containing nuclei tagged with a distinct affinity reagent binding region was demonstrated.

Methods and Results:

Antibodies

GFP (Invitrogen A11122) and FLAG (Sigma F7425).

DNA Constructs

DNA constructs encoding the nuclear tagging fusion proteins were constructed according to the general design described in Example 6, above. Briefly, a polynucleic acid encoding a synthetic polypeptide linker with the sequence LAAASGGGGSGGGGSLAAASEFSAAALSGGGGSGGGGSAAAL (SEQ ID NO:89), was inserted into the reading frames of D. melanogaster endogenous genes for klarcicht (“klar”; containing a KASH family member domain). The amino acid sequence for the unmodified klar protein is set forth herein as SEQ ID NO:95, and is encoded by the nucleic acid sequence set forth herein as SEQ ID NO:94. The KASH domain comprises amino acids 512 to 567 of the polypeptide sequence (SEQ ID NO:95). The nucleic acid encoding the linker was inserted in the klar-encoding reading frame such that the linker would appear between amino acids 495 and 496, which is N-terminal to the KASH domain. See the general scheme illustrated in FIG. 19A (in context of the mouse Nesprin-3 gene. A polynucleic acid encoding two copies of the super-folder GFP variant was then cloned into the centrally located EcoRI site of the linker (corresponding to the amino acids EF in the linker, underlined in the recitation above). As an alternative for each construct, a polynucleic acid encoding Sun-tdTomato that carried a restriction site at its 3′ end allowing the addition of a C-terminal epitope tag (FLAG). Finally, the DNA construct was truncated to remove amino acids 1-164 (the N-terminal end) of the native klarcicht protein, and a methionine was inserted before amino acid 165. Therefore, the modified N-terminal end became MVTDSNG, etc. This truncation was performed because a domain in the N-terminal portion of the native klarcicht protein causes it to bind to the Microtubule Organizing Center (MTOC), which could cause potential problems in making the protein accessible to the affinity reagents. See Fischer, J. A., et al., “Drosophila Klarsicht Has Distinct Subcellular Localization Domains for Nuclear Envelope and Microtubule Localization in the Eye,” Genetics 168(3):1385-1393, 2004.

Similarly, the linker was inserted into the reading frames of C. elegans endogenous genes for Unc-84 (containing a SUN family member domain), and Unc-83 (containing a KASH family member domain). The amino acid sequence for the unmodified Unc-84 protein is set forth herein as SEQ ID NO:97, and is encoded by the nucleic acid sequence set forth herein as SEQ ID NO:96. The SUN domain comprises amino acids 971 to 1108 of the polypeptide sequence (SEQ ID NO:96). The nucleic acid encoding the linker was inserted in the Unc-84-encoding reading frame such that the linker would appear C-terminal to the SUN domain. See the general scheme illustrated in FIG. 18A-1/A-2 (in context of the mouse Sun-1 gene). The amino acid sequence for the unmodified Unc-83 protein is set forth herein as SEQ ID NO:99, and is encoded by the nucleic acid sequence set forth herein as SEQ ID NO:98. The nucleic acid encoding the linker was inserted in the Unc-83-encoding reading frame such that the linker would appear N-terminal to the KASH domain. See the general scheme illustrated in FIG. 19A (in context of the mouse Nesprin-3 gene). A polynucleic acid encoding two copies of the super-folder GFP variant was then cloned into the centrally located EcoRI site of the linker (corresponding to the amino acids EF in the linker, underlined in the recitation above). As an alternative for each construct, a polynucleic acid encoding Sun-tdTomato that carried a restriction site at its 3′ end allowing the addition of a C-terminal epitope tag (FLAG).

As illustrated generally in FIGS. 18B and 19B (in context of the mouse Sun-1 and Nesprin-3 genes, respectively), this nuclear tagging fusion protein design results in the positioning of the affinity reagent binding region of the protein between the INM and ONM when using the SUN domain, and outside the ONM when using the KASH domain.

The nucleic acid constructs encoding the nuclear tagging fusion proteins incorporate the GAL4/UAS expression system. Briefly, the promoter regions of the reading frames incorporate an upstream activation sequence (UAS) that can be bound by Gal4. Gal4 is a yeast-derived transcription factor protein that can initiate gene transcription upon binding to the UAS in the promoter region. Many transgenic D. melanogaster lines are available that express Gal4 in various specific cell lineages, some of which are used as described below.

Nuclear Tagging Fusion Protein Expression in D. melanogaster Larvae

The described constructs encoding the klar-, Unc-82-, and Unc-83-based nuclear tagging fusion proteins were expressed in cultured cell lines, as generally described above in Example 6. Furthermore, klar-, Unc-82-, and Unc-83-based nuclear tagging fusion proteins were expressed in the ventral nerve cord (VNC) of the 3^{rd} instar of D. melanogaster larvae, driven by the GAL4/UAS system. Localization of the fusion protein tags was assessed using fluorescence based microscopy to visualize the GFP and tdTomato tags. The larvae were also DAPI-stained to establish the location of the nucleic acid within the observed cells. The images were overlayed, with congruent fluorescent signals indicating the localization of the tagging fusion proteins to the nuclear membranes.

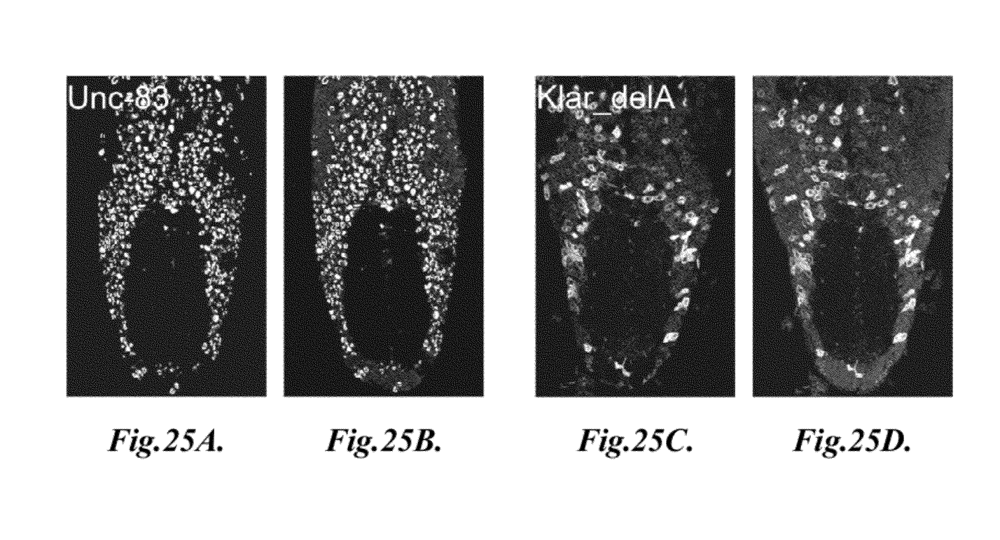

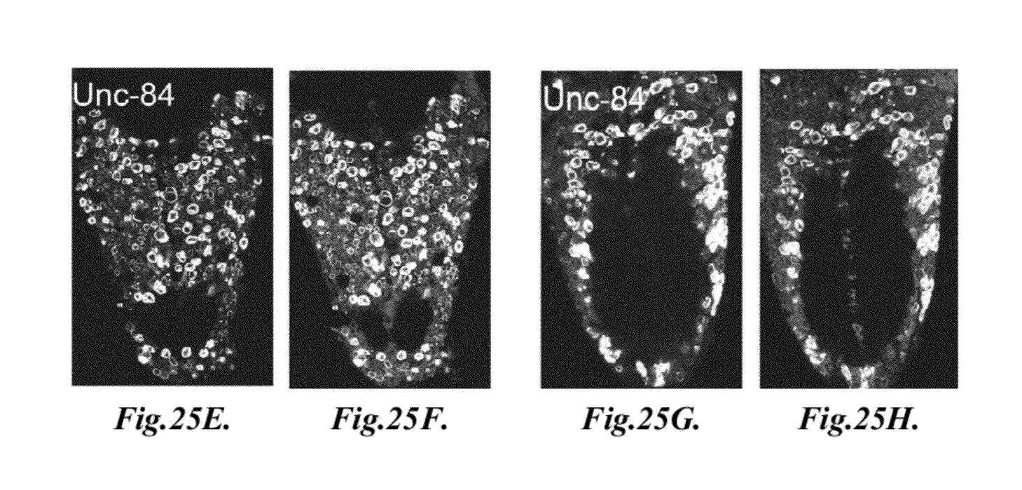

After a preliminary confirmation that the nuclear tagging fusion proteins were expressed and localize to the nucleus in tissue culture cells, the klar-, Unc-82-, and Unc-83-based nuclear tagging fusion proteins were shown to also tag nuclei in vivo. FIGS. 25A-H are fluorescence micrographs of the ventral nerve cord (VNC) of 3^{rd} instar of D. melanogaster larvae. As indicated by the fluorescent signal, the nuclear tagging fusion protein incorporating GFP and tdTomato with the C. elegans SUN domain protein, Unc-84, clearly localized to the periphery of the nucleus. See FIGS. 25E and 25G, respectively. FIGS. 25F and 25H illustrate the images reflected in FIGS. 25E and 25G, respectively, merged with the corresponding DAPI stain of the larvae, indicating the presence of DNA in the VNC. Furthermore, FIG. 25C illustrates the nuclear localization of the tagging fusion protein incorporating GFP and the deletion of the D. melanogaster KASH domain protein, klar. FIG. 25D illustrates the image reflected in FIG. 25C merged with the corresponding DAPI stain of the larvae, indicating the overall presence of DNA in the VNC. Finally, FIG. 25A illustrates the nuclear localization of the tagging fusion protein incorporating GFP and the C. elegans KASH domain protein, Unc-83. As for all illustrated embodiments, the tagging fusion proteins localized to the periphery of the nuclei. However, as is evident from the fluorescence micrograph in FIG. 25A, the nuclei tagged with the UC-83-GFP fusion protein were generally smaller, indicating that expression of the tagging protein results in growth retardation and may be lethal over time. FIG. 25B illustrates the image reflected in FIG. 25A merged with the corresponding DAPI stain of the larvae, indicating the overall presence of DNA in the VNC. Based on these findings, the Unc-84 based tags were analyzed in more detail as described below.

Expression of the Unc-84-2XGFP in Cell Lineages of the D. melanogaster Brain

Expression of the Unc-84-2XGFP nuclear tagging fusion protein was induced in female D. melanogaster flies using the GAL4/UAS system in fly lineages with specific Gal4 expression in fruitless neurons, Kenyon cells of the mushroom body, antennal lobe subgroup cells, and octopaminergic neurons. The cell type-specific expression of the nuclear tagging fusion proteins was assessed using fluorescence microscopy. Images of the frontal and ventral views of each brain were collected.

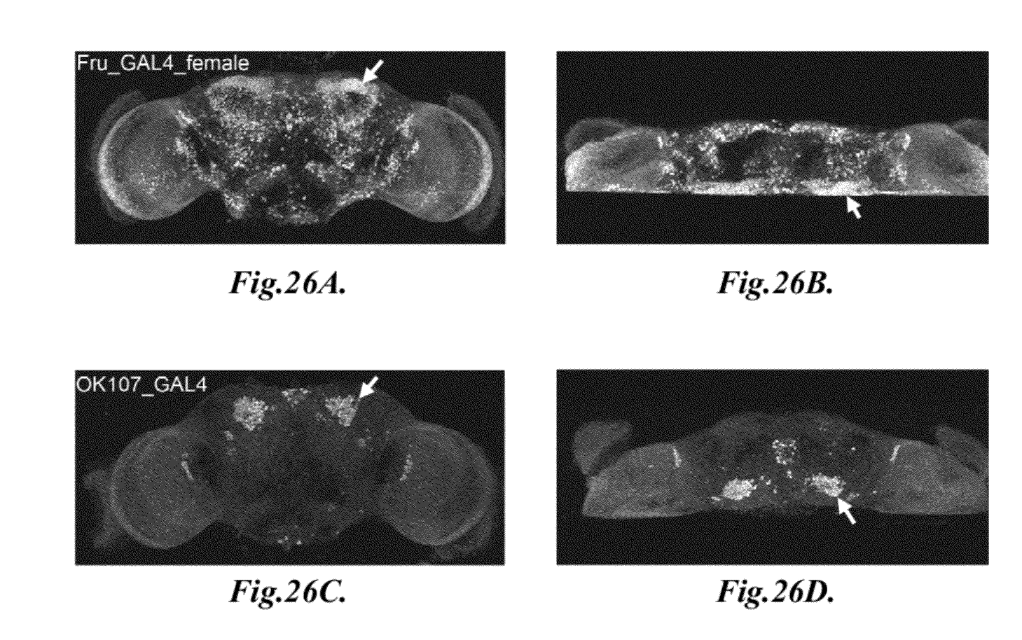

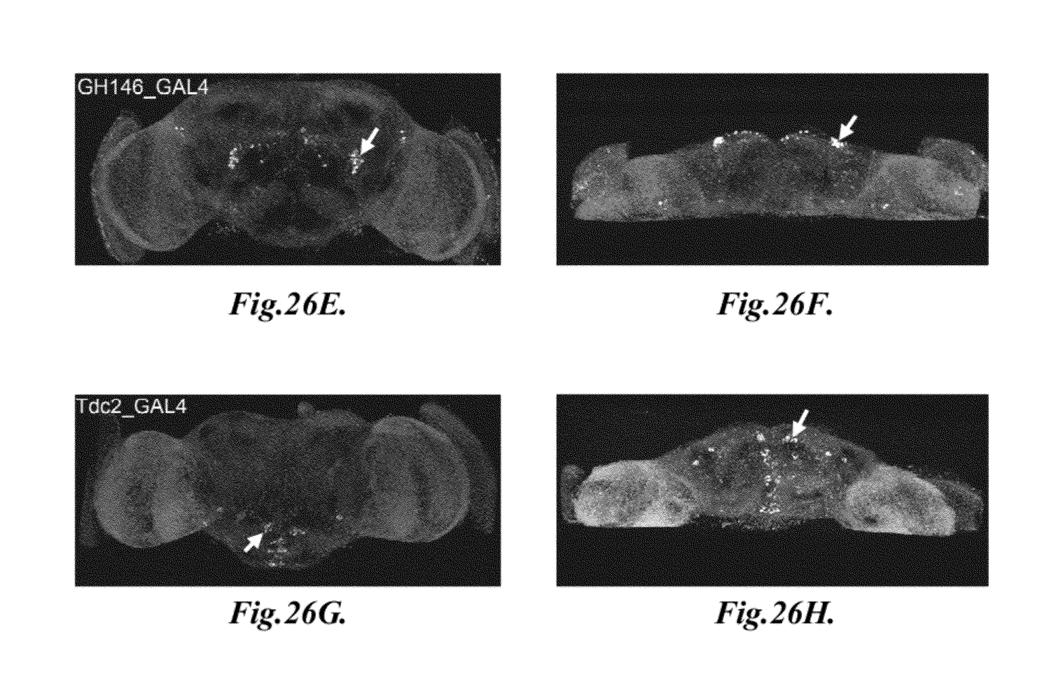

Use of the GAL4/UAS expression system in D. melanogaster afforded the opportunity to assess cell-type specific expression of the nuclear tagging fusion proteins in various distinct cell-types of interest (neuronal lineages) while using the same expression construct. The fluorescence signals in FIGS. 26A and B illustrate the expression of the Unc-84-2XGFP nuclear tagging fusion protein in fruitless neurons (frontal and ventral views, respectively). The fluorescence signals in FIGS. 26C and D illustrate the expression of the Unc-84-2XGFP nuclear tagging fusion protein in Kenyon cells of the mushroom body (frontal and ventral views, respectively). The fluorescence signals in FIGS. 26E and F illustrate the expression of the Unc-84-2XGFP nuclear tagging fusion protein in a sub-population of cells in the antennal lobe (frontal and ventral views, respectively). Finally, the fluorescence signals in FIGS. 26A and B illustrate the expression of the Unc-84-2XGFP nuclear tagging fusion protein in octopaminergic neurons (frontal and ventral views, respectively). In aggregate, these figures illustrate that the nucleic acid construct encoding the nuclear tagging fusion proteins can be appropriately expressed in a specific cell-type of interest through the use of a promoter that is specific for expression in the cell-type of interest.

Immunocapture of Tagged D. melanogaster Nuclei

A mixture of nuclei tagged with either GFP or tdTomato was prepared from either transfected DmBg3-C2 cells or 3rd instar larval neurons. In both experiments, the GFP and tdTomato tagged nuclei were prepared separately, mixed together, and then subjected to immunocapture by magnetic beads that were pre-adsorbed to either an anti-GFP or anti-Flag antibody, as generally described in Example 6.

The initial mixture (i.e., “input”) contained both red and green fluorescently labeled-tagged nuclei in a 1:1 mixture. In the first experiment, beads coupled to anti-GFP antibody effectively separated the mixture into a bead bound population of green nuclei and an unbound population of red nuclei (not shown). The converse experiment, where the beads were loaded with an anti-Flag antibody yielded a bead bound population of red nuclei and an unbound population of green nuclei (not shown). It is noted that the anti-Flag bead capture typically worked less efficiently than the GFP capture, as indicated by a higher rate of red nuclei appearing in the wash population.

Conclusion:

These results demonstrate that the INTACT method can incorporate additional members of the SUN and KASH domain families to serve as nuclear envelope targeting regions. It is noteworthy that, in the Drosophila system described herein, SUN and KASH domains derived from C. elegans functioned to localize the tagging fusion proteins to the nuclei, illustrating the power of these domains to function as nuclear envelope targeting regions across animal phyla. Furthermore, this example provides additional evidence that nuclei tagged according to the INTACT method can be purified from a mixture of nuclei, to facilitate subsequent analysis of the chromatin contained therein.

While the preferred embodiment of the invention has been illustrated and described, it will be appreciated that various changes can be made therein without departing from the spirit and scope of the invention.

1. A vector for selectively labeling nuclei in a cell type of interest comprising a nucleic acid sequence encoding a fusion polypeptide comprising (a) a nuclear envelope targeting region and (b) an affinity reagent binding region.

2. The vector of claim 1, wherein the vector further comprises a cell type specific promoter operatively linked to the nucleic acid sequence encoding the fusion polypeptide.

3. The vector of claim 1, wherein the encoded affinity reagent binding region comprises a biotin ligase accepting site.

4. The vector of claim 3, wherein the vector further comprises a nucleic acid sequence encoding biotin ligase capable of ligating biotin to the biotin ligase accepting site in the fusion polypeptide.

5. The vector of claim 4, wherein expression of at least one of the fusion polypeptide or biotin ligase polypeptide is controlled by a cell type specific promoter.

6. The vector of claim 1, wherein the encoded affinity reagent binding region comprises an epitope recognized by an immunological capture reagent.

7. The vector of claim 6, wherein the epitope is selected from the group consisting of FLAG, HA, MYC, GST, HIS, VSVg, V5, HSV, and AU1.

8. The vector of claim 1, wherein the encoded fusion protein comprises a visualization tag region.

9. The vector of claim 1, wherein the affinity reagent binding region encodes a fluorescent protein.

10. The vector of claim 1, wherein the nuclear envelope targeting region and the affinity reagent binding region of the encoded recombinant fusion polypeptide are separated by a spacer region.

11. The vector of claim 1, wherein the encoded nuclear envelope targeting region comprises a KASH domain.

12. The vector of claim 11, wherein the KASH domain comprises a polypeptide sequence with at least 90% identity to at least one of (i) the sequence from amino acid residue 947 to amino acid residue 975 of SEQ ID NO:91; and (ii) the sequence from amino acid residue 512 to amino acid residue 567 of SEQ ID NO:95.

13. The vector of claim 1, wherein the encoded nuclear envelope targeting region comprises a SUN domain.

14. The vector of claim 13, wherein the SUN domain comprises a polypeptide sequence with at least 90% identity to at least one of (i) the sequence from amino acid residue 771 to amino acid residue 911 of SEQ ID NO:93; or (ii) the sequence from amino acid residue 971 to amino acid residue 1108 of SEQ ID NO:97.

15. The vector of claim 1, wherein the nuclear envelope targeting region is encoded by a nucleic acid sequence comprising a nucleic acid sequence with at least 90% identity to SEQ ID NO:1.

16. The vector of claim 3, wherein the encoded biotin ligase accepting site has an amino acid sequence with at least 90% identity to one of SEQ ID NO:6 or SEQ ID NO:88.

17. The vector of claim 2, wherein the cell type of interest is derived from a mammal.

18. The vector of claim 2, wherein the cell type of interest is derived from a plant.

19. The vector of claim 2, wherein the cell type of interest is derived from a nematode.

20. The vector of claim 2, wherein the cell type of interest is derived from an arthropod.

21. The vector of claim 4, wherein the encoded biotin ligase comprises an amino acid sequence with at least 90% identity to SEQ ID NO:12.

22. The vector of claim 1, wherein the vector is a plasmid.

23. The vector of claim 1, wherein the vector is a viral vector.

24. A cell comprising the vector of claim 1.

25. The cell of claim 24, wherein the cell is in a tissue.

26. The cell of claim 24, wherein the cell is in culture.

27. The cell of claim 24, wherein the cell is part of a transgenic organism.

28. A kit for selectively labeling nuclei in a cell type of interest, the kit comprising:
(a) a vector comprising a first expression cassette comprising a nucleic acid sequence encoding a fusion polypeptide comprising:
(i) a nuclear envelope targeting region; and
(ii) an affinity reagent binding region; and
(b) a capture molecule capable of specifically binding to the affinity binding region, or a modified form thereof.

29. The kit of claim 28, wherein the first expression cassette is adapted to receive a cell-type specific promoter operatively linked to the sequence encoding the fusion protein.

30. The kit of claim 28, wherein the affinity reagent binding region comprises a biotin ligase accepting site.

31. The kit of claim 28, further comprising a second expression cassette comprising a nucleic acid sequence encoding a biotin ligase polypeptide.

32. The kit of claim 31, wherein the biotin ligase accepting site comprises an amino acid sequence with at least 90% identity to SEQ ID NO:6.

33. The kit of claim 28, wherein the capture molecule is bound to a magnetic particle.

34. The kit of claim 28, wherein the affinity reagent binding region comprises an epitope recognized by an antibody, and wherein the capture molecule is an antibody that specifically binds the epitope.

35. The kit of claim 28, wherein the capture molecule binds to biotin.

36. The kit of claim 28, wherein the nuclear envelope targeting region comprises a KASH domain or a SUN domain.

37. The kit of claim 36, wherein the affinity reagent binding region encodes a fluorescent protein.

38. A method for selectively isolating nuclei from a cell type of interest present in a plurality of cells wherein at least a portion of the cells recombinantly express a fusion polypeptide comprising (i) a nuclear envelope targeting region and (ii) an affinity reagent binding region, wherein at least one of the fusion polypeptide or a molecule that modifies the fusion protein is under the control of a promoter specific to the cell type of interest, the method comprising:
(a) lysing the plurality of cells under conditions suitable to generate a cell lysate comprising a plurality of intact nuclei;
(b) contacting the cell lysate with a capture molecule that specifically binds to the affinity reagent binding region, or a modified form thereof, under conditions suitable to bind the nuclei comprising the fusion polypeptide; and
(c) isolating the nuclei bound to the capture molecule.

39. The method of claim 38, wherein the affinity reagent binding region comprises a biotin ligase accepting site, and wherein at least the portion of the cells further express a biotin ligase polypeptide capable of attaching biotin to the biotin ligase accepting site.

40. The method of claim 38, wherein the affinity reagent binding region comprises an epitope recognized by an antibody.

41. The method of claim 38, wherein the nuclei of the cell type of interest are isolated from a mixture of multiple cell types obtained from at least one of a plant, a nematode, an arthropod or a mammal.

42. The method of claim 41, wherein nuclei are isolated from a mammal.

43. The method of claim 42, wherein the nuclear envelope targeting region of the fusion polypeptide comprises a KASH or SUN domain.

44. The method of claim 38, wherein the method further comprises permeabilizing the cells and subjecting nucleic acids therein to biochemical manipulation before the cell lysis of step (a).

45. The method of claim 38, wherein the method further comprises introducing a viral vector encoding the fusion polypeptide into the mammal.

46. The method of claim 45, wherein the cell type of interest is post-mitotic neurons.

47. The method of claim 38, wherein the method further comprises extracting nucleic acids from the isolated nuclei.

48. The method of claim 38, wherein the method further comprises performing gene expression analysis on the isolated nuclei.

49. The method of claim 38, wherein the method further comprises performing analysis of the chromatin structure of the isolated nuclei.

50. A method of generating in vivo biotinylated nuclei in a cell type of interest comprising recombinantly co-expressing in the cell:
(a) a fusion polypeptide comprising (i) a nuclear envelope targeting region; and (ii) a biotin ligase accepting site; and
(b) a biotin ligase;
wherein the co-expression of the recombinant fusion polypeptide and the biotin ligase produces biotinylated nuclei in the cell type of interest.

51. The method of claim 50, wherein the expression of at least one of the fusion polypeptide and the biotin ligase is under the control of a promoter specific to the cell type of interest.

52. The method of claim 50, wherein the nucleic acid sequences encoding the fusion polypeptide and biotin ligase are present on the same vector, and wherein the co-expressing comprises introducing one or more copies of the vector encoding the fusion polypeptide and biotin ligase into the cell type of interest, or a progenitor of the cell type of interest.

53. The method of claim 50, wherein the nucleic acid sequences encoding the fusion polypeptide and biotin ligase are present on separate vectors, and wherein the co-expressing comprises introducing one or more copies of the vector encoding the fusion polypeptide and introducing one or more copies of the vector encoding biotin ligase into the cell type of interest, or a progenitor of the cell type of interest.

54. The method of claim 50, wherein the fusion protein further comprises a visualization tag.

55. The method of claim 50, wherein the cell type of interest is in a mixture of multiple cell types.

56. The method of claim 55, wherein the mixture is a cell culture.

57. The method of claim 55, wherein the mixture is a tissue.

58. The method of claim 50, wherein the cell type of interest is present in a plant, nematode, arthropod, or mammal.

59. The method of claim 50, wherein the cell type of interest is derived from the root cell epidermis of A. thaliana.

60. The method of claim 50, wherein the method further comprises isolating biotinylated nuclei from the cells using a capture molecule that specifically binds to biotin.

61. A method of selectively isolating nuclei from a cell type of interest wherein at least a portion of the cells co-express (i) a recombinant fusion polypeptide comprising a nuclear envelope targeting region and a biotin ligase accepting site, and (ii) a biotin ligase, wherein expression of at least one of the recombinant fusion polypeptide or the biotin ligase is under the control of a promoter that is specific for the cell type of interest, and wherein the co-expression of the recombinant fusion polypeptide and the biotin ligase selectively produces biotinylated nuclei in the cell type of interest, the method comprising:
(a) lysing the plurality of cells from the mixture under conditions suitable to generate a cell lysate comprising a plurality of intact nuclei;
(b) contacting the cell lysate with a capture molecule that specifically binds to biotin under conditions suitable to bind the biotinylated nuclei; and
(c) isolating the biotinylated nuclei bound to the capture molecule.

62. The method of claim 61, wherein the capture molecule is bound to a magnetic particle.

63. The method of claim 61, wherein the capture molecule is selected from the group consisting of: streptavidin or a fragment thereof, avidin or a fragment thereof, and an anti-biotin antibody or a fragment thereof.

64. The method of claim 61, wherein the cell type of interest is in a mixture of multiple cell types.

65. The method of claim 64, wherein the mixture is a cell culture.

66. The method of claim 64, wherein the mixture is a tissue.

67. The method of claim 64, wherein the method further comprises extracting nucleic acids from the isolated biotinylated nuclei.

68. The method of claim 64, wherein the method further comprises performing gene expression analysis on the isolated nucleic acids.

69. The method of claim 64, wherein the method further comprises performing analysis of the chromatin structure of the nucleic acids.

70. A method of visually tagging nuclei in a cell type of interest comprising introducing a vector comprising a nucleic acid sequence encoding a fusion polypeptide comprising (a) a nuclear envelope targeting region and (b) a fluorescent protein, into the cell type of interest.

71. The method of claim 70, wherein the cell type of interest is eukaryotic.

72. The method of claim 71, wherein the nuclear envelope targeting region selectively targets the outer nuclear membrane.

73. The method of claim 72, wherein the nuclear envelope targeting region comprises a KASH domain.

74. The method of claim 71, wherein the nuclear envelope targeting region selectively targets the inner nuclear membrane.

75. The method of claim 74, wherein the nuclear envelope targeting region comprises a SUN domain.

76. The method of claim 71, wherein the cell type of interest is a neuron.

77. The method of claim 70, wherein the vector is a viral vector.

78. The method of claim 70, wherein the cell type of interest is present in a mammal.

79. The method of claim 78, wherein the mammal is a mouse.

80. The method of claim 70, further comprising isolating the tagged nuclei.

81. The method of claim 80, wherein the tagged nuclei are present in eukaryotic cells and are isolated under conditions that preserve both the outer nuclear membrane and the inner nuclear membrane.

82. The method of claim 80, wherein the tagged nuclei are present in eukaryotic cells and the cells are permeabilized prior to isolation of the tagged nuclei.


SEQUENCE LISTING (99 sequences)

SEQ ID NO: 1 - DNA, 333 bp, Artificial sequence
  Synthetic
atggatcattcagcgaaaaccacacagaaccgtgttttgtcagtgaagatgtggccaccgagtaagagtacccgtctcatgcttgttgagcggatgaccaagaacattaccaccccttccatcttctccaggaagtacggtcttttgtctgttgaagaggctgagcaagacgccaagcgcattgaagatttggcctttgctactgccaacaaacacttccagaacgagcctgatggtgatggcacttctgctgttcacgtctatgctaaagaatccagcaagctcatgcttgatgtcatcaaacgtggtccacaggaagaatccgaggttgag

SEQ ID NO: 2 - protein, 111 aa, Artificial sequence
  Synthetic Construct
MDHSAKTTQNRVLSVKMWPPSKSTRLMLVERMTKNITTPSIFSRKYGLLSVEEAEQDAKRIEDLAFATANKHFQNEPDGDGTSAVHVYAKESSKLMLDVIKRGPQEESEVE

SEQ ID NO: 3 - DNA, 738 bp, Artificial sequence
  Synthetic
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagctgcggccgctgcc

SEQ ID NO: 4 - protein, 246 aa, Artificial sequence
  Synthetic Construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGAAAAA

SEQ ID NO: 5 - DNA, 72 bp, Artificial sequence
  Synthetic
atggctggtggacttaacgatatcttcgaagctcagaagattgaatggcatgaggatactggtggatcttga

SEQ ID NO: 6 - protein, 23 aa, Artificial sequence
  Synthetic Construct
MAGGLNDIFEAQKIEWHEDTGGS

SEQ ID NO: 7 - DNA, 45 bp, Artificial sequence
  Synthetic
ggacttaacgatatcttcgaagctcagaagattgaatggcatgag

SEQ ID NO: 8 - protein, 15 aa, Artificial sequence
  Synthetic Construct
GLNDIFEAQKIEWHE

SEQ ID NO: 9 - DNA, 1149 bp, Artificial sequence
  Synthetic
atgaatcattcagcgaaaaccacacagaaccgtgttttgtcagtgaagatgtggccaccgagtaagagtacccgtctcatgcttgttgagcggatgaccaagaacattaccaccccttccatcttctccaggaagtacggtcttttgtctgttgaagaggctgagcaagacgccaagcgcatcgaagatttggcctttgctactgccaacaaacacttccagaacgagcctgatggtgatggcacttctgctgttcacgtctatgctaaagaatccagcaagctcatgcttgatgtcatcaaacgtggtccacaggaagaatccgaggttgaggcggccgctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggagctgcggccgctgccatggctggtggacttaacgatatcttcgaagctcagaagattgaatggcatgaggatactggtggatcttga

SEQ ID NO: 10 - protein, 382 aa, Artificial sequence
  Synthetic Construct
MNHSAKTTQNRVLSVKMWPPSKSTRLMLVERMTKNITTPSIFSRKYGLLSVEEAEQDAKRIEDLAFATANKHFQNEPDGDGTSAVHVYAKESSKLMLDVIKRGPQEESEVEAAAVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGAAAAAMAGGLNDIFEAQKIEWHEDTGGS

SEQ ID NO: 11 - DNA, 966 bp, Escherichia coli
  CDS (1)..(966)
atgaaggataacaccgtgccactgaaattgattgccctgttagcgaacggtgaatttcactctggcgagcagttgggtgaaacgctgggaatgagccgggcggctattaataaacacattcagacactgcgtgactggggcgttgatgtctttaccgttccgggtaaaggatacagcctgcctgagcctatccagttacttaatgctaaacagatattgggtcagctggatggcggtagtgtagccgtgctgccagtgattgactccacgaatcagtaccttcttgatcgtatcggagagcttaaatcgggcgatgcttgcattgcagaataccagcaggctggccgtggtcgccggggtcggaaatggttttcgccttttggcgcaaacttatatttgtcgatgttctggcgtctggaacaaggcccggcggcggcgattggtttaagtctggttatcggtatcgtgatggcggaagtattacgcaagctgggtgcagataaagttcgtgttaaatggcctaatgacctctatctgcaggatcgcaagctggcaggcattctggtggagctgactggcaaaactggcgatgcggcgcaaatagtcattggagccgggatcaacatggcaatgcgccgtgttgaagagagtgtcgttaatcaggggtggatcacgctgcaggaagcggggatcaatctcgatcgtaatacgttggcggccatgctaatacgtgaattacgtgctgcgttggaactcttcgaacaagaaggattggcaccttatctgtcgcgctgggaaaagctggataattttattaatcgcccagtgaaacttatcattggtgataaagaaatatttggcatttcacgcggaatagacaaacagggggctttattacttgagcaggatggaataataaaaccctggatgggcggtgaaatatccctgcgtagtgcagaaaaataa

SEQ ID NO: 12 - protein, 321 aa, Escherichia coli
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 13 - DNA, 1177 bp, Artificial sequence
  Synthetic
aaccaacaatttgaacctgagaaatcgtttgcaactggcgagaagtctttgtctgaaactcgatgttctagtacaagacgaagagactggtttgtccgagatggtgcgattgcgaatcttgggtacaagagccattagagatcgacttctcaggaaaaaaatattttagggtttaaacataactttcttctacttcacccactcgagagaaacgtcaatgttggaaattgaatctttgcttaatcgtgtacaagtgaaatcagaagagaaaggtgagaaaatgaagtgtagagagatgtgtgtttaaggaagaagaagaggcgcgctcgagtgtagaagaacgaagaagaagaagaatagtagtgaaggaaaagagagaaaaaggccattgggccagaagtgaacaaggcccaattatgggttcaatggtcgtttgggccgcataaaacaatatttagcatagaggctaaagtcaaagtatatagaagagaaaaaaatatatataacatgttcatcatgaaaacttcaacggtccaatgatctcccgaccaaagttgagctcatatgcaacaaacacaaataaatatagatttatatgtattatcaaggattaaaactatatataggtttgaaaaaaatgggcacgacgtgatagcatcaaagtcatctatttcaagacgtgatcagcctaaagaccaaaagaaaatatcaaatattacccaacttttgtgtttttcctcaacctcataaacccccccaatcctcctcactcttccctatcctcttcatttttcccaatcatcaaacacaagattagagttcgattctccaaagaaaatatggtaagttcctacatttaatcttctgatgtagatgatgatgatgcattgattctgtttatgttctgattgtcaacatgtgaaatttatatgtggatggtgtggttgaatatatatggttttgtgttatttggttttggaagtgtaacacatgaataggtttcatgtatatattgacttcattaagaatgatgttgtggacatatggtttatatgctatgatcctaaatcacaatctaagttttatggtaactggttaggaagatgtatagaatggaaacagcttaatatataaataaatgactacgtgtgtgtgtgtgtgtgttgtgcaggctaat

SEQ ID NO: 14 - DNA, 3708 bp, Artificial sequence
  Synthetic
tttccctcggccaccggcggtacaaacggcgtcgtttcgcggattagaaaaacgttctgtcttgatatcatttacttcattctaagcggctttggtctgaattttttatatacaaggcctgtctccgtttttgtaaagggaaaacagtaggatccattttagcctctgtaagtaacaatattgggcccctaaaagcccacccattttggggccagcaaaccaaggcaccctcggttccgcacgctcgctaagacgctaacctatgcatatgttgatatgttttttctcttccttttggtatgaatcttgatttgttttgatactcatgatgtacattcgtattctcttacgtattgtaaaccatcctatttcagatcacgattatatctttacatttacatttttcatttttatttctgtttgaatgttacaatttactagtagagttattcattaaaatactacaactggtatacagaaatgtaatttgagtgataaattatatgaaataattaagtaatatatgtgatatttatggatccaaacaaaaactaattactggttcattttctattttagatgtaagcaaaatgtgtaagattcaaggtatatatatatcccaatatacgtatatatgtggtactcactagctagtagctctctcacaactgtgtcttttggttttcatcagctgatcctctccaactaactatccatcttttgttttgcggttggacttggaggtaccaagaatattagcaacgtacgactcgtatggtatcatttcctttgtacaaaaagtgaatatcaaaatgcattgtattaattatatataagttagtatatggagttagttgtcctcactgtctttatctggcgcaatctcctatgccatcattccctcttcacacgtacgtgtgcacactcgatgtcacatttgtataaacacgtttgcttttagcgtgagatcatcaccatattccatttttggtgggtcagttctctttctagatagttatttgtaaggacgtgaattaaaagggatcgtcgtcacttgttgagataaaagaaaagatatatggtcagtttctgcatcttggaatcaacttaagggttgtcttaattaattttgatagaccctactttaaaaattaattagttgctttcattggccctcaataagaaaagccaaaaaagaaagaagactggtcttggaagtttgccaacacgggtaatagattaatggtgaaaagggcgaatttttttacccaaaaccctaattaagtagaagtattaatcgagagcaaaaaagagagagagagtcagtagccaaaaggaatgaatggaagaaagaaaaaggaatctctataggcagcatatattcaagtaattaattaaagtagatagatagagcaaaaggagaggttaggaggcattaattaattatttaagagcatgtggtgaatgtaaatgtttatggttgcttccctctctatacattatgtatctacctttcctaactaacaattccctaggccgtacgacgactaacaaagaaaaaaacaaaagaaactgataaagcttttgaattgtagataaatcatctgctacagttataccattatatatcttattaaagacctaagtttccttcactatacgtcttcgtccatttacgtacgtattatacggacggtttaagctactatatctatattgttaacaatgtaactgttgagatatatcttgcaataatatgtcatggtgtatgcatacgataatatgaatcaatgtttgaaatcttgacgtgcccgtgatacaataagatgatcaaaatttcaaattttgtcaaatattaaaacaacatacacatacacatgtgtccaggtggcattataaaatgtatatatggtggatatagagagagagggagatgcgtatagtgaataggaaagtaagtaataaagagagggtggaggaattggaaaggggttggaggcaaacccataaagagcattcatttccttttaaggtcgctgaaattaatgagtaacgatcggtcaatgcctctcgctgacctttttctttttttacaacaacaaataaaaataaaataaatttcgacgtctctttccgctgctgaattacatttgttgaattaattttctctgcttacgtacgtcttctaaactttctctatccgaattcttttttaactttctaacttatattcaacaactcttctttcctgcctttaccgttagtctaattgttttcctaatactgctacgtacatacccctactatactagtcagtgtattagattcgattgggattaatccaggaatatagatatcccattagtttttataaaaatattggaagaggacaagtctcaagcaatttagggttccatgtagcgctgcaatatactgttagtaactctctcttacccatatattgtatatgctaattcttatcaaatatatatatatgcttctcccagagtcccagtttcctataatcctgacgcaattatactaatagagccaagtttacataataaagtatatatgattaatagatagggtttcttattaagccatatcttaaattaagatgtgatgatagcgttttgtataagttaccaattgtttgaaagaagagatcatcacaataataaatcataagtagtagtatatagtaataaataaatacacaagtcataataagagtaatgagaggataattaaggagggaagaagaaagcagaaaatgcggttggagaattaggtgctaaaagttagttgagtccatctcagtatctaacggtcaactctctctctctctagagaaaacaattaagaaatctgacatacacatatgtctctctctctctctctctctagtctatacacacaattcaattaaagaagagacagagaagttcgtcttttttgtttttatacccttaaatcaatcatgcaattgtaacccttccttcttattctcattccttccccccctgtctacagtaatctatagcaacgccattatgtactacttttaacggataatttgctcatgtttcaatatggcttcattgtatatatgttcaagttcttctcaatcctttatatcattccaacataattcatattaaagttagtagctgaaattggaaggctgatatattttccataattcaaatttgaattttgctcatcatatatatatgtatatattaaaaatcgaatattaagaagaaaaatgaagtcgatcgatggctgccaatgctgtagctggccatgttttaaactactcaattgtcggattgaagtatagccaaaatatataaaaccgtaaaaggactaaatataataatataataggtattaattaattaaaactaattaattataaaagaagcacctaaaagtcaagagcagtagagaaatggaagaaatatctgaaaaacgaccgcttatatatatatgtatcattggaattgaagaggctatatatatatatatatatatatatatcgatcttagcttatatattaattgaaagtacattttggtgtataagtaattaaagaagaaagaaaaaaagagagataatatataaggaagaaggagtgcgaggagaagagggaagagatcataattaagcaaagaagctagctagggacaggatttccatgg

SEQ ID NO: 15 - DNA, 1444 bp, Artificial sequence
  Synthetic
gacaaaatttagaacgaacttaattatgatctcaaatacattgatacatatctcatctagatctaggttatcattatgtaagaaagttttgacgaatatggcacgacaaaatggctagactcgatgtaattggtatctcaactcaacattatacttataccaaacattagttagacaaaatttaaacaactattttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaaaatggctgaggctgatgatattcaaccaatcgtgtgtgac

SEQ ID NO: 16 - DNA, 711 bp, Artificial sequence
  Synthetic
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 17 - protein, 236 aa, Artificial sequence
  Synthetic Construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 18 - DNA, 1001 bp, Artificial sequence
  Synthetic
caaacgttatagtgtggacaccaatttggcttcgtacttcttactcgttcccgccccgccccgacaccattcgaatatatattgtcagttgttctgtttgtcgtcgtgatgagaggtgcgtgcgtgtgcgcgcgcgctctcccaacttttcggaccattttgcgggctttatactgcaatttggctcatttcttctccttttttttccacaggcttattttgtactctcgagttgacagcctttttcggtataggttggaagtagtccattaaagtagatgtttcttaagaatttatcggcttagcatcgatttcgacctagttagatgccacgtttatgcgcgaagcggccgcgtcgcggcgagaatttcagtgcggttttacgggattatccattaaaatcaggaaaatcagcagaaaacttcttaacaaaactaatttttttagaaaagaatttagaaatacgtagtcttgaataattcagcaactttttcaaattttatgagaatttcgcaatttcctacacctcctatttcatcatcacttccgaaatcctacgttatcgatcgaacacgcgacgcgaccgcagcgcacctactaagcgcgcgcatttcgaccgtgacgcgaccgcaacgcggtggtgtcagggtgcgcgcgctttttcaatcatatgtggtgcggtcgattccttcattcatccttctgtatgcagctgctcatcagtttattgagcatctgctgtaccaattcgtactttaaattaaatattaagagtacattttctgtgtgctctgtatataaatgtctagaataaactgcaaacaagaattcttggctccatctttccctgaatcaatatcgatcgatagtgacacgcgacgcgaccgcaacgcgtagggccatttcatattgattcttcttctactcttccttcttctccttctgctctcacatttcctcactattccaaccattctcaattctcaatttccagaacaaca

SEQ ID NO: 19 - DNA, 1000 bp, Artificial sequence
  Synthetic
gctccatcaccaattctcgaagcacttttaatattcatttaatccaacttgtaccataatcctcgtatttttccatcccgaaaattgatttttgcgatcgaaccgccgatctcatcaccacttttctctccgttctttatgtcttattttattcgtaaattgctgaatttcatgttgaactccaacaaaaaaaaataaagtttcgtgtgcttcagtagtctagcaacatcatatgaacgcctagcaactatattccacgccgccgcagttttgaattgaattcactaaattcatgctctgaattcacaaaaaaaatgtttcttcgagtcattggaaatagtaaaaaacgccagcgactatttatcagtaagttttaaaagggtctcgtcgcgaaattcacattaaaaatattaattttctgtatgtacctttaaaattactgtagaaaatctatcagatgggtcttggcgcgcaaaacgcaaaaaataaattgatttcattgcatgctttttccgcgccgagacccatctaacctaaatttttcaaaaaggtgtgcgcctttaaagattactgtagtttcgaacttgtgtagttgcggagttttcatcgatttttttgtaaataaatagttttattttagtttttgaagaaataaatattttttcaacgaacaactatatgaaaaatcgataaaaatcccgcaattttttggaaaattctgtgatggataataccaaattcagatgtttccttcttttttctcatcattgggcttcttgttctcggtgtcttcacaatcaaaaagaacacctatatcaccttcgattcatgggatctagcacaagagattgctcagaacgcgacggatgcagcgatggaaaactcttctgtaagtttttcacggaattctggcctccctcataaatcgaaatggcagagtttgccgaactaggccattttgagtccgcgagattttgtgtaggtttacggcgcgttgcgt

SEQ ID NO: 20 - DNA, 2413 bp, Artificial sequence
  Synthetic
agatctctaaaagttacataaaattgaaagttttgtggggaaattgttttaaaacacgcctatggtaatacgagaacataaaattctgagaatgcgtattacacaacatattcgacgcgcaaaatataactatatctcgtagcgaaaactacagtaatccttaaatgactactgtagcgattgtgtcgatttacgggctcgattctcggaaaaaaaaatcatttttcaaattttgacagcgatattcaatttttcttcgttttttcgtatttctcagtcatttttgtgcttatttttaatattttattcattaataaattaatttaattgaaaaacgtccagacgtatttttacatttcccgtctatttccgtaataggcggttaataacgtagaaatacgtctgtatgagtttactcatcaaaatgacacgaatattggacgtttttaaatcaaattaatttattaataggtaaaatattaaaaaataaacacaaaaatgacaaaggaataggaaaacacgaagagaaattgagtatcgctgtcaaaattcgaaaaacgtggatttttccaagaatcgagcccgtaaatcgacacattcgctacagtagtcatttaaggattattgtagttttcgctacgagatactttgcgcgccaaatatgttgtgaaatacgcattctcagaattttatgttcccgtaataccacaatgaccgaatatcataataaaaaaattcaaaaacaatttctagattttatatgattttttgaaaattgaaaaaatctcagttttcacctaattatatttgaattaccgccaattgaactcgttcgttggagcgcgcttgcattattttcattaattaattttattaattttcattatttcactgattttcttcatttttgaggttttttttatcgggaaatgaaagaaatatacaagaaaaatgcaaaatgtttattaaaaactgaaattagtatttctcggagttttaggcattttcagttactttttaataaagattttgcatttttcttgtatatttctttcattttccgattaaaaaaccccaaaaatgaagaaaatcagtgaaataatgaaaattaattcaaataactaatgaaaataatgcaagcgcgctccaaccaacgagttcaattggcggtaattcaaatataattaggtgaaaactgagattttttcaattttcaaaaaatcatataacatctagacaatttttttgaatttttttatcatgatattcggtcattgtgtccccataggcatgttttaaagcaatttccacctttaaataaaatcgagaaaaaatggcgtcaaaagacatatgtaaaagctgtttctctttgtatattcacacagaatatagcccgattttggaggtgaaaaaccggtttttttgtattctttacgccacataaagtgatatggaagagaaaatcgacatatttatgtacttggagtacgagtacattggagaaaaagggtgaactactggaaaatctgggaaatttcagaaaaaaattccgaaaaatcttttttgctgaaaaaaaaatttgttcgaaatttgggaaatactggaaaattttcatcaaaattaaagcgatttttaacgtttttgcttgggagatactatttgtaattttaatgtttggtgaggacaacagaaatgaccttcagcgtacggaaaacgattaaaagacacatttgaatcgaaaatagcgtgaaaaacatgaaaatatcggaaaaatagctttaaaattggatttgaaagcaaaaaatgtatcaaaaatcgagaaaaatgcttgaaaaagttgaaaaactcaaacaaaaaatcaaaaaaggtttttttaaaaatctgtgagtatcgcaacgaaagcacaacttgaggcatgcgcctttaaaccaaccgtaaccaaattcggaggcaaaaaaagatttctcgctgttttttcagttttaaaatcacttttcacgtttcttttcgcagtaattcgtttgcaaaacatctgaaaaaattttattttataagaaattatataaaaatcgctataaattgagtttttgcccccaaattcagcacggagcacttctcaattaaatataattttattttttaacattgaaagctttcataaaattgagctttgtttgtaaataatcagtgaaaaaacacagaaatcttcaaaaaataaagaaaaagtttttaaaaatctgtgagtcccgcaacgaaaataaaactttaggcatgcgcctttaaaccaaccgtaaccaaattcggcggcaaaaatggatttctcgccgttttttcagttttacaatcactttttaggtttctttttgcagtatttcgtttcccaaacaattaaaaatcaaattttcttttccag

SEQ ID NO: 21 - DNA, 900 bp, Artificial sequence
  Synthetic
ttttgccgtattttccatattttgttttgtatatttatccactcaccccctctctttgtcctgtgaatgaacttgtgccaaaaaagccaaaaataatttactttttttaaagacatattaaaacttgaaaacttggaacaatacggtgcgaaagcgaaacagagacagaaaaatgaacaaagagatccgtgaaaagtgaacgatgatttaggcttcttgttgggcggcgtggatgagagtgatgttgtctcctttgagaagaattctgccgatcttgttgcgtcccttcgtcttcatgttcacctcctcggcttcgtcgaacacgacattcatgaactcgtcgaatccgatgatgtagccctcgagacggtgggtgacatcctcgtacaaccagatttggacccgagtgcggttttggagatatctgaaatttgggaaattgttttctactgattagtgaaaaaatattacctgaagatgaggttcacaggctgaaccatcactttgttgagctttctcgtcgacatttttgcgggatgatctgtaaaaatcaatatattttttgaaagacacaaatttttaagataaatatcaagaaaactcgattttagcagctatttaagctcaaaaactacaatttatggctttaaaacataatttttcacattttacataaatttcaacgaaaaaaaatgccaaaaaacttcaaatttgagcagaaatcggctattttctcgacaaaaataatttcgaaaattcgttcaaaaaagatgagtacgcaagcaccgtgacgcacagttgcggactttgtaaaccgtacccctgcataaaaaggcggaattttgaatttaataaaatgcagtttagaacaaagaaaagcacggagagaattgtgataac

SEQ ID NO: 22 - DNA, 3270 bp, Artificial sequence
  Synthetic
atgagcgatcagaagccggtaagtgaatcttttcgaatgttgctcaacatgattcacggtatttcagaatatggggcgcattgtcgccagcgtcgtggatgtgcagcagttgatgacaatgcagtttgactcgcttatcaaaagcatggactcgttgaaggtatgcgattcatgaaaaactcaatttaatacttatttttcacagattgagcaccagaccggcgtcacccagcttcgcgatgatattcgtcgatccgaggatcggtttcagaagcagttgtccgatttgagcactaatcacggaaaagagctcgagagacttcatcaaattatacacactcttcttgctcgtgatgcgaatcctctcggctcaatgattcctccacagcagcttcagcaacaacagcaaatgttgattcttcagcgacaaatggagatggctcatgtgcaggcagcacaagctcaagctcatgctcacgctcaagctcaagcccaagcacaggcccaaagccaagttatggctaatttgctcaatgctgcgaagccagctattcccgttacacagccactggtcgccactacagcacaagcaaaatcgacagttccagcttctggagttattgcaccaaaggtaacaaattttcgggttttagttccagcaattaaaaattcaatatttttcagacatctccaccagaagttgtaattccacctgctaagccaacattctctactccaacgcctgcagttgttccaaagccagccaccaccggattctcattcggcgggaccaatccagctacttcaatcttcggtaaaaagcctgaaactgcttccccagtcgtggttccagcagctaaagatgatgaagacgaagaacatgatgaggattacgagccagagggcgagttcaagccagtcattccactgccagatcttgtcgaggtgaaaactggagaagaaggagagcaaaccatgttctgcaatcgttcaaaactctacatttatgccaacgagacgaaggaatggaaggagcgaggaaccggcgagctgaaggttctctacaacaaggacaagaagtcgtggagagttgttatgcgacgagaccaggtgaccatttcggaggagacactcccttcaaatattgagaatggaaccgaagaattgtgttggaatattcaggttattccaattacagcttttttcacatttttcggcgttttcctcaacagaaagatcttaatttaaatttaaataccttaatttagcattaaaataaattacaggttctcaaagtctgtgccaatttcccaattctcggctcgatgaccattcaacaaatgaaatccaacgagaaggcctacacatggttctgcgaggacttttccgaagatcagccagctcacgtgaagctttccgctcgtttcgctaacgtcgatatcgctggagagtttaagactttgttcgagaaggctgtcgctgaggcaaaatcatcgagcaacgctggaaagaccatcgacaaggaaatcaagccggcggctgaggtgaagaaggaagtcaagcaagaagttgtgattccatcaaacaacaaaccggaagagactggatttggagatcagttcaagccaaaaccaggatcttgggaatgccctggatgctatgttacttgcaaagctgacgagattgaatgtgcctgttgtgggacatcgaaagacggatctgtcaaagagaaaaatatattctctaagtaaatattctatttgagttatcttaaaattaaaaatttaaatttgcagaccttcgatcctccagcctgccccaggaactccaaaagtgacattcggattcggagcatctgctccagccaaggaacctcttgctcaaacatctcagttcggtggatcactcagcggatctccaagcactagcagtagcattttcggtggtggaacaccaaagggaacaagcgttttcggtggaggtgctgctaatactccaactttctcattcaacaagccagctgccgctgtcaatgcaactaccccatcgttcaatttcaacaatcctgcagcttcaactgcttctccggcaacctctacgactccaggaaattcattgttcggaggtggactctccaagactgaatctacagcttcttcaactactactccgtcgtttatgttcgccaagaattctgaaagtgccttcccaaagccaactttctcattcggaaagcaacagactccatcaacgactgcaccagctaaacaagaagaaaataaacagtccgagacgccaaaatccgtgttcggtagtggattcacgtctggaggtgcaacattcgctgccctttcagccaactctgccaaatctggctccattttcgacgcagcaaatgtcaagaaggcccaagaagaacttgctgctcagaagaaggcaagcattttcgggagcaagaacaccacgttgaacacgacttctgcaacttcacacgatggtgatgagacgaatgaggatggagatggggagtatgagccagaagtcgagtttaagccagttattcctcttccggatttggttgaggtgaaaactggtgaagaggacgaggaggtcatgttctccgccagatgcaagctttataagtactattcggatctcaaggagaataaggaacgtggactcggagatattaaggtcagatttaggattttatatcaaaatgagccagcttggcatgttcttttgatagttttcattaaaatttagtgataaaacgtagaaaataacttatttcagttgaaacagttaagaaaagtgctagttttgcgcacttttcaactgttttgcaagtattttctatgttttttcactaaattttaatgaaaactattaagaaaacaactgatttttgactcatagaggctaaatttgagaaaaataatttattatttcagctcctgaaaagcaatgacaacaaatatcgaatcgtgatgcgtcgtgagcaagtccataagctctgtgccaatttccgtatcgaaaagtcgatgaagctcagtccgaagccaaacttgccaaatgtgctcacatttatgtgccaggtagcaatttttaatttctttttatataaaaatgtactataaattccttatttttcaggacttctcagaagacgcctccaacgctgatccagccatcttcaccgccaaattcaaggacgaggcgaccgccggagcattcaagacggctgttcaggatgctcaatcgaagatg

SEQ ID NO: 23 - protein, 860 aa, Artificial sequence
  Synthetic
MSDQKPNMGRIVASVVDVQQLMTMQFDSLIKSMDSLKIEHQTGVTQLRDDIRRSEDRFQKQLSDLSTNHGKELERLHQIIHTLLARDANPLGSMIPPQQLQQQQQMLILQRQMEMAHVQAAQAQAHAHAQAQAQAQAQSQVMANLLNAAKPAIPVTQPLVATTAQAKSTVPASGVIAPKTSPPEVVIPPAKPTFSTPTPAVVPKPATTGFSFGGTNPATSIFGKKPETASPVVVPAAKDDEDEEHDEDYEPEGEFKPVIPLPDLVEVKTGEEGEQTMFCNRSKLYIYANETKEWKERGTGELKVLYNKDKKSWRVVMRRDQVLKVCANFPILGSMTIQQMKSNEKAYTWFCEDFSEDQPAHVKLSARFANVDIAGEFKTLFEKAVAEAKSSSNAGKTIDKEIKPAAEVKKEVKQEVVIPSNNKPEETGFGDQFKPKPGSWECPGCYVTCKADEIECACCGTSKDGSVKEKNIFSKPSILQPAPGTPKVTFGFGASAPAKEPLAQTSQFGGSLSGSPSTSSSIFGGGTPKGTSVFGGGAANTPTFSFNKPAAAVNATTPSFNFNNPAASTASPATSTTPGNSLFGGGLSKTESTASSTTTPSFMFAKNSESAFPKPTFSFGKQQTPSTTAPAKQEENKQSETPKSVFGSGFTSGGATFAALSANSAKSGSIFDAANVKKAQEELAAQKKASIFGSKNTTLNTTSATSHDGDETNEDGDGEYEPEVEFKPVIPLPDLVEVKTGEEDEEVMFSARCKLYKYYSDLKENKERGLGDIKLLKSNDNKYRIVMRREQVHKLCANFRIEKSMKLSPKPNLPNVLTFMCQDFSEDASNADPAIFTAKFKDEATAGAFKTAVQDAQSKM

SEQ ID NO: 24 - DNA, 4134 bp, Artificial sequence
  Synthetic
atgagcgatcagaagccggtaagtgaatcttttcgaatgttgctcaacatgattcacggtatttcagaatatggggcgcattgtcgccagcgtcgtggatgtgcagcagttgatgacaatgcagtttgactcgcttatcaaaagcatggactcgttgaaggtatgcgattcatgaaaaactcaatttaatacttatttttcacagattgagcaccagaccggcgtcacccagcttcgcgatgatattcgtcgatccgaggatcggtttcagaagcagttgtccgatttgagcactaatcacggaaaagagctcgagagacttcatcaaattatacacactcttcttgctcgtgatgcgaatcctctcggctcaatgattcctccacagcagcttcagcaacaacagcaaatgttgattcttcagcgacaaatggagatggctcatgtgcaggcagcacaagctcaagctcatgctcacgctcaagctcaagcccaagcacaggcccaaagccaagttatggctaatttgctcaatgctgcgaagccagctattcccgttacacagccactggtcgccactacagcacaagcaaaatcgacagttccagcttctggagttattgcaccaaaggtaacaaattttcgggttttagttccagcaattaaaaattcaatatttttcagacatctccaccagaagttgtaattccacctgctaagccaacattctctactccaacgcctgcagttgttccaaagccagccaccaccggattctcattcggcgggaccaatccagctacttcaatcttcggtaaaaagcctgaaactgcttccccagtcgtggttccagcagctaaagatgatgaagacgaagaacatgatgaggattacgagccagagggcgagttcaagccagtcattccactgccagatcttgtcgaggtgaaaactggagaagaaggagagcaaaccatgttctgcaatcgttcaaaactctacatttatgccaacgagacgaaggaatggaaggagcgaggaaccggcgagctgaaggttctctacaacaaggacaagaagtcgtggagagttgttatgcgacgagaccaggtgaccatttcggaggagacactcccttcaaatattgagaatggaaccgaagaattgtgttggaatattcaggttattccaattacagcttttttcacatttttcggcgttttcctcaacagaaagatcttaatttaaatttaaataccttaatttagcattaaaataaattacaggttctcaaagtctgtgccaatttcccaattctcggctcgatgaccattcaacaaatgaaatccaacgagaaggcctacacatggttctgcgaggacttttccgaagatcagccagctcacgtgaagctttccgctcgtttcgctaacgtcgatatcgctggagagtttaagactttgttcgagaaggctgtcgctgaggcaaaatcatcgagcaacgctggaaagaccatcgacaaggaaatcaagccggcggctgaggtgaagaaggaagtcaagcaagaagttgtgattccatcaaacaacaaaccggaagagactggatttggagatcagttcaagccaaaaccaggatcttgggaatgccctggatgctatgttacttgcaaagctgacgagattgaatgtgcctgttgtgggacatcgaaagacggatctgtcaaagagaaaaatatattctctaagtaaatattctatttgagttatcttaaaattaaaaatttaaatttgcagaccttcgatcctccagcctgccccaggaactccaaaagtgacattcggattcggagcatctgctccagccaaggaacctcttgctcaaacatctcagttcggtggatcactcagcggatctccaagcactagcagtagcattttcggtggtggaacaccaaagggaacaagcgttttcggtggaggtgctgctaatactccaactttctcattcaacaagccagctgccgctgtcaatgcaactaccccatcgttcaatttcaacaatcctgcagcttcaactgcttctccggcaacctctacgactccaggaaattcattgttcggaggtggactctccaagactgaatctacagcttcttcaactactactccgtcgtttatgttcgccaagaattctgaaagtgccttcccaaagccaactttctcattcggaaagcaacagactccatcaacgactgcaccagctaaacaagaagaaaataaacagtccgagacgccaaaatccgtgttcggtagtggattcacgtctggaggtgcaacattcgctgccctttcagccaactctgccaaatctggctccattttcgacgcagcaaatgtcaagaaggcccaagaagaacttgctgctcagaagaaggcaagcattttcgggagcaagaacaccacgttgaacacgacttctgcaacttcacacgatggtgatgagacgaatgaggatggagatggggagtatgagccagaagtcgagtttaagccagttattcctcttccggatttggttgaggtgaaaactggtgaagaggacgaggaggtcatgttctccgccagatgcaagctttataagtactattcggatctcaaggagaataaggaacgtggactcggagatattaaggtcagatttaggattttatatcaaaatgagccagcttggcatgttcttttgatagttttcattaaaatttagtgataaaacgtagaaaataacttatttcagttgaaacagttaagaaaagtgctagttttgcgcacttttcaactgttttgcaagtattttctatgttttttcactaaattttaatgaaaactattaagaaaacaactgatttttgactcatagaggctaaatttgagaaaaataatttattatttcagctcctgaaaagcaatgacaacaaatatcgaatcgtgatgcgtcgtgagcaagtccataagctctgtgccaatttccgtatcgaaaagtcgatgaagctcagtccgaagccaaacttgccaaatgtgctcacatttatgtgccaggtagcaatttttaatttctttttatataaaaatgtactataaattccttatttttcaggacttctcagaagacgcctccaacgctgatccagccatcttcaccgccaaattcaaggacgaggcgaccgccggagcattcaagacggctgttcaggatgctcaatcgaagatgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagacctgcataatggcttcttctcttcgtcagatcctcgactctcagaagatggagtggcgttctaacgctggaggatctgcggccgcagattacaaggatcacgatggcgattacaaggatcacgatatcgattacaaggatgatgatgataagtaa

SEQ ID NO: 25 - protein, 1147 aa, Artificial sequence
  Synthetic
MSDQKPNMGRIVASVVDVQQLMTMQFDSLIKSMDSLKIEHQTGVTQLRDDIRRSEDRFQKQLSDLSTNHGKELERLHQIIHTLLARDANPLGSMIPPQQLQQQQQMLILQRQMEMAHVQAAQAQAHAHAQAQAQAQAQSQVMANLLNAAKPAIPVTQPLVATTAQAKSTVPASGVIAPKTSPPEVVIPPAKPTFSTPTPAVVPKPATTGFSFGGTNPATSIFGKKPETASPVVVPAAKDDEDEEHDEDYEPEGEFKPVIPLPDLVEVKTGEEGEQTMFCNRSKLYIYANETKEWKERGTGELKVLYNKDKKSWRVVMRRDQVLKVCANFPILGSMTIQQMKSNEKAYTWFCEDFSEDQPAHVKLSARFANVDIAGEFKTLFEKAVAEAKSSSNAGKTIDKEIKPAAEVKKEVKQEVVIPSNNKPEETGFGDQFKPKPGSWECPGCYVTCKADEIECACCGTSKDGSVKEKNIFSKPSILQPAPGTPKVTFGFGASAPAKEPLAQTSQFGGSLSGSPSTSSSIFGGGTPKGTSVFGGGAANTPTFSFNKPAAAVNATTPSFNFNNPAASTASPATSTTPGNSLFGGGLSKTESTASSTTTPSFMFAKNSESAFPKPTFSFGKQQTPSTTAPAKQEENKQSETPKSVFGSGFTSGGATFAALSANSAKSGSIFDAANVKKAQEELAAQKKASIFGSKNTTLNTTSATSHDGDETNEDGDGEYEPEVEFKPVIPLPDLVEVKTGEEDEEVMFSARCKLYKYYSDLKENKERGLGDIKLLKSNDNKYRIVMRREQVHKLCANFRIEKSMKLSPKPNLPNVLTFMCQDFSEDASNADPAIFTAKFKDEATAGAFKTAVQDAQSKMTCIMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKMASSLRQILDSQKMEWRSNAGGSAAADYKDHDGDYKDHDIDYKDDDDK

SEQ ID NO: 26 - DNA, 66 bp, Artificial sequence
  Synthetic
gattacaaggatcacgatggcgattacaaggatcacgatatcgattacaaggatgatgatgataag

SEQ ID NO: 27 - protein, 22 aa, Artificial sequence
  Synthetic Construct
DYKDHDGDYKDHDIDYKDDDDK

SEQ ID NO: 28 - DNA, 2100 bp, Artificial sequence
  Synthetic
atgtccacctttaacttcgccagcatggccgcccaactgggccaggagcagggaatatcattcgagaacaaggtgctttcctggaacacagctgccgatggtaagtttgggtgacgctttgggtactccatgcagtggctatattgaggctttttacagtccaggatgtggtggatgcccttaacaaacagaccaccgtgcactatctgaatctggacgggaacacactgggcgttgaggccgccaaggcgattggtgagggtctgaagcgtcatccagagtttcggaaggcgctgtggaagaacatgtttactggtcgtctcatatcggagattccggaggcactcaagcacctgggagccgcgctaattgtcgcgggcgccaaactgacagtcctggatctcagcgacaatgccttaggaccgaatggcatgcgaggcttagaggagttactgcgatccccggtctgctactcgctgcaggagctgctgctgtgcaattgtggccttggtcccgagggcggtagtatgctgtcccgggctctgatcgatctgcatgccaatgccaacaaggcgggcttcccgctccagctgcgtgtgttcataggttcgcgcaatcgtctcgaggatgccggtgctacggaaatggcaaccgcattccaaaccctcaagaccttcgaggagattgttctggagcaaaactccatttacatcgaaggcgtcgaggcccttgccgaatccttcaagcataatcctcatctacgagtgctaaacatgaacgacaatactctaaagtccgagggagctgaaaaaatagctgaggctcttcccttcttgccactgtgagtcggttataaagtttttgatgagggagagtgagtgagtgttatgtgatatctttttgctttgcaggctgcgtgaaatgagctttggagactgcctgatcaaaactaatggcgcctaccacttcggtgaggctctggagagaggaaacgaacgactggaagttatcgacttaggttttaacgaaatcaacagcgacggcggcttggtgttggtgaatgctatgggaaacaagcccaagctacgcatcttgaatctagatggcaatagctttggagaagaaggcagcgagaagataatcagcgagatgagtaagttgccaactgctgccgcactgcaaccgtttcagcaccaggaagaggaggatttggaagatgaataccaggctgacaagcaggacgcagattacgaagaggaagaggaagtacacgagcacgccaacgatactaccgaagaagcagatgaggatagcgagggcgacgaggacgacgaggaagacgagggagacgaggagtacagcaacgtcgcggaggagactgcctatgtcactacgaatgcctacacgaccaaggttagtttatacttcggagtactgtgcaaatggtaacagcaattaatcgaattatctttaattttaaattttcacagctttttaacgacacaaccaactcgatggccagcgaaacttttgcggtcgcgaacaagacgatcagccaaaaatgcactccagagaagttctgtttgagccagaaaccctgctcccaggaagatttcgattcgctagatatggataacaaacttgaggctttgcagtcgattgtcaacgtaagatattctcaataattagaatgcaattagagtaacgtaacatttttgtacagcaattcaccggcgacaaccatttgctactgctcgtcttcaccaccttgaagtgcgcgcatttgtcgcaatcctcgaaagctgcgttggatctggccgtctccttgtaccaggccacctttgactatgccatcaagacaaagcaggagacacgtgtactcaactatgtactgatgcagctccgtttgttgccctgcaaggaggtattccattcggactacgatgtcaagaactgtcgatttgctcttcgcgaggctctcaagcaaccaacgtttgccaacgacaacattaagaattcctttaagactttcctggagggtgcggagtcgtaaagagaattagagaagagattacctttatttcccactttccgatttgt

SEQ ID NO: 29 - protein, 596 aa, Artificial sequence
  Synthetic
MSTFNFASMAAQLGQEQGISFENKVLSWNTAADVQDVVDALNKQTTVHYLNLDGNTLGVEAAKAIGEGLKRHPEFRKALWKNMFTGRLISEIPEALKHLGAALIVAGAKLTVLDLSDNALGPNGMRGLEELLRSPVCYSLQELLLCNCGLGPEGGSMLSRALIDLHANANKAGFPLQLRVFIGSRNRLEDAGATEMATAFQTLKTFEEIVLEQNSIYIEGVEALAESFKHNPHLRVLNMNDNTLKSEGAEKIAEALPFLPLLREMSFGDCLIKTNGAYHFGEALERGNERLEVIDLGFNEINSDGGLVLVNAMGNKPKLRILNLDGNSFGEEGSEKIISEMSKLPTAAALQPFQHQEEEDLEDEYQADKQDADYEEEEEVHEHANDTTEEADEDSEGDEDDEEDEGDEEYSNVAEETAYVTTNAYTTKLFNDTTNSMASETFAVANKTISQKCTPEKFCLSQKPCSQEDFDSLDMDNKLEALQSIVNQFTGDNHLLLLVFTTLKCAHLSQSSKAALDLAVSLYQATFDYAIKTKQETRVLNYVLMQLRLLPCKEVFHSDYDVKNCRFALREALKQPTFANDNIKNSFKTFLEGAES

SEQ ID NO: 30 - DNA, 2967 bp, Artificial sequence
  Synthetic
atggattacaaggatcacgatggcgattacaaggatcacgatatcgattacaaggatgatgatgataaggcggccgctatggcttcttctcttcgtcagatcctcgactctcagaagatggagtggcgttctaacgctggaggatctgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggggcccatgcatggcatgtccacctttaacttcgccagcatggccgcccaactgggccaggagcagggaatatcattcgagaacaaggtgctttcctggaacacagctgccgatggtaagtttgggtgacgctttgggtactccatgcagtggctatattgaggctttttacagtccaggatgtggtggatgcccttaacaaacagaccaccgtgcactatctgaatctggacgggaacacactgggcgttgaggccgccaaggcgattggtgagggtctgaagcgtcatccagagtttcggaaggcgctgtggaagaacatgtttactggtcgtctcatatcggagattccggaggcactcaagcacctgggagccgcgctaattgtcgcgggcgccaaactgacagtcctggatctcagcgacaatgccttaggaccgaatggcatgcgaggcttagaggagttactgcgatccccggtctgctactcgctgcaggagctgctgctgtgcaattgtggccttggtcccgagggcggtagtatgctgtcccgggctctgatcgatctgcatgccaatgccaacaaggcgggcttcccgctccagctgcgtgtgttcataggttcgcgcaatcgtctcgaggatgccggtgctacggaaatggcaaccgcattccaaaccctcaagaccttcgaggagattgttctggagcaaaactccatttacatcgaaggcgtcgaggcccttgccgaatccttcaagcataatcctcatctacgagtgctaaacatgaacgacaatactctaaagtccgagggagctgaaaaaatagctgaggctcttcccttcttgccactgtgagtcggttataaagtttttgatgagggagagtgagtgagtgttatgtgatatctttttgctttgcaggctgcgtgaaatgagctttggagactgcctgatcaaaactaatggcgcctaccacttcggtgaggctctggagagaggaaacgaacgactggaagttatcgacttaggttttaacgaaatcaacagcgacggcggcttggtgttggtgaatgctatgggaaacaagcccaagctacgcatcttgaatctagatggcaatagctttggagaagaaggcagcgagaagataatcagcgagatgagtaagttgccaactgctgccgcactgcaaccgtttcagcaccaggaagaggaggatttggaagatgaataccaggctgacaagcaggacgcagattacgaagaggaagaggaagtacacgagcacgccaacgatactaccgaagaagcagatgaggatagcgagggcgacgaggacgacgaggaagacgagggagacgaggagtacagcaacgtcgcggaggagactgcctatgtcactacgaatgcctacacgaccaaggttagtttatacttcggagtactgtgcaaatggtaacagcaattaatcgaattatctttaattttaaattttcacagctttttaacgacacaaccaactcgatggccagcgaaacttttgcggtcgcgaacaagacgatcagccaaaaatgcactccagagaagttctgtttgagccagaaaccctgctcccaggaagatttcgattcgctagatatggataacaaacttgaggctttgcagtcgattgtcaacgtaagatattctcaataattagaatgcaattagagtaacgtaacatttttgtacagcaattcaccggcgacaaccatttgctactgctcgtcttcaccaccttgaagtgcgcgcatttgtcgcaatcctcgaaagctgcgttggatctggccgtctccttgtaccaggccacctttgactatgccatcaagacaaagcaggagacacgtgtactcaactatgtactgatgcagctccgtttgttgccctgcaaggaggtattccattcggactacgatgtcaagaactgtcgatttgctcttcgcgaggctctcaagcaaccaacgtttgccaacgacaacattaagaattcctttaagactttcctggagggtgcggagtcgtaaagagaattagagaagagattacctttatttcccactttccgatttgt

SEQ ID NO: 31 - protein, 885 aa, Artificial sequence
  Synthetic
MDYKDHDGDYKDHDIDYKDDDDKAAAMASSLRQILDSQKMEWRSNAGGSVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKGPMHGMSTFNFASMAAQLGQEQGISFENKVLSWNTAADVQDVVDALNKQTTVHYLNLDGNTLGVEAAKAIGEGLKRHPEFRKALWKNMFTGRLISEIPEALKHLGAALIVAGAKLTVLDLSDNALGPNGMRGLEELLRSPVCYSLQELLLCNCGLGPEGGSMLSRALIDLHANANKAGFPLQLRVFIGSRNRLEDAGATEMATAFQTLKTFEEIVLEQNSIYIEGVEALAESFKHNPHLRVLNMNDNTLKSEGAEKIAEALPFLPLLREMSFGDCLIKTNGAYHFGEALERGNERLEVIDLGFNEINSDGGLVLVNAMGNKPKLRILNLDGNSFGEEGSEKIISEMSKLPTAAALQPFQHQEEEDLEDEYQADKQDADYEEEEEVHEHANDTTEEADEDSEGDEDDEEDEGDEEYSNVAEETAYVTTNAYTTKLFNDTTNSMASETFAVANKTISQKCTPEKFCLSQKPCSQEDFDSLDMDNKLEALQSIVNQFTGDNHLLLLVFTTLKCAHLSQSSKAALDLAVSLYQATFDYAIKTKQETRVLNYVLMQLRLLPCKEVFHSDYDVKNCRFALREALKQPTFANDNIKNSFKTFLEGAES

SEQ ID NO: 32 - DNA, 1379 bp, Artificial sequence
  Synthetic
tgtgctgagggcagtaaatcatattgctcatcatgtccaagctcctaagtccaggtagattttggacagggcaaaaccctgttggtggtttttctaaggggaccatttcgagtcctgggttttgctattacctaagccggcgatcggcgatctgcgatcggagatcttcgatcgtggttttttccagcggaagttcgcgctctgcattaatcgggtatttttggtggccccggcaggcaaacagataattatatccggaaatttgacttttcgctcgtatttttctggattttcggagctccgagcggcattcgcctgcgattttctcggtacgtgtgtgtgggaattcactaattaggcataatgaaaccttttcgtggagttcccctcggttagggttgtggatttgcacgctttacgatggttggcaactaactcgatattatatatgcttacatctaaatgaatttgatacccgatttatgtggattttcgtgtttttgatcaggggagattcattgcgtcttatttttttttttacaaaaagaatataacttatttccatatcatttaaagtttttaagttaaattcttgtctaaacctaaactaacgctgcaaacaacaacattcaatgaagtttactaaatgttctattgggaattgcggaacaaaatcttttctacaagaatgcatttttcaggaaatgcttttatgtaaaaacataatttatcattactctgactgcactttttcaaaacttagaaactctgtcctatgaattcccgtcgatccaaagatattctcaatcccctttttgaatcaacaagtaaaatatttcaaaaattgccgacaattcccctcgtattccccgtcgcgcatcccaacacgcatacttcccaggcattttcccaaatcgagagaaaacccaaagaataacccaagagaaacagaaaaatccagagcgtcgagtcaaggctctcttcaatttagctttgaatttgctgtattttcgttttgcagccgccgctgccgctcgagaaaatcgaaatcccccgccgcctgacgtcatacctgccgatgccgcagcttccgccattgagtgggagcgggatggcaagacaagcgagcgagcgggacgacgatagagcggcggcagcgaatggccgtcgagcagccgcaaaatgtcaatttgagcaatggccggaaggatcctgcgtcagttgcgttccgtaagtgcgtgcgagcagatcgatccagcaaaacgcgggcgtgaagaatatctacggagttatacagttccgaaataagaatatattgttagataccacaaattctaactgaagaagtgcgctaaaaagccaagcaagatcaccaaatg

SEQ ID NO: 33 - DNA, 22 bp, Artificial sequence
  Synthetic
cagacaagctgttgggttcctg

SEQ ID NO: 34 - DNA, 24 bp, Artificial sequence
  Synthetic
aagttgttggacactaaggatcgg

SEQ ID NO: 35 - DNA, 23 bp, Artificial sequence
  Synthetic
accaccgtgtcctgaatcatctc

SEQ ID NO: 36 - DNA, 20 bp, Artificial sequence
  Synthetic
tttgtgtcacgggtgcgtag

SEQ ID NO: 37 - DNA, 22 bp, Artificial sequence
  Synthetic
aagactccaacgctggtggttg

SEQ ID NO: 38 - DNA, 22 bp, Artificial sequence
  Synthetic
tcctttggcatggcactcttcg

SEQ ID NO: 39 - DNA, 23 bp, Artificial sequence
  Synthetic
aggacgacaaacattgcaaaggc

SEQ ID NO: 40 - DNA, 24 bp, Artificial sequence
  Synthetic
tcttcttcatgccttgagaactcg

SEQ ID NO: 41 - DNA, 22 bp, Artificial sequence
  Synthetic
tgtggcgaattaactgcttccc

SEQ ID NO: 42 - DNA, 24 bp, Artificial sequence
  Synthetic
tccgagaatgtaatccacctgacg

SEQ ID NO: 43 - DNA, 23 bp, Artificial sequence
  Synthetic
acaagaacacccgtaggacaagc

SEQ ID NO: 44 - DNA, 22 bp, Artificial sequence
  Synthetic
cgccgtttcaaccaccactatc

SEQ ID NO: 45 - DNA, 23 bp, Artificial sequence
  Synthetic
ctcccagctatgaacacaaaggc

SEQ ID NO: 46 - DNA, 23 bp, Artificial sequence
  Synthetic
agtttacctttggcgatggagtg

SEQ ID NO: 47 - DNA, 22 bp, Artificial sequence
  Synthetic
agacggtcagggtgcacttatc

SEQ ID NO: 48 - DNA, 24 bp, Artificial sequence
  Synthetic
cgcttgtgatttcttgttgctctg

SEQ ID NO: 49 - DNA, 22 bp, Artificial sequence
  Synthetic
cgaagacgcaatggcgagaatc

SEQ ID NO: 50 - DNA, 22 bp, Artificial sequence
  Synthetic
tagcgacacgaaggagagcaag

SEQ ID NO: 51 - DNA, 22 bp, Artificial sequence
  Synthetic
agctgatgcttccttgggatgg

SEQ ID NO: 52 - DNA, 23 bp, Artificial sequence
  Synthetic
tgggttggtcttgcaatgagtcg

SEQ ID NO: 53 - DNA, 22 bp, Artificial sequence
  Synthetic
tgctgagagagttggagctcag

SEQ ID NO: 54 - DNA, 22 bp, Artificial sequence
  Synthetic
agaggagcatcctgctgctttg

SEQ ID NO: 55 - DNA, 24 bp, Artificial sequence
  Synthetic
ccttctcacagccagagaaggttg

SEQ ID NO: 56 - DNA, 24 bp, Artificial sequence
  Synthetic
cgggagaacaacggtcatatcctc

SEQ ID NO: 57 - DNA, 22 bp, Artificial sequence
  Synthetic
acaacgcctgcgttatgaatgg

SEQ ID NO: 58 - DNA, 22 bp, Artificial sequence
  Synthetic
acctcccacgtcaattgttgcc

SEQ ID NO: 59 - DNA, 23 bp, Artificial sequence
  Synthetic
tcatttgcgctctaggagttgtc

SEQ ID NO: 60 - DNA, 24 bp, Artificial sequence
  Synthetic
tggtggagagctatctgtgtatgg

SEQ ID NO: 61 - DNA, 23 bp, Artificial sequence
  Synthetic
tgcgttagctgaaggcagttctc

SEQ ID NO: 62 - DNA, 22 bp, Artificial sequence
  Synthetic
aagtgaagtcctcgcagaaccc

SEQ ID NO: 63 - DNA, 22 bp, Artificial sequence
  Synthetic
tcccttggcgttgtacggatac

SEQ ID NO: 64 - DNA, 22 bp, Artificial sequence
  Synthetic
tccgacgctaaagagctccaag

SEQ ID NO: 65 - DNA, 22 bp, Artificial sequence
  Synthetic
tgcacgagttccgtcttgagtg

SEQ ID NO: 66 - DNA, 24 bp, Artificial sequence
  Synthetic
tgcacaagacccagtcttccttag

SEQ ID NO: 67 - DNA, 24 bp, Artificial sequence
  Synthetic
cgctaatctcctccatgctaaccg

SEQ ID NO: 68 - DNA, 23 bp, Artificial sequence
  Synthetic
agccaatcctctcgtaactcctc

SEQ ID NO: 69 - DNA, 23 bp, Artificial sequence
  Synthetic
tgacttgaaacctgctcatgtcg

SEQ ID NO: 70 - DNA, 24 bp, Artificial sequence
  Synthetic
acaagtgcttcttccaaagccttc

SEQ ID NO: 71 - DNA, 22 bp, Artificial sequence
  Synthetic
gatttacgccgctggtccattg

SEQ ID NO: 72 - DNA, 22 bp, Artificial sequence
  Synthetic
tcagtaagtgggtggtgcagtc

SEQ ID NO: 73 - DNA, 22 bp, Artificial sequence
  Synthetic
agatacggatgtggctcggaac

SEQ ID NO: 74 - DNA, 23 bp, Artificial sequence
  Synthetic
agacatgcagcttcatcgtaggc

SEQ ID NO: 75 - DNA, 23 bp, Artificial sequence
  Synthetic
tctccagcaaagccatgggaatc

SEQ ID NO: 76 - DNA, 23 bp, Artificial sequence
  Synthetic
agcttcgaagactcaccagcaag

SEQ ID NO: 77 - DNA, 23 bp, Artificial sequence
  Synthetic
ttcacagcttcttccttcagagc

SEQ ID NO: 78 - DNA, 24 bp, Artificial sequence
  Synthetic
ccaacgagctccttatctcctttc

SEQ ID NO: 79 - DNA, 23 bp, Artificial sequence
  Synthetic
ggtttgcgtttctgacgaagagc

SEQ ID NO: 80 - DNA, 24 bp, Artificial sequence
  Synthetic
tggtgcaacggtaatagcttctgg

SEQ ID NO: 81 - DNA, 22 bp, Artificial sequence
  Synthetic
agagcattgggaaggcatgctg

SEQ ID NO: 82 - DNA, 24 bp, Artificial sequence
  Synthetic
tctctcccttctcaacaccactcc

SEQ ID NO: 83 - DNA, 24 bp, Artificial sequence
  Synthetic
tgaagtggagccatggtttaggac

SEQ ID NO: 84 - DNA, 23 bp, Artificial sequence
  Synthetic
ggtcaacgcagtctttgtgcatc

SEQ ID NO: 85 - DNA, 22 bp, Artificial sequence
  Synthetic
ggttcccaacaccaacaagacg

SEQ ID NO: 86 - DNA, 22 bp, Artificial sequence
  Synthetic
actgatcctgcacctcccaatc

SEQ ID NO: 87 - DNA, 69 bp, Artificial sequence
  Synthetic
atggcttcttctcttcgtcagatcctcgactctcagaagatggagtggcgttctaacgctggaggatct

SEQ ID NO: 88 - protein, 23 aa, Artificial sequence
  Synthetic Construct
MASSLRQILDSQKMEWRSNAGGS

SEQ ID NO: 89 - protein, 42 aa, Artificial sequence
  Synthetic
LAAASGGGGSGGGGSLAAASEFSAAALSGGGGSGGGGSAAAL

SEQ ID NO: 90 - DNA, 5436 bp, Mus musculus
  CDS (293)..(3220)
ggaggaccagcattgttgaggggtcccacagctaggcgtggctccagactgagggcgtatcttcacatagagttccaatttagagactccagcctacagacaaacatttgggaaggatcatgtttttctgagtttcttctggacaggtggaagctttcttatcctggggccaaccctgcctgtctggaaccctgtgcttaataattcctgtccctgtggatgtgtgggctggagagtcttccttgtttcctccgtgctggagacaagcgaccgggaagccctcgcaggtgcgatgacgcagcagccccaggaggactttgagaggagcgtggaggatgcccaggcctggatgaaggtgatacaggagcagcttcaggtcaatgacaacacgaaggggccccgagcggccctggaggcaaggcttcgagagacagagaaaatctgccagctggagtctgaaggaatggtgaaggtggaactggtcctgcgggcggcggaggccctcttggcaacttgccaggagggccagaaacctgagatcctggcccggctgagggatatcaagtctcagtgggaggagacggtcacctacatgacccactgccacagtcgcatcgagtgggtgtggctgcactggagtgagtacctgctggcccaggatgagttttaccgctggttccagaagatggtggtcgcactggagccccccgtggagctgcagctgggcttgaaggagaagcaatggcagctgagccacgcccaggtgctgctgcacaacgtggacaatcaggctgtgctcctggacaggctgttggaggaggcgggctccctgttcagcaggatcggagaccccagcgtggatgaagatgcccagaagaggatgaaggctgagtacgatgccgtgaaggccagagcccagcgcagggtggacctcctggcccaggtggcccaggaccatgagcagtaccgggaggacgtgaatgagttccagctgtggctgaaggcggtggtggagaaggtgcacagctgtctggggcggaactgcaagctggccacagaacttcgtctctctacgctgcaggacatcgccaaggatttccctaggggtgaggagtctctgaaaagattggaggaacaggctgtgggtgtcattcaaaacacctctcccttgggtgcagagaagatctcaggggagctggaggagatgcggggtgtcctggagaagctgagagtcctctggaaagaggaggaagggaggctgcggggcctgctccagtccaggggggactgtgagcagcagatccaacagctggaggcagaactgggagacttcaagaaaagccttcagaggctggcccaggagggcttggagcccacggtgaagacagccacagaggatgagctggtggcccagtggaggctgttctcggggactcgggctgcactggcttcagaggaaccccgtgtagaccggttacaaactcaactgaagaaacttgtcaccttcccggacctgcagtcactctctgacagcgtggtagccaccattcaggaataccaaagtatgaaggggaagaataccaggctccacaatgcgacccgggcagagctgtggcagcgtttccagcggcccctaaatgacctgcagctgtggaaggccctggcccagaggctcctggacatcacggccagcctgcctgacctggcctccattcacacctttctaccccagattgaggcggccctcacggaaagctctcgcctgaaagagcagctggcgatgctgcagctgaagaccgacctgctgggcagcatctttggccaggagagagcagccaccctcctggagcaggtgacaagttctgtgagggacagagacctactgcataacagccttcttcagcggaagagcaaacttcagagcctgcttgttcagcacaaggactttggggtggcttttgatcccctaaacaggaagctcctagacctccaggccaggatccaagcagagaaagggcttccgagggaccttcctggaaagcaggtccagctcctaaggttgcaggggctgcaggaagaggggctggatctggggacacagatcgaggctgtgaggcctcttgcccatgggaactctaagcaccagcagaaagtagaccagatctcctgtgaccagcaagccctgcagaggtccctggaggatctcgtggacaggtgtcagcagaacgtacgggaacattgtaccttcagtcacaggctgtcggagctgcagctatggatcaccatggccacacagacattagagtcacaccaaggggatgtgcgtctgtgggatgctgagtcccaagaggctggactcgagacgctgctgtctgaaatcccagagaaagaggtccaggtgtccctgctccaagcactgggccagcttgtgatgaagaagtcttccccagaaggggcaaccatggtccaggaggagctgaggaagctgatggagtcttggcaggccctgcggctgctagaggagaacatgctgagtctcatgagaaaccagcagctgcagaggacagaggtggacacggggaagaagcaggtgttcaccaacaacatcccaaaggccggctttctcatcaaccctcaggaccccattcccaggagacagcatggggcaaacccactggaaggacacgacctccctgaagatcatccccagctcctgagggactttgaacagtggctgcaggcagaaaactccaagctacgtagaatcatcacaatgagagtggccacagccaaggacttgaggaccagagaggtgaagctgcaggagctggaggcccgaatcccagaaggccagcacctctttgagaacctgcttcgtctcaggccggcaagggacccctccaacgagctggaagatctgcgctaccggtggatgctgtacaagtccaagctcaaggactctggccacctgctgaccgagagttctccgggggagctgactgcattccagaagagtcggaggcagaagcggtggagtccctgctctctcctacagaaagcatgccgtgtggcactgccattgcagctgttgctcctgctctttctgctgctgctgttcctgctgccggccggcgaggaggagcgcagctgcgccctggccaacaacttcgcccgctcctttgcgctcatgcttcggtacaatggccccccgcccacctagcccactgggcgcacaggtgaccttctgcagtccctcagaggctggctgctgggggcccagtagccgatctcacacccagagcagccttagaccaaatgctgcctctgtttccagaacaaacttgcctttttctacgatgctgcttctgtctatttatactaaaatgtgtactgtgtgtggttagggaatatgtacagaatttttatatgccatgtctgtacatgctcagtgtagataacttcagatacaatgtctctttgtgcctatggaagcccaaaacagtgggtttctctagtgggatttgtggcacttgtgccttagcctcctggaatccctgggggagagggaaaaaaaaaaatcacccgttactttgatgacagcctgttaaccagaaatccagatgttgctgaagaagcctctgagtggttaccatagccacacagaattggagtcctgggacttgagtatttggctagttttaaagtcacacagacttgggccgtgggctagataaagagaagccgtgatggtgggccactggcttagccagagcagtgagacaagatgcgtgccagagggatgccaagagagcaacaagaagagctgtaaagatggagcccaatggagcaccacaaaatgatggtacggagcacccaaccctgcacagcagccactgataacacagaccatggaggaatgcccatgactatgactgtgaccgtgaccgtgaccatgaccgtgactgcgatcccacgtcttcagaagtggctgggttttatttgcatgttttctccaggagaaagaaaaagatgaacttgactttttattctcatgcataagggtgaagtggcctgtgtcagtcctgggaggcccgtgccactgtacacagctgttgagctgtctgcagcctagggcatgagctgagagcagagtggaaatgcttgcccaccaggccaggaggtccaccctaagcagagaggagaaactcaggccatctgaaggggcctctcttcccttccaagaagggactgtgcaggctggtgggtctacaaggctgggcaggtcccacattcccccatgggcctacggctcacagtctgaccccaggcaggagcaggcaaaggcagctccaggcacaggtgggaggcatctgggtgattccaggttatgcccaaggctggacacataaccagggaattctgggatgattcctaagtgctttaaaactggatcgggttcaccctaaaccgacacttttttttttaacctttagaaaatacttaacactccctttttccagatgggattacacactcctttggagtgtgtgtgctcgcaggcctgatcaccagctcaccatcatcagtgtcacagacccagagaggcactttaaatacagcaaatactgtagccctaagagggactggactcccaggtcaccgcactgatgcacatgcaagctggcatcaggctgtatcagaggcagctttcatcttagcatggtacccagaaaaggttggacatcagcaaacaggaaagcagtgtcttggggctcgtgaccctgactttgtctgtaaccccagtcacaaagccctctctgcacctggccactttgctgggaaatcagggactggtgaaccctgacctcacttagacccaggtagccgagccctgatctcagacccagatagcagagccctgatgctcactggccaagaaagaccactggtccaccactccatcagaactgtggcaagggccatcaatacttctgtaaccacttttccatttcaaaagtaaaaaacctgaggcccagagaggggaagtaactcacccatagccacacagcaagaacctatacagccagaaatggactgataccaggtttattagagcaaacctccccacattttccttcccaggaaaaatgaacagggctcgactttggagcacttgcctaacaggtgtgagtcctgagttccatgacctgaattgaagagggattgggggagagctgtttgctacgaatgtgggcgtgtctcctgataaatactggggagggtcaaacttcaatacctgggtgggggtggggcagggtgggagcctggctgtgtttttaacgacaatgacactgtggattgcttgttgtgtaacattttttttaacagaggggactgatatttttattactttcatatgaatatagctgcaatatg

SEQ ID NO: 91 - protein, 975 aa, Mus musculus
MTQQPQEDFERSVEDAQAWMKVIQEQLQVNDNTKGPRAALEARLRETEKICQLESEGMVKVELVLRAAEALLATCQEGQKPEILARLRDIKSQWEETVTYMTHCHSRIEWVWLHWSEYLLAQDEFYRWFQKMVVALEPPVELQLGLKEKQWQLSHAQVLLHNVDNQAVLLDRLLEEAGSLFSRIGDPSVDEDAQKRMKAEYDAVKARAQRRVDLLAQVAQDHEQYREDVNEFQLWLKAVVEKVHSCLGRNCKLATELRLSTLQDIAKDFPRGEESLKRLEEQAVGVIQNTSPLGAEKISGELEEMRGVLEKLRVLWKEEEGRLRGLLQSRGDCEQQIQQLEAELGDFKKSLQRLAQEGLEPTVKTATEDELVAQWRLFSGTRAALASEEPRVDRLQTQLKKLVTFPDLQSLSDSVVATIQEYQSMKGKNTRLHNATRAELWQRFQRPLNDLQLWKALAQRLLDITASLPDLASIHTFLPQIEAALTESSRLKEQLAMLQLKTDLLGSIFGQERAATLLEQVTSSVRDRDLLHNSLLQRKSKLQSLLVQHKDFGVAFDPLNRKLLDLQARIQAEKGLPRDLPGKQVQLLRLQGLQEEGLDLGTQIEAVRPLAHGNSKHQQKVDQISCDQQALQRSLEDLVDRCQQNVREHCTFSHRLSELQLWITMATQTLESHQGDVRLWDAESQEAGLETLLSEIPEKEVQVSLLQALGQLVMKKSSPEGATMVQEELRKLMESWQALRLLEENMLSLMRNQQLQRTEVDTGKKQVFTNNIPKAGFLINPQDPIPRRQHGANPLEGHDLPEDHPQLLRDFEQWLQAENSKLRRIITMRVATAKDLRTREVKLQELEARIPEGQHLFENLLRLRPARDPSNELEDLRYRWMLYKSKLKDSGHLLTESSPGELTAFQKSRRQKRWSPCSLLQKACRVALPLQLLLLLFLLLLFLLPAGEEERSCALANNFARSFALMLRYNGPPPT

SEQ ID NO: 92 - DNA, 4080 bp, Mus musculus
  CDS (51)..(2792)
gacgaggcctgaggcggcggcgcgaggcagcatggtctgagacggtgaacatggacttttctcggctgcacacgtacaccccaccccagtgtgtgccggagaacactggctacacttacgcactcagttctagttactcgtcggatgctctggattttgaaactgagcacaagttggaacctgtatttgactctccaaggatgtcccgccgcagcttgcgtctggtcacaacagcttcgtacagcagtggggacagccaggctattgattcgcacattagcaccagcagggccacccccgccaaggggagagaaaccaggacagtcaaacagagaagaagtgcaagcaagccagcttttagtatcaaccacctgtcagggaagggcttgtcctcgagcacaagccatgacagctcttgcagcctgcggagtgccacggtgctgcggcaccctgtgctagatgagtccctgattcgtgagcagaccaaagtggaccacttctggggtctcgatgatgatggtgaccttaaaggtggaaataaagctgccactcagggaaatggtgaactggcagcagaggtggcgagcagcaatggatacacttgccgtgactgcaggatgctctcagcgcgcactgacgcactcacagcccactctgccatccacgggaccacctccagggtgtactccagagacaggactctcaaaccacgcggagtgtccttttacctggataggactctgtggctggccaagtccacctcctcatcctttgcatcatttatagttcaacttttccaagtggttttaatgaagctcaattttgaaacttacaaattgaaaggctatgaatccagagcttatgaatcacagagctatgagacaaagagccatgagtcagaagcccatctcggtcactgtgggaggatgactgccggagaactttccagagtggacggggagtccctgtgcgatgactgtaaggggaagaagcaccttgagatacacacagccacccactcgcaactgccccagccacacagggtggccggggccatggggcgcctctgcatctatacaggtgacctcttggttcaagcactgcgaaggactagagctgccgggtggtctgtggccgaggccgtgtggtcggtgctctggctggctgtctctgctccagggaaggcagcctcgggaaccttctggtggctagggagcggctggtaccaatttgttactttgatttcttggctgaatgtctttcttcttaccaggtgccttcgaaatatttgcaaggtttttgtcttgctcctcccactcctacttttactaggtgctggtgtctccctgtggggccagggaaacttcttctcactcctaccagtgctgaactggacggccatgcagccaacacagagggtggacgattccaagggcatgcatagacctggccctcttcccccgagcccacctccaaaggttgatcacaaggcttcccagtggcctcaggagagtgacatggggcagaaggtagcttctttgagtgcgcagtgccacaaccatgatgagagacttgcagagctgacagtcctgcttcagaaactacagatacgggtagaccaagtggatgacggcagggaagggctgtcactgtgggtcaagaatgtggttggacagcacctgcaggagatgggcaccatagaaccacctgatgctaagactgacttcatgactttccaccatgaccatgaagtgcgtctctccaacttggaagatgttcttagaaaactgacagaaaaatctgaggctatccagaaggagctggaagaaaccaagctgaaagcaggcagcagggatgaagagcagcccctccttgaccgtgtgcagcacctagaactggaactgaacctgttgaagtcacagctgtcagactggcagcatctgaagaccagctgtgagcaggctggggcccgcatccaggagactgtgcagctcatgttctctgaggatcagcagggcggttccctcgagtggctattagagaagctttcttctcggttcgtgagcaaggatgagctgcaggtgctcttacatgaccttgagctgaaactgctgcagaatatcacacaccacatcaccgtgacaggacaggccccgacatccgaggctattgtgtctgccgtgaatcaggcagggatttcaggaatcacagaagcgcaagcacatatcattgtgaacaatgctctgaagctgtactcccaagacaagacggggatggtggactttgctctggagtctggaggtggcagcatcctaagcactcggtgctctgagacctatgagaccaagacggcactgctgagcctgtttggggtcccactgtggtacttctcacagtcacctcgagtggtgatccagcccgacatctacccagggaattgctgggcgttcaaaggttcccaggggtacctggtggtgcggttgtccatgaagatctacccaaccacattcaccatggaacacattccaaagacactatcacccactggtaacatctccagtgcccccaaagactttgcagtctatggactggaaacggagtatcaagaagaggggcagcctctgggacggttcacctatgaccaggaaggagactcactccagatgttccacacactggaaagacctgaccaagccttccagatagtagagctccgggtcctgtccaactggggccaccctgagtacacttgcctctaccggttccgagtccacggagagcccatccagtagagcactccagccatgtacatgtctgtatataccaagacgccataacactggaactcattgagaaagagagcacgtgagcataggaacttgtcagccacccttcaataaatgtgctgtggtcctgaggacgtgctgagtcctctgaggctgtgcgtgtgttctcaaagcccaaaagccggggaggcttttttaattcatttgaatgtatgattctcggacactccttttgacttagggatttgcttgaatgcataaagtcaagaagcagataggctggcaggtttccaggctcctgacataaaggctgagagctctgcattggccatcctctggatgtcaagaaggcaaagtcccatctgctaaacccacgtttctttcagggaaaactgttgctctgtcttcagtctgaccccttccaggttccctggtggggagaggctatgtcatagactgtctgctttgagctgggactcctctgattccccaggggagtagatcgggctttgcagaagagcagtggggtgtgcacaccatgaatggtgctcttgggtctcagccaggccgttcctggcagatcacagaatggacaggaaccttgagaagggatgctctgctgtccttgagctatgagtctctgctgaagggggaacactaaggtggtgctagtggctgccactcttggtctttatcacagcagcattctgtgctgcccttgggtttgcctctacaccattatcagcccttctcactgattcaaatttgggtatatttaatatttagacttttgtgagtagactttataactgatacatcatctggacacttaaaagtgtttagatgtcttacctaaagaattgttaagtttcattggttagacagccttggcagaatacaaagctcacctgctggggctcagagttctgggatttctcgcatgttgcaagcagcccttaagacagtggcccaaagcacatcccactccatagggttagagccactgcctgtgtggccaggttgtgtgggatgcacacattttgcatactcaaggttacatgcagaagtcagaatttcctatattaaactaaattggggaattgggggtggagatatttcaaatatttatttttaaagatgcaagataggactttgtgcaatgtatttttgtaaatgcttttcaaaacacttgtctttggtagtgcttctactgccactaaattaagatgctattgagatgtttaaataaaagagttaatttttaaaagtgaaaaaaaaaaaaaaaaaaaaaaaa

SEQ ID NO: 93 - protein, 913 aa, Mus musculus
MDFSRLHTYTPPQCVPENTGYTYALSSSYSSDALDFETEHKLEPVFDSPRMSRRSLRLVTTASYSSGDSQAIDSHISTSRATPAKGRETRTVKQRRSASKPAFSINHLSGKGLSSSTSHDSSCSLRSATVLRHPVLDESLIREQTKVDHFWGLDDDGDLKGGNKAATQGNGELAAEVASSNGYTCRDCRMLSARTDALTAHSAIHGTTSRVYSRDRTLKPRGVSFYLDRTLWLAKSTSSSFASFIVQLFQVVLMKLNFETYKLKGYESRAYESQSYETKSHESEAHLGHCGRMTAGELSRVDGESLCDDCKGKKHLEIHTATHSQLPQPHRVAGAMGRLCIYTGDLLVQALRRTRAAGWSVAEAVWSVLWLAVSAPGKAASGTFWWLGSGWYQFVTLISWLNVFLLTRCLRNICKVFVLLLPLLLLLGAGVSLWGQGNFFSLLPVLNWTAMQPTQRVDDSKGMHRPGPLPPSPPPKVDHKASQWPQESDMGQKVASLSAQCHNHDERLAELTVLLQKLQIRVDQVDDGREGLSLWVKNVVGQHLQEMGTIEPPDAKTDFMTFHHDHEVRLSNLEDVLRKLTEKSEAIQKELEETKLKAGSRDEEQPLLDRVQHLELELNLLKSQLSDWQHLKTSCEQAGARIQETVQLMFSEDQQGGSLEWLLEKLSSRFVSKDELQVLLHDLELKLLQNITHHITVTGQAPTSEAIVSAVNQAGISGITEAQAHIIVNNALKLYSQDKTGMVDFALESGGGSILSTRCSETYETKTALLSLFGVPLWYFSQSPRVVIQPDIYPGNCWAFKGSQGYLVVRLSMKIYPTTFTMEHIPKTLSPTGNISSAPKDFAVYGLETEYQEEGQPLGRFTYDQEGDSLQMFHTLERPDQAFQIVELRVLSNWGHPEYTCLYRFRVHGEPIQ

SEQ ID NO: 94 - DNA, 3035 bp, Drosophila melanogaster
  CDS (493)..(2196)
cgcgttccaatcggttgttctctgtttcgctgtctgtaattatattttaacgtgcttccgcgaaattgtgtgaagtaatcccactagaatcccgcataattccgccaaaacaactgtgataaaacaaaactatgccctctaaagtaacttaggccagtaaaatccaacttagaagtgtccattcataaagttcccaaatcgcgtgctttttttgccctgcgtataagagttcctgtttgttataagagtttcgcggaaatttcgatagttttcaatgccgaattgttcaagttacagtgtatatagtgtatatacttccataatccctgcaagtgtgtgcttcgaaaaacgaaattgaaatggtttaaaacacaaattgattacaaaaattgatttcaagtggcacatcgagcgaaagtgggtcactcaggggttgaagagagcttttcccgccgaggtctgccaggggaaaactccaaaacccagaccacaatggggaaagtcagcgtggccgtccagacggatatatccgaaatgctttcggccacttcttccaccaacacatcacgctcgtcatcggagcaagatctgctcggcgcctgggaggatctacttagctggagcgagaatgcctccgctgcccgcaaattgcagcaggagatgagtgtgttgaagagctcgctgcagcgcctgggagacaagccaactccagagctcctcgatacggagccggccatccaaatagcagtggaggcgctcaagttggagcagacgcaactgaccagctataggaccaacatgctgcgtcttaacgcctccgtccacagttggcttaccaagcaggagcggcgactgcagagcgccttggaggaacaggagcagcaacaggagtccgaacaactcaagcagcaaaaacttgttgaagaggagaagggagctgacgttcagaaggagctggcgtctaccggagcagtggccatcacggtcaccgacagcaatggtaatcaggtggaggcactagccacaggagaagcctccacctccacgccagcctgggatgtgcacacgctgatgtcatcggagcaggagttccataagcatctgaagaacgaagtgagcgacatgtacagttcctgggatgaggcggatgccagaatcaacacgcaactggaaatgcttaccaactcgcttattgcctggaggcagttggagtccggtttgagtgagttccaattggctttgggccaagataggggcactctgaagggtcttgaaggagctctggacaagggacaagccacaccagtcgagctggcccagaatgtaaaactggtggccaagctgctgtccgaaaaggttcacgtaagccaggagcaacttttggccgtccagcagcacctggaccccaaccacatctaccacattacgaagtttacagcctcaaatggctcgctatcagattccggcatctctgatggaggagctacttctgatggtggcctgtccgagagggagcgacgactgggagttcttcgacgcctagcaaagcagttggaactagctctggcgccgggaagcgaagccatgcgttctattgctgcccgaatggaaagtgccgaggccgacctgaagcacctgcagaacacctgtagagacttaattgttcgcactgcagctagccaccagcagaagcagcagatccagcaaaatcagacccagcaggtgtcaccgaaggccaatggacacattaagaagcaggccgcaaagggcaaggcagaaccccagtcgcctggcagacgtggtaaaggagctcgaaaggcgcgtcaggccaagaaggctggagaggatcagcaagtagaggagccaagtctcagcccagaacagcagaagatggtcctgaagcaactcaagaccttgacaagtggcgatggtggggacgacccctctgacgatccctcgttgctcttcaacctggaaagctccgaggaagatggagagggagcggatcctgcacagacctctaagcgagggtgggcgtggcgcatcgcgagggcggcagtgcctatgcaggtggccctgttcacaatcttctgcgctgcctgcctgatgcaacccaactgctgcgacaatctgaacaacctgtccatgagcttcacgccgcagctccgctatatccgtggaccacctccgatctgaggacagagcgcgactaattaagcaggccgctaatcgttgtaggttaccgttacgttatgttaaggcatttaacagctgtaactagagttaaccagcgcaaccagccacttaggcagagccactaagagctcgcctgtggcgcgcgttttccgcgctcgaggttttacaatcccgaacagagcgctctcctccgaaaggtgcgacacccagaggcagcccaattttatatgtctaatttaggtagcgagcgcgaaaggaacgcaatttatagtaaggatgtatttgttatttcggttaatttcctacgagtttaggttgctgttcgattgaacgctgaatatgtacttaggcgaggagcagacgatgcccagccacgtgtaaactgccgcataaaggaacaaaggggcggatcgtcccccagattcttacccagtaaatatgatacaaacacaatgatcaattgagtggcgtttttgtatacttttttgtttgaaaaacactaaagtgcctacaaatgcgaactgtgaacgaggagctaccagcagatgacaaaggatccgtcgaggagtccaaacgagagtatgaatccaaggcaccgaagcaaacagaagctaaaaagccaacagcactaaggagctagcaatggagctcaccagaacagcgatgaagaatcagagtttatatttttatacgcaacgcaactatacacaatcagtgcggagacaacgaatcttcagccaacacacatcatacgttttatgtctattatattaataggataaaccttacacttattttgcaagaaaacaaaaattcaacaaaaaaaaaaaaaacgaaaa

SEQ ID NO: 95 - protein, 567 aa, Drosophila melanogaster
MGKVSVAVQTDISEMLSATSSTNTSRSSSEQDLLGAWEDLLSWSENASAARKLQQEMSVLKSSLQRLGDKPTPELLDTEPAIQIAVEALKLEQTQLTSYRTNMLRLNASVHSWLTKQERRLQSALEEQEQQQESEQLKQQKLVEEEKGADVQKELASTGAVAITVTDSNGNQVEALATGEASTSTPAWDVHTLMSSEQEFHKHLKNEVSDMYSSWDEADARINTQLEMLTNSLIAWRQLESGLSEFQLALGQDRGTLKGLEGALDKGQATPVELAQNVKLVAKLLSEKVHVSQEQLLAVQQHLDPNHIYHITKFTASNGSLSDSGISDGGATSDGGLSERERRLGVLRRLAKQLELALAPGSEAMRSIAARMESAEADLKHLQNTCRDLIVRTAASHQQKQQIQQNQTQQVSPKANGHIKKQAAKGKAEPQSPGRRGKGARKARQAKKAGEDQQVEEPSLSPEQQKMVLKQLKTLTSGDGGDDPSDDPSLLFNLESSEEDGEGADPAQTSKRGWAWRIARAAVPMQVALFTIFCAACLMQPNCCDNLNNLSMSFTPQLRYIRGPPPI

SEQ ID NO: 96 - DNA, 3685 bp, Caenorhabditis elegans
  CDS (13)..(3348)
gtttgaggtactatggctcccgcaacggaagccgacaacaacttcgacacccatgaatggaaatcggaattcgcatccacacgctctggacgcaattctccaaacatttttgcaaaagttcgccggaagcttctcctgactccaccagttcgaaacgccagatcgccacgtcttaccgaagaagagctggatgctttgacaggcgacttaccatacgcaaccaactacacatacgcatacagcaaaatctacgatccatccttgccggaccactgggaagtgccaaaccttggtggtactacttcaggatcactctctgagcaggagcactggtcagcggccagtctcagcagacagcttctctatatcctccgtttccccgtctaccttgttcttcacgtcatcacctacattttggaagctttctaccacgtcatcaagatcactagcttcaccatctgggactacctgttgtatttggtgaaactcgcgaaaactcgttactacgcctaccaagatcatcgtcgccgtacagctctcattcgcaaccggcaagagccattctccactaaggctgctcgttctattcgtcgattctttgagatccttgtctacgtcgtgcttactccttacagaatgctcacaagaagtaacaatggcgtggaacagtaccagtaccgttcgatcaaggatcaattggaaaatgagagagctagcagaatgacgacaagatctcaaacattggaaagaagccgcaagtttgatggattatcgaaatcaccagcacgccgagcagctccagcctttgtgaagactagtacaattaccagaatcactgccaaggtgttctcgagctctccattcggagaaggaacgtccgaaaatataaccccgactgttgtgactactagaacagtgaagcaacgctcagttaccccaagattccgccaaacccgtgccactcgtgaagctataactcgagcactcgatactccggaactcgaaatcgacacaccactctccacatatggacttcgaagccgaggactgagtcatctgaatactcctgaaccaacttttgacattggtcatgctgctgcaacttccacgcctttgttcccacaagaaacttacaattatcaatacgaagaagcgacaggaaataagattaaaactgcattcacttggctaggttacttgatattgttcccgttctttgcggcacgacatgtatggtatacgttctacgattatggaaagagtgcctacatgaagctgaccaattatcagcaagcgccaatggagactattcatgtcagagatatcaacgaaccggcaccaagttcatcagatgttcatgatgctgttggtgtttcttggagaattcgaattgccgatttcttgagctcattcgtagcaacaatcgttgaagcgcatcaagtggtatttgcaatgttcaaaggaggaattgttgagacagtttcctattttggaggactatttgctggtcttaccgataagaaatcatcaaagttctcgtggtgtcaaattctcggtctacttctggctcttctcttcgccatctttctccttggattcctgacatctgacaacacagcaataagagttaaagaaattaccaaagataagaatgcatctaagaagtcggaaggatccctcccagctgtgccaatctggatttcagctgcaaatcacgttaaacattacacatggatggtgaaggaatttgttgtagatattgcatttgacacgtacaactatggaaagtcgacgattggtagacttggcactactccacgttatgcttgggacctgattgcaagcggatgtggcgctgttggaaatggcttaaaatctgtgctctcatcgagttttcgattcatcgatttttgtgctggaaagctattttactatggctcagatgggttcttgtcagcaaacaagtctatcggaacctttttcaacggttgctacgagaccttgtacaacggatgcacagcaattgttggccatacaaagagcttcatctacaatgcttcaaatgctgtttacaactttttctcaactatctttgccggtctcttaaacttttctacttcttcccaaaactccattctttctcttctcaagtcatttggcaccggaatcactaacattttttataacttcatttatgcaccaatcgctggagtgttcaactttgctggtgataactacatgtatttcttcaatgaggtagcggcagtctttggaaaagtgtacaactccgtggtttccgtgctcaaaactgtaattaactggattctcttcctcattgcctacccattcagtttgtgcactcgtgcttggattcgcatcagccaatatgctccagaagatgttgttcaagtgattccaattccacaagctattaccccaactccggatgtggagcgtattgttgaagagccactgagaaaagtcaccgatgtggaggacgaagaactagtgataattcccgcccccgcacctaaacctatcccagtcccagcgccaactccggccccagtaattatccatcagactaacgttgttgagactgttgacaaagatgctatcattaaggaggtaacggagaagcttcgcgccgagttgtccgcccaattccagcaagagcttagcgcaaagtttgagcaaaactacaacacaattattgagcaactgaaaatggaaaacaccaacattcaatatgataagaatcatttggaagctatcatccgtcaaatgatctacgagtatgacacggataaaactgggaaagttgactatgccctggagagctcaggtggagctgttgtgtcaacaagatgctcggagacgtacaaaagctacacaaggctggaaaagttttgggatatcccaatctactatttccattactctccaagagttgtcattcagagaaattccaaatccctgtttcctggggaatgctggtgcttcaaagaatcccgtggctacattgctgtcgagctgtctcatttcattgatgtttctagcatcagctatgagcacattggatcagaagttgctccagaagggaaccggtcgagtgctccaaagggagtcctcgtttgggcttacaagcagattgacgacctgaactcgagagttttgattggcgactacacttatgatcttgatggcccgccacttcaattcttccttgccaagcacaaacccgattttcctgtcaagtttgtggagctcgaggtgacaagcaattacggagctccgttcacatgtctctaccgccttcgtgttcatggaaaagttgttcaagtttaatttattttgttaatcttgtttttatggtcaaattttcaatttccattgatctgattccgaaatgtttcaatttcacccctctcctgccaatttttcaaatcacaattcattttcccaaatttttccagttcccttgttatatttttagcgcgtgtccattttttttcaaagtatagaatatcattacattttcatgcagtttaccggtctcctgaatacttggcctgaatatatttgaatgcaaaaccaatcactctctatttctctgcagcctttaccccctgatctcaaaatactttatcgattttcataaattatttcaatatcaaaaaaaaaa

SEQ ID NO: 97 - protein, 1111 aa, Caenorhabditis elegans
MAPATEADNNFDTHEWKSEFASTRSGRNSPNIFAKVRRKLLLTPPVRNARSPRLTEEELDALTGDLPYATNYTYAYSKIYDPSLPDHWEVPNLGGTTSGSLSEQEHWSAASLSRQLLYILRFPVYLVLHVITYILEAFYHVIKITSFTIWDYLLYLVKLAKTRYYAYQDHRRRTALIRNRQEPFSTKAARSIRRFFEILVYVVLTPYRMLTRSNNGVEQYQYRSIKDQLENERASRMTTRSQTLERSRKFDGLSKSPARRAAPAFVKTSTITRITAKVFSSSPFGEGTSENITPTVVTTRTVKQRSVTPRFRQTRATREAITRALDTPELEIDTPLSTYGLRSRGLSHLNTPEPTFDIGHAAATSTPLFPQETYNYQYEEATGNKIKTAFTWLGYLILFPFFAARHVWYTFYDYGKSAYMKLTNYQQAPMETIHVRDINEPAPSSSDVHDAVGVSWRIRIADFLSSFVATIVEAHQVVFAMFKGGIVETVSYFGGLFAGLTDKKSSKFSWCQILGLLLALLFAIFLLGFLTSDNTAIRVKEITKDKNASKKSEGSLPAVPIWISAANHVKHYTWMVKEFVVDIAFDTYNYGKSTIGRLGTTPRYAWDLIASGCGAVGNGLKSVLSSSFRFIDFCAGKLFYYGSDGFLSANKSIGTFFNGCYETLYNGCTAIVGHTKSFIYNASNAVYNFFSTIFAGLLNFSTSSQNSILSLLKSFGTGITNIFYNFIYAPIAGVFNFAGDNYMYFFNEVAAVFGKVYNSVVSVLKTVINWILFLIAYPFSLCTRAWIRISQYAPEDVVQVIPIPQAITPTPDVERIVEEPLRKVTDVEDEELVIIPAPAPKPIPVPAPTPAPVIIHQTNVVETVDKDAIIKEVTEKLRAELSAQFQQELSAKFEQNYNTIIEQLKMENTNIQYDKNHLEAIIRQMIYEYDTDKTGKVDYALESSGGAVVSTRCSETYKSYTRLEKFWDIPIYYFHYSPRVVIQRNSKSLFPGECWCFKESRGYIAVELSHFIDVSSISYEHIGSEVAPEGNRSSAPKGVLVWAYKQIDDLNSRVLIGDYTYDLDGPPLQFFLAKHKPDFPVKFVELEVTSNYGAPFTCLYRLRVHGKVVQV

SEQ ID NO: 98 - DNA, 3811 bp, Caenorhabditis elegans
  CDS (136)..(3261)
ccgtgatcatattgcgcagatgcgtaattgatcgtccacccctaccagtacagtatcccacgtctggatacaaacttctctgatttttttatttttattcctggacaccgaaagtaacccgaaaacaggttacgaatggacgtaatggactcattctcggaggtcgagatgcccaatgatatatcatcggaggaccatcttttgaaggtcatcgaatcgtcggctgaggaagtggatatctttttggagaactgctcctcgctctacaatttaatcctcgactcgttgcacaacctcacctcaaaaacgatatcatgtgaatgtctcgacgaaatgacatctacacttgaaaaatcggcaaagaaaattctcgccgaacggcccgaggccgagaattcggtccttctccgccttaacacaatatgctgcgcaatggatcaactacgtgtccagcacaattcacgaatgatgagcggtgccgattcggacacagcaagctcagcacgtagttccacgtcatcatcgacaggcgaaatgcgtttgtggttacacgaggtcgagagaagactcgaaataaatgagaaacggattcgagtggagccaaacttgcagctgttgctctctgatcaacaggctctacaacttgaaatacaacacgaaggtcaactattggttaatcgactcaacaaacaaattaaagacgatcacgacagcgactcgtcagaagaagagaaacggaaaacttgtgtcgatgcgatcagaaaacggtggcatactatctacttgaatagtttgtctctagtctgcagaattgaagagcttattaaccatcagcaagcatcagaagactcggaaagtgatccggacctcgtcggaccaccgatcaaacgggctcgcattcgaactgttggtcacctgacggcttctgatacggaagagtctgaagctgacgaggaagacagacatagtcagactgaaactgtggtcactgaagacgataacgttcttccatttgcggagaacgagtacgaaagtattatggatggaagagtaactgtcgattcgtgtacctcttcgtccgaagaccagatggttgagcagtcgacgaacaaaaaatgggagagtgttctacaagacgtcggctattcaagcggagagaattcaattcatgaagctttgaacacatgtgctgatcatttagttcctgaaaccagtgacatgcgacgaaaacgcatcgaatgctccccggtcaaagccttttatcggactgttcagttggaagacatgtcggatctagaagtgactaaagccatcaatcacgatgtagaagaagaaccaaacttgtctgattcgatgtatgtcaaccatgattccacgttcttggccactcaaaaccttccagaatatgacgaagtaatggctttaatggatgatgatgatctaccaatggacatgtcaatgacagaatcattcaatacaaaatggcgggaaattcatggacagaagaagccattgcgacgtgcatctcgaccaagtcgtgaacaaatgaatttgattgccaagagctcgtgcgacgcatcctctgaagactcgtccgaaggagagaatcaaacgaatttggaagatgatccggagatgatgagtgtatcattcaactctgcccaattcgacacatcctccccattgaagcgccaacgatcggctcgcggactcaagaatgcttccttcctttacgacagtctcgaaatggacggatcattctgctcgacacgttccgagatgcttccaccgtgcaaaacgagatcccttgcacgccgcaagcttcgagttcgcagaatgccacgtagcatgagtgacggagagcaattgggcgttgtgagcagtaaaccagaaggaatgatgactccaatgatccgtgtttccccaccttctacaccagtccgccgacttctcaggaagctcgacgagcagattagaaaccgggatagtgacacggcgccagaacatagtgatgctgcacaagcctatgaatgggatgagtataatccaccacaaaaggacgactcaatttccgaccgccacattcagacaatgactgatatctccgatcaattgatgaatattgatgatgatttcgcagagcattttggaacttccagtgcaattcgactgatcgaagaatcgaaatcacatttgagagttgtcctaaaggctcttgaggagagtgactcgaatattccacagctctcaaattttgagctgatagcccgctcaaacctccgccaggtcgatgaggctttaaaaattcaatctggaaaccaaccatcatttttggaaaccagcactctccaggatctccgatcagaatgggctaatttatatgaatcgattcgcagcccgtttgctcgaattatgcatcaagtcaaaaagtttgcagcgacattgcaagaagtttcatcgatggcttctcttggtgacgttgatattcgttcaaaagaggacgtggcaaagacattggacgctgtgacagctattgaacgacgtctgagcagtgaaaggcaagaattgagagacctcttagcttcatcaagcttccgagacgtcgccaaagatctttcctgtgaatttgaatccgtctctgaaggatacgacgacgctgtcgacaaaattggaaaaatggctcactcattatcacaagtgaaaggagaatgggatgcttggaatagcagacaaaatgatatcagaaatgcaatggttcgtattgaatcccatctgaaagagggtcaaatggacaataaaatgattgcggatgagatggagttgtgtcaggaaagaatgaacagtctagaaactatgtgcaactacttgacagcctcgttgggatcaattcaaaatgaatccaactcaaagaatctgccagattttaaggcggaactctcgatttacagcaatgcgttggccaggctgaaggatagattcaacgacatgatccgagtgccaactcccccaacagtccaattccaccctccggagccacttccatctttggctcgaagcatgacaactcaaacagctgaaatggaatcggaaactgaaaatgagccactcacaattgcggaggcaatctcttcatctcgtctcatcaaattcacatttgccctttctcttctggcagcgctcgcagcgattttctattatcacgtgttcggaaaaccatttggtccgcatgtaacctatgtgaatggaccaccaccggtttaactgaatcatcagtattctgattgaaatccctttttgtttctatttttatgttgtaatctctgacccgggtaggtacaaaattaaaccagtagattcgattgattgtttttatattttaaatttctgaaattaaacatttaatttcaaaaaagaaaaaaattgttttttaaaaatagaaatgtaggactagatactcaatattaatcacttaccctacagtttctttcgaatatccgaaaatcttccaaactttccccccaaaaaaatctccgtgttcaggtatttgtttcattctctctcttttttgtttctccactcatattgagaatattttatatctccacttaaatcggtgactttcctgtatatatgatgatttatttgacaaaaaatgttatctgaatattctcatctgaaaagagaatccacttttgatgtcagttattttatttacgaactaccttttggttttatcctattttgttttgaaatttgatttatgtgcattcgttaataaaaattaatatatgccaaaaaaaaaaaaaaaaaa

SEQ ID NO: 99 - protein, 1041 aa, Caenorhabditis elegans
MDVMDSFSEVEMPNDISSEDHLLKVIESSAEEVDIFLENCSSLYNLILDSLHNLTSKTISCECLDEMTSTLEKSAKKILAERPEAENSVLLRLNTICCAMDQLRVQHNSRMMSGADSDTASSARSSTSSSTGEMRLWLHEVERRLEINEKRIRVEPNLQLLLSDQQALQLEIQHEGQLLVNRLNKQIKDDHDSDSSEEEKRKTCVDAIRKRWHTIYLNSLSLVCRIEELINHQQASEDSESDPDLVGPPIKRARIRTVGHLTASDTEESEADEEDRHSQTETVVTEDDNVLPFAENEYESIMDGRVTVDSCTSSSEDQMVEQSTNKKWESVLQDVGYSSGENSIHEALNTCADHLVPETSDMRRKRIECSPVKAFYRTVQLEDMSDLEVTKAINHDVEEEPNLSDSMYVNHDSTFLATQNLPEYDEVMALMDDDDLPMDMSMTESFNTKWREIHGQKKPLRRASRPSREQMNLIAKSSCDASSEDSSEGENQTNLEDDPEMMSVSFNSAQFDTSSPLKRQRSARGLKNASFLYDSLEMDGSFCSTRSEMLPPCKTRSLARRKLRVRRMPRSMSDGEQLGVVSSKPEGMMTPMIRVSPPSTPVRRLLRKLDEQIRNRDSDTAPEHSDAAQAYEWDEYNPPQKDDSISDRHIQTMTDISDQLMNIDDDFAEHFGTSSAIRLIEESKSHLRVVLKALEESDSNIPQLSNFELIARSNLRQVDEALKIQSGNQPSFLETSTLQDLRSEWANLYESIRSPFARIMHQVKKFAATLQEVSSMASLGDVDIRSKEDVAKTLDAVTAIERRLSSERQELRDLLASSSFRDVAKDLSCEFESVSEGYDDAVDKIGKMAHSLSQVKGEWDAWNSRQNDIRNAMVRIESHLKEGQMDNKMIADEMELCQERMNSLETMCNYLTASLGSIQNESNSKNLPDFKAELSIYSNALARLKDRFNDMIRVPTPPTVQFHPPEPLPSLARSMTTQTAEMESETENEPLTIAEAISSSRLIKFTFALSLLAALAAIFYYHVFGKPFGPHVTYVNGPPPV